(19) United States
(12) Patent Application Publication
(10) Pub. No.: US 2025/0215110 A1
(43) Pub. Date: Jul. 3, 2025

(54) ACTIVATABLE MULTISPECIFIC MOLECULES AND METHODS OF USE THEREOF

(71) Applicant: CytomX Therapeutics, Inc., South San Francisco, CA (US)

(72) Inventors: Madan M. PAIDHUNGAT, San Francisco, CA (US); Sayantan MITRA, Mountain View, CA (US); Ellaine Anne Mariano FOX, San Carlos, CA (US); Veena VINOD, Daly City, CA (US); Sonali Arun PATIL, San Francisco, CA (US); Leila M. BOUSTANY, Redwood City, CA (US)

(21) Appl. No.: 18/852,825

(22) PCT Filed: Mar. 31, 2023
(86) PCT No.: PCT/US2023/065191

§ 371 Date: Sep. 30, 2024

Related U.S. Application Data
(60) Provisional application No. 63/326,692, filed on Apr. 1, 2022.

(51) Int. Cl. C07K 16/32; A61K 39/00; A61P 35/00; C07K 16/28

(52) U.S. Cl. CPC C07K 16/32; A61P 35/00; C07K 16/2809; A61K 2039/505; C07K 2317/31; C07K 2317/52; C07K 2317/55; C07K 2317/569; C07K 2317/92; C07K 2319/50

(57) ABSTRACT

Activatable proteins comprising a first target binding protein (TB1) that specifically binds to a first target; a second target binding protein (TB2) that specifically binds to a second target, wherein the TB2 is directly or indirectly coupled to the TB1; a first masking moiety (MM1) inhibiting the binding of the TB1 to the first target and coupled to the TB1 via a cleavable moiety, directly or indirectly, e.g., via one or more linkers or other components; a second masking moiety (MM2) inhibiting the binding of the TB2 to the second target and coupled either to the TB1 or to the TB2 via a cleavable moiety, directly or indirectly; and a half-life extending moiety (EM) coupled either to the TB1 or to the TB2 via a cleavable moiety, directly or indirectly. The activatable proteins are configured such that when the cleavable moieties are cleaved, the resulting activated protein has a shorter half life, a higher binding affinity for the first target, and a higher binding affinity for the second target as compared to the intact activatable protein.

### CROSS-REFERENCE TO RELATED APPLICATION

This application is a 35 U.S.C. 371 National Phase Entry Application of PCT/US2023/065191, filed Mar. 31, 2023, which claims the benefit of U.S. Provisional Application No. U.S. 63/326,692, filed Apr. 1, 2022, which is incorporated herein by reference in its entirety.

### SEQUENCE LISTING

The Sequence Listing filed with this application by EFS, which is entitled “4862-122PCT.xml,” was created on Mar. 30, 2023 and is 639,463 bytes in size, is hereby incorporated by reference in its entirety.

### TECHNICAL FIELD

The present disclosure relates to the field of biotechnology, and more specifically, to activatable multispecific molecules.

### BACKGROUND

Antibody-based therapies have provided proven effective treatments for various diseases. However, in some cases, toxicities due to broad target expression have limited their therapeutic effectiveness. In addition, antibody-based therapeutics have exhibited other limitations such as rapid clearance from the circulation following administration.

Activatable antibodies have advanced the effort to broaden the therapeutic index of antibody-based therapies. These molecules are administered as an activatable prodrug that is activated in vivo at or near the desired site of action. This mechanism of action can lead to an increase in the therapeutic index of the parental antibody.

However, there is a continued need for other strategies for increasing the therapeutic index of antibody-based therapeutics.

### SUMMARY OF THE INVENTION

The present disclosure provides an activatable protein and related compositions and methods.

In one aspect, the present disclosure provides an activatable protein comprising: a first target-binding domain (TB1) that specifically binds to a first target; a second target-binding domain (TB2) that specifically binds to a second target, wherein the TB2 is coupled to the TB1; a first masking moiety (MM1) coupled to the TB1 via a first cleavable moiety (CM1), wherein the MM1 inhibits the binding of the TB1 to the first target; a second masking moiety (MM2) that inhibits the binding of the TB2 to the second target; a second cleavable moiety (CM2); and a half-life extending moiety (EM) coupled, either directly or indirectly, to the MM1 or the MM2, wherein the components of the activatable protein are configured such that upon cleavage of the CM1 and CM2, the resulting activated protein comprises the TB1 and TB2, but does not comprise the MM1, the MM2, and the EM. As used herein and unless otherwise stated, components of the activatable molecule that are “coupled” may be coupled either via a direct covalent linkage or indirect covalent linkage, e.g., via one or more linking peptides (also referred to as “linkers”), cleavable moieties, or other components of the activatable protein.

In one aspect, the present disclosure provides an activatable protein comprising: a first antigen-binding domain (AB1) that specifically binds to a first target, wherein the AB1 comprises a first heavy chain variable domain (HVD1) and a first light chain variable domain (LVD1); a second antigen-binding domain (AB2) that specifically binds to a second target, wherein the AB2 comprises a second heavy chain variable domain (HVD2) and a second light chain variable domain (LVD2), and the AB2 is directly or indirectly coupled to a C-terminus of the HVD1 or to a C-terminus of the LVD1; a first masking moiety (MM1) coupled to the AB1 via a first cleavable moiety (CM1) (either directly or indirectly, e.g., via one or more linkers), wherein the MM1 inhibits the binding of the AB1 to the first target; a half-life extending moiety (EM) directly or indirectly coupled to a second masking moiety (MM2), wherein the EM is coupled to the AB1 or to the AB2 via a second cleavable moiety (CM2) (either directly or indirectly, e.g., via one or more linkers or other components of the activatable protein), and wherein the MM2 inhibits the binding of the AB2 to the second target.

In one aspect, the present disclosure provides an activatable protein comprising: a first antigen-binding domain (AB1) that specifically binds to a first target, wherein the AB1 comprises a first heavy chain variable domain (HVD1) and a first light chain variable domain (LVD1); a second antigen-binding domain (AB2) that specifically binds to a second target, wherein the AB2 comprises a second heavy chain variable domain (HVD2) and a second light chain variable domain (LVD2), and the AB2 is directly or indirectly coupled to a C-terminus of the HVD1 or the LVD1; a first masking moiety (MM1) coupled to the AB1 via a first cleavable moiety (CM1) (either directly or indirectly, e.g., via one or more linkers), wherein the MM1 inhibits the binding of the AB1 to the first target; and a half-life extending moiety (EM) directly or indirectly coupled to a second masking moiety (MM2), wherein the EM and the MM2 are coupled either to the AB1 or to the AB2 via a second cleavable moiety (CM2) (directly or indirectly), and wherein the MM2 inhibits the binding of the AB2 to the second target.

In one aspect, the present disclosure provides an activatable protein comprising: a first antigen-binding domain (AB1) that specifically binds to a first target, wherein the AB1 comprises a first heavy chain variable domain (HVD1) and a first light chain variable domain (LVD1); a second antigen-binding domain (AB2) that specifically binds to a second target, wherein the AB2 comprises a second heavy chain variable domain (HVD2) and a second light chain variable domain (LVD2), and the AB2 is directly or indirectly coupled to a C-terminus of the HVD1 or the LVD1; a first masking moiety (MM1) coupled to the AB1 via a first cleavable moiety (CM1) (either directly or indirectly, e.g., via one or more linkers), wherein the MM1 inhibits the binding of the AB1 to the first target; and a half-life extending moiety (EM) comprising a dimer of a first half-life extending moiety (EM1) and a second half-life extending moiety (EM2), wherein the EM1 is coupled to the AB1 via a second cleavable moiety (CM2) (either directly or indirectly, e.g., via one or more linkers), and wherein the EM2 is directly or indirectly coupled to a second masking moiety (MM2), wherein the MM2 inhibits the binding of the AB2 to the second target.

In one aspect, the present disclosure provides an activatable protein comprising: a first target-binding domain (TB1) that specifically binds to a first target; a second target-binding domain (TB2) that specifically binds to a second target, wherein the TB2 is directly or indirectly coupled to the TB1; a first masking moiety (MM1) coupled to the TB1 via a first cleavable moiety (CM1) (either directly or indirectly, e.g., via one or more linkers), wherein the MM1 inhibits the binding of the TB1 to the first target; a half-life extending moiety (EM) and a second masking moiety (MM2) coupled to the TB1 or to the TB2 via a second cleavable moiety (CM2) (either directly or indirectly, e.g., via one or more linkers), wherein the MM2 inhibits the binding of the TB2 to the second target, wherein the components of the activatable molecule are configured such that cleavage of the CM1 and the CM2 releases the MM1, the MM2, and the EM from the TB1 and TB2 (as applicable), and wherein optionally the TB1 is an antigen-binding molecule (AB1) comprising a HVD1 and an LVD1, and optionally the TB2 is an antigen-binding molecule (AB2) comprising a HVD2 and an LVD2.

In one aspect, the present disclosure provides an activatable protein comprising: a first antigen-binding domain (AB1) that specifically binds to a first target, wherein the AB1 comprises a first heavy chain variable domain (HVD1) and a light chain variable domain (LVD1); a second antigen-binding domain (AB2) that specifically binds to a second target, wherein the AB2 comprises a second heavy chain variable domain (HVD2) and a second light chain variable domain (LVD2), and the AB2 is coupled, either directly or indirectly (e.g., via a linker), to a C-terminus of the HVD1 or the LVD1; a first masking moiety (MM1) coupled to the AB1 via a first cleavable moiety (CM1), (either directly or indirectly, e.g., via a linker), wherein the MM1 inhibits the binding of the AB1 to the first target; a second masking moiety (MM2) coupled to the AB2 via a second cleavable moiety (CM2) (either directly or indirectly, e.g., via a linker), wherein the MM2 inhibits the binding of the AB2 to the second target; and a half-life extending moiety (EM) coupled, either directly or indirectly, to the MM1 or the MM2.

In another aspect, the present disclosure provides an activatable protein comprising: a first antigen-binding domain (AB1) that specifically binds to a first target, wherein the AB1 comprises a first heavy chain variable domain (HVD1) and a first light chain variable domain (LVD1); a second antigen-binding domain (AB2) that specifically binds to a second target, wherein the AB2 comprises a second heavy chain variable domain (HVD2) and a second light chain variable domain (LVD2), and the AB2 is directly or indirectly coupled to an N-terminus of the HVD1 or to an N-terminus of the LVD1; a first masking moiety (MM1) coupled to the AB1 via a first cleavable moiety (CM1) and optionally one or more linkers, wherein the MM1 inhibits the binding of the AB1 to the first target; a second masking moiety (MM2) coupled to the AB2 via a second cleavable moiety (CM2) and optionally one or more linkers, wherein the MM2 inhibits the binding of the AB2 to the second target; a half-life extending moiety (EM) coupled to a C-terminus of the HVD1 or to a C-terminus of the LVD1 via a third cleavable moiety (CM3) and optionally one or more linkers.

In some embodiments, the EM is a dimer formed by a first fragment crystallizable (Fc) domain and a second Fc domain. In some embodiments, the protein comprises at least a first polypeptide and a second polypeptide.

In some embodiments, the first polypeptide comprises, in order from N-terminus to C-terminus, the MM1, the CM1, and the VLD1 (with one or more optional linkers between the elements). In some embodiments, the second polypeptide comprises the VHD1, the VHD2, the VLD2, the CM2, the MM2 and a first Fc domain, and wherein the activatable protein further comprises a third polypeptide comprising a second Fc domain. In some embodiments, the second polypeptide comprises, in order from N-terminus to C-terminus, the VHD1, the VHD2, the VLD2, the CM2, the MM2, and a first Fc domain. In some embodiments, the second polypeptide comprises, in order from N-terminus to C-terminus, the VHD1, the CM2, the MM2, and a first Fc domain. In some embodiments, the second polypeptide comprises, in order from N-terminus to C-terminus, the VHD1, the CM2, and a first Fc domain. In some embodiments, the first polypeptide comprises the MM1, the CM1, and the VLD1, the VHD2, and the VLD2. In some embodiments, the first polypeptide comprises, in order from N-terminus to C-terminus, the MM1, the CM1 the VLD1, the VHD2, and the VLD2. In some embodiments, the first polypeptide comprises, in order from N-terminus to C-terminus, the MM1, the CM1 the VLD1, the VLD2, and the VHD2. In some embodiments, the protein comprises a third polypeptide, and wherein the third polypeptide comprises a second Fc domain and the MM2. In each of the foregoing embodiments, and unless otherwise stated, the polypeptide may comprise, e.g., one or more optional linkers between each of the elements listed.

In some embodiments, the MM2 is linked to the C-terminus of the second Fc domain via a linking peptide. In some embodiments, the MM2 is linked to the N-terminus of the second Fc domain via a linking peptide (also referred to as a “linker”). In some embodiments, the second polypeptide further comprises a linker (L1) between the MM2 and the first Fc domain. In some embodiments, L1 is a peptide having a length of 5 to 30, 6 to 29, 7 to 28, 8 to 27, 9 to 26, 10 to 25, 11, 12, 13, 14, 15, 16, 17, 18, 19, 20, 21, 22, 23, or 24 amino acids. In the disclosed structural arrangements in the foregoing paragraphs and throughout this disclosure, one or more linkers may optionally be present between the elements. Further, this disclosure also contemplates and includes activatable proteins in which any one or more of the disclosed elements optionally directly abut each other such that there are no linkers or other amino acid sequences between the elements.

In some embodiments, the first Fc domain is a Fc domain hole mutant and the second Fc domain is a Fc domain knob mutant. In some embodiments, the Fc domain hole mutant comprises a sequence of SEQ ID NO: 2 and the Fc domain knob mutant comprises a sequence of SEQ ID NO: 1.

In some embodiments, the first target or epitope is a tumor associated antigen. In some embodiments, the tumor associated antigen is human epidermal growth factor receptor 2 (HER2). In some embodiments, the AB1 is a Fab of trastuzumab. In some embodiments, the HVD1 comprises a sequence of SEQ ID NO: 27 and the LVD1 comprises a sequence of SEQ ID NO: 17. In some embodiments, AB2 is: an immune effector cell engaging scFv; a leukocyte engaging scFv; a T-cell engaging scFv; a NK-cell engaging scFv; a macrophage engaging scFv; or a mononuclear cell engaging scFv. In some embodiments, AB2 is or is derived from an anti-CD3 epsilon scFv or an anti-CTLA-4 scFv. In some embodiments, the AB2 is or is derived from an anti-CD3 epsilon scFv. In some embodiments, the HVD2 comprises a sequence of SEQ ID NO: 30 and the LVD2 comprises a sequence of SEQ ID NO: 31.

In some embodiments, AB1 is or is derived from an anti-HER2 antibody. In some embodiments, AB1 is a scFv and the activatable protein is an activatable bi-specific T-cell engager (BiTE) or a dual-affinity retargeting antibody (DART). In some embodiments, AB1 is a Fragment antigen binding (Fab). In some embodiments, the second target is a co-stimulatory molecule. In some embodiments, the co-stimulatory molecule is CD3.

In some embodiments, each of the CM1 and the CM2 comprises a substrate for the same protease. In some embodiments, the CM1 and the CM2 comprise substrates for different proteases. In some embodiments, each of the CM1 and the CM2 independently comprises a substrate for a protease selected from the group consisting of ADAMS, ADAMTS, ADAM8, ADAM9, ADAM10, ADAM12, ADAM15, ADAM17/TACE, ADAMDEC1, ADAMTS1, ADAMTS4, ADAMTS5, Aspartate proteases, BACE, Renin, Aspartic cathepsins, Cathepsin D, Cathepsin E, Caspases, Caspase 1, Caspase 2, Caspase 3, Caspase 4, Caspase 5, Caspase 6, Caspase 7, Caspase 8, Caspase 9, Caspase 10, Caspase 14, Cysteine cathepsins, Cathepsin B, Cathepsin C, Cathepsin K, Cathepsin L, Cathepsin S, Cathepsin V/L2, Cathepsin X/Z/P, Cysteine proteinases, Cruzipain, Legumain, Otubain-2, KLKs, KLK4, KLK5, KLK6, KLK7, KLK8, KLK10, KLK11, KLK13, KLK14, Metallo proteinases, Meprin, Neprilysin, PSMA, BMP-1, MMPs, MMP1, MMP2, MMP3, MMP7, MMP8, MMP9, MMP10, MMP11, MMP12, MMP13, MMP14, MMP15, MMP16, MMP17, MMP19, MMP20, MMP23, MMP24, MMP26, MMP27, Serine proteases, activated protein C, Cathepsin A, Cathepsin G, Chymase, coagulation factor proteases, FVIIa, FIXa, FXa, FXIa, FXIIa, Elastase, Granzyme B, Guanidinobenzoatase, HtrA1, Human Neutrophil Elastase, Lactoferrin, Marapsin, NS3/4A, PACE4, Plasmin, PSA, tPA, Thrombin, Tryptase, uPA, Type II Transmembrane, Serine Proteases, TTSPs, DESC1, DPP-4, FAP, Hepsin, Matriptase-2, MT-SP1/Matriptase, TMPRSS2, TMPRSS3, and TMPRSS4.

In some embodiments, the MM1 and the MM2 are each independently 2 to 40 amino acids in length. In some embodiments, the MM1 and the MM2 are each independently 4 to 30 amino acids in length. In some embodiments, the heavy chain variable region of the AB2 is directly or indirectly coupled to a C-terminus of the heavy chain fragment of the AB1, an N-terminus of the MM2 coupled to a C-terminus of a light chain variable region of the AB2 via the CM2 (either directly or indirectly, e.g., via one or more linkers), and the EM comprises a dimer of a first Fc domain and a second Fc domain, and a C-terminus of the MM2 is directly or indirectly coupled to an N-terminus of the first Fc domain of the EM. In some embodiments, the heavy chain variable region of the AB2 is directly or indirectly coupled to a C-terminus of the light chain fragment of the AB1, an N-terminus of the MM2 is coupled to a C-terminus of the heavy chain fragment of the AB1 via the CM2 (either directly or indirectly, e.g., via one or more linkers), and the EM comprises a dimer of a first Fc domain and a second Fc domain, and a C-terminus of the MM2 is directly or indirectly coupled to an N-terminus of the first Fc domain of the EM. In some embodiments, the heavy chain variable region of the AB2 is directly or indirectly coupled to a C-terminus of the light chain fragment of the AB1, the EM comprises a dimer of a first Fc domain and a second Fc domain, and an N-terminus of the first Fc domain is coupled to a C-terminus of the heavy chain fragment of the AB1 via the CM2 (either directly or indirectly, e.g., via one or more linkers), and an N-terminus of the MM2 is directly or indirectly coupled to an C-terminus of the second Fc domain. In some embodiments, the heavy chain variable region of the AB2 is directly or indirectly coupled to a C-terminus of the light chain fragment of the AB1, the EM comprises a dimer of a first Fc domain and a second Fc domain, and an N-terminus of the first Fc domain is coupled to a C-terminus of the heavy chain fragment of the AB1 via the CM2 (either directly or indirectly, e.g., via one or more linkers), an a C-terminus of the MM2 is directly or indirectly coupled to an N-terminus of the second Fc domain.

In some embodiments, the activatable protein further comprises a linker between the MM2 and the first or second Fc domain directly or indirectly coupled to the MM2. In some embodiments, the MM1 comprises a sequence of SEQ ID NO: 40 and the MM2 comprises a sequence of any one of SEQ ID NO: 34-37, or 66-70. In some embodiments, the MM1 has a dissociation constant for binding to the AB1 that is greater than a dissociation constant of the AB1 for binding to the first target or epitope, and the MM2 has a dissociation constant for binding to the AB2 that is greater than a dissociation constant of the AB2 for binding to the second target or epitope. In some embodiments, the activated molecule has a shorter half-life compared to a counterpart molecule that is the same as the activated molecule but comprising the EM. In some embodiments, the activated molecule has a higher target-binding activity compared to a counterpart molecule that is the same as the activated molecule but comprising the EM. In some embodiments, the activated molecule has a higher target-binding activity compared to the activatable molecule.

In some embodiments, the second polypeptide further comprises a linker (L2) between the MM2 and the AB2. In some embodiments, L2 is 5 to 30, 6 to 29, 7 to 28, 8 to 27, 9 to 26, 10 to 25, 11, 12, 13, 14, 15, 16, 17, 18, 19, 20, 21, 22, 23, 24, 25, 26, or 27 amino acids in length. In some embodiments, the second polypeptide further comprises a linker (L3) between the AB2 and the AB1. In some embodiments, L3 is 5 to 30, 6 to 29, 7 to 28, 8 to 27, 9 to 26, 10 to 25, 11, 12, 13, 14, 15, 16, 17, 18, 19, 20, 21, 22, 23, 24, 25, 26, or 27 amino acids in length. In general, in each embodiment herein, unless otherwise stated, a polypeptide may comprise one or more optional linkers between each of the elements listed, and such linkers may be 1 to 30, 6 to 29, 7 to 28, 8 to 27, 9 to 26, 10 to 25, 1, 2, 3, 4, 5, 6, 7, 8, 9, 10, 11, 12, 13, 14, 15, 16, 17, 18, 19, 20, 21, 22, 23, 24, 25, 26, or 27 amino acids in length.

In another aspect, the present disclosure provides a composition comprising the activatable protein herein and a carrier. In some embodiments, the composition is a pharmaceutical composition, wherein the carrier is a pharmaceutically acceptable carrier.

In another aspect, the present disclosure provides a container, vial, syringe, injector pen, or kit comprising at least one dose of the composition herein.

In another aspect, the present disclosure provides a nucleic acid comprising a sequence encoding the second polypeptide herein.

In another aspect, the present disclosure provides a vector comprising the nucleic acid herein.

In another aspect, the present disclosure provides a cell comprising the nucleic acid or the vector herein.

In another aspect, the present disclosure provides a conjugated activatable protein comprising the activatable protein herein conjugated to an agent. In some embodiments, the agent is a therapeutic agent, an antineoplastic agent, a toxin, a diagnostic agent, a therapeutic macromolecule, a targeting moiety, or a detectable moiety. In some embodiments, the agent is conjugated to the antibody via a linker. In some embodiments, the linker is a cleavable linker. In some embodiments, the linker is a non-cleavable linker.

In another aspect, the present disclosure provides a method of treating a subject in need thereof comprising administering to the subject a therapeutically effective amount of the activatable protein, the composition, or the conjugated activatable protein herein. In some embodiments, the subject has been identified or diagnosed as having a cancer.

In another aspect, the present disclosure provides a method of producing an activatable protein, comprising: culturing a cell herein in a culture medium under a condition sufficient to produce the activatable protein; and recovering the activatable protein from the cell or the culture medium. In some embodiments, the method further comprises isolating the activatable protein recovered from the cell or the culture medium. In some embodiments, isolating the activatable protein is performed using a protein purification tag and/or size exclusion chromatography. In some embodiments, the method further comprises formulating the activatable protein into a pharmaceutical composition.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIGS. 1-4 show configurations of exemplary activatable molecules. The molecules are designed such that the activated molecules resulting from the activation of the activatable molecules do not comprise half-life extending moieties and thus have a shorter half-life than counterpart molecules that are the same as the activated molecules but comprising the half-life extending moieties.

FIG. 5 is a schematic of an illustrative activatable (dually masked) bispecific antibody according to some embodiments in the present disclosure before and after activation by a protease. On the left side, an activatable dually masked protein is schematically illustrated. The broken lines between elements 501 and 505, and between elements 502 and 503, indicate a cleavable moiety. On the right side, an activated protein is schematically illustrated. The activated bispecific antibody does not comprise masking moieties and thus has increased binding affinity for its targets relative to the activatable bispecific antibody. The activated bispecific antibody also does not comprise a half-life extending moiety and thus has a shorter half-life than the activatable bispecific antibody.

FIG. 6A is a schematic of the components of an exemplary dually masked bispecific activatable antibody having a masked Fab fragment that that recognizes Her2, a masked scFV component that recognizes CD3, and a half-life extending moiety comprising a pair of knob and hole Fc domains. FIG. 6B is a schematic showing the components of three polypeptides that encode an exemplary dually masked bispecific activatable antibody as illustrated in FIG. 6A. The broken lines indicate a cleavable moiety.

FIG. 7A is an image of an SDS-PAGE gel run under reducing conditions. The gel was loaded as follows: (1) dually masked bispecific activatable antibody with 20 GG CD3 mask (ProC1446, SEQ ID NO: 21); (2) product of ProC1446 and uPA (ProC1446+uPA); (3) dually masked bispecific activatable antibody with MN15a CD3 mask (ProC1447, SEQ ID NO: 22); (4) product of ProC1447 and uPA (ProC1447+uPA); (5) dually masked bispecific activatable antibody with MN15b CD3 mask (ProC1448, SEQ ID NO: 23); and (6) product of ProC1448 and uPA (ProC1448+uPA). FIG. 7B is a table summarizing the expected molecular weights of the components of each activatable antibody construct before and after protease activation.

FIG. 8 provides the results of an ELISA binding assay to determine the ability of the activatable and activated molecules to bind CD3 antigen bound to the plate: unmasked reference bispecific molecule (ProC531), dually masked activatable bispecific molecule with 20 GG CD3 mask (ProC1446, SEQ ID NO: 21), product of ProC1446 and uPA (ProC1446+uPA), dually masked molecule with MN15a mask (ProC1447, SEQ ID NO: 22), product of ProC1447 and uPA (ProC1447+uPA), dually masked molecule with MN15b mask (ProC1448, SEQ ID NO: 23), product of ProC1448 and uPA (ProC1448+uPA). The results show that the respective CD3 masks between the anti-CD3 scFv and the Fc region showed attenuation of binding of the anti-CD3 scFv to the CD3 antigen coated on the plate. Treatment of the dually masked activatable bispecific antibodies with protease uPA resulted in CD3 antigen binding equivalent to that of the unmasked reference bispecific molecule ProC531.

FIGS. 9A-9C provide the results of a HER2-dependent cytotoxic assay to determine the in vitro potency of the dually masked activatable bispecific antibodies (FIG. 9A: ProC1446; FIG. 9B: ProC1447; FIG. 9C: ProC1448). The results show that the protease-treated (activated) bispecific molecules of the present disclosure were more active than a monovalent, unmasked bispecific antibody control having the same HER2 and CD3 binding domains, but arranged in a different format (“ProC306”).

FIGS. 10-11 show configurations of exemplary activatable molecules. The molecules comprise dually masked activatable bispecific antibodies having an EM coupled to the C-terminus via a third cleavable moiety. The molecules are designed such that the activated molecules resulting from the activation of the activatable molecules do not comprise half-life extending moieties and thus have a shorter half-life than counterpart molecules that are the same as the activated molecules but comprising the half-life extending moieties.

FIG. 12 is a schematic of an illustrative activatable (dually masked) bispecific antibody according to some embodiments in the present disclosure before and after activation by a protease. On the left side, an activatable dually masked protein is schematically illustrated. On the right side, an activated protein is schematically illustrated. The activated bispecific antibody does not comprise masking moieties and thus has increased binding affinity for its targets relative to the activatable bispecific antibody. The activated bispecific antibody also does not comprise a half-life extending moiety and thus has a shorter half-life than the activatable bispecific antibody. The broken lines indicate a cleavable moiety.

FIGS. 13A-13B provide the binding results of masked, activatable short half-life antibodies, ProC1446 (SHL1), ProC3007 (SHL2), ProC3008 (SHL2), and masked antibody, ProC1441 (1/2 TCB, not an activatable short half-life antibody) and unmasked (ProC1963 (SHL1, no mask or Fc), ProC1965 (SHL2, no mask or Fc), and ProC306) anti-CD3, anti-HER2 bispecific antibodies, as well as secondary antibody (“Sec only”, negative control) to NCI-N87 and SKOV3 cells (i.e., HER2 binding), respectively. FIG. 13C provides the binding results of the same molecules to Jurkat cells (i.e., CD3 binding).

FIGS. 14A-14B provide the results of a cytotoxicity assay showing the dose-response for ProC1963 (SHL1), ProC1965 (SHL2), and ProC306 at the indicated concentrations using NCI-N87 cells (FIG. 14A) and SKOV3 cells (FIG. 14B).

FIGS. 15A-15B provide the results of a cytotoxicity assay showing the dose-response for ProC1963, ProC1965, ProC1446, ProC3007 and ProC3008 at the indicated concentrations using NCI-N87 cells (FIG. 15A) and SKOV3 cells (FIG. 15B).

FIGS. 16A-16D provide the results of cytotoxicity assays. FIGS. 16A-16B show the dose-response for ProC1963, ProC1965, ProC3007, ProC3008, ProC306, and ProC1441 using NCI-N87 cells (FIG. 16A) and SKOV3 cells (FIG. 16B). FIGS. 16C-16D show the dose-response for ProC1963, ProC1965, ProC1446, ProC306, and ProC1441 uisng NCI-N87 cells (FIG. 16C) and SKOV3 cells (FIG. 16D).

FIG. 17 provides the results of an in vivo tumor growth assay using a NCI-N87 xenograft model. The plot shows tumor volume versus days post initial treatment with ProC1965, ProC3007, ProC3008, and ProC1441 administered at the indicated doses in milligrams per kilogram (mpk).

The figures provided herein are for illustrative purposes only and are not necessarily drawn to scale.

### DETAILED DESCRIPTION

Provided herein are activatable molecules (e.g., activatable proteins such as activatable antibodies and other activatable therapeutic or activatable diagnostic proteins) that have relatively low binding activity and a structure that includes a half-life extending moiety (EM). When activated by exposure to certain activating conditions (e.g., when the activatable molecule is delivered to a tumor), the resulting activated molecule has greater binding activity and a shorter half-life as compared to the activatable molecule. In one aspect, the activatable molecules may be activatable therapeutic macromolecules. In some aspects, the activatable therapeutic macromolecules may be activatable antibodies or any other desired protein, e.g., a therapeutic protein.

In general, an activatable molecule herein may include one or more target-binding domains (TBs), one or more masking moieties (MMs) that reduce, inhibit or interfere with the binding of the TBs to their targets, one or more cleavable moieties (CMs) that couple the one or more MMs to the one or more TBs, and one or more half-life extending moieties (EMs) coupled to the TBs via one or more CMs. The coupling of two components in a polypeptide may be direct or indirect. When the two components are coupled directly, the amino acid residue at the C-terminus of a component forms a peptide bond with the amino acid residue at the N-terminus of the other component. When the two components are coupled indirectly, there is a stretch of amino acids between the two components. In some examples, the two components of a polypeptide may be indirectly coupled via one or more other components in the polypeptide, i.e., the one or more other components are between the two coupled components. For indirectly coupling or linking via another component, the one or more other components may be a linker, TB(s) (e.g., AB(s)), CM(s), MM(s), or any combination thereof.

A CM is a polypeptide that comprises a substrate for a sequence-specific protease, e.g., a protease that is present in higher amounts (or present in an active state in higher amounts) in the environment of a diseased tissue such as a tumor than in healthy tissue. The MMs and the EMs of an activatable molecule described herein may be released from the TBs by cleaving the CMs, creating an activated molecule. The activated molecule exhibits greater binding affinity for its target compared to a counterpart activatable molecule comprising the MM(s). Free of the EM, the activated molecule may have a shorter half-life compared to a counterpart molecule that is the same as the activated molecule but comprising the EM. The activated molecule may have reduced toxicities and reduced off-target effects compared to a counterpart molecule that is the same as the activated molecule but comprising the EM.

In some embodiments, the activatable molecules may be a dually masked bispecific target-binding molecule. In some aspects, such molecules may comprise at least two target-binding proteins and at least two masking moieties, each of the masking moieties inhibiting the binding of a target-binding protein to its target. For example, the activatable molecules may comprise a first target-binding protein (TB1) that specifically binds to a first target, a first masking moiety (MM1) inhibiting the binding of TB1 and the first target, a cleavable moiety (CM1) positioned between MM1 and TB1, a second target-binding protein (TB2) that specifically binds to a second target, a second masking moiety (MM2) inhibiting the binding of TB2 and the second target, a cleavable moiety (CM2) positioned between MM2 and TB2, and an EM coupled to the TB1 or the TB2 via a cleavable moiety (CM). In some embodiments, the EM may be coupled to a TB via a CM that also couples a MM to the TB. In some embodiments, the EM may be coupled to a TB via a CM that is different from the CM1 and CM2 (e.g., a third CM or “CM3”). In the activated state, the EM may be released from the activated molecule. The activated molecule (comprising the TB1 and TB2 but not the MM1, MM2 or EM) thus has a shorter half-life compared to a reference molecule comprising the TB1, TB2, and EM, but not the MM1 or MM2. The activated molecule (comprising the TB1 and TB2 but not the MM1, MM2, and EM) has a higher target-binding activity compared to a reference molecule comprising the TB1, TB2, and EM, but not the MM1 or MM2.

Also provided herein are related compositions, kits, nucleic acids, vectors, and recombinant cells, as well as related methods, including methods of using and methods of producing any of the activatable molecules (e.g., activatable antibodies and other proteins) described herein.

### Definitions

Unless otherwise defined, all technical and scientific terms used herein have the same meaning as commonly understood by one of ordinary skill in the art to which this invention belongs. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

The term “a” and “an” refers to one or more (i.e., at least one) of the grammatical object of the article. By way of example, “a cell” encompasses one or more cells.

As used herein, the terms “about” and “approximately,” when used to modify an amount specified in a numeric value or range, indicate that the numeric value as well as reasonable deviations from the value known to the skilled person in the art. For example ±20%, ±10%, or ±5%, are within the intended meaning of the recited value where appropriate.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 0.01 to 2.0” should be interpreted to include not only the explicitly recited values of about 0.01 to about 2.0, but also include individual values and sub-ranges within the indicated range. Thus, included in this numerical range are individual values such as 0.5, 0.7, and 1.5, and sub-ranges such as from 0.5 to 1.7, 0.7 to 1.5, and from 1.0 to 1.5, etc. Furthermore, such an interpretation should apply regardless of the breadth of the range or the characteristics being described. Additionally, it is noted that all percentages are computed on the basis of weight, unless specified otherwise.

In understanding the scope of the present disclosure, the terms “including” or “comprising” and their derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of,” as used herein, is intended to specify the presence of the stated features, elements, components, groups, integers, and/or steps as well as those that do not materially affect the basic and novel characteristic(s) of features, elements, components, groups, integers, and/or steps. It is understood that reference to any one of these transition terms (i.e. “comprising,” “consisting,” or “consisting essentially”) provides direct support for replacement to any of the other transition term not specifically used. For example, amending a term from “comprising” to “consisting essentially of” or “consisting of” would find direct support due to this definition for any elements disclosed throughout this disclosure. Based on this definition, any element disclosed herein or incorporated by reference may be included in or excluded from the claimed invention.

As used herein, a plurality of compounds, elements, or steps may be presented in a common list for convenience. However, these lists should be construed as though each member of the list is individually identified as a separate and unique member. Thus, no individual member of such list should be construed as a defacto equivalent of any other member of the same list solely based on their presentation in a common group without indications to the contrary.

The term “exemplary” is used herein to mean serving as an example, instance, or illustration. Any aspect or design described herein as “exemplary” is not necessarily to be construed as preferred or advantageous over other aspects or designs. Rather, use of the word exemplary is intended to present concepts in a more concrete fashion.

Furthermore, certain molecules, constructs, compositions, elements, moieties, excipients, disorders, conditions, properties, steps, or the like may be discussed in the context of one specific embodiment or aspect or in a separate paragraph or section of this disclosure. It is understood that this is merely for convenience and brevity, and any such disclosure is equally applicable to and intended to be combined with any other embodiments or aspects found anywhere in the present disclosure and claims, which all form the application and claimed invention at the filing date. For example, a list of constructs, molecules, method steps, kits, or compositions described with respect to a construct, composition, or method is intended to and does find direct support for embodiments related to constructs, compositions, formulations, and methods described in any other part of this disclosure, even if those method steps, active agents, kits, or compositions are not re-listed in the context or section of that embodiment or aspect.

### Activatable Molecules

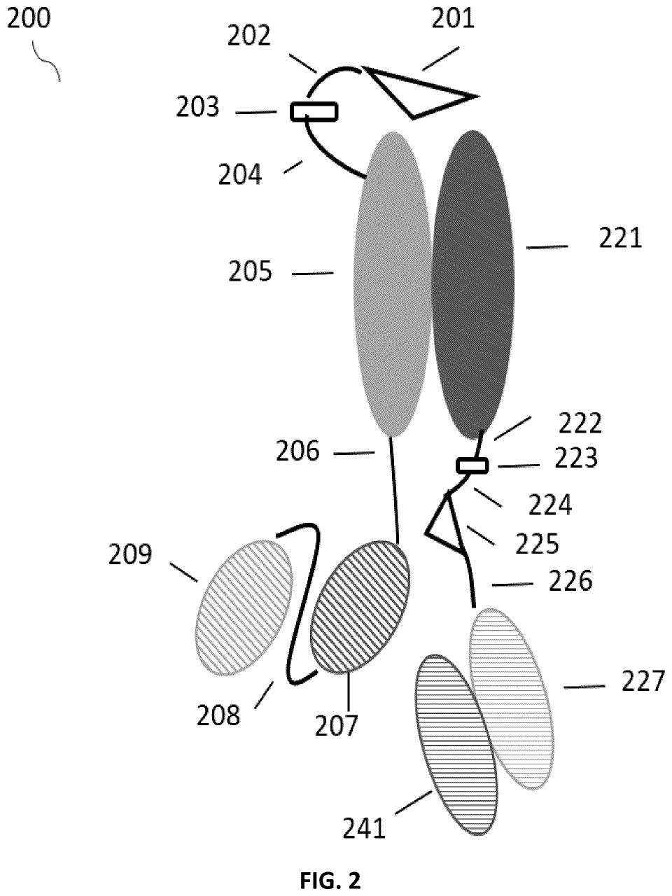

In one aspect, the activatable molecules provided herein may be activatable target-binding proteins (TBs), for example, activatable antibodies or another protein that specifically binds to a target. In some embodiments, the activatable molecule comprises a TB (e.g., an antigen-binding protein (AB)) that specifically binds to a target; a cleavable moiety (CM) directly covalently linked to (also referred to as “directly coupled to”) or indirectly covalently linked to (also referred to as “indirectly coupled to”) to the TB (e.g., AB), wherein the CM is positioned between the TB and a masking moiety (MM) that reduces, inhibits, or interferes with the binding of the TB (e.g., AB) to its target(s), and one or more half-life extending moieties (EMs) coupled to the TB (e.g., AB) via one or more CMs. The MMs and the EMs may be released from the TBs by cleaving the CMs, producing the activated molecule. In some embodiments, the activatable molecule may comprise a first antigen-binding protein (AB1) that specifically binds to a first target, a first masking moiety (MM1) inhibiting the binding of AB1 to the first target and coupled to the AB1 via a first cleavable moiety (CM1), a second antigen-binding protein (AB2) that specific binds to a second target (AB2), a second masking moiety (MM2) inhibiting the binding of AB2 to the second target and coupled either to the AB1 or to the AB2 via a second cleavable moiety (CM2), and an EM coupled either to the AB1 or to the AB2 via a cleavable moiety. In some aspects, the EM may be coupled to the AB1 via the same cleavable moiety (CM1) that couples the MM1 to the ABL. In some aspects, the EM may be coupled to the AB2 via the same cleavable moiety (CM2) that couples the MM2 to the AB2. In some aspects, the EM may be coupled to AB1 via the same cleavable moiety (CM2) that couples the MM2 to the AB1 (see, e.g., FIG. 2). In some aspects, the EM may be coupled to the AB1 or the AB2 via a third cleavable moiety (CM3). In each of the foregoing aspects, the elements of the activable molecule may be coupled directly, or coupled indirectly via one more optional linkers between the elements. In the activated state, the EM may be released from the activatable protein resulting in an activated protein that comprises the AB1 and AB2 but not the MM1, MM2 or EM, wherein the activated protein has a shorter half-life compared to a reference antibody comprising the AB1, AB2, and EM, but not the MM1 or MM2.

In some embodiments, activatable proteins provide for reduced toxicity and/or off-target side effects that could otherwise result from binding of the TB (e.g., AB) at non-treatment sites if the TB were not masked or otherwise inhibited from binding to the target. In the activatable state, the MM may interfere with the binding of the TB to its target molecule.

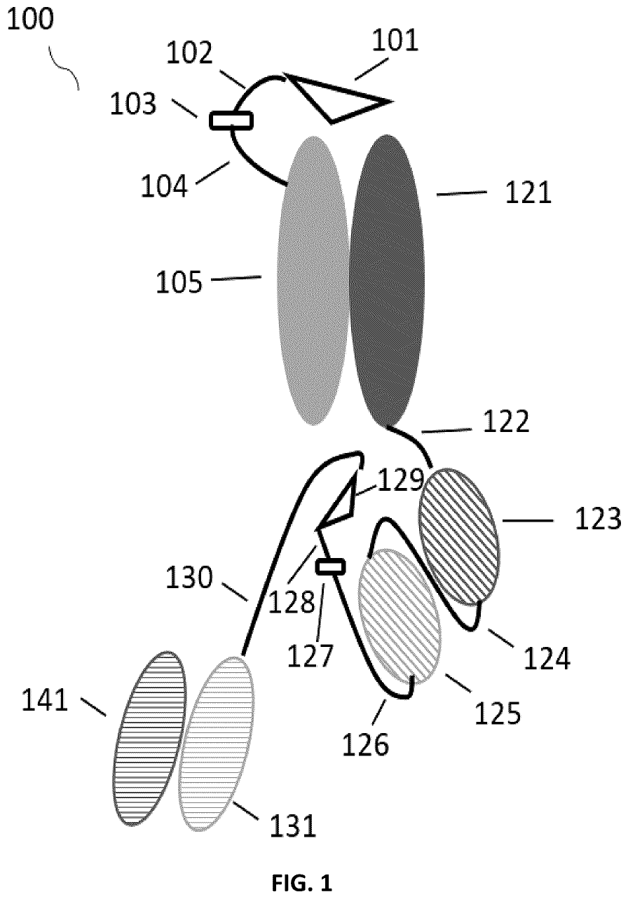

In some embodiments, the activatable protein comprises: a first antigen-binding protein (AB1) that specifically binds to a first target, wherein the AB1 comprises antibody or a fragment thereof comprising a heavy chain fragment and a light chain fragment; a second antigen-binding protein (AB2) that specifically binds to a second target, wherein the AB2 comprises a single chain fragment variable (scFv) comprising a heavy chain variable region and a light chain variable region, and the AB2 is coupled to C-terminus of the heavy chain fragment or the light chain fragment of the AB1; a first masking moiety (MM1) coupled to the AB1 via a first cleavable moiety (CM1) and inhibiting the binding of the AB1 to the first target when the activatable protein is in an uncleaved state; a second masking moiety (MM2) coupled to the AB2 and inhibiting the binding of the AB2 to the second target when the activatable protein is in the uncleaved state; and a half-life extending moiety (EM) coupled to a component of the AB1 or the AB2 via a second cleavable moiety (CM2). In some examples, the AB1 may be a Fab. In some examples, the AB1 may be a scFv. In some aspects, the EM is coupled to the AB1 or to the AB2 through a masking moiety, e.g., a EM-MM-CM-AB or AB-CM-MM-EM structure, optionally with one or more linkers between one or more of the components. As used herein, the symbol “-” in a structure formula indicates directly or indirectly coupling of two components (e.g., optional linkers may be present between the components). Structural configurations of the molecules of the present disclosure are described in detail below and depicted in, e.g., FIGS. 1-6.

As used herein, the terms “activatable protein” and “activatable target-binding protein” (e.g., an “activatable antibody”) and either of the foregoing together with the terms “intact,” “uncleaved” and/or “inactive” are used interchangeably to refer to a protein that comprises at least one set of MM, CM, and TB and which exhibits attenuated binding to a biological target as compared to the binding of a counterpart “activated” protein comprising the same TB to the same biological target (such as, for example, an activated antibody). It will be apparent to the ordinarily skilled artisan that exposure of the activatable protein to a CM-specific protease may generate an “activated” protein in which the MM is not reducing, inhibiting, or interfering with binding between the TB (e.g., AB) and its target. In some embodiments, cleavage of the CM by the appropriate protease may result in release of the MM. In some embodiments, cleavage of the CM by the appropriate protease may result in release of the EM. The terms “activated protein”, “activated target-binding protein” (e.g., “activated antibody”), “cleaved activatable protein”, and “cleaved activatable target-binding protein” (e.g., “cleaved activatable antibody”) refer interchangeably herein to the TB-containing cleavage product that is generated after exposure of the activatable protein to a CM-specific protease. As used throughout this disclosure, descriptions relating to activatable antibodies should be construed to also be applicable to activatable target-binding proteins.

As used herein, the term “masking moiety” and “MM” are used interchangeably to refer to a peptide or protein that, when positioned proximal to a TB (e.g., an AB), interferes with binding of the TB to the biological target. The terms “cleavable moiety” and “CM” are used interchangeably herein to refer to a peptide that comprises a substrate for a sequence-specific protease. In an activatable protein, the CM is positioned relative to the MM and TB, such that cleavage results in a molecule that is capable of binding to the biological target of the TB. Thus, the activatable protein exhibits a reduction in binding to the biological target as compared to the activated protein. In some embodiments, an activatable protein may be designed by selecting a TB of interest and constructing the remainder of the activatable protein so that the MM provides for masking of the TB or reduction of binding of the TB to its target. Structural design criteria can be taken into account to provide for this functional feature.

The activatable protein may be a multispecific (e.g., bispecific, trispecific, tetraspecific, and other multispecific activatable proteins) activatable protein that is capable of binding to multiple distinct antigens when activated. In some embodiments, the multispecific activatable protein may be multivalent, e.g., comprising multiple target-binding sites regardless of whether the binding sites recognize the same or different antigens or epitopes. In some embodiments, the activatable protein may be monospecific, e.g. capable of binding to only one antigen when activated.

In some embodiments, the activatable protein is bispecific. The term “bispecific” means that the activatable protein, when activated, is able to specifically bind to two distinct targets. Typically, an activatable bispecific activatable protein comprises two TBs, a first TB and a second TB, each of which is capable of specifically binding to a different target (i.e., a first target and a second target, respectively) after activation. In some embodiments, after activation, the resulting bispecific target binding molecule may be capable of simultaneously binding two targets, e.g., two target proteins expressed on two distinct cells.

In some embodiments, the activatable protein may comprise an AB1 capable of binding to a molecule on the surface of a cell associated with a disease (e.g., a tumor cell) and an AB2 capable of binding to a molecule on the surface of an immune cell. When activated, such bispecific activatable protein may simultaneously bind to an immune cell and a cell associated with a disease (e.g., a tumor cell), thus activating the immune cell and crosslinking the activated immune cell to the cell associated with the disease. In some embodiments, the activatable protein may be formulated as part of a pro-Bispecific T Cell Engager (pro-BiTE) molecule, pro-Chimeric Antigen Receptor (pro-CAR) modified T cell, or other engineered receptor or other immune effector cell, such as a CAR modified NK cell.

In some examples, the activatable protein may be an activatable T cell-engaging bispecific antibody (TCB) or a fragment thereof. For example, the activatable protein may comprise an AB1 targeting a cell associated with a disease and an AB2 targeting a T cell receptor.

The present disclosure includes activatable proteins in various structural configurations described herein. Exemplary configurations of activatable proteins are provided below. The N- to C-terminal order of the TB, MM, CM, and EM may be reversed within an activatable protein. The CM and MM may overlap in amino acid sequence, e.g., such that the CM sequence recognized by the sequence-specific protease is at least partially contained within the MM. For example, various structural configurations of an activatable antigen-binding protein in which the AB1 is an antigen-binding fragment (Fab) and the AB2 is a single chain fragment variable are contemplated, and can be represented by the formulas below (in order from an amino (N) terminal region to carboxyl (C) terminal region). In the formulas below, “:” separates two different polypeptides; “Fab_L” and “Fab_H” are the light and heavy chain fragments, respectively, of the Fab (“Fab_L” comprises the variable light chain region (VL) and the light chain constant region; “Fab_H” comprises the variable heavy chain region (VH) and the CH1 region); “VL*” and “VH*” are the light and heavy chain variable regions of the scFv. Further, as used herein and unless otherwise stated, each dash (-) between the components of the activatable molecule represents either a direct linkage or indirect linkage via one or more linkers.
(MM1-CM1-Fab_L):(Fab_H-VH*-VL*-CM2-MM2-EM)
(MM1-CM1-Fab_L):(Fab_H-VH*-VL*-CM2-EM-MM2)
Fab_L:(MM1-CM1-Fab_H-VH*-VL*-CM2-MM2-EM)
Fab_L:(MM1-CM1-Fab_H-VH*-VL*-CM2-EM-MM2)
(MM1-CM1-Fab_L-VH*-VL*-CM2-MM2-EM):Fab_H
(MM1-CM1-Fab_L-VH*-VL*-CM2-EM-MM2):Fab_H
(Fab_L-VH*-VL*-CM2-MM2-EM):(MM1-CM1-Fab_H)
(Fab_L-VH*-VL*-CM2-EM-MM2):(MM1-CM1-Fab_H)
(MM1-CM1-Fab_L-VH*-VL*):(Fab_H-CM2-MM2-EM)
(MM1-CM1-Fab_L-VH*-VL*):(Fab_H-CM2-EM-MM2)
(Fab_L-VH*-VL*):(MM1-CM1-Fab_H-CM2-MM2-EM)
(Fab_L-VH*-VL*):(MM1-CM1-Fab_H-CM2-EM-MM2)
(MM1-CM1-Fab_L-CM2-MM2-EM):(Fab_H-VH*-VL*)
(MM1-CM1-Fab_L-CM2-EM-MM2):(Fab_H-VH*-VL*)
(Fab_L-CM2-MM2-EM):(MM1-CM1-Fab_H-VH*-VL*)
(Fab_L-CM2-EM-MM2):(MM1-CM1-Fab_H-VH*-VL*)
(MM1-CM1-Fab_L):(MM2-CM2-VH*-VL*-Fab_H-CM3-EM)
(MM1-CM1-Fab_H):(MM2-CM2-VH*-VL*-Fab_L-CM3-EM)
(MM2-CM2-VH*-VL*-Fab_L):(MM1-CM1-Fab_H-CM3-EM)
(MM2-CM2-VH*-VL*-Fab_H):(MM1-CM1-Fab_L-CM3-EM)

In any of the configurations, the activatable protein may comprise one or more linkers between any two of the components. For example, the activatable protein may comprise a linker between the MM1 and the CM1, a linker between the CM1 and the Fab_L, a linker between the Fab_H and the VH*, a linker between the VH* and the VL*, a linker between the VL* and the CM2, a linker between the CM2 and the MM2, a linker between the MM2 and the EM, a linker between the Fab_L and the VH*, a linker between the CM1 and the Fab_H, or any combination of thereof.

In some embodiments, the EM may comprise two or more moieties (e.g., a pair of Fe domains). For example, the EM may be a protein complex comprising two moieties EM1 and EM2. In such cases, examples of such activatable proteins can be represented by the formulae below (in order from an amino (N) terminal region to carboxyl (C) terminal region):
(MM1-CM1-Fab_L):(Fab_H-VH*-VL*-CM2-MM2-EM1):EM2
(MM1-CM1-Fab_L):(Fab_H-VH*-VL*-CM2-EM1-MM2):EM2
Fab_L:(MM1-CM1-Fab_H-VH*-VL*-CM2-MM2-EM1):EM2
Fab_L:(MM1-CM1-Fab_H-VH*-VL*-CM2-EM1-MM2):EM2
(MM1-CM1-Fab_L):(Fab_H-VH*-VL*-CM2-EM1):(MM2-EM2)
(MM1-CM1-Fab_L):(Fab_H-VH*-VL*-CM2-EM1):(EM2-MM2)
Fab_L:(MM1-CM1-Fab_H-VH*-VL*-CM2-EM1):(MM2-EM2)
Fab_L:(MM1-CM1-Fab_H-VH*-VL*-CM2-EM1):(EM2-MM2)
(MM1-CM1-Fab_L-VH*-VL*-CM2-MM2-EM1):Fab_H:EM2
(MM1-CM1-Fab_L-VH*-VL*-CM2-EM1-MM2):Fab_H:EM2
(Fab_L-VH*-VL*-CM2-MM2-EM1):(MM1-CM1-Fab_H):EM2
(Fab_L-VH*-VL*-CM2-EM1-MM2):(MM1-CM1-Fab_H):EM2
(MM1-CM1-Fab_L-VH*-VL*-CM2-EM1):Fab_H:MM2-EM2
(MM1-CM1-Fab_L-VH*-VL*-CM2-EM1):Fab_H:EM2-MM2
(Fab_L-VH*-VL*-CM2-EM1):(MM1-CM1-Fab_H):MM2-EM2
(Fab_L-VH*-VL*-CM2-EM1):(MM1-CM1-Fab_H):EM2-MM2
(MM1-CM1-Fab_L-VH*-VL*):(Fab_H-CM2-MM2-EM1):EM2
(MM1-CM1-Fab_L-VH*-VL*):(Fab_H-CM2-EM1-MM2):EM2
(Fab_L-VH*-VL*):(MM1-CM1-Fab_H-CM2-MM2-EM1):EM2
(Fab_L-VH*-VL*):(MM1-CM1-Fab_H-CM2-EM1-MM2):EM2
(MM1-CM1-Fab_L-VH*-VL*):(Fab_H-CM2-EM1):MM2-EM2
(MM1-CM1-Fab_L-VH*-VL*):(Fab_H-CM2-EM1):EM2-MM2
(Fab_L-VH*-VL*):(MM1-CM1-Fab_H-CM2-EM1):MM2-EM2
(Fab_L-VH*-VL*):(MM1-CM1-Fab_H-CM2-EM1):EM2-MM2
(MM1-CM1-Fab_L-CM2-MM2-EM1):(Fab_H-VH*-VL*):EM2
(MM1-CM1-Fab_L-CM2-EM1-MM2):(Fab_H-VH*-VL*):EM2
(Fab_L-CM2-MM2-EM1):(MM1-CM1-Fab_H-VH*-VL*):EM2
(Fab_L-CM2-EM1-MM2):(MM1-CM1-Fab_H-VH*-VL*):EM2
(MM1-CM1-Fab_L-CM2-EM1):(Fab_H-VH*-VL*):MM2-EM2
(MM1-CM1-Fab_L-CM2-EM1):(Fab_H-VH*-VL*):EM2-MM2
(Fab_L-CM2-EM1):(MM1-CM1-Fab_H-VH*-VL*):MM2-EM2
(Fab_L-CM2-EM1):(MM1-CM1-Fab_H-VH*-VL*):EM2-MM2
(MM1-CM1-Fab_L):(MM2-CM2-VH*-VL*-Fab_H-CM3-EM1):EM2
(MM1-CM1-Fab_H):(MM2-CM2-VH*-VL*-Fab_L-CM3-EM1):EM2
(MM2-CM2-VH*-VL*-Fab_L):(MM1-CM1-Fab_H-CM3-EM1):EM2
(MM2-CM2-VH*-VL*-Fab_H):(MM1-CM1-Fab_L-CM3-EM):EM2

In some embodiments, the EM1 and EM2 may be two fragment crystallizable (Fc) domains. The two Fc domain may form a dimer as the half-extending moiety. In some examples, the EM1 and EM2 may be two identical Fc domains and thus may form a homodimer. In some constructs, EM1 and EM2 comprise Fc domains having two different amino acid sequences that together form a heterodimer. In some examples, the two Fc domains may be a Fc domain hole mutant and a Fc domain knob mutant and may form a heterodimer. In any of these configurations, the activatable protein may include one or more linkers between any two of the components. For example, the activatable protein may comprise a linker between the MM1 and the CM1, a linker between the CM1 and the Fab_L, a linker between the Fab_H and the VH*, a linker between the VH* and the VL*, a linker between the VL* and the CM2, a linker between the CM2 and the MM2, a linker between the MM2 and the EM, a linker between the Fab_L and the VH*, a linker between the CM1 and the Fab_H, a linker between the CM2 and the EM1, a linker between the MM2 and the EM1, a linker between the MM2 and the EM2, or any combination of thereof.

FIGS. 1-4 show exemplary configurations of the activatable molecules disclosed herein. In these non-limiting examples, the activatable molecules comprise an AB1 that may be a Fab, an AB2 that may be a scFv, an EM that is a dimer formed by two Fc domains, a MM1 coupled to the AB1 via a CM1 and capable of interfering with the binding of the AB1 and its target, a MM2 capable of interfering with the binding of the AB2 and its target, and a CM2 between a Fc domain of the EM and the AB1 or AB2. It will be appreciated that the activatable molecule structures exemplified in FIGS. 1-4 can analogously be applied to molecules in which the AB1 and the AB2 are antigen-binding proteins other than a Fab and an scFv. Likewise, the activatable molecule structures exemplified in FIGS. 1-4 can analogously be applied to molecules in which the AB1 and the AB2 are replaced by a TB1 and TB2, respectively, that may be target-binding proteins that do not necessarily comprise an antigen binding domain.

FIG. 1 shows an exemplary activatable protein 100 comprising three polypeptides. The first polypeptide, in order from an amino (N) terminal region to carboxyl (C) terminal region, comprises the MM1 101, an optional linker 102, the CM1 103, an optional linker 104, and the AB1's light chain fragment 105. The second polypeptide, in order from an amino (N) terminal region to carboxyl (C) terminal region, comprises the AB1's heavy chain fragment 121, a linker 122, the AB2's heavy chain variable region 123, a linker 124, the AB2's light chain variable region 125, a linker 126, the CM2 127, an optional linker 128, the MM2 129, a linker 130, and the EM's first Fc domain 131. The third polypeptide comprises the EM's second Fc domain 141. In an alternative exemplary configuration, in FIG. 1, 105 is the AB1's heavy chain fragment and 121 is the AB1's light chain fragment. In an alternative exemplary configuration, in FIG. 1, 123 is the AB2's light chain variable region and 125 is the AB2's heavy chain variable region. In an alternative exemplary configuration, in FIG. 1, the EM's first Fc domain 131 is a Fc domain hole mutant and the EM's second Fc domain 141 is a Fc domain knob mutant. In an alternative exemplary configuration, in FIG. 1, the EM's first Fc domain 131 is a Fc domain knob mutant and the EM's second Fc domain 141 is a Fc domain hole mutant.

FIG. 2 shows another exemplary activatable protein 200 comprising three polypeptides. The first polypeptide, in order from an amino (N) terminal region to carboxyl (C) terminal region, comprises the MM1 201, an optional linker 202, the CM1 203, an optional linker 204, the AB1's light chain fragment 205, a linker 206, the AB2's heavy chain variable region 207, a linker 208, and the AB2's light chain variable region 209. The second polypeptide, in order from an amino (N) terminal region to carboxyl (C) terminal region, comprises the AB1's heavy chain fragment 221, an optional linker 222, the CM2 223, an optional linker 224, the MM2 225, a linker 226, and the EM's first Fc domain 227. The third polypeptide comprises the EM's second Fc domain 241. In an alternative exemplary configuration, in FIG. 2, 205 is the AB1's heavy chain fragment and 221 is the AB1's light chain fragment. In an alternative exemplary configuration example, in FIG. 2, 207 is the AB2's light chain variable region and 209 is the AB2's heavy chain variable region. In an alternative exemplary configuration example, in FIG. 2, the EM's first Fc domain 227 is a Fc domain hole mutant and the EM's second Fc domain 241 is a Fc domain knob mutant. In an alternative exemplary configuration example, in FIG. 2, the EM's first Fc domain 227 is a Fc domain knob mutant and the EM's second Fc domain 241 is a Fc domain hole mutant.

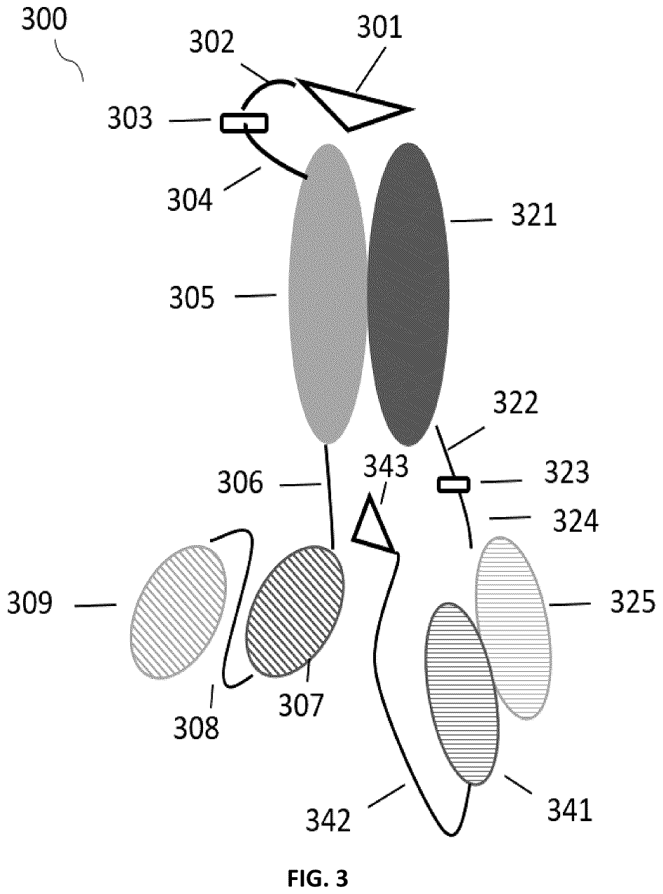

FIG. 3 shows another exemplary activatable protein 300 comprising three polypeptides. The first polypeptide, in order from an amino (N) terminal region to carboxyl (C) terminal region, comprises the MM1 301, an optional linker 302, the CM1 303, an optional linker 304, the AB1's light chain fragment 305, a linker 306, the AB2's heavy chain variable region 307, a linker 308, and the AB2's light chain variable region 309. The second polypeptide, in order from an amino (N) terminal region to carboxyl (C) terminal region, comprises the AB1's heavy chain fragment 321, an optional linker 322, the CM2 323, an optional linker 324, and the EM's first Fc domain 325. The third polypeptide, in order from an amino (N) terminal region to carboxyl (C) terminal region, comprises the EM's second Fc domain 341, a linker 342, and the MM2 343. In an alternative exemplary configuration, in FIG. 3, 305 is the AB1's heavy chain fragment and 321 is the AB1's light chain fragment. In an alternative exemplary configuration, in FIG. 3, 307 is the AB2's light chain variable region and 309 is the AB2's heavy chain variable region. In an alternative exemplary configuration, in FIG. 3, the EM's first Fc domain 325 is a Fc domain hole mutant and the EM's second Fc domain 341 is a Fc domain knob mutant. In an alternative exemplary configuration, in FIG. 3, the EM's first Fc domain 325 is a Fc domain knob mutant and the EM's second Fc domain 341 is a Fc domain hole mutant.

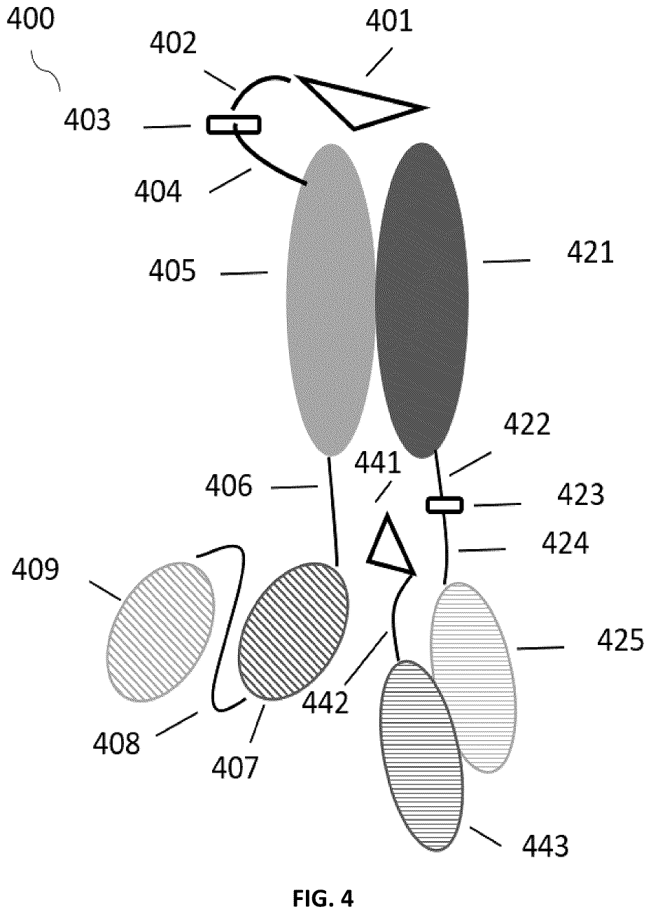

FIG. 4 shows another exemplary activatable protein 400 comprising three polypeptides. The first polypeptide, in order from an amino (N) terminal region to carboxyl (C) terminal region, comprises the MM1 401, an optional linker 402, the CM1 403, an optional linker 404, the AB1's light chain fragment 405, a linker 406, the AB2's heavy chain variable region 407, a linker 408, and the AB2's light chain variable region 409. The second polypeptide, in order from an amino (N) terminal region to carboxyl (C) terminal region, comprises the AB1's heavy chain fragment 421, an optional linker 422, the CM2 423, an optional linker 424, and the EM's first Fc domain 425. The third polypeptide, in order from an amino (N) terminal region to carboxyl (C) terminal region, comprises the MM2 441, a linker 442, and the EM's second Fc domain 443. In an alternative exemplary configuration, in FIG. 4, 405 is the AB1's heavy chain fragment and 421 is the AB1's light chain fragment. In an alternative exemplary configuration, in FIG. 4, 407 is the AB2's light chain variable region and 409 is the AB2's heavy chain variable region. In an alternative exemplary configuration, in FIG. 4, the EM's first Fc domain 425 is a Fc domain hole mutant and the EM's second Fc domain 443 is a Fc domain knob mutant. In an alternative exemplary configuration, in FIG. 4, the EM's first Fc domain 425 is a Fc domain knob mutant and the EM's second Fc domain 443 is a Fc domain hole mutant.

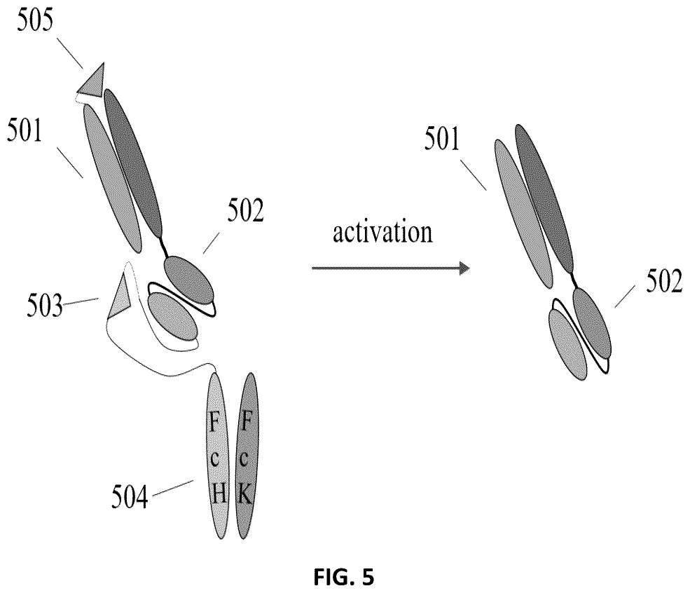

FIG. 5 shows an exemplary activatable bispecific antibody that comprises an Fab component (501) that binds a first target; a first prodomain (505) comprising a CM1 and an MM1 that masks the Fab component; an scFv component (502) that binds a second target; a second prodomain (503) comprising a CM2 and an MM2 that masks the scFv component; and an EM comprising a pair of knob and hole Fc domains (504). Upon activation, the CM1 is cleaved releasing the MM1, and the CM2 is cleaved releasing both the MM2 and the Fc domain (504) from the activated bispecific antibody. The activated bispecific antibody lacking the Fc domain has a relatively short half-life compared to its parent activatable bispecific antibody.

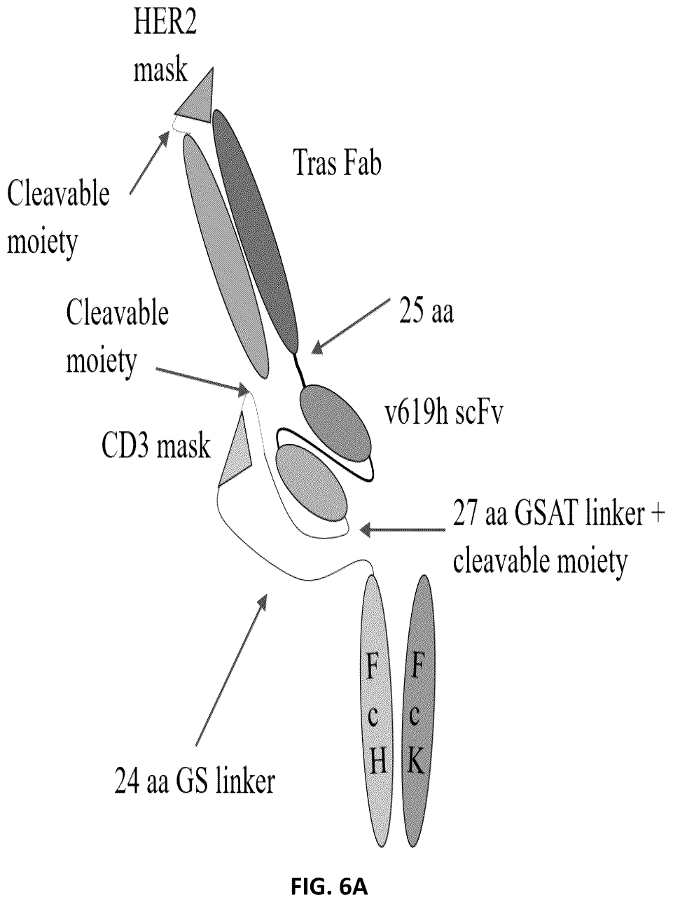

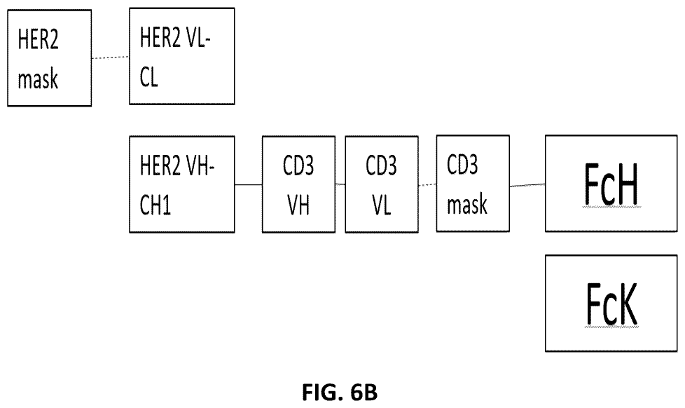

FIG. 6A shows an exemplary activatable bispecific antibody targeting Her2 and CD3. In this example, the activatable bispecific antibody comprises three polypeptides. The first polypeptide comprises, in order from N- to C-terminus: a heavy chain fragment of the Fab of trastuzumab (an anti-HER2 antibody), a linker with 25 amino acids, an anti-CD3 scFv, a GSAT linker with 27 amino acids, a CM1, a MM1 for masking the anti-CD3 scFv, a GS linker with 24 amino acids, and a Fc domain hole mutant. The second polypeptide comprises a MM2 for making the Fab, a CM2, and a light chain fragment of the Fab. The third polypeptide comprises a Fc domain knob mutant. Examples of the activatable bispecific antibody with the configuration in FIG. 6 may comprise a first polypeptide comprising a sequence of any one of SEQ ID NOs: 21-24, a second polypeptide comprising a sequence of SEQ ID NO: 18, and third polypeptide comprising a sequence of SEQ ID NO: 1. FIG. 6B is a schematic representation of the three polypeptides that form the activatable bispecific antibody shown in FIG. 6A.

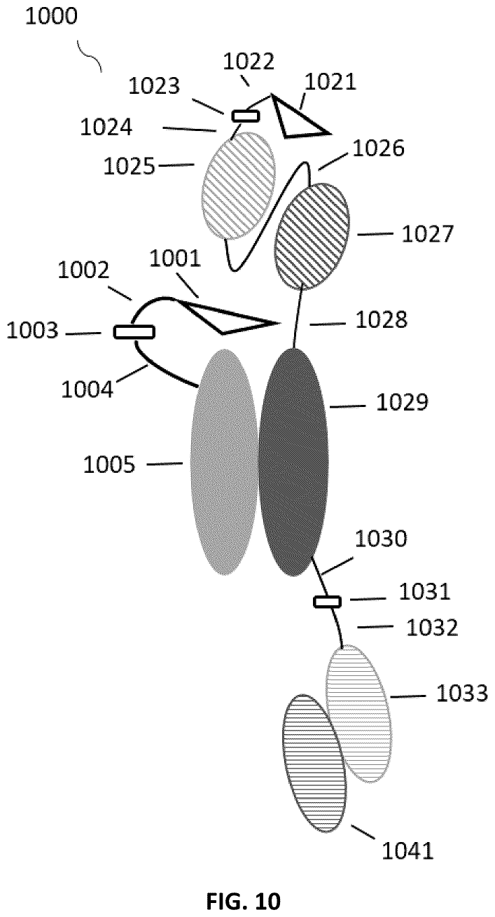

FIG. 10 shows another exemplary activatable protein 1000 comprising three polypeptides. The first polypeptide, in order from an amino (N) terminal region to carboxyl (C) terminal region, comprises the MM1 1001, an optional linker 1002, the CM1 1003, an optional linker 1004, and the AB1's light chain fragment 1005. The second polypeptide, in order from an amino (N) terminal region to carboxyl (C) terminal region, comprises the MM2 1021, an optional linker 1022, the CM2 1023, an optional linker 1024, the AB2's heavy chain variable region 1025, a linker 1026, the AB2's light chain variable region 1027, a linker 1028, the AB1's heavy chain fragment 1029, an optional linker 1030, a third cleavable moiety (CM3) 1031, an optional linker 1032, and a first domain of the EM (EM1) 1033. The third polypeptide comprises a second domain of the EM (EM2) 1041. In an alternative exemplary configuration, in FIG. 10, 1005 is the AB1's heavy chain fragment and 1029 is the AB1's light chain fragment. In an alternative exemplary configuration, in FIG. 10, 1025 is the AB2's light chain variable region and 1027 is the AB2's heavy chain variable region. In an alternative exemplary configuration, in FIG. 10, the first domain of the EM (EM1) 1033 is a Fc domain hole mutant and the second domain of the EM (EM2) 1041 is a Fc domain knob mutant. In an alternative exemplary configuration, in FIG. 10, the EM1 1033 is a Fc domain knob mutant and the EM2 1041 is a Fc domain hole mutant.

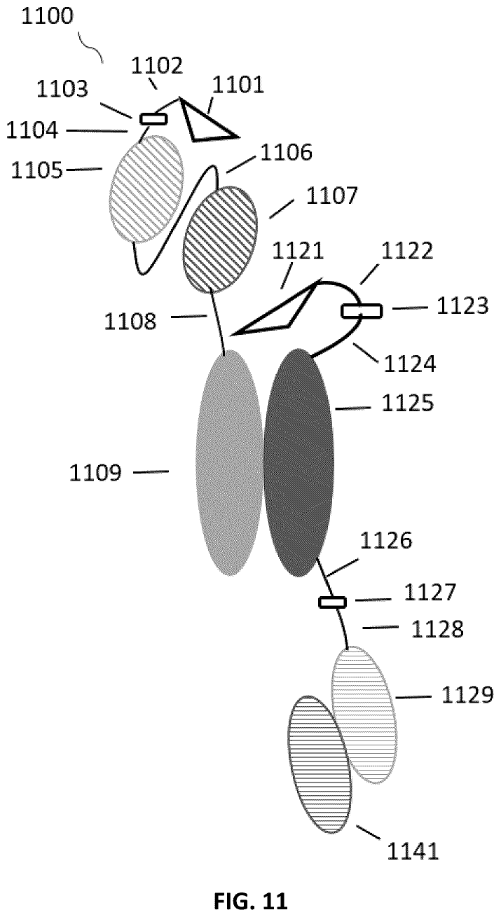

FIG. 11 shows another exemplary activatable protein 1100 comprising three polypeptides. The first polypeptide, in order from an amino (N) terminal region to carboxyl (C) terminal region, comprises the MM2 1101, an optional linker 1102, the CM2 1103, an optional linker 1104, the AB2's heavy chain variable region 1105, a linker 1106, the AB2's light chain variable region 1107, a linker 1108, and the AB1's light chain fragment 1109. The second polypeptide, in order from an amino (N) terminal region to carboxyl (C) terminal region, comprises the MM1 1121, an optional linker 1122, the CM1 1123, an optional linker 1124, the AB1's heavy chain fragment 1125, an optional linker 1126, a third cleavable moiety (CM3) 1127, an optional linker 1128, and a first domain of the EM (EM1) 1129. The third polypeptide comprises a second domain of the EM (EM2) 1141. In an alternative exemplary configuration, in FIG. 11, 1109 is the AB1's heavy chain fragment and 1125 is the AB1's light chain fragment. In an alternative exemplary configuration, in FIG. 11, 1105 is the AB2's light chain variable region and 1107 is the AB2's heavy chain variable region. In an alternative exemplary configuration, in FIG. 11, the EM1 1129 is a Fc domain hole mutant and the EM2 1141 is a Fc domain knob mutant. In an alternative exemplary configuration, in FIG. 11, the EM1 1129 is a Fc domain knob mutant and the EM2 1141 is a Fc domain hole mutant.

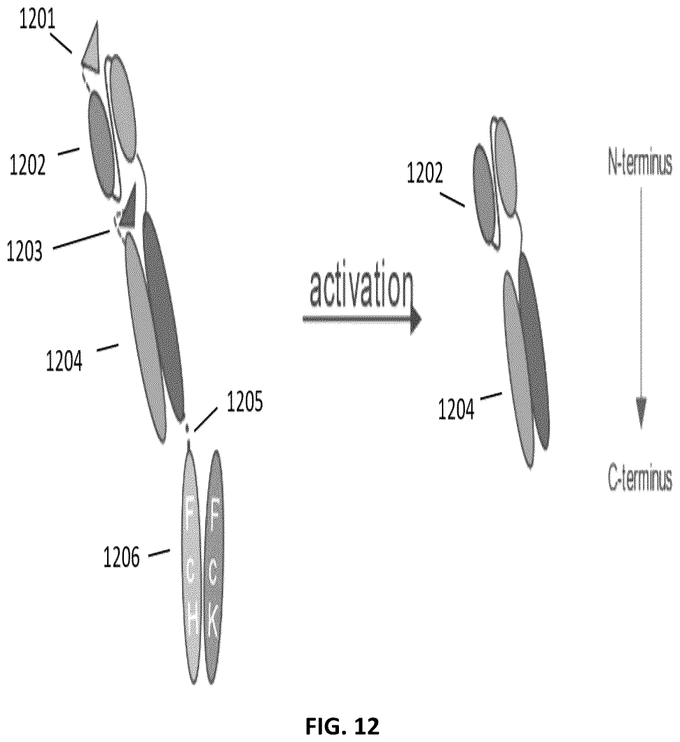

FIG. 12 shows an exemplary activatable bispecific antibody that comprises an Fab component (1204) that binds a first target; a first prodomain (1203) comprising a CM1 (broken line) and an MM1 (triangle) that masks the Fab component; an scFv component (1202) that binds a second target; a second prodomain (1201) comprising a CM2 (broken line) and an MM2 (triangle) that masks the scFv component; an EM comprising a pair of knob and hole Fc domains (1206); and a third cleavable moiety (CM3) (1205) between the EM and the Fab. Upon activation, the CM1 is cleaved releasing the MM1, and the CM2 is cleaved releasing the MM2, and the CM3 is cleaved releasing the EM (1206) from the activated bispecific antibody. The activated bispecific antibody lacking the EM has a relatively short half-life compared to its parent activatable bispecific antibody.

In some embodiments, the activated protein resulting from the activation of the activatable protein of the present disclosure is not attached to the EM. Such activated proteins may have a shorter half-life compared to the activatable protein. Such activated proteins may have a shorter half-life compared to a counterpart protein that is the same as the activated protein but comprising the EM. The term “half-life” as used herein is the time it takes for the concentration of a molecule or a complex of molecules to reach 50% of its original concentration in an environment. In some examples, the environment may be serum and the half-life is serum half-life, which is the time it takes for the concentration of a molecule or a complex of molecules to reach 50% of its original concentration in serum (e.g., in the circulation of a subject). In some examples, an activated protein comprising the AB1 and AB2 but not the MM1, MM2 or EM (i.e., resulting from the activation of the activatable protein) may have a shorter half-life compared to a counterpart protein that is the same as the activated protein but comprising the EM, i.e., the half-life of the activated molecule (AB1-AB2) is shorter than the half-life of the counterpart protein (AB1-AB2-EM).

For example, the activated protein resulting from the activation of the activatable protein herein may have a half-life (e.g., serum half-life) of less than 15 days, 14 days, 13 days, 12 days, 11 days, 10 days, 9 days, 8 days, 7 days, 6 days, 5 days, 4 days, 3 days, 2 days, 1 day, 23 hours, 22 hours, 21 hours, 20 hours, 19 hours, 18 hours, 17 hours, 16 hours, 15 hours, 14 hours, 13 hours, 12 hours, 11 hours, 10 hours, 9 hours, 8 hours, 7 hours, 6 hours, 5 hours, 4 hours, or 3 hours. In one example, the activated protein resulting from the activation of the activatable protein herein may have a half-life (e.g., serum half-life) of less than or equal to 5, 4, 3, or 2 days. In some examples, the activated protein resulting from the activation of the activatable protein herein may have a half-life (e.g., serum half-life) that is up to 90%, 80%, 70%, 60%, 50%, 40%, 30%, 20%, 10%, or 5% of the half-life (e.g., serum half-life) of a counterpart protein that is the same as the activated protein but comprising the EM.

In some embodiments, activated proteins resulting from the activation of the activatable protein herein (i.e., activated proteins that are not attached to the EM or MMs) may have a higher target binding activity compared to a counterpart protein that is the same as the activated protein but comprising the EM attached thereto In some examples, an activated protein comprising the TB1 and TB2 but not the MM1, MM2 or EM has a level of target-binding activity that is greater than that of a counterpart protein that is the same as the activated protein but comprising EM (i.e., TB1-TB2-EM). For example, the activated protein resulting from the activation of the activatable protein disclosed herein may have a target-binding activity that is at least 10%, 20%, 30%, 40%, 50%, 60%, 70%, 80%, 90%, 100%, 3-fold, 4-fold, 6-fold, 8-fold, 10-fold, 20-fold, 30-fold, 40-fold, 50-fold, 60-fold, 70-fold, 80-fold, 90-fold, 100-fold, 200-fold, 300-fold, 400-fold, or 500-fold greater than the target-binding activity of a counterpart protein that is the same as the activated protein but comprising EM.

In some embodiments, the activatable protein (prior to activation) may be characterized by a target-binding activity that is less than a control level of the target-binding activity of the TB without the MM coupled to it, either directly or indirectly. For example, in some embodiments, the activatable protein is characterized by at least a 2, 4, 6, 8, 10, 20, 30, 40, 50, 60, 70, 80, 90, 100, 200, 300, 400, 500, 600, 700, 800, 900, 1000, 2000, 5000, or 10000 fold reduction in targeting binding activity as compared to the control level of the target-binding activity of the TB without the MM coupled to it.

### Target-Binding Proteins

An activatable protein according to the present disclosure may include one or more target-binding proteins (TBs). In some examples, the activatable protein may be multispecific. For example, the activatable protein may comprise multiple TBs, each having specificity for a different epitope on the same target. In some examples, the TBs in an activatable protein herein may bind to different targets, e.g., targets on different types of cells. This way, in the activated protein resulting from the activation of the activatable protein disclosed herein, the TBs may co-localize the different types of cells. In some examples of multispecific activatable proteins of the present disclosure, one of the TBs binds to a target on an immune cell and another of the TBs binds to a cell associated with a disease. By targeting and co-localizing the immune cell and the cell associated with the disease, the activated protein may provide a targeted treatment for the disease.

In some embodiments, the target-binding proteins (TBs) may be antigen-binding proteins (ABs). In some embodiments, the AB may be an antibody or a fragment thereof, e.g., a monoclonal antibody, single chain antibody, Fab fragment, F(ab′)₂ fragment, single-chain variable fragment (scFv), diabody (a noncovalent dimer of scFv), single chain antibody (scab), a VHH, a domain antibody (dAb) or single domain antibody (nanobody, e.g., single domain heavy chain antibody, single domain light chain antibody). A single domain antibody may be an antibody fragment that is a single monomeric variable antibody domain. A single domain antibody may have similar affinity to antigens as a corresponding full-length antibody. In some embodiments, the AB may be a full-length antibody. In some embodiments, the AB may be an immunologically active fragment. In some embodiments, the AB may be an antigen-binding fragment (“Fab”). In one example, the activatable protein comprises a Fab as a first AB and a scFv as a second AB. In some embodiments, the AB may be a scFv. In some embodiments, the AB may be a mouse, other rodent, chimeric, humanized or fully human monoclonal antibody. The present disclosure includes structures having combinations of one or more polypeptides comprising any of the domains listed above, e.g., one or more of SDA, Fv, ScFv, Fab, scFab, VHH, and dAb, with one or more selected from SDA, Fv, scFv, Fab, VHH, scFab, and dAb.

The term “antibody” is used herein in its broadest sense and includes certain types of immunoglobulin molecules that include one or more antigen-binding domains that specifically bind to an antigen or epitope. The term “antibody” specifically includes, e.g., intact antibodies (e.g., intact immunoglobulins), antibody fragments, bispecific, and multi-specific antibodies. One example of an antibody is an antigen-binding domain formed by a V_{H}-V_{L} dimer. Additional examples of an antibody are described herein. Additional examples of an antibody are known in the art.

A “light chain” consists of one variable domain (VL) and one constant domain (CL). There are two different light chain types or classes termed kappa or lambda.

A “heavy chain” consists of one variable domain (VH) and three constant region domains (CH1, CH2, CH3). There are five main heavy-chain classes or isotypes, some of which have several subtypes, and these determine the functional activity of an antibody molecule. The five major classes of immunoglobulin are immunoglobulin M (IgM), immunoglobulin D (IgD), immunoglobulin G (IgG), immunoglobulin A (IgA), and immunoglobulin E (IgE). IgG is by far the most abundant immunoglobulin and has several subclasses (IgG1, 2, 3, and 4 in humans).

A “fragment antigen binding” (Fab) contains a complete light chain paired with the VH domain and the CH1 domain of a heavy chain. A F(ab′)₂ fragment is formed when an antibody is cleaved by pepsin below the hinge region, in which case the two fragment antigen-binding domains (Fabs) of the antibody molecule remain linked. A F(ab′)₂ fragment contains two complete light chains paired with the two VH and CH1 domains of the heavy chains joined together by the hinge region. A “fragment crystallizable” (Fc) fragment (also referred to herein as Fc domain) corresponds to the paired CH2 and CH3 domains and is the part of the antibody molecule that interacts with effector molecules and cells. The functional differences between heavy-chain isotypes lie mainly in the Fc fragment. A “single chain Fv” (scFv) contains only the variable domain of a light chain (VL) linked by a stretch of synthetic peptide to a variable domain of a heavy chain (VH). The name single-chain Fv is derived from Fragment variable. A “hinge region” or “interdomain” is flexible amino acid stretch that joins or links the Fab fragment to the Fc domain. A “synthetic hinge region” is an amino acid sequence that joins or links a Fab fragment to an Fc domain.

“Prodomain” refers to a polypeptide that has a portion that inhibits antigen binding referred to as a masking moiety (MM) and a portion containing a protease cleavable substrate referred to as a cleavable moiety (CM) that when linked to a target-binding protein (TB) (e.g., antigen-binding protein (AB) such as an antibody or antigen binding fragment thereof), functions to inhibit antigen binding by the. The prodomain may include a linker peptide (L1) between the MM and the CM. The prodomain may also include a linker peptide (L2) at the prodomain's carboxyl terminus to facilitate the linkage of the prodomain to the antibody. In certain embodiments, a prodomain comprises one of the following formulae (wherein the formula below represents an amino acid sequence in an N- to C-terminal direction):(MM)-(CM), (MM)-L1-(CM), (MM)-(CM)-L2, or (MM)-L1-(CM)-L2.

The TB (e.g, an AB) specifically binds to a target. As used herein, the terms “specific binding,” “immunological binding,” and “immunological binding properties” refer to the non-covalent interactions of the type that occur between an immunoglobulin molecule and an antigen for which the immunoglobulin is specific. The strength or affinity of immunological binding interactions can be expressed in terms of the dissociation constant (K_{d}) of the interaction, wherein a smaller K_{d} represents a greater affinity. Immunological binding properties of selected polypeptides may be quantified using methods well known in the art. One such method entails measuring the rates of antigen-binding site/antigen complex formation and dissociation, wherein those rates depend on the concentrations of the complex partners, the affinity of the interaction, and geometric parameters that equally influence the rate in both directions. Thus, both the “on rate constant” (Kₒₙ) and the “off rate constant” (K_{off}) can be determined by calculation of the concentrations and the actual rates of association and dissociation. (See Nature 361:186-87 (1993)). The ratio of K_{off}/Kₒₙ enables the cancellation of all parameters not related to affinity, and is equal to the dissociation constant K_{d}. (See, generally, Davies et al. (1990) Annual Rev Biochem 59:439-473). A TB or antibody binding domain (AB) of the present disclosure is said to “specifically bind” or “immunospecifically bind” to the target, when the dissociation constant (K_{d}) is ≤100 μM, in some embodiments ≤1 μM, in some embodiments 100 nM, in some embodiments 10 nM, and in some embodiments ≤100 pM to about 1 pM, as measured by assays such as radioligand binding assays or similar assays known to those skilled in the art.

The target of the TB (e.g., AB) may be a protein or other type of molecules. Example targets of a TB include cell surface receptors and secreted binding proteins (e.g., growth factors), soluble enzymes, structural proteins (e.g. collagen, fibronectin) and the like. In some examples, the target of a TB may be a protein associated with a disease (e.g., cancer) in a subject.

In some embodiments, a TB in the activatable protein may bind to a target that is a molecule on or inside a cell associated with a disease. For example, a TB in the activatable protein may bind to a tumor cell. In such cases, the TB may bind to a tumor associated antigen. As used herein, the term “tumor associated antigen” means any antigen including a protein, glycoprotein, ganglioside, carbohydrate, lipid that is associated with cancer. Such antigen may be expressed on tumor cells (e.g., malignant cells) or in the tumor microenvironment such as on tumor-associated blood vessels, extracellular matrix, mesenchymal stroma, or immune infiltrates. In some embodiments, the tumor associated antigen that is the target of the AB may be human epidermal growth factor receptor 2 (HER2). For example, the AB may be trastuzumab or a fragment thereof, e.g., the Fab of trastuzumab.

In some embodiments, an AB in the activatable protein may bind to at target that is a molecule on an immune cell and/or capable of activating the immune cell. In some examples, the target of the AB may be a co-stimulatory molecule, which is a cell surface molecule other than antigen receptors or ligands thereof required for a highly efficient immune response. Examples of the co-stimulatory molecules that may be the target of the AB include a component of T cell receptor (TCR), CD3 zeta, CD3 gamma, CD3 delta, and CD3 epsilon.

In some examples, the AB may bind to a co-stimulatory molecule expressed on the surface of a T lymphocyte, e.g., a cytotoxic T lymphocyte, which is capable of inducing T cell activation upon interaction with an antigen binding molecule. The interaction of an antigen binding molecule with an activating T cell antigen may induce T cell activation by triggering the signaling cascade of the T cell receptor complex. When activated, the AB may bind to such co-stimulatory molecule to activate T cells. “T cell activation” as used herein refers to one or more cellular response of a T lymphocyte, particularly a cytotoxic T lymphocyte, selected from: proliferation, differentiation, cytokine secretion, cytotoxic effector molecule release, cytotoxic activity, and expression of activation markers. The T cell activating bispecific antigen binding molecules of the invention are capable of inducing T cell activation.

In some examples, the AB may bind to CD3. For example, the CD3 may be the epsilon subunit of CD3, e.g., the sequence of NCBI RefSeq no. NP_000724.1. In some examples, the AB may be an anti-CD3 scFv. The anti-CD3 scFv may comprise one or more sequences of SEQ ID NOs: 1-9, 143-145, 149, and 150 of US20190135943, which is incorporated by reference herein in its entirety. Such sequences include, for example, the following:

Anti-CD3 variant v12
Light Chain Variable Domain LV12
QTVVTQEPSFSVSPGGTVTLTCRSSTGAVTTSNYANWVQQTPGQAPRGLIGGTNKRAP
GVPDRFSGSILGNKAALTITGAQADDESDYYCALWYSNLWVFGGGTKLTVL (SEQ ID
NO: 47)
Heavy Chain Variable Domain HV12,
EVQLVESGGGLVQPGGSLRLSCAASGFTFSTYAMNWVRQAPGKGLEWVARIR
SKYNNYATYYADSVKDRFTISRDDSKNSLYLQMNSLKTEDTAVYYCVRHGNFGNSY
VSWFAYWGQGTLVTVSS (SEQ ID NO: 48)
LV12-L3-HV12 wherein L3 is GGGGSGGGGSGGGGS (SEQ ID NO: 28)
QTVVTQEPSFSVSPGGTVTLTCRSSTGAVTTSNYANWVQQTPGQAPRGLIGGT
NKRAPGVPDRFSGSILGNKAALTITGAQADDESDYYCALWYSNLWVFGGGTKLTVL
[GGGGSGGGGSGGGGS]EVQLVESGGGLVQPGGSLRLSCAASGFTFSTYAMNWVRQA
PGKGLEWVARIRSKYNNYATYYADSVKDRFTISRDDSKNSLYLQMNSLKTEDTAVY
YCVRHGNFGNSYVSWFAYWGQGTLVTVSS (SEQ ID NO: 49)
Anti-CD3 variant v16
Light Chain Variable Domain LV12
Sequence provided above
Heavy Chain Variable Domain HV20
EVQLVESGGGLVQPGGSLKLSCAASGFTFSTYAMNWVRQASGKGLEWVGRIR
SKYNNYATYYADSVKDRFTISRDDSKNTAYLQMNSLKTEDTAVYYCTRHGNFGNSY
VSWFAYWGQGTLVTVSS (SEQ ID NO: 50)
LV12-L3-HV20
QTVVTQEPSFSVSPGGTVTLTCRSSTGAVTTSNYANWVQQTPGQAPRGLIGGT
NKRAPGVPDRFSGSILGNKAALTITGAQADDESDYYCALWYSNLWVFGGGTKLTVL
[GGGGSGGGGSGGGGS]EVQLVESGGGLVQPGGSLKLSCAASGFTFSTYAMNWVRQA
SGKGLEWVGRIRSKYNNYATYYADSVKDRFTISRDDSKNTAYLQMNSLKTEDTAVY
YCTRHGNFGNSYVSWFAYWGQGTLVTVSS (SEQ ID NO: 51)
Anti-CD3 variant v19
Light Chain Variable Domain LV19
QAVVTQEPSLTVSPGGTVTLTCRSSTGAVTTSNYANWVQQKPGQAPRGLIGGTNKRA
PGTPARFSGSLIGGKAALTLSGAQPEDEAEYYCALWYSNLWVFGGGTKLTVL (SEQ
ID NO: 52)
Heavy Chain Variable Domain HV20
Sequence provided above
LV19-L3-HV20
QAVVTQEPSLTVSPGGTVTLTCRSSTGAVTTSNYANWVQQKPGQAPRGLIGGT
NKRAPGTPARFSGSLIGGKAALTLSGAQPEDEAEYYCALWYSNLWVFGGGTKLTVL
[GGGGSGGGGSGGGGS]EVQLVESGGGLVQPGGSLKLSCAASGFTFSTYAMNWVRQA
SGKGLEWVGRIRSKYNNYATYYADSVKDRFTISRDDSKNTAYLQMNSLKTEDTAVY
YCTRHGNFGNSYVSWFAYWGQGTLVTVSS (SEQ ID NO: 53)
Anti-CD3 variant v26
Light Chain Variable Domain LV19
Sequence provided above
Heavy Chain Variable Domain HV12
Sequence provided above
LV19-L3-HV12
QAVVTQEPSLTVSPGGTVTLTCRSSTGAVTTSNYANWVQQKPGQAPRGLIGGT
NKRAPGTPARFSGSLIGGKAALTLSGAQPEDEAEYYCALWYSNLWVFGGGTKLTVL
[GGGGSGGGGSGGGGS]EVQLVESGGGLVQPGGSLRLSCAASGFTFSTYAMNWVRQA
PGKGLEWVARIRSKYNNYATYYADSVKDRFTISRDDSKNSLYLQMNSLKTEDTAVY
YCVRHGNFGNSYVSWFAYWGQGTLVTVSS (SEQ ID NO: 54)

Exemplary CDR sequences of CD3-binding antibodies include the following:

| Name | CD3 Ab CDR Sequences | SEQ ID NO: |
|---|---|---|
| SP34L1 | RSSTGAVTTSNYAN | SEQ ID NO: 7 |
| SP34L2 | GTNKRAP | SEQ ID NO: 8 |
| SP34L3 | ALWYSNLWV | SEQ ID NO: 9 |
| SP34H1 | TYAMN | SEQ ID NO: 10 |
| SP34H2 | RIRSKYNNYATYYADSVKD | SEQ ID NO: 11 |
| SP34H3 | HGNFGNSYVSWFAY | SEQ ID NO: 12 |

Additional examples of anti-CD3 ABs include the following:

v619_HLc

| | | |
|---|---|---|
| VH CDR1 | SEQ ID NO: 10 | TYAMN |
| VH CDR2 | SEQ ID NO: 11 | RIRSKYNNYATYYADSVKD |
| VH CDR3 | SEQ ID NO: 13 | HGNFGNSYVSWWAY |
| VL CDR1 | SEQ ID NO: 14 | RSSTGAVTTSNYPN |
| VL CDR2 | SEQ ID NO: 8 | GTNKRAP |
| VL CDR3 | SEQ ID NO: 15 | VLWYSNRWV |
| Heavy chain | SEQ ID NO: 30 | EVQLVESGGGLVQPGGSLKLSCAASGFTFSTY |
| variable | | AMNWVRQASGKGLEWVGRIRSKYNNYATY |
| domain | | YADSVKDRFTISRDDSKNTAYLQMNSLKTED |
| | | TAVYYCVRHGNFGNSYVSWWAYWGQGTLV |
| | | TVSS |
| Light chain | SEQ ID NO: 16 | QTVVTQEPSLTVSPGGTVTLTCRSSTGAVTTS |
| variable | | NYPNWVQQKPGQAPRGLIGGTNKRAPGTPAR |
| domain | | FSGSLLGGKAALTLSGVQPEDEAEYYCVLWY |
| | | SNRWVFGGGTKLTVL |
| scFv | SEQ ID NO: 19 | EVQLVESGGGLVQPGGSLKLSCAASGFTFSTY |
| | | AMNWVRQASGKGLEWVGRIRSKYNNYATY |
| | | YADSVKDRFTISRDDSKNTAYLQMNSLKTED |
| | | TAVYYCVRHGNFGNSYVSWWAYWGQGTLV |
| | | TVSSGGGGSGGGGSGGGGSQTVVTQEPSLTV |
| | | SPGGTVTLTCRSSTGAVTTSNYPNWVQQKPG |
| | | QAPRGLIGGTNKRAPGTPARFSGSLLGGKAAL |
| | | TLSGVQPEDEAEYYCVLWYSNRWVFGGGTK |
| | | LTVL |

v619_HLh

| | | |
|---|---|---|
| VH CDR1 | SEQ ID NO: 10 | TYAMN |
| VH CDR2 | SEQ ID NO: 11 | RIRSKYNNYATYYADSVKD |
| VH CDR3 | SEQ ID NO: 13 | HGNFGNSYVSWWAY |
| VL CDR1 | SEQ ID NO: 20 | GSSTGAVTTSNYVN |
| VL CDR2 | SEQ ID NO: 8 | GTNKRAP |
| VL CDR3 | SEQ ID NO: 15 | VLWYSNRWV |
| Heavy chain | SEQ ID NO: 30 | EVQLVESGGGLVQPGGSLKLSCAASGFTFSTY |
| variable | | AMNWVRQASGKGLEWVGRIRSKYNNYATY |
| domain | | YADSVKDRFTISRDDSKNTAYLQMNSLKTED |
| | | TAVYYCVRHGNFGNSYVSWWAYWGQGTLV |
| | | TVSS |
| Light chain | SEQ ID NO: 25 | QTVVTQEPSLTVSPGGTVTLTCGSSTGAVTTS |
| variable | | NYVNWVQQKPGQAPRGLIGGTNKRAPGTPA |
| domain | | RFSGSLIGGKAALTLSGAQPEDEAEYYCVLW |
| | | YSNRWVFGGGTKLTVL |
| scFv | SEQ ID NO: 26 | EVQLVESGGGLVQPGGSLKLSCAASGFTFSTY |
| | | AMNWVRQASGKGLEWVGRIRSKYNNYATY |
| | | YADSVKDRFTISRDDSKNTAYLQMNSLKTED |
| | | TAVYYCVRHGNFGNSYVSWWAYWGQGTLV |
| | | TVSSGGGGSGGGGSGGGGSQTVVTQEPSLTV |
| | | SPGGTVTLTCGSSTGAVTTSNYVNWVQQKPG |
| | | QAPRGLIGGTNKRAPGTPARFSGSLIGGKAAL |
| | | TLSGAQPEDEAEYYCVLWYSNRWVFGGGTK |
| | | LTVL |

v619-HLh-CC4

| | | |
|---|---|---|
| VH CDR1 | SEQ ID NO: 10 | TYAMN |
| VH CDR2 | SEQ ID NO: 11 | RIRSKYNNYATYYADSVKD |
| VH CDR3 | SEQ ID NO: 12 | HGNFGNSYVSWFAY |
| VL CDR1 | SEQ ID NO: 20 | GSSTGAVTTSNYVN |
| VL CDR2 | SEQ ID NO: 8 | GTNKRAP |
| VL CDR3 | SEQ ID NO: 15 | VLWYSNRWV |
| Heavy chain | SEQ ID NO: 55 | EVQLVESGGGLVQPGGSLKLSCAASGFTFST |
| variable | | YAMNWVRQASGKGLEWVGRIRSKYNNYAT |
| domain | | YYADSVKDRFTISRDDSKNTAYLQMNSLKT |
| | | EDTAVYYCVRHGNFGNSYVSWFAYWGCGT |
| | | LVTVSS |
| Light chain | SEQ ID NO: 56 | QTVVTQEPSLTVSPGGTVTLTCGSSTGAVTT |
| variable | | SNYVNWVQQKPGCAPRGLIGGTNKRAPGTP |
| domain | | ARFSGSLIGGKAALTLSGAQPEDEAEYYCVL |
| | | WYSNRWVFGGGTKLTVL |
| scFv | SEQ ID NO: 57 | EVQLVESGGGLVQPGGSLKLSCAASGFTFST |
| | | YAMNWVRQASGKGLEWVGRIRSKYNNYAT |
| | | YYADSVKDRFTISRDDSKNTAYLQMNSLKT |
| | | EDTAVYYCVRHGNFGNSYVSWFAYWGCGT |
| | | LVTVSSGGGGSGGGGSGGGGSQTVVTQEPS |
| | | LTVSPGGTVTLTCGSSTGAVTTSNYVNWVQ |
| | | QKPGCAPRGLIGGTNKRAPGTPARFSGSLIG |
| | | GKAALTLSGAQPEDEAEYYCVLWYSNRWV |
| | | FGGGTKLTVL |

v619_Hh8

| | | |
|---|---|---|
| VH CDR1 | SEQ ID NO: 10 | TYAMN |
| VH CDR2 | SEQ ID NO: 11 | RIRSKYNNYATYYADSVKD |
| VH CDR3 | SEQ ID NO: 13 | HGNFGNSYVSWWAY |
| VL CDR1 | SEQ ID NO: 20 | GSSTGAVTTSNYVN |
| VL CDR2 | SEQ ID NO: 8 | GTNKRAP |
| VL CDR3 | SEQ ID NO: 39 | VLWYSNLWV |
| Heavy chain | SEQ ID NO: 30 | EVQLVESGGGLVQPGGSLKLSCAASGFTFST |
| variable | | YAMNWVRQASGKGLEWVGRIRSKYNNYAT |
| domain | | YYADSVKDRFTISRDDSKNTAYLQMNSLKT |
| | | EDTAVYYCVRHGNFGNSYVSWWAYWGQG |
| | | TLVTVSS |
| Light chain | SEQ ID NO: 58 | QTVVTQEPSLTVSPGGTVTLTCGSSTGAVTT |
| variable | | SNYVNWVQQKPGQAPRGLIGGTNKRAPGTP |
| domain | | ARFSGSLIGGKAALTLSGAQPEDEAEYYCVL |
| | | WYSNLWVFGGGTKLTVL |
| scFv | SEQ ID NO: 59 | EVQLVESGGGLVQPGGSLKLSCAASGFTFST |
| | | YAMNWVRQASGKGLEWVGRIRSKYNNYAT |
| | | YYADSVKDRFTISRDDSKNTAYLQMNSLKT |
| | | EDTAVYYCVRHGNFGNSYVSWWAYWGQG |
| | | TLVTVSSGGGGSGGGGSGGGGSQTVVTQEP |
| | | SLTVSPGGTVTLTCGSSTGAVTTSNYVNWV |
| | | QQKPGQAPRGLIGGTNKRAPGTPARFSGSLI |
| | | GGKAALTLSGAQPEDEAEYYCVLWYSNLW |
| | | VFGGGTKLTVL |

v619_HLp2

| | | |
|---|---|---|
| VH CDR1 | SEQ ID NO: 10 | TYAMN |
| VH CDR2 | SEQ ID NO: 11 | RIRSKYNNYATYYADSVKD |
| VH CDR3 | SEQ ID NO: 13 | HGNFGNSYVSWWAY |
| VL CDR1 | SEQ ID NO: 60 | RSSTGAVTTSNYVN |
| VL CDR2 | SEQ ID NO: 8 | GTNKRAP |
| VL CDR3 | SEQ ID NO: 61 | ILWYSNRWV |
| Heavy chain | SEQ ID NO: 30 | EVQLVESGGGLVQPGGSLKLSCAASGFTFST |
| variable | | YAMNWVRQASGKGLEWVGRIRSKYNNYAT |
| domain | | YYADSVKDRFTISRDDSKNTAYLQMNSLKT |
| | | EDTAVYYCVRHGNFGNSYVSWWAYWGQG |
| | | TLVTVSS |
| Light chain | SEQ ID NO: 62 | QTVVTQEPSLTVSPGGTVTLTCRSSTGAVTT |
| variable | | SNYVNWVQQKPGQAPRGLIGGTNKRAPGTP |
| domain | | ARFSGSLIGGKAALTLSGAQPEDEAEYYCIL |
| | | WYSNRWVFGGGTKLTVL |
| scFv | SEQ ID NO: 63 | EVQLVESGGGLVQPGGSLKLSCAASGFTFST |
| | | YAMNWVRQASGKGLEWVGRIRSKYNNYAT |
| | | YYADSVKDRFTISRDDSKNTAYLQMNSLKT |
| | | EDTAVYYCVRHGNFGNSYVSWWAYWGQG |
| | | TLVTVSSGGGGSGGGGSGGGGSQTVVTQEP |
| | | SLTVSPGGTVTLTCRSSTGAVTTSNYVNWV |
| | | QQKPGQAPRGLIGGTNKRAPGTPARFSGSLI |
| | | GGKAALTLSGAQPEDEAEYYCILWYSNRWV |
| | | FGGGTKLTVL |

v619_Hh13

| | | |
|---|---|---|
| VH CDR1 | SEQ ID NO: 10 | TYAMN |
| VH CDR2 | SEQ ID NO: 11 | RIRSKYNNYATYYADSVKD |
| VH CDR3 | SEQ ID NO: 13 | HGNFGNSYVSWWAY |
| VL CDR1 | SEQ ID NO: 60 | RSSTGAVTTSNYVN |
| VL CDR2 | SEQ ID NO: 8 | GTNKRAP |
| VL CDR3 | SEQ ID NO: 15 | VLWYSNRWV |
| Heavy chain | SEQ ID NO: 30 | EVQLVESGGGLVQPGGSLKLSCAASGFTFST |
| variable | | YAMNWVRQASGKGLEWVGRIRSKYNNYAT |
| domain | | YYADSVKDRFTISRDDSKNTAYLQMNSLKT |
| | | EDTAVYYCVRHGNFGNSYVSWWAYWGQG |
| | | TLVTVSS |
| Light chain | SEQ ID NO: 64 | QTVVTQEPSLTVSPGGTVTLTCRSSTGAVTT |
| variable | | SNYVNWVQQKPGQAPRGLIGGTNKRAPGTP |
| domain | | ARFSGSLIGGKAALTLSGAQPEDEAEYYCVL |
| | | WYSNRWVFGGGTKLTVL |
| scFv | SEQ ID NO: 65 | EVQLVESGGGLVQPGGSLKLSCAASGFTFST |
| | | YAMNWVRQASGKGLEWVGRIRSKYNNYAT |
| | | YYADSVKDRFTISRDDSKNTAYLQMNSLKT |
| | | EDTAVYYCVRHGNFGNSYVSWWAYWGQG |
| | | TLVTVSSGGGGSGGGGSGGGGSQTVVTQEP |
| | | SLTVSPGGTVTLTCRSSTGAVTTSNYVNWV |
| | | QQKPGQAPRGLIGGTNKRAPGTPARFSGSLI |
| | | GGKAALTLSGAQPEDEAEYYCVLWYSNRW |
| | | VFGGGTKLTVL |

In some embodiments, the activatable protein herein may comprise an AB 1 that binds to a tumor associated antigen and an AB2 that binds to a co-stimulatory molecule. In one example, the activatable protein may comprise an AB 1 that binds to HER2 and an AB2 that binds to CD3. In a particular example, the activatable protein may comprise an AB 1 that is an anti-HER2 Fab (e.g., Fab of trastuzumab) and an AB2 that is an anti-CD3 scFv.

In some embodiments, the AB2 may bind to a target that is antigen on any immune effector cells. Examples of immune effector cells include leukocytes, T cells, natural killer (NK) cells, macrophages, mononuclear cells, and myeloid mononuclear cells. In some examples, the activatable protein may comprise an immune effector cell engaging bispecific activatable antibody, which crosslinks an immune effector cell with another cell (e.g., a cell associated with a disease such as cancer or infection).

The activatable protein may comprise a leukocyte cell-engaging bispecific activatable antibody, a T cell engaging bispecific activatable antibody, a NK cell-engaging bispecific activatable antibody, a macrophage cell-engaging bispecific activatable antibody, a mononuclear cell-engaging bispecific activatable antibody, or a myeloid mononuclear cell-engaging bispecific activatable antibody. In one example, the activatable antibody may comprise a T cell engaging bispecific antibody.

### Half-Life Extending Moieties

The activatable protein may comprise a half-life extending moiety (EM). In the activatable protein, the EM may be coupled to a TB or a component thereof in the activatable protein via a CM. Upon activation of the activatable protein, the EM may be cleaved off from the TB. In some embodiments, for example, a CM is positioned at a location between the C-terminus of the TB and the N-terminus of the EM. In certain of these embodiments, a CM is positioned at a location between the C-terminus of the TB and the N-terminus of the EM, and an MM is positioned at a location that is C-terminal relative to the CM, and either N-terminal or C-terminal relative to the EM (e.g., from N-terminus to C-terminus, TB-CM-EM, TB-CM-EM-MM, TB-CM-MM-EM, etc., wherein each “-” independently indicates direct or indirect (e.g., via a linker) coupling) (see, e.g., FIGS. 1 and 2). In some embodiments, the EM may be a dimer, e.g., a pair of Fc domains of an immunoglobulin. In such embodiments, a first polypeptide may comprise the TB, the CM, and a first Fc domain, and a second polypeptide may comprise the MM and the second Fc domain, and the two polypeptides are covalently linked via one or more disulfide bonds between the first and second Fc domains. In such embodiments, the MM may be positioned at either the N-terminus or the C-terminus of the second Fc domain (see, e.g., FIGS. 3 and 4), and cleavage of the CM on the first polypeptide results in release of the MM and the EM (e.g., both of the Fc domains) from the activated protein. Thus, in some embodiments, activated proteins resulting from the activation of the activatable protein do not comprise the EM. In some examples, the half-life extending moiety may be a serum half-life extending moiety, i.e., capable of extending the half-life of the molecule attached to the EM in serum.

In some examples, the EM may comprise a fragment crystallizable region (Fc domain) of an antibody. For example, the EM may be the Fc domain of an IgG (e.g., IgG1, IgG2, or IgG4). In some examples, the EM may comprise a dimer formed by two Fc domains. The Fc domain may be a wild type Fc domain or a mutant thereof. For example, the EM may comprise a dimer formed by two Fc domain mutants. In such cases, the two Fc domain mutants may comprise a Fc domain hole mutant and a Fc domain knob mutant. The knob and hole mutants may interact with each other to facilitate the dimerization of the two Fc domains. In some embodiments, the knob and hole mutants may comprise one or more amino acid modifications within the interface between two Fe domains (e.g., in the CH3 domain). In one example, the modifications comprise amino acid substitution T366W and optionally the amino acid substitution S354C in one of the antibody heavy chains, and the amino acid substitutions T366S, L368A, Y407V and optionally Y349C in the other one of the antibody heavy chains (numbering according to EU numbering system). An example of the Fc domain knob mutant comprise a sequence of SEQ ID NO: 1. An example of the Fc domain hole mutant comprise a sequence of SEQ ID NO: 2.

Examples of the Fc domain mutants also include those described in U.S. Pat. Nos. 7,695,936, which is incorporated herein by reference in its entirety. In one example, the modifications comprise amino acid substitution T366Y in one IgG Fc domain, and the amino acid substitutions Y407T in the other IgG Fc domain. In one example, the modifications comprise amino acid substitution T366W in one IgG Fc domain, and the amino acid substitutions Y407A in the other IgG Fc domain. In one example, the modifications comprise amino acid substitution F405A in one IgG Fc domain, and the amino acid substitutions T394W in the other IgG Fc domain. In one example, the modifications comprise amino acid substitution T366Y and F405A in one IgG Fc domain, and the amino acid substitutions T394W and Y407T in the other IgG Fc domain. In one example, the modifications comprise amino acid substitution T366W and F405W in one IgG Fc domain, and the amino acid substitutions T394S and Y407A in the other IgG Fc domain. In one example, the modifications comprise amino acid substitution F405W and Y407A in one IgG Fc domain, and the amino acid substitutions T366W and T394S in the other IgG Fc domain. In one example, the modifications comprise amino acid substitution F405W in one IgG Fc domain, and the amino acid substitutions T394S in the other IgG Fc domain. The mutation positions in the Fc domains are numbered according to EU numbering system. The IgG Fc domain may comprise a sequence of SEQ ID NOs: 3-6 (IgG1, IgG2, IgG3 or IgG4). In these sequences, amino acids 1-107 correspond to EU numbering 341-447.

In some examples, the Fc domains mutants may have reduced effector function. Examples of such Fc domains include those disclosed in in US20190135943, which incorporated herein by reference in its entirety.

Further examples of EMs include immunoglobulin (e.g., IgG), serum albumin (e.g., human serum albumin (HSA), hexa-hat GST (glutathione S-transferase) glutathione affinity, Calmodulin-binding peptide (CBP), Strep-tag, Cellulose Binding Domain, Maltose Binding Protein, S-Peptide Tag, Chitin Binding Tag, Immuno-reactive Epitopes, Epitope Tags, E2Tag, HA Epitope Tag, Myc Epitope, FLAG Epitope, AU1 and AU5 Epitopes, Glu-Glu Epitope, KT3 Epitope, IRS Epitope, Btag Epitope, Protein Kinase-C Epitope, and VSV Epitope.

In some embodiments, the serum half-life of the activatable protein may be longer than that of a counterpart protein that is the same as the activatable protein but not having the half-life extending moiety. In some embodiments, the serum half-life of the activatable protein may be longer than that of the activated protein. In some embodiments, the serum half-life of the activatable protein is at least 15 days, 12 days, 11 days, 10 days, 9 days, 8 days, 7 days, 6 days, 5 days, 4 days, 3 days, 2 days, 1 day, 20, 18 hours, 16 hours, 14 hours, 12 hours, 10 hours, 8 hours, 6 hours, 4 hours, 3 hours, 2 hours, or 1 hour when administered to an organism.

### Masking Moieties (MMs)

The activatable proteins herein may comprise one or more masking moieties (MMs) capable of interfering with the binding of the TBs to the target. A masking moiety in an activatable molecule “masks” or reduces or otherwise inhibits the binding of the activatable macromolecule to its target and/or epitope. In some embodiments, the coupling or modifying of a target-binding protein (TB) (e.g., an AB or other therapeutic or diagnostic protein) with an MM may inhibit the ability of the TB to specifically bind its target and or epitope by means of inhibition known in the art (e.g., structural change, competition for antigen-binding domain, and the like). In some embodiments, the coupling or modifying of a TB with an MM may effect a structural change that reduces or inhibits the ability of the TB to specifically bind its target and or epitope. In some embodiments, the coupling or modifying of a protein comprising an antigen-binding domain with a MM sterically blocks, reduces or inhibits the ability of the antigen-binding domain to specifically bind its target and or epitope. An MM may be coupled to a TB (e.g., an AB) by a CM, either directly or indirectly (e.g., via one or more linkers described herein).

Alternatively, a MM interfering with the target binding of a TB may be coupled to a component of the activatable protein that is not the TB. For example, as exemplified in FIG. 2, an activatable protein may comprise a TB1 and a TB2, and the MM interfering with the TB2 may be coupled to the TB1. In another example, as exemplified in FIGS. 3 and 4, an activatable protein may comprise a TB1, a TB2, and an EM, and the MM interfering with the TB2 may be coupled to the EM. In either case, in the tertiary or quaternary structure of the activatable structure, the MM may be in a position (e.g., proximal to the TB to be masked) that allows the MM to mask the TB.

In some embodiments, a MM may interact with the TB, thus reducing or inhibiting the interaction between the TB and its binding partner. In some embodiments, the MM may comprise at least a partial or complete amino acid sequence of a naturally occurring binding partner of the TB. For example, the MM may be a fragment of a naturally occurring binding partner. The fragment may retain no more than 95%, 90%, 80%, 75%, 70%, 60%, 50%, 40%, 30%, 25%, or 20% nucleic acid or amino acid sequence homology to the naturally occurring binding partner. In some embodiments, the MM may be a cognate polypeptide of the TB (e.g., AB). For example, the MM may comprise a sequence of a TB's epitope or a fragment thereof. The term “naturally occurring” as used herein as applied to an object refers to the fact that an object can be found in nature. For example, a polypeptide or polynucleotide sequence that is present in an organism (including viruses) that can be isolated from a source in nature and that has not been intentionally modified by man in the laboratory or otherwise is naturally occurring.

In some embodiments, the MM may comprise an amino acid sequence that is not naturally occurring or does not contain the amino acid sequence of a naturally occurring binding partner or target protein. In certain embodiments, the MM is not a natural binding partner of the TB. In some embodiments, the MM does not comprise a subsequence of more than 4, 5, 6, 7, 8, 9 or 10 consecutive amino acid residues of a natural binding partner of the TB. The MM may be a modified binding partner for the TB which contains amino acid changes that decrease affinity and/or avidity of binding to the TB. In some embodiments the MM may contain no or substantially no nucleic acid or amino acid homology to the TB's natural binding partner. In other embodiments the MM is no more than 5%, 10%, 15%, 20%, 25%, 30%, 35%, 40%, 45%, 50%, 55%, 60%, 65%, 70%, 75%, or 80% similar to the natural binding partner of the TB.

In some embodiments, the MM may not specifically bind to the TB (or other activatable protein), but interfere with target-binding protein's (e.g., AB's) binding to its binding partner through non-specific interactions such as steric hindrance. For example, the MM may be positioned in the activatable protein such that the tertiary or quaternary structure of the activatable protein allows the MM to mask the AB through charge-based interaction, thereby holding the MM in place to interfere with binding partner access to the TB.

In some embodiments, the MM may have a dissociation constant for binding to the target-binding protein (e.g., AB) that is no more than the dissociation constant of the TB to the target. In some embodiments, the MM may not interfere or compete with the TB for binding to the target in a cleaved state.

The structural properties of the MMs may be selected according to factors such as the minimum amino acid sequence required for interference with protein binding to target, the target protein-protein binding pair of interest, the size of the TB, the presence or absence of linkers, and the like.

In some embodiments, the MM may be unique for the coupled TB. Examples of MMs include MMs that were specifically screened to bind a binding domain of the TB, e.g., AB, or fragment thereof (e.g., affinity masks). Methods for screening MMs to obtain MMs unique for the TB and those that specifically and/or selectively bind a binding domain of a binding partner/target are provided herein and can include protein display methods.

As used herein, the term “masking efficiency” or “ME” refers to the activity (e.g., EC50) of the activatable protein divided by the activity of a control target-binding protein (e.g., antibody), wherein the control target-binding protein (e.g., antibody) may be either the cleavage product of the activatable protein (i.e., the activated protein) or the target-binding protein (e.g., antibody or fragment thereof) used as the TB of the activatable protein. An activatable protein having a reduced level of target binding activity may have a masking efficiency that is greater than 10. In some embodiments, the activatable proteins described herein may have a masking efficiency that is greater than 10, 100, 1000, or 5000.

In some embodiments, the MM may be a peptide of about 2 to 50 amino acids in length. For example, the MM may be a peptide of from 2 to 40, from 2 to 30, from 2 to 20, from 2 to 10, from 5 to 15, from 10 to 20, from 15 to 25, from 20 to 30, from 25 to 35, from 30 to 40, from 35 to 45, from 40 to 50 amino acids in length. For example, the MM may be a peptide with 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids in length. In some examples, the MM may be a polypeptide of more than 50 amino acids in length, e.g., 100, 200, 300, 400, 500, 600, 700, 800, or more amino acids.

In some embodiments, the activatable protein with an TB and an interfering MM, in the presence of the target of an TB, there is no binding or substantially no binding of the TB to the target, or no more than 0.001%, 0.01%, 0.1%, 1%, 2%, 3%, 4%, 5%, 6%, 7%, 8%, 9%, 10%, 15%, 20%, 25%, 30%, 35%, 40%, or 50% binding of the TB to its target, as compared to the binding of a counterpart antibody without the interfering MM, for at least 0.1, 0.5, 1, 2, 4, 6, 8, 12, 28, 24, 30, 36, 48, 60, 72, 84, 96 hours, or 5, 10, 15, 30, 45, 60, 90, 120, 150, 180 days, or 1, 2, 3, 4, 5, 6, 7, 8, 9, 10, 11, or 12 months when measured in vivo or in a masking efficiency assay, or in an in vitro immunoabsorbant assay, e.g., as described in US20200308243A1. For example, the ability of a MM to inhibit binding of an activatable protein to its binding partner at therapeutically relevant concentrations and times can be measured. For this measurement, an immunoabsorbant assay (MEA, Mask Efficiency Assay) to measure the time-dependent binding of an activatable protein binding to its binding partner has been developed and described in US20200308243A1, the entirety of which is incorporated herein by reference.

The binding affinity of the TB towards the target or binding partner with an interfering MM may be at least 5, 10, 25, 50, 100, 250, 500, 1,000, 2,500, 5,000, 10,000, 50,000, 100,000, 500,000, 1,000,000, 5,000,000, 10,000,000, 50,000,000 times lower than the binding affinity of the TB towards its binding partner without an interfering MM, or between 5-10, 10-100, 10-1,000, 10-10,000, 10-100,000, 10-1,000,000, 10-10,000,000, 100-1,000, 100-10,000, 100-100,000, 100-1,000,000, 100-10,000,000, 1,000-10,000, 1,000-100,000, 1,000-1,000,000, 1000-10,000,000, 10,000-100,000, 10,000-1,000,000, 10,000-10,000,000, 100,000-1,000,000, or 100,000-10,000,000 times lower than the binding affinity of the TB towards its binding partner when there is no interfering MM.

The dissociation constant (K_{d}) of the MM towards the TB (e.g., AB) it masks, may be greater than the K_{d} of the TB (e.g., AB) towards the target. The K_{d} of the MM towards the masked TB may be at least 5, 10, 25, 50, 100, 250, 500, 1,000, 2,500, 5,000, 10,000, 100,000, 1,000,000 or even 10,000,000 times greater than the K_{d} of the TB towards the target. Conversely, the binding affinity of the MM towards the masked TB may be lower than the binding affinity of the TB towards the target. The binding affinity of MM towards the TB may be at least 5, 10, 25, 50, 100, 250, 500, 1,000, 2,500, 5,000, 10,000, 100,000, 1,000,000 or even 10,000,000 times lower than the binding affinity of the TB towards the target.

In some embodiments, the MMs may contain genetically encoded or genetically non-encoded amino acids. Examples of genetically non-encoded amino acids are but not limited to D-amino acids, β-amino acids, and γ-amino acids. In specific embodiments, the MMs contain no more than 50%, 40%, 30%, 20%, 15%, 10%, 5% or 1% of genetically non-encoded amino acids.

In some embodiments, once released from the activatable protein and in a free state, the MM may have a biological activity or a therapeutic effect, such as binding capability. For example, the free MM may bind with the same or a different binding partner. In certain embodiments, the free MM may exert a therapeutic effect, providing a secondary function to the compositions disclosed herein. In some embodiments, once uncoupled from the activatable protein and in a free state, the MM may advantageously not exhibit biological activity. For example, in some embodiments the MM in a free state does not elicit an immune response in the subject.

Suitable MMs may be identified and/or further optimized through a screening procedure from a library of candidate activatable proteins having variable MMs. For example, a TB and a CM may be selected to provide for a desired enzyme/target combination, and the amino acid sequence of the MM can be identified by the screening procedure described below to identify a MM that provides for an activatable phenotype. For example, a random peptide library (e.g., of peptides comprising 2 to 40 amino acids or more) may be used in the screening methods disclosed herein to identify a suitable MM.

In some embodiments, MMs with specific binding affinity for a TB (e.g., AB) may be identified through a screening procedure that includes providing a library of peptide scaffolds comprising candidate MMs wherein each scaffold is made up of a transmembrane protein and the candidate MM. The library may then be contacted with an entire or portion of a protein such as a full length protein, a naturally occurring protein fragment, or a non-naturally occurring fragment containing a protein (also capable of binding the binding partner of interest), and identifying one or more candidate MMs having detectably bound protein. The screening may be performed by one more rounds of magnetic-activated sorting (MACS) or fluorescence-activated sorting (FACS), as well as determination of the binding affinity of MM towards the AB and subsequent determination of the masking efficiency, e.g., as described in WO2009025846 and US20200308243A1, which are incorporated herein by reference in their entireties.

In some embodiments, a MM may be selected for use with a specific protein, antibody or antibody fragment. For example, suitable MM for use with an AB that binds to an epitope may comprise the sequence of the epitope. In an example when the activatable comprising an AB1 that is an anti-HER2 Fab and an AB2 that is an anti-CD3 scFv, a MM1 (for masking the AB1) may comprise a sequence in HER2 that the AB1 binds to and a MM2 (for masking the AB2) may comprise a sequence in CD3 that the AB2 binds to. In other embodiments, the MM may not comprise a sequence of the natural binding partner of the TB. In some examples, suitable MM1 for masking the anti-HER2 Fab comprise the sequence of ALICCSDVSGLCRWC (SEQ ID NO: 40). In some examples, suitable MM2 for masking the anti-CD3 scFv include MMs comprising the sequences of GYLWGCEWNCGGITT (SEQ ID NO: 34), NAFRCWWDPPCQPMT (SEQ ID NO: 35), ARGLCWWDPPCTHDL (SEQ ID NO: 36), or NHSLCYWDPPCEPST (SEQ ID NO: 37). In some examples, suitable MM2 for masking the anti-CD3 scFv include MMs comprising the sequences of MMYCGGNEVLCGPRV (SEQ ID NO: 66), GYRWGCEWNCGGITT (SEQ ID NO: 67), MMYCGGNEIFCEPRG (SEQ ID NO: 68), GYGWGCEWNCGGSSP (SEQ ID NO: 69), or MMYCGGNEIFCGPRG (SEQ ID NO: 70).

Additional suitable MMs are disclosed in WO2021207657, WO2021142029, WO2021061867, WO2020252349, WO2020252358, WO2020236679, WO2020176672, WO2020118109, WO2020092881, WO2020086665, WO2019213444, WO2019183218, WO2019173771, WO2019165143, WO2019075405, WO2019046652, WO2019018828, WO2019014586, WO2018222949, WO2018165619, WO2018085555, WO2017011580, WO2016179335, WO2016179285, WO2016179257, WO2016149201, and WO2016014974, which are incorporated herein by reference in their entireties.

### Cleavable Moieties (CMs)

The activatable protein may comprise one or more cleavable moieties (CMs) as defined above.

In some embodiments, the activatable protein may comprise a CM between a TB (e.g., AB) and a MM. The activatable protein may further comprise a CM between a TB and an EM. In some examples, a CM between the TB and the MM is also between the TB and the EM (see, e.g., FIG. 1, in which CM 127 is between TB 123/125 and MM 129, and CM 127 also is between TB 123/125 and EM 131/141.). In such cases, the cleavage of the CM may release both the MM and the EM from the TB. In some examples, the CM is positioned between a first TB (TB1) and an MM (MM2) that binds a second TB (TB2) (see, e.g., FIG. 2, in which CM 223 is between a first TB (TB 221) and MM 225, wherein MM 225 is a masking moiety that inhibits the binding of a second TB (TB 207/209); see also FIG. 3, in which CM 323 is positioned between a first TB (TB 321) and MM 343, and MM 343 is a masking moiety that inhibits the binding of a second TB (TB 307/309) to its target). In certain examples, a CM between the TB and the MM is not between the TB and the EM. In such cases, the activatable protein may comprise a first CM between the TB and the MM and a second CM between the TB and the EM. In some examples, the activatable protein may have three CMs: a first CM between a first TB and a first MM, a second CM between a second TB and a second MM, and a third CM between an EM and the first or second TB (see, e.g., FIGS. 10 and 11). The activation of the activatable protein may cleave both CMs so the MM and the EM are both released from the EM.

The CM and the TB of the activatable proteins may be selected so that the TB comprises a binding moiety for a given target, and the CM comprises a substrate for one or more proteases, where the one or more proteases is/are co-localized with the target in a tissue (e.g., at a treatment site or diagnostic site in a subject). In some embodiments, the activatable proteins may find particular use where, for example, one or more proteases capable of cleaving a site in the CM, is present at relatively higher levels (or is more active) in target-containing tissue of a treatment site or diagnostic site than in tissue of non-treatment sites (for example in healthy tissue).

In some embodiments, the CMs herein may comprise substrates for proteases that have been reported in a cancer, or in a number of cancers. See, e.g., La Roca et al., British J. Cancer 90(7):1414-1421, 2004. Substrates suitable for use in the CM components employed herein include those which are more prevalently found in cancerous cells and tissue. Thus, in certain embodiments, the CM may comprise a substrate for a protease that is more prevalently found in diseased tissue associated with a cancer. Examples of the cancers include gastric cancer, breast cancer, osteosarcoma, esophageal cancer, breast cancer, a HER2-positive cancer, Kaposi sarcoma, hairy cell leukemia, chronic myeloid leukemia (CML), follicular lymphoma, renal cell cancer (RCC), melanoma, neuroblastoma, basal cell carcinoma, cutaneous T-cell lymphoma, nasopharyngeal adenocarcimoa, ovarian cancer, bladder cancer, BCG-resistant non-muscle invasive bladder cancer (NMIBC), endometrial cancer, pancreatic cancer, non-small cell lung cancer (NSCLC), colorectal cancer, esophageal cancer, gallbladder cancer, glioma, head and neck carcinoma, uterine cancer, cervical cancer, or testicular cancer, and the like. In some embodiments, the CM components comprise substrates for protease(s) that is/are more prevalent in tumor tissue. For example, the protease(s) may be produced by a tumor in a subject.

In some embodiments, the activatable protein may comprise a first CM between the MM and the TB (e.g., AB), and a second CM between the EM and the same or a different TB. In an activated state, both CMs may be cleaved so that the MM and the EM are released from the TB(s). In some examples, the first and the second CMs may comprise the substrates of the same protease. In some examples, the first and the second CMs may comprise the substrates of different proteases. In some examples, the first and the second CMs may comprise or consist of the same sequence. In some examples, the first and the second CMs may comprise or consist of different sequences.

The second CM may be at a position in the activatable protein where its cleavage facilitates dissociation of the EM from the TB). In some examples, the second CM may be between the C-terminus of the TB (or a component thereof if the TB comprises multiple polypeptides) and the N terminus of the MM, where the C-terminus of the MM is coupled to the N-terminus of the EM (or a component thereof if the EM comprises multiple polypeptides). In some examples, the second CM may be between the N-terminus of the TB (or a component thereof if the TB comprises multiple polypeptides) and the C-terminus of the MM, where the N-terminus of the MM is coupled to the C-terminus of the EM (or a component thereof if the EM comprises multiple polypeptides). In some examples, the second CM may be between the C-terminus of the TB (or a component thereof if the TB comprises multiple polypeptides) and the N-terminus of the EM (or a component thereof if the EM comprises multiple polypeptides), where the C-terminus of the EM (or a component thereof if the EM comprises multiple polypeptides) is coupled to the N-terminus of the MM. In some examples, the second CM may be between the N-terminus of the TB (or a component thereof if the TB comprises multiple polypeptides) and the C-terminus of the EM (or a component thereof if the EM comprises multiple polypeptides), where the N-terminus of the EM (or a component thereof if the EM comprises multiple polypeptides) is coupled to the C-terminus of the MM. In these examples, the MM may be a masking moiety of the TB or a different TB (e.g., on the same or another polypeptide) in the activatable protein.

Suitable CMs for use in the activatable protein herein include any of the protease substrates that are known the art. In some examples, the CM may comprise a substrate of a serine protease (e.g., u-type plasminogen activator (uPA, also referred to as urokinase), matriptase (also referred to herein as MT-SP1 or MTSP1). In some examples, the CM may comprise a substrate of a matrix metalloprotease (MMP). In some examples, the CM may comprise a substrate of cysteine protease (CP) (e.g., legumain).

In some embodiments, the CM may comprise a substrate for a disintegrin and a metalloproteinase (ADAM) or a disintegrin and metalloproteinase with a thrombospondin motifs (ADAMTS)(e.g., ADAM8, ADAM9, ADAM10, ADAM12, ADAM15, ADAM17/TACE, ADEMDEC1, ADAMTS1, ADAMTS4, ADAMTS5), an aspartate protease (e.g. BACE, Renin), an aspartic cathepsin (e.g., Cathepsin D, Cathepsin E), Caspase (e.g., Caspase 1, Caspase 2, Caspase 3, Caspase 4, Caspase 5, Caspase 6, Caspase 7, Caspase 8, Caspase 9, Caspase 10, Caspase 14), cysteine cathepsin (e.g., Cathepsin A, Cathepsin B, Cathepsin C, Cathepsin G, Cathepsin K, Cathepsin L, Cathepsin S, Cathepsin V/L2, Cathepsin X/Z/P), a cysteine proteinase (e.g., Cruzipain, Legumain, Otubain-2), a Chymase, DESC1, DPP-4, FAP, an Elastase, FVIIa, FiXA, FXa, FXIa, FXIIa, Granzyme B, Guanidinobenzoatase, Hepsin, HtrA1, a Human Neutrophil Elastase, a KLK (e.g., KLK4, KLK5, KLK6, KLK7, KLK8, KLK10, KLK11, KLK13, KLK14), a metallo proteinase (e.g., Meprin, Neprilysin, PSMA, BMP-1), Lactoferrin, Marapsin, Matriptase-2, MT-SP1/Matriptase, NS3/4A, PACE4, Plasmin, PSA, a MMP (e.g., MMP1, MMP2, MMP3, MMP7, MMP8, MMP9, MMP10, MMP11, MMP12, MMP13, MMP14, MMP15, MMP16, MMP17, MMP19, MMP20, MMP23, MMP24, MMP26, MMP27), TMPRSS2, TMPRSS3, TMPRSS4, tPA, Thrombin, Tryptase, and uPA.

In some embodiments, the protease substrate in the CM may comprise a polypeptide sequence that is not substantially identical (e.g., no more than 90%, 80%, 70%, 60%, or 50% identical) to any polypeptide sequence that is naturally cleaved by the same protease. In some embodiments, CM may be or comprise a sequence of LSGRSDDH (SEQ ID NO: 33) or ISSGLLSGRSDNH (SEQ ID NO: 41). In some embodiments, the CM may be or comprise a sequence or encompassed by the consensus of sequence of any one of the sequences in the following table:

| | SEQ ID |
|---|---|
| Sequences | NO |
| AANALAHGLF | 99 |
| AANL | 100 |
| AANLGSGGSS | 101 |
| AAPRS | 102 |
| AAPRSF | 103 |
| AARGPAIH | 104 |
| AAYHLVSQ | 105 |
| AFPDMRSVRS | 106 |
| AFQALRM | 107 |
| AFRHLR | 108 |
| AGLGISST | 109 |
| AGLGVVER | 110 |
| AGPR | 111 |
| AHGL | 112 |
| AHGLF | 113 |
| AHQALRM | 114 |
| AIPRVRLFDV | 115 |
| ALAHG | 116 |
| ALAHGL | 117 |
| ALAHGLF | 118 |
| ALAHGLFAPRSF | 119 |
| ALAHGLESGRSAN | 120 |
| ALAHGLPTFVHL | 121 |
| ALGLLRLP | 122 |
| ALPSVKMVSE | 123 |
| ALRAP | 124 |
| ANQALRM | 125 |
| ANQALRMA | 126 |
| APPLVKSMVV | 127 |
| APPSFKLVNA | 128 |
| APRS | 129 |
| APRSALAHGLF | 130 |
| APRSF | 131 |
| AQFVLTEG | 132 |
| AQNLLGMV | 133 |
| ARGP | 134 |
| ARGPS | 135 |
| ARGPSF | 136 |
| ARGPSFK | 137 |
| ASGLLRFP | 138 |
| ASPTMKTVGL | 139 |
| AVGLLAPP | 140 |
| AVGLLAPPGGLSGRSANI | 141 |
| AVGLLAPPGGLSGRSANP | 142 |
| AVGLLAPPGGLSGRSDDH | 143 |
| AVGLLAPPGGLSGRSDIH | 144 |
| AVGLLAPPGGLSGRSDNH | 145 |
| AVGLLAPPGGLSGRSDNI | 146 |
| AVGLLAPPGGLSGRSDNP | 147 |
| AVGLLAPPGGLSGRSDQH | 148 |
| AVGLLAPPGGLSGRSDTH | 149 |
| AVGLLAPPGGLSGRSDYH | 150 |
| AVGLLAPPGGLSGRSNI | 151 |
| AVGLLAPPGGLSGRSNIG | 152 |
| AVGLLAPPGGLSGRSNIGS | 153 |
| AVGLLAPPGGTSTSGRSANP | 154 |
| RG | |
| AVGLLAPPSGRSANPRG | 155 |
| AVGLLAPPTSGRSANPRG | 156 |
| AVPKVRVVPE | 157 |
| CGPPLGR | 158 |
| CSPPLGR | 159 |
| DEVDGSGGSS | 160 |
| DEXXXC(A/S) | |
| DISHWRRS | 161 |
| DLAHPLL | 162 |
| DLPLVKSLPS | 163 |
| DLXXT(A/S) | |
| DRLSGRSANHKK | 164 |
| DRLSGRSDNHKK | 165 |
| DRPEMKSLSG | 166 |
| DRPKVKTMDF | 167 |
| DVAQFVLT | 168 |
| DVPPMKTLRP | 169 |
| DWLYWMGI | 170 |
| DWLYWMSI | 171 |
| DWLYWPGI | 172 |
| DWLYWPSI | 173 |
| EAPKVKALPK | 174 |
| EHPRVKVVSE | 175 |
| EKPRMKLFQG | 176 |
| EPQALAMS | 177 |
| EQPEVKMVKG | 178 |
| ERPGVKSLVL | 179 |
| ESLPVVAV | 180 |
| ESPVMKSMAL | 181 |
| ESRRW | 182 |
| ESRRWM | 183 |
| ESRRWMP | 184 |
| ETPSVKTMGR | 185 |
| ETPSVKTMGRSS | 186 |
| FPRPLGITGL | 187 |
| FRLLDWQW | 188 |
| GCGPPLGR | 189 |
| GCSPPLGR | 190 |
| GFPHMKTFQH | 191 |
| GFPHMKTFQHSS | 192 |
| GGGPPLGR | 193 |
| GGPPLGR | 194 |
| GGQPSGMWGW | 195 |
| GGSIDGR | 196 |
| GGSPPLGR | 197 |
| GGWHTGRN | 198 |
| GIAGQ | 199 |
| GLGTPRGLFA | 200 |
| GLPTFV | 201 |
| GLPTFVH | 202 |
| GLPTFVHL | 203 |
| GLPTFVHLPRQV | 204 |
| GLSGRSDNHGSS | 205 |
| GPEGLRVG | 206 |
| GPLGIAGI | 207 |
| GPLNGRSDNHKA | 208 |
| GPLNGRSDNHKK | 209 |
| GPLNGRSDNHKR | 210 |
| GPLNGRSDNHQA | 211 |
| GPLNGRSDNHQK | 212 |
| GPLNGRSDNHQR | 213 |
| GPLNGRSDNHRA | 214 |
| GPLNGRSDNHRK | 215 |
| GPLNGRSDNHRR | 216 |
| GPLSGRSDNHKA | 217 |
| GPLSGRSDNHKK | 218 |
| GPLSGRSDNHKR | 219 |
| GPLSGRSDNHQA | 220 |
| GPLSGRSDNHQK | 221 |
| GPLSGRSDNHQR | 222 |
| GPLSGRSDNHRA | 223 |
| GPLSGRSDNHRK | 224 |
| GPLSGRSDNHRR | 225 |
| GPPLGR | 226 |
| GPQGIAGQ | 227 |
| GPQGLLGA | 228 |
| GPRSFG | 229 |
| GPRSFGL | 230 |
| GPSHLVLT | 231 |
| GPTN | 232 |
| GPTNALAHGLF | 233 |
| GRSML | 234 |
| GRSMLL | 235 |
| GRSMLLG | 236 |
| GRSMLLGG | 237 |
| GRSMLLGP | 238 |
| GRSMLLGS | 239 |
| GRSMLLP | 240 |
| GRSMLLPG | 241 |
| GRSMLLPP | 242 |
| GRSMLLPS | 243 |
| GRSMLLS | 244 |
| GRSMLLSG | 245 |
| GRSMLLSP | 246 |
| GRSMLLSS | 247 |
| GRSMLM | 248 |
| GRSMLMG | 249 |
| GRSMLMGG | 250 |
| GRSMLMGP | 251 |
| GRSMLMGS | 252 |
| GRSMLMP | 253 |
| GRSMLMPG | 254 |
| GRSMLMPP | 255 |
| GRSMLMPS | 256 |
| GRSMLMS | 257 |
| GRSMLMSG | 258 |
| GRSMLMSP | 259 |
| GRSMLMSS | 260 |
| GSGPPLGR | 261 |
| GSPPLGR | 262 |
| GSSPPLGR | 263 |
| GTGRGPSWVGSS | 264 |
| PTNL | 265 |
| PTNLGSGGSS | 266 |
| PVGYTSSL | 267 |
| PVPRLKLIKD | 268 |
| PVQPIGPQ | 269 |
| QALAMSAI | 270 |
| QFQALRM | 271 |
| QGPMFKSLWD | 272 |
| QGRAITFI | 273 |
| QHQALRM | 274 |
| QNQALRIA | 275 |
| QNQALRM | 276 |
| QNQALRMA | 277 |
| QNQALRMAGGSGGSLSGRS | 278 |
| DNH | |
| QNQALRMAGGSGGSLSGRS | 279 |
| GNH | |
| QSRRVP | 280 |
| QSRRVPL | 281 |
| QSRRVPV | 282 |
| QTRRVP | 283 |
| QTRRVPL | 284 |
| QTRRVPV | 285 |
| QYIVSRSA | 286 |
| RALRAP | 287 |
| REPFMKSLPW | 288 |
| RFPLKV | 289 |
| RFPSLKSFPL | 290 |
| RFPYGVW | 291 |
| RFYRNQFF | 292 |
| RGPA | 293 |
| RGPAFNPM | 294 |
| RGPATPIM | 295 |
| RGPKLYW | 296 |
| RHLAKL | 297 |
| RIGRSDNH | 298 |
| RKMPNITV | 299 |
| RKSSIIIRMRDVVL | 300 |
| RKTVQHWW | 301 |
| RLGRSDNN | 302 |
| RMHLRSLG | 303 |
| RPLARAGI | 304 |
| RPLARAGL | 305 |
| RPLNGRSDNHKA | 306 |
| RPLNGRSDNHKK | 307 |
| RPLNGRSDNHKR | 308 |
| RPLNGRSDNHQA | 309 |
| RPLNGRSDNHQK | 310 |
| RPLNGRSDNHQR | 311 |
| RPLNGRSDNHRA | 312 |
| RPLNGRSDNHRK | 313 |
| RPLNGRSDNHRR | 314 |
| RPLSGRSDNHKA | 315 |
| RPLSGRSDNHKK | 316 |
| RPLSGRSDNHKR | 317 |
| RPLSGRSDNHQA | 318 |
| RPLSGRSDNHQK | 319 |
| RPLSGRSDNHQR | 320 |
| RPLSGRSDNHRA | 321 |
| RPLSGRSDNHRK | 322 |
| RPLSGRSDNHRR | 323 |
| RPSPMWAY | 324 |
| RRHDGLRA | 325 |
| RRHDGLRS | 326 |
| RSLVFAPI | 327 |
| RSPSRLKC | 328 |
| VAGRSMRP | 329 |
| VAPQLKSLVP | 330 |
| VAQFVLTE | 331 |
| VHMPLGFLGP | 332 |
| VHMPLGFLGPGGLSGRSDN | 333 |
| H | |
| VHMPLGFLGPGGTSTSGRS | 334 |
| ANPRG | |
| VLPELRSVFS | 335 |
| VLSKQMSF | 336 |
| VPAGRRS | 337 |
| VPAGRRSL | 338 |
| VPRQ | 339 |
| VPRQV | 340 |
| VSRSA | 341 |
| VVPEGRRS | 342 |
| WATPRPMR | 343 |
| WDHPISLL | 344 |
| XXQAR(A/V)X | |
| YDPZVKVVLA | 345 |
| YGAGLGVV | 346 |
| YIVSRSA | 347 |
| YKKFVGSL | 348 |
| YVPRVKALEM | 349 |
| HMMQYARH | 350 |
| HTGRSGAL | 351 |
| HVPRQ | 352 |
| HVPRQV | 353 |
| HVPRQVAPRSF | 354 |
| HVPRQVLSGRS | 355 |
| HVPRQVLSGRSAN | 356 |
| HWHLGPPT | 357 |
| IANLLSMV | 358 |
| IDGR | 359 |
| IEGR | 360 |
| ILNLLSMV | 361 |
| ILPRSPAF | 362 |
| IPFSWSRF | 363 |
| IQNLLSMV | 364 |
| ISSGL | 365 |
| ISSGLL | 366 |
| ISSGLLS | 367 |
| ISSGLLSGRSANI | 368 |
| ISSGLLSGRSANP | 369 |
| ISSGLLSGRSANPRG | 370 |
| ISSGLLSGRSDDH | 371 |
| ISSGLLSGRSDIH | 372 |
| ISSGLLSGRSDNH | 41 |
| ISSGLLSGRSDNI | 373 |
| ISSGLLSGRSDNP | 374 |
| ISSGLLSGRSDQH | 375 |
| ISSGLLSGRSDTH | 376 |
| ISSGLLSGRSDYH | 377 |
| ISSGLLSGRSGNH | 378 |
| ISSGLLSGRSNI | 379 |
| ISSGLLSGRSNIG | 380 |
| ISSGLLSGRSNIGS | 381 |
| ISSGLLSS | 382 |
| ISSGLLSSGGSGGSLSGRSDNH | 383 |
| ISSGLLSSGGSGGSLSGRSGNH | 384 |
| ISSGLSS | 385 |
| IVSRSA | 386 |
| KGLTGRSDRHQA | 387 |
| KGPKVKVVTL | 388 |
| KNLYGRSENNGN | 389 |
| KRMPVQFL | 390 |
| LAAPLGLL | 391 |
| LAHG | 392 |
| LAHGL | 393 |
| LAHGLF | 394 |
| LAPLGLQRR | 395 |
| LARAG | 396 |
| LARAGI | 397 |
| LARAGL | 398 |
| LKAAPRWA | 399 |
| LKAAPRWF | 400 |
| LKAAPVWA | 401 |
| LKAAPVWF | 402 |
| LKGRSYYY | 403 |
| LLAPSHRA | 404 |
| LLEALRAL | 405 |
| LLESLRAL | 406 |
| LLLPAHGG | 407 |
| LLLPLLGS | 408 |
| LLNALRAL | 409 |
| LLNSLRAL | 410 |
| LLQALRAL | 411 |
| LLQSLRAL | 412 |
| LLSALRAL | 413 |
| LLSSLRAL | 414 |
| LNGRSDNH | 415 |
| LPAGLLL | 416 |
| LPAGLLLR | 417 |
| LPAHLVLL | 418 |
| LPAHLVLV | 419 |
| LPGGLSPW | 420 |
| LPSHLVLL | 421 |
| LPSHLVLV | 422 |
| LPTFV | 423 |
| LPTFVH | 424 |
| LPTFVHL | 425 |
| LRSGW | 426 |
| LSGR | 427 |
| LSGRS | 428 |
| LSGRSA | 429 |
| LSGRSALAHGLF | 430 |
| LSGRSAN | 431 |
| LSGRSANI | 432 |
| LSGRSANP | 433 |
| LSGRSD | 434 |
| LSGRSDD | 435 |
| LSGRSDDH | 33 |
| LSGRSDI | 436 |
| LSGRSDIH | 437 |
| LSGRSDN | 438 |
| LSGRSDNH | 439 |
| LSGRSDNHGGAVGLLAPP | 440 |
| LSGRSDNHGGSGGSISSGLLSS | 441 |
| LSGRSDNHGGSGGSQNQALR | 442 |
| MA | |
| LSGRSDNHGGVHMPLGFLGP | 443 |
| LSGRSDNI | 444 |
| LSGRSDNP | 445 |
| LSGRSDQ | 446 |
| LSGRSDQH | 447 |
| LSGRSDT | 448 |
| LSGRSDTH | 449 |
| LSGRSDY | 450 |
| LSGRSDYH | 451 |
| LSGRSENH | 452 |
| LSGRSG | 453 |
| LSGRSGN | 454 |
| LSGRSGNH | 455 |
| LSGRSGNHGGSGGSISSGLLSS | 456 |
| LSGRSGNHGGSGGSQNQALR | 457 |
| MA | |
| LSGRSGNP | 458 |
| LSGRSVTQ | 459 |
| LSQARWRK | 460 |
| LTFPTYIF | 461 |
| LTFPTYWF | 462 |
| LTGRSDRH | 463 |
| LTGRSGA | 464 |
| LTGRSGA | 465 |
| LYAAPRWA | 466 |
| LYAAPRWF | 467 |
| LYAAPVWA | 468 |
| LYAAPVWF | 469 |
| LYGRSENN | 470 |
| MDAFLESS | 471 |
| MGLFSEAG | 472 |
| MGPWFM | 473 |
| MIAPVAYR | 474 |
| MLRSGW | 475 |
| MLRSGWR | 476 |
| MLRSGWRG | 477 |
| MLRSGWRL | 478 |
| MLRSGWRS | 479 |
| MTFPTYIF | 480 |
| MTFPTYWF | 481 |
| NHRIGRSDNHRR | 482 |
| NMPSFKLVTG | 483 |
| NTLSGRSENHSG | 484 |
| NTLSGRSGNHGS | 485 |
| NZPRVRLVLP | 486 |
| PAGLWLDP | 487 |
| PAGRR | 488 |
| PAGRRS | 489 |
| PAGRRSL | 490 |
| PASLWYTQ | 491 |
| PESRRWMP | 492 |
| PFHLSR | 493 |
| PHGFFQ | 494 |
| PLARAGI | 495 |
| PLARAGL | 496 |
| PLGL | 497 |
| PLGLAG | 498 |
| PLGLWA | 499 |
| PLGVRGK | 500 |
| PLTGRSGG | 501 |
| PLTGRSGGGGSS | 502 |
| PPLGR | 503 |
| PPPDMKLFPG | 504 |
| PPPEVRSFSV | 505 |
| PPPVLKLLEW | 506 |
| PPSIARSDNLAN | 507 |
| PQHRIVSF | 508 |
| PRFKIIGG | 509 |
| PRFRIIGG | 510 |
| PRPFVKSVDQ | 511 |
| PRQV | 512 |
| PRSF | 513 |
| PSPPVKMMPE | 514 |
| PTNGGSGGSS | 515 |
| RVPKVKVMLD | 516 |
| SAGFSLPA | 517 |
| SAPAVESE | 518 |
| SAPYFRMMDM | 519 |
| SARGPSRW | 520 |
| SCGPPLGR | 521 |
| SCSPPLGR | 522 |
| SGGPLGVR | 523 |
| SGGPPLGR | 524 |
| SGPPLGR | 525 |
| SGRS | 526 |
| SGRSA | 527 |
| SGRSAN | 528 |
| SGRSANI | 529 |
| SGRSANP | 530 |
| SGRSANPRG | 531 |
| SGRSD | 532 |
| SGRSDD | 533 |
| SGRSDDH | 534 |
| SGRSDI | 535 |
| SGRSDIH | 536 |
| SGRSDN | 537 |
| SGRSDNI | 538 |
| SGRSDNP | 539 |
| SGRSDQ | 540 |
| SGRSDQH | 541 |
| SGRSDT | 542 |
| SGRSDTH | 543 |
| SGRSDY | 544 |
| SGRSDYH | 545 |
| SGRSG | 546 |
| SGRSGN | 547 |
| SGRSGNH | 548 |
| SGSPPLGR | 549 |
| SIARSDNL | 550 |
| SISSGLLSGRSDNI | 551 |
| SNPFKY | 552 |
| SPLPLRVP | 553 |
| SPLTGRSG | 554 |
| SPPLGR | 555 |
| SRRVP | 556 |
| SRRVPL | 557 |
| SRRVPV | 558 |
| SSGPPLGR | 559 |
| SSPPLGR | 560 |
| SSRGPAYL | 561 |
| SSRHRRALD | 562 |
| SSSFDKGKYKKGDDA | 563 |
| SSSFDKGKYKRGDDA | 564 |
| SSSPPLGR | 565 |
| STFPFGMF | 566 |
| STVFHM | 567 |
| SVHHLI | 568 |
| SVSGLLSH | 569 |
| SVSGLLSS | 570 |
| SVSGLRSH | 571 |
| SVSGLRSS | 572 |
| TARG | 573 |
| TARGP | 574 |
| TARGPALAHGLF | 575 |
| TARGPS | 576 |
| TARGPSF | 577 |
| TARGPSFK | 578 |
| TARGPSW | 579 |
| TARGPSW | 580 |
| TARGPVPRQV | 581 |
| TFVH | 582 |
| TGLSGRSVTQTS | 583 |
| TGRGPSWV | 584 |
| TLRLGRSDNNKN | 585 |
| TLSGLRSP | 586 |
| TSGRSANP | 587 |
| TSGRSGNP | 588 |
| TSLSGRSANPRG | 589 |
| TSLSGRSGNPRG | 590 |
| TSSGLRSP | 591 |
| TSTSGRSANPRG | 592 |
| TSTSGRSANPRGGGAVGLLAP | 593 |
| P | |
| TSTSGRSANPRGGGVHMPLGF | 594 |
| LGP | |
| TSTSGRSGNPRG | 595 |
| TVSGLRSP | 596 |

Examples of CMs also include those described in WO 2010/081173, WO2021207669, WO2021207657, WO2021142029, WO2021061867, WO2020252349, WO2020252358, WO2020236679, WO2020176672, WO2020118109, WO2020092881, WO2020086665, WO2019213444, WO2019183218, WO2019173771, WO2019165143, WO2019075405, WO2019046652, WO2019018828, WO2019014586, WO2018222949, WO2018165619, WO2018085555, WO2017011580, WO2016179335, WO2016179285, WO2016179257, WO2016149201, WO2016014974, which are incorporated herein by reference in their entireties for all purposes.

In some embodiments, the CM may be or comprise a combination, a C-terminal truncation variant, or an N-terminal truncation variant of the example sequences discussed above. Truncation variants of the aforementioned amino acid sequences that are suitable for use in a CM may be any that retain the recognition site for the corresponding protease. These include C-terminal and/or N-terminal truncation variants comprising at least 3 contiguous amino acids of the above-described amino acid sequences, or at least 4, 5, 6, 7, 8, 9, or 10 amino acids of the foregoing amino acid sequences that retain a recognition site for a protease. In certain embodiments, the truncation variant of the above-described amino acid sequences may be an amino acid sequence corresponding to any of the above, but that is C- and/or N-terminally truncated by 1 to 10 amino acids, 1 to 9 amino acids, 1 to 8 amino acids, 1 to 7 amino acids, 1 to 6 amino acids, 1 to 5 amino acids, 1 to 4 amino acids, or 1 to 3 amino acids, and which: (1) has at least three amino acid residues; and (2) retains a recognition site for a protease. In some of the foregoing embodiments, the truncated CM is an N-terminally truncated CM. In some embodiments, the truncated CM is a C-terminally truncated CM. In some embodiments, the truncated C is a C- and an N-terminally truncated CM.

In some embodiments, the CM may comprise a total of 3 amino acids to 25 amino acids. In some embodiments, the CM may comprise a total of 3 to 25, 3 to 20, 3 to 15, 3 to 10, 3 to 5, 5 to 25, 5 to 20, 5 to 15, 5 to 10, 10 to 25, 10 to 20, 10 to 15, 15 to 25, 15 to 20, or 20 to 25 amino acids.

In some embodiments, the CM may be specifically cleaved by at least a protease at a rate of about 0.001-1500×10⁴ M⁻¹S⁻¹ or at least 0.001, 0.005, 0.01, 0.05, 0.1, 0.5, 1, 2.5, 5, 7.5, 10, 15, 20, 25, 50, 75, 100, 125, 150, 200, 250, 500, 750, 1000, 1250, or 1500×10⁴ M⁻¹S⁻¹. The rate may be measured as substrate cleavage kinetics (k_{cat}/Kₘ) as disclosed in WO2016118629.

### Linkers

The activatable protein may comprise one or more linkers. The linkers may comprise a stretch of amino acid sequence that link two components in the activatable protein. The linkers may be non-cleavable by any protease. In some embodiments, one or more linkers (e.g., flexible linkers) may be introduced into the activatable protein to provide flexibility at one or more of the junctions between domains, between moieties, between moieties and domains, or at any other junctions where a linker would be beneficial. In some embodiments, where the activatable protein is provided as a conformationally constrained construct, a flexible linker may be inserted to facilitate formation and maintenance of a structure in the uncleaved activatable protein. Any of the linkers described herein may provide the desired flexibility to facilitate the inhibition of the binding of a target, or to facilitate cleavage of a CM by a protease. In some embodiments, linkers included in the activatable protein may be all or partially flexible, such that the linker can include a flexible linker as well as one or more portions that confer less flexible structure to provide for a desired activatable protein. Some linkers may include cysteine residues, which may form disulfide bonds and reduce flexibility of the construct.

In some embodiments, a linker coupled to a MM may have a length that allows the MM to be in a position in the tertiary or quaternary to effectively mask a TB, e.g., proximal to the TB to be masked) that allows the MM to mask the TB.

In most instances, linker length may be determined by counting, in a N- to C-direction, the number of amino acids from the N-terminus of the linker adjacent to the C-terminal amino acid of the preceding component, to the C-terminus of the linker adjacent to the N-terminal amino acid of the following component (i.e., where the linker length does not include either the C-terminal amino acid of the preceding component or the N-terminal amino acid of the following component).

In some embodiments, a linker may include a total of 1 to 50, 1 to 40, 1 to 30, 1 to 25 (e.g., 1 to 24, 1 to 22, 1 to 20, 1 to 18, 1 to 16, 1 to 15, 1 to 14, 1 to 12, 1 to 10, 1 to 8, 1 to 6, 1 to 5, 1 to 4, 1 to 3, 1 to 2, 2 to 25, 2 to 24, 2 to 22, 2 to 20, 2 to 18, 2 to 16, 2 to 15, 2 to 14, 2 to 12, 2 to 10, 2 to 8, 2 to 6, 2 to 5, 2 to 4, 2 to 3, 4 to 25, 4 to 24, 4 to 22, 4 to 20, 4 to 18, 4 to 16, 4 to 15, 4 to 14, 4 to 12, 4 to 10, 4 to 8, 4 to 6, 4 to 5, 5 to 25, 5 to 24, 5 to 22, 5 to 20, 5 to 18, 5 to 16, 5 to 15, 5 to 14, 5 to 12, 5 to 10, 5 to 8, 5 to 6, 6 to 25, 6 to 24, 6 to 22, 6 to 20, 6 to 18, 6 to 16, 6 to 15, 6 to 14, 6 to 12, 6 to 10, 6 to 8, 8 to 25, 8 to 24, 8 to 22, 8 to 20, 8 to 18, 8 to 16, 8 to 15, 8 to 14, 8 to 12, 8 to 10, 10 to 25, 10 to 24, 10 to 22, 10 to 20, 10 to 18, 10 to 16, 10 to 15, 10 to 14, 10 to 12, 12 to 25, 12 to 24, 12 to 22, 12 to 20, 12 to 18, 12 to 16, 12 to 15, 12 to 14, 14 to 25, 14 to 24, 14 to 22, 14 to 20, 14 to 18, 14 to 16, 14 to 15, 15 to 25, 15 to 24, 15 to 22, 15 to 20, 15 to 18, 15 to 16, 16 to 25, 16 to 24, 16 to 22, 16 to 20, 16 to 18, 18 to 25, 18 to 24, 18 to 22, 18 to 20, 20 to 25, 20 to 24, 20 to 22, 22 to 25, 22 to 24, or 24 to 25 amino acids). In some embodiments, the linker may include a total of 1, 2, 3, 4, 5, 6, 7, 8, 9, 10, 11, 12, 13, 14, 15, 16, 17, 18, 19, 20, 21, 22, 23, 24, or 25 amino acids.

In some embodiments, a linker may be rich in glycine (Gly or G) residues. In some embodiments, the linker may be rich in serine (Ser or S) residues. In some embodiments, the linker may be rich in glycine and serine residues. In some embodiments, the linker may have one or more glycine-serine residue pairs (GS) (e.g., 1, 2, 3, 4, 5, 6, 7, 8, 9, or 10 or more GS pairs).

In some embodiments, the linker may have one or more Gly-Gly-Gly-Ser (GGGS) sequences (e.g., 1, 2, 3, 4, 5, 6, 7, 8, 9, or 10 or more GGGS sequences). In some embodiments, the linker may have one or more Gly-Gly-Gly-Gly-Ser (GGGGS) sequences (e.g., 1, 2, 3, 4, 5, 6, 7, 8, 9, or 10 or more GGGGS sequences). In some embodiments, the linker may have one or more Gly-Gly-Ser-Gly (GGSG) sequences (e.g., 1, 2, 3, 4, 5, 6, 7, 8, 9, or 10 or more GGSG sequences). Examples of the linkers may include glycine polymers (G)n, glycine-serine polymers (including, for example, (GS)n, (GGS)n, (GSGGS)n and (GGGS)n, where n is an integer of at least one), glycine-alanine polymers, alanine-serine polymers, and other flexible linkers known in the art. Glycine and glycine-serine polymers may be relatively unstructured, and therefore may be able to serve as a neutral link between components. Glycine accesses significantly more phi-psi space than even alanine, and is much less restricted than residues with longer side chains (see Scheraga, Rev. Computational Chem. 11173-142 (1992)). Example flexible linkers include one of or a combination of one or more of: GGSG (SEQ ID NO: 71), GGSGG (SEQ ID NO: 72), GSGSG (SEQ ID NO: 73), GSGGG (SEQ ID NO: 74), GGGSG (SEQ ID NO: 75), GSSSG (SEQ ID NO: 76), GSSGGSGGSGG (SEQ ID NO: 77), GGGS (SEQ ID NO: 78), GGGSGGGS (SEQ ID NO: 79), GGGSGGGSGGGS (SEQ ID NO: 80), GGGGSGGGGSGGGGS (SEQ ID NO: 81), GGGGSGGGGSGGGGSGGGGSGGGGS (SEQ ID NO: 82), GGGGSGGGGS (SEQ ID NO: 83), GGGGS (SEQ ID NO: 84), GS, GGGGSGS (SEQ ID NO: 85), GGGGSGGGGSGGGGSGS (SEQ ID NO: 86), GGSLDPKGGGGS (SEQ ID NO: 87), PKSCDKTHTCPPCPAPELLG (SEQ ID NO: 88), SKYGPPCPPCPAPEFLG (SEQ ID NO: 89), GKSSGSGSESKS (SEQ ID NO: 90), GSTSGSGKSSEGKG (SEQ ID NO: 91), GSTSGSGKSSEGSGSTKG (SEQ ID NO: 92), GSTSGSGKPGSGEGSTKG (SEQ ID NO: 93), GSTSGSGKPGSSEGST (SEQ ID NO: 94), GGGSSGGS (SEQ ID NO: 95), GGGGSGGGGSS (SEQ ID NO: 96), GGGSSGGSGGSSGGS (SEQ ID NO: 97), and GSTSGSGKPGSSEGST (SEQ ID NO: 98).

Examples of linkers may further include a sequence that is at least 70% identical (e.g., at least 72%, at least 74%, at least 75%, at least 76%, at least 78%, at least 80%, at least 82%, at least 84%, at least 85%, at least 86%, at least 88%, at least 90%, at least 92%, at least 94%, at least 95%, at least 96%, at least 97%, at least 98%, at least 99%, or 100% identical to the example linkers described herein. An ordinarily skilled artisan will recognize that design of an activatable proteins can include linkers that are all or partially flexible, such that the linker can include a flexible linker as well as one or more portions that confer less flexible structure to provide for a desired activatable proteins structure.

In some embodiments, an activatable protein may include one, two, three, four, five, six, seven, eight, nine, or ten linker sequence(s) (e.g., the same or different linker sequences of any of the exemplary linker sequences described herein or known in the art). In some embodiments, a linker may comprise sulfo-SIAB, SMPB, and sulfo-SMPB, wherein the linkers react with primary amines sulfhydryls.

### Conjugation Agents

In some aspects, the activatable molecules (e.g., activatable proteins such as activatable antibodies) may further comprise one or more additional agents, e.g., a targeting moiety to facilitate delivery to a cell or tissue of interest, a therapeutic agent (e.g., an antineoplastic agent such as chemotherapeutic or anti-neoplastic agent), a toxin, or a fragment thereof. The additional agents may be conjugated to the activatable antibodies. The term “agent” is used herein to denote a chemical compound, a mixture of chemical compounds, a biological macromolecule, or an extract made from biological materials.

In some embodiments, the activatable protein may be conjugated to a cytotoxic agent, e.g., a toxin (e.g., an enzymatically active toxin of bacterial, fungal, plant, or animal origin, or fragments thereof) or a radioactive isotope.

Examples of cytotoxic agents that can be conjugated to the activatable proteins include: dolastatins and derivatives thereof (e.g., auristatin E, AFP, monomethyl auristatin D (MMAD), monomethyl auristatin F (MMAF), monomethyl auristatin E (MMAE), desmethyl auristatin E (DMAE), auristatin F, desmethyl auristatin F (DMAF), dolastatin 16 (DmJ), dolastatin 16 (Dpv), auristatin derivatives (e.g., auristatin tyramine, auristatin quinolone), maytansinoids (e.g., DM-1, DM-4), maytansinoid derivatives, duocarmycin, alpha-amanitin, turbostatin, phenstatin, hydroxyphenstatin, spongistatin 5, spongistatin 7, halistatin 1, halistatin 2, halistatin 3, halocomstatin, pyrrolobenzimidazoles (PBI), cibrostatin6, doxaliform, cemadotin analogue (CemCH2-SH), Pseudomonas toxin A (PES8) variant, Pseudomonase toxin A (ZZ-PE38) variant, ZJ-101, anthracycline, doxorubicin, daunorubicin, bryostatin, camptothecin, 7-substituted campothecin, 10, 11-difluoromethylenedioxycamptothecin, combretastatins, debromoaplysiatoxin, KahaMide-F, discodermolide, and Ecteinascidins.

Examples of enzymatically active toxins that can be conjugated to the activatable proteins include: diphtheria toxin, exotoxin A chain from Pseudomonas aeruginosa, ricin A chain, abrin A chain, modeccin A chain, alpha-sarcin, Aleuriies fordii proteins, dianfhin proteins, Phytoiaca Americana proteins (e.g., PAPI, PAPII, and PAP-8), Momordica charantia inhibitor, curcin, crotirs, Sapaonaria officinalis inhibitor, geionin, mitogeliin, restrictocin, phenomycin, neomycin, and tricothecenes.

Examples of anti-neoplastics that can be conjugated to the activatable proteins include: adriamycin, cerubidine, bleomycin, alkeran, velban, oncovin, fluorouracil, methotrexate, thiotepa, bisantrene, novantrone, thioguanine, procarabizine, and cytarabine.

Examples of antivirals that can be conjugated to the activatable proteins include: acyclovir, vira A, and symmetrel. Examples of antifungals that can be conjugated to the activatable proteins include: nystatin. Examples of detection reagents that can be conjugated to the activatable proteins include: fluorescein and derivatives thereof, fluorescein isothiocyanate (FITC). Examples of antibacterials that can be conjugated to the activatable proteins include: aminoglycosides, streptomycin, neomycin, kanamycin, amikacin, gentamicin, and tobramycin. Examples of 3beta,16beta,17alpha-trihydroxycholest-5-en-22-one 16-O-(2-O-4-methoxybenzoyl-beta-D-xylopyranosyl)-(1→3)-(2-O-acetyl-alpha-L-arabinopyranoside) (OSW-1) that can be conjugated to the activatable proteins include: s-nitrobenzyloxycarbonyl derivatives of 06-benzylguanine, toposisomerase inhibitors, hemiasterlin, cephalotaxine, homoharringionine, pyrrol obenzodiazepine dimers (PBDs), functionalized pyrrolobenzodiazepenes, calcicheamicins, podophyiitoxins, taxanes, and vinca alkoids. Examples of radiopharmaceuticals that can be conjugated to the activatable proteins include: ¹²³I, ⁸⁹Zr, ¹²⁵I, ¹³¹I, ^{99m}Tc, ²⁰¹Tl, ⁶²Cu, ¹⁸F, ⁶⁸Ga, ¹³N, ¹⁵O, ³⁸K, ⁸²Rb, ¹¹¹In, ¹³³Xe, ¹¹C, and ^{99m}Tc (Technetium). Examples of heavy metals that can be conjugated to the activatable proteins include: barium, gold, and platinum. Examples of anti-mycoplasmals that can be conjugated to the activatable proteins include: tylosine, spectinomycin, streptomycin B, ampicillin, sulfanilamide, polymyxin, and chloramphenicol.

In some embodiments, the activatable protein may comprise a signal peptide. If comprising multiple polypeptides, the activatable protein may comprise multiple signal peptides, e.g., one signal peptide for each of the multiple polypeptides. A signal peptide may be a peptide (e.g., 10-30 amino acids long) present at a terminus (e.g., the N-terminus or C-terminus) of a newly synthesized proteins that are destined toward the secretory pathway. In some embodiments, the signal peptide may be conjugated to the activatable protein via a spacer. In some embodiments, the spacer may be conjugated to the activatable protein in the absence of a signal peptide.

Those of ordinary skill in the art will recognize that a large variety of possible agents may be conjugated to any of the activatable proteins described herein. The agents may be conjugated to another component of the activatable protein by a conjugating moiety. Conjugation may include any chemical reaction that binds the two molecules so long as the activatable protein and the other moiety retain their respective activities. Conjugation may include many chemical mechanisms, e.g., covalent binding, affinity binding, intercalation, coordinate binding, and complexation. In some embodiments, the binding may be covalent binding. Covalent binding may be achieved either by direct condensation of existing side chains or by the incorporation of external bridging molecules. Many bivalent or polyvalent linking agents may be useful in conjugating any of the activatable proteins described herein. For example, conjugation may include organic compounds, such as thioesters, carbodiimides, succinimide esters, glutaraldehyde, diazobenzenes, and hexamethylene diamines. In some embodiments, the activatable proteins may include, or otherwise introduce, one or more non-natural amino acid residues to provide suitable sites for conjugation.

In some embodiments, an agent and/or conjugate may be attached by disulfide bonds (e.g., disulfide bonds on a cysteine molecule) to the antigen-binding domain. Since many cancers naturally release high levels of glutathione, a reducing agent, glutathione present in the cancerous tissue microenvironment can reduce the disulfide bonds, and subsequently release the agent and/or the conjugate at the site of delivery.

In some embodiments, when the conjugate binds to its target in the presence of complement within the target site (e.g., diseased tissue (e.g., cancerous tissue)), the amide or ester bond attaching the conjugate and/or agent to the linker is cleaved, resulting in the release of the conjugate and/or agent in its activated form. These conjugates and/or agents when administered to a subject, may accomplish delivery and release of the conjugate and/or the agent at the target site (e.g., diseased tissue (e.g., cancerous tissue)). These conjugates and/or agents may be effective for the in vivo delivery of any of the conjugates and/or agents described herein.

In some embodiments, the conjugating moiety may be uncleavable by enzymes of the complement system. For example, the conjugate and/or agent is released without complement activation since complement activation ultimately lyses the target cell. In such embodiments, the conjugate and/or agent is to be delivered to the target cell (e.g., hormones, enzymes, corticosteroids, neurotransmitters, or genes). Furthermore, the conjugating moiety may be mildly susceptible to cleavage by serum proteases, and the conjugate and/or agent is released slowly at the target site.

In some embodiments, the conjugate and/or agent may be designed such that the conjugate and/or agent is delivered to the target site (e.g., disease tissue (e.g., cancerous tissue)) but the conjugate and/or agent is not released.

In some embodiments, the conjugate and/or agent may be attached to an antigen-binding domain either directly or via amino acids (e.g., D-amino acids), peptides, thiol-containing moieties, or other organic compounds that may be modified to include functional groups that can subsequently be utilized in attachment to antigen-binding domains by methods described herein.

In some embodiments, an activatable protein may include at least one point of conjugation for an agent. In some embodiments, all possible points of conjugation are available for conjugation to an agent. In some embodiments, the one or more points of conjugation may include sulfur atoms involved in disulfide bonds, sulfur atoms involved in interchain disulfide bonds, sulfur atoms involved in interchain sulfide bonds but not sulfur atoms involved in intrachain disulfide bonds, and/or sulfur atoms of cysteine or other amino acid residues containing a sulfur atom. In such cases, residues may occur naturally in the protein construct structure or may be incorporated into the protein construct using methods including site-directed mutagenesis, chemical conversion, or mis-incorporation of non-natural amino acids.

The present disclosure also provides methods and materials for preparing an activatable protein with one or more conjugated agents. In some embodiments, an activatable protein may be modified to include one or more interchain disulfide bonds. For example, disulfide bonds may undergo reduction following exposure to a reducing agent such as, without limitation, TCEP, DTT, or β-mercaptoethanol. In some cases, the reduction of the disulfide bonds may be only partial. As used herein, the term partial reduction refers to situations where an activatable protein is contacted with a reducing agent and a fraction of all possible sites of conjugation undergo reduction (e.g., not all disulfide bonds are reduced). In some embodiments, an activatable protein may be partially reduced following contact with a reducing agent if less than 99%, (e.g., less than 98%, 97%, 96%, 95%, 90%, 85%, 80%, 75%, 70%, 65%, 60%, 55%, 50%, 45%, 40%, 35%, 30%, 25%, 20%, 15%, 10% or 5%) of all possible sites of conjugation are reduced. In some embodiments, the activatable protein having a reduction in one or more interchain disulfide bonds may be conjugated to a drug reactive with free thiols.

The present disclosure also provides methods and materials for conjugating a therapeutic agent to a particular location on an activatable protein. In some embodiments, an activatable protein may be modified so that the therapeutic agents can be conjugated to the activatable protein at particular locations on the activatable protein. For example, an activatable protein may be partially reduced in a manner that facilitates conjugation to the activatable protein. In such cases, partial reduction of the activatable protein may occur in a manner that conjugation sites in the activatable protein are not reduced. In some embodiments, the conjugation site(s) on the activatable protein may be selected to facilitate conjugation of an agent at a particular location on the protein construct. Various factors can influence the “level of reduction” of the activatable protein upon treatment with a reducing agent. For example, without limitation, the ratio of reducing agent to activatable protein, length of incubation, incubation temperature, and/or pH of the reducing reaction solution can require optimization in order to achieve partial reduction of the activatable protein with the methods and materials described herein. Any appropriate combination of factors (e.g., ratio of reducing agent to activatable protein, the length and temperature of incubation with reducing agent, and/or pH of reducing agent) may be used to achieve partial reduction of the activatable protein (e.g., general reduction of possible conjugation sites or reduction at specific conjugation sites).

An effective ratio of reducing agent to activatable protein can be any ratio that at least partially reduces the A activatable protein in a manner that allows conjugation to an agent (e.g., general reduction of possible conjugation sites or reduction at specific conjugation sites). In some embodiments, the ratio of reducing agent to activatable protein may be in a range from about 20:1 to 1:1, from 10:1 to 1:1, from 9:1 to 1:1, from 8:1 to 1:1, from 7:1 to 1:1, from 6:1 to 1:1, from 5:1 to 1:1, from 4:1 to 1:1, from 3:1 to 1:1, from 2:1 to 1:1, from 20:1 to 1:1.5, from 10:1 to 1:1.5, from 9:1 to 1:1.5, from 8:1 to 1:1.5, from 7:1 to 1:1.5, from 6:1 to 1:1.5, from 5:1 to 1:1.5, from 4:1 to 1:1.5, from 3:1 to 1:1.5, from 2:1 to 1:1.5, from 1.5:1 to 1:1.5, or from 1:1 to 1:1.5.

An effective incubation time and temperature for treating an activatable protein with a reducing agent may be any time and temperature that at least partially reduces the activatable protein in a manner that allows conjugation of an agent to an activatable protein (e.g., general reduction of possible conjugation sites or reduction at specific conjugation sites). In some embodiments, the incubation time and temperature for treating an activatable protein may be in a range from about 1 hour at 37° C. to about 12 hours at 37° C. (or any subranges therein).

An effective pH for a reduction reaction for treating an activatable protein with a reducing agent can be any pH that at least partially reduces the activatable protein in a manner that allows conjugation of the activatable protein to an agent (e.g., general reduction of possible conjugation sites or reduction at specific conjugation sites).

When a partially-reduced activatable protein is contacted with an agent containing thiols, the agent may conjugate to the interchain thiols in the activatable protein. An agent can be modified in a manner to include thiols using a thiol-containing reagent (e.g., cysteine or N-acetyl cysteine). For example, the activatable protein can be partially reduced following incubation with reducing agent (e.g., TEPC) for about 1 hour at about 37° C. at a desired ratio of reducing agent to activatable protein. An effective ratio of reducing agent to activatable protein may be any ratio that partially reduces at least two interchain disulfide bonds located in the activatable protein in a manner that allows conjugation of a thiol-containing agent (e.g., general reduction of possible conjugation sites or reduction at specific conjugation sites).

In some embodiments, an activatable protein may be reduced by a reducing agent in a manner that avoids reducing any intrachain disulfide bonds. In some embodiments of, an activatable protein may be reduced by a reducing agent in a manner that avoids reducing any intrachain disulfide bonds and reduces at least one interchain disulfide bond.

In some embodiments, the agent (e.g., agent conjugated to an activatable protein) may be a detectable moiety such as, for example, a label or other marker. For example, the agent may be or include a radiolabeled amino acid, one or more biotinyl moieties that can be detected by marked avidin (e.g., streptavidin containing a fluorescent marker or enzymatic activity that can be detected by optical or calorimetric methods), one or more radioisotopes or radionuclides, one or more fluorescent labels, one or more enzymatic labels, and/or one or more chemiluminescent agents. In some embodiments, detectable moieties may be attached by spacer molecules. In some embodiments, the detectable label may include an imaging agent, a contrasting agent, an enzyme, a fluorescent label, a chromophore, a dye, one or more metal ions, or a ligand-based label. In some embodiments, the imaging agent may comprise a radioisotope. In some embodiments, the radioisotope may be indium or technetium. In some embodiments, the contrasting agent may comprise iodine, gadolinium or iron oxide. In some embodiments, the enzyme may comprise horseradish peroxidase, alkaline phosphatase, or β-galactosidase. In some embodiments, the fluorescent label may comprise yellow fluorescent protein (YFP), cyan fluorescent protein (CFP), green fluorescent protein (GFP), modified red fluorescent protein (mRFP), red fluorescent protein tdimer2 (RFP tdimer2), HCRED, or a europium derivative. In some embodiments, the luminescent label may comprise an N-methylacrydium derivative. In some embodiments, the label may comprise an Alexa Fluor® label, such as Alex Fluor® 680 or Alexa Fluor® 750. In some embodiments, the ligand-based label may comprise biotin, avidin, streptavidin or one or more haptens.

Further examples of detectable labels also include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

In some embodiments, the agent may be conjugated to the activatable protein using a carbohydrate moiety, sulfhydryl group, amino group, or carboxylate group. In some embodiments, the agent may be conjugated to the activatable protein via a linker and/or a CM described herein. In some embodiments, the agent may be conjugated to a cysteine or a lysine in the activatable protein. In some embodiments, the agent may be conjugated to another residue of the activatable protein, such as those residues disclosed herein.

In some embodiments, a variety of bifunctional protein-coupling agents may be used to conjugate the agent to the activatable protein including N-succinimidyl-3-(2-pyridyldithiol) propionate (SPDP), iminothiolane (IT), bifunctional derivatives of imidoesters (e.g., dimethyl adipimidate HCL), active esters (e.g., disuccinimidyl suberate), aldehydes (e.g., glutareldehyde), bis-azido compounds (e.g., bis (p-azidobenzoyl) hexanediamine), bis-diazonium derivatives (e.g., bis-(p-diazoniumbenzoyl)-ethylenediamine), diisocyanates (e.g., tolyene 2,6-diisocyanate), and bis-active fluorine compounds (e.g., 1,5-difluoro-2,4-dinitrobenzene). For example, a ricin immunotoxin can be prepared as described in Vitetta et al., Science 238: 1098 (1987). In some embodiments, a carbon-14-labeled 1-isothiocyanatobenzyl-3-methyldiethylene triaminepentaacetic acid (MX-DTPA) chelating agent can be used to conjugate a radionucleotide to the activatable protein. (See, e.g., WO94/11026).

Suitable conjugation moieties include those described in the literature. (See, for example, Ramakrishnan, S. et al., Cancer Res. 44:201-208 (1984) describing use of MBS (M-maleimidobenzoyl-N-hydroxysuccinimide ester). See also, U.S. Pat. No. 5,030,719, describing use of halogenated acetyl hydrazide derivative coupled to an activatable protein by way of an oligopeptide. In some embodiments, suitable conjugation moieties include: (i) EDC (1-ethyl-3-(3-dimethylamino-propyl) carbodiimide hydrochloride; (ii) SMPT (4-succinimidyloxycarbonyl-alpha-methyl-alpha-(2-pridyl-dithio)-toluene (Pierce Chem. Co., Cat. (21558G); (iii) SPDP (succinimidyl-6 [3-(2-pyridyldithio) propionamido]hexanoate (Pierce Chem. Co., Cat #21651G); (iv) Sulfo-LC-SPDP (sulfosuccinimidyl 6 [3-(2-pyridyldithio)-propianamide]hexanoate (Pierce Chem. Co. Cat. #2165-G); and (v) sulfo-NHS (N-hydroxysulfo-succinimide: Pierce Chem. Co., Cat. #24510) conjugated to EDC. Additional example conjugation moieties include SMCC, sulfo-SMCC, SPDB, and sulfo-SPDB.

The conjugation moieties described above may contain components that have different attributes, thus leading to conjugates with differing physio-chemical properties. For example, sulfo-NHS esters of alkyl carboxylates are more stable than sulfo-NHS esters of aromatic carboxylates. NHS-ester containing linkers are less soluble than sulfo-NHS esters. Further, the SMPT contains a sterically-hindered disulfide bond, and can form conjugates with increased stability. Disulfide linkages, are in general, less stable than other linkages because the disulfide linkage is cleaved in vitro, resulting in less conjugate available. Sulfo-NHS, in particular, can enhance the stability of carbodimide couplings. Carbodimide couplings (such as EDC) when used in conjunction with sulfo-NHS, forms esters that are more resistant to hydrolysis than the carbodimide coupling reaction alone.

Those of ordinary skill in the art will recognize that a large variety of possible moieties can be coupled to the activatable protein of the disclosure. (See, for example, “Conjugate Vaccines”, Contributions to Microbiology and Immunology, J. M. Cruse and R. E. Lewis, Jr (eds), Carger Press, New York, (1989), the entire contents of which are incorporated herein by reference). In general, an effective conjugation of an agent (e.g., cytotoxic agent) to an activatable protein can be accomplished by any chemical reaction that will bind the agent to the activatable protein while also allowing the agent and the activatable protein to retain functionality.

### Nucleic Acids and Vectors

In some aspects, the present disclosure further provides nucleic acids comprising sequences that encode the activatable molecules (e.g., activatable antibodies) herein, or components or fragment thereof. The nucleic acids may comprise coding sequences for the TBs, the CMs, the MMs, the EM and the linker(s) in an activatable protein. In cases where the activatable protein comprises multiple polypeptides (e.g., multiple TBs on different polypeptides, or a TB comprises multiple polypeptides), the nucleic acid may comprise coding sequences for the multiple polypeptides. In some examples, the coding sequences for one of the polypeptides are comprised in a nucleic acid, and the coding sequences for another one of the polypeptides are comprised in another nucleic acid. In some examples, the coding sequences for two or more of the multiple polypeptides are comprised in the same nucleic acid. The present disclosure includes a polynucleotide encoding a protein as described herein or a portion thereof, and use of such polynucleotides to produce the proteins and/or for therapeutic purposes. Such polynucleotides may include DNA and RNA molecules (e.g., mRNA, self-replicating RNA, self-amplifying mRNA, etc.) that encode a protein as defined herein. The present disclosure includes compositions comprising such polynucleotides. In some aspects, such compositions may be used therapeutically or prophylactically.

Unless otherwise specified, a “nucleic acid sequence encoding a protein” includes all nucleotide sequences that are degenerate versions of each other and thus encode the same amino acid sequence. The term “nucleic acid” refers to a deoxyribonucleic acid (DNA) or ribonucleic acid (RNA), or a combination thereof, in either a single- or double-stranded form. Unless specifically limited, the term encompasses nucleic acids containing known analogues of natural nucleotides that have similar binding properties as the reference nucleotides. Unless otherwise indicated, a particular nucleic acid sequence also implicitly encompasses complementary sequences as well as the sequence explicitly indicated. In some embodiments, the nucleic acid is DNA. In some embodiments, the nucleic acid is RNA.

The term “N-terminally positioned” when referring to a position of a first domain or sequence relative to a second domain or sequence in a polypeptide primary amino acid sequence means that the first domain is located closer to the N-terminus of the polypeptide primary amino acid sequence. In some embodiments, there may be additional sequences and/or domains between the first domain or sequence and the second domain or sequence. The term “C-terminally positioned” when referring to a position of a first domain or sequence relative to a second domain or sequence in a polypeptide primary amino acid sequence means that the first domain is located closer to the C-terminus of the polypeptide primary amino acid sequence. In some embodiments, there may be additional sequences and/or domains between the first domain or sequence and the second domain or sequence.

Modifications may be introduced into a nucleotide sequence by standard techniques known in the art, such as site-directed mutagenesis and polymerase chain reaction (PCR)-mediated mutagenesis. Conservative amino acid substitutions are ones in which the amino acid residue is replaced with an amino acid residue having a similar side chain. Families of amino acid residues having similar side chains have been defined in the art. These families include: amino acids with acidic side chains (e.g., aspartate and glutamate), amino acids with basic side chains (e.g., lysine, arginine, and histidine), non-polar amino acids (e.g., alanine, valine, leucine, isoleucine, proline, phenylalanine, methionine, and tryptophan), uncharged polar amino acids (e.g., glycine, asparagine, glutamine, cysteine, serine, threonine and tyrosine), hydrophilic amino acids (e.g., arginine, asparagine, aspartate, glutamine, glutamate, histidine, lysine, serine, and threonine), hydrophobic amino acids (e.g., alanine, cysteine, isoleucine, leucine, methionine, phenylalanine, proline, tryptophan, tyrosine, and valine). Other families of amino acids include: aliphatic-hydroxy amino acids (e.g., serine and threonine), amide family (e.g., asparagine and glutamine), alphatic family (e.g., alanine, valine, leucine and isoleucine), and aromatic family (e.g., phenylalanine, tryptophan, and tyrosine).

The present disclosure further provides vectors and sets of vectors comprising any of the nucleic acids described herein. One skilled in the art will be capable of selecting suitable vectors or sets of vectors (e.g., expression vectors) for making any of the activatable proteins described herein, and using the vectors or sets of vectors to express any of the activatable proteins described herein. For example, in selecting a vector or a set of vectors, the type of cell may be selected such that the vector(s) may need to be able to integrate into a chromosome of the cell and/or replicate in it. Example vectors that can be used to produce an activatable protein are also described herein. As used herein, the term “vector” refers to a polynucleotide capable of inducing the expression of a recombinant protein (e.g., a first or second monomer) in a cell (e.g., any of the cells described herein). A “vector” is able to deliver nucleic acids and fragments thereof into a host cell, and includes regulatory sequences (e.g., promoter, enhancer, poly(A) signal). Exogenous polynucleotides may be inserted into the expression vector in order to be expressed. The term “vector” also includes artificial chromosomes, plasmids, retroviruses, and baculovirus vectors.

Methods for constructing suitable vectors that comprise any of the nucleic acids described herein, and suitable for transforming cells (e.g., mammalian cells) are well-known in the art. See, e.g., Sambrook et al., Eds. “Molecular Cloning: A Laboratory Manual,” 2^{nd} Ed., Cold Spring Harbor Press, 1989 and Ausubel et al., Eds. “Current Protocols in Molecular Biology,” Current Protocols, 1993.

Examples of vectors include plasmids, transposons, cosmids, and viral vectors (e.g., any adenoviral vectors (e.g., pSV or pCMV vectors), adeno-associated virus (AAV) vectors, lentivirus vectors, and retroviral vectors), and any Gateway@vectors. A vector may, for example, include sufficient cis-acting elements for expression; other elements for expression may be supplied by the host mammalian cell or in an in vitro expression system. Skilled practitioners will be capable of selecting suitable vectors and mammalian cells for making any activatable protein described herein.

In some embodiments, the activatable protein may be made biosynthetically using recombinant DNA technology and expression in eukaryotic or prokaryotic species.

### Cells

In some aspects, the present disclosure provides recombinant host cells comprising any of the vectors or nucleic acids described herein. The cells may be used to produce the activatable molecules (e.g., activatable antibodies) described herein. In some embodiments, the cell may be an animal cell, a mammalian cell (e.g., a human cell), a rodent cell (e.g., a mouse cell, a rat cell, a hamster cell, or a guinea pig cell), a non-human primate cell, an insect cell, a bacterial cell, a fungal cell, or a plant cell. In some embodiments, the cell may be a eukaryotic cell. As used herein, the term “eukaryotic cell” refers to a cell having a distinct, membrane-bound nucleus. Such cells may include, for example, mammalian (e.g., rodent, non-human primate, or human), insect, fungal, or plant cells. In some embodiments, the eukaryotic cell is a yeast cell, such as Saccharomyces cerevisiae. In some embodiments, the eukaryotic cell is a higher eukaryote, such as mammalian, avian, plant, or insect cells. Non-limiting examples of mammalian cells include Chinese hamster ovary (CHO) cells and human embryonic kidney cells (e.g., HEK293 cells). In some embodiments, the cell may be a prokaryotic cell.

Methods of introducing nucleic acids and vectors (e.g., any of the vectors or any of the sets of vectors described herein) into a cell are known in the art. Examples of methods that can be used to introducing a nucleic acid into a cell include: lipofection, transfection, calcium phosphate transfection, cationic polymer transfection, viral transduction (e.g., adenoviral transduction, lentiviral transduction), nanoparticle transfection, and electroporation.

In some embodiments, the introducing step includes introducing into a cell a vector (e.g., any of the vectors or sets of vectors described herein) including a nucleic acid encoding the monomers that make up any activatable protein described herein.

### Compositions and Kits

The present disclosure also provides compositions and kits comprising the activatable molecules (e.g., activatable antibodies) described herein. The compositions and kits may further comprise one or more excipients, carriers, reagents, instructions needed for the use of the activatable proteins.

In some embodiments, the compositions may be pharmaceutical compositions, which comprise the activatable proteins, derivatives, fragments, analogs and homologs thereof. The pharmaceutical compositions may comprise the activatable protein and a pharmaceutically acceptable carrier. As used herein, the term “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Suitable examples of such carriers or diluents include water, saline, ringer's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition may be formulated to be compatible with its intended route of administration. Examples of routes of administration include parenteral, e.g., intravenous, intradermal, subcutaneous, oral (e.g., inhalation), transdermal (e.g., topical), transmucosal, and rectal administration. Solutions or suspensions used for parenteral, intradermal, or subcutaneous application may include one or more of the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH may be adjusted with acids or bases, such as hydrochloric acid or sodium hydroxide. The parenteral preparation can be enclosed in ampoules, disposable syringes or multiple dose vials made of glass or plastic. In some, any of the activatable proteins described herein are prepared with carriers that protect against rapid elimination from the body, e.g., sustained and controlled release formulations, including implants and microencapsulated delivery systems. Biodegradable, biocompatible polymers can be used, e.g., ethylene vinyl acetate, polyanhydrides, polyglycolic acid, collagen, polyorthoesters, polylactic-co-glycolic acid, and polylactic acid. Methods for preparation of such pharmaceutical compositions and formulations are apparent to those skilled in the art. For example, the activatable proteins may be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles, and nanocapsules) or in macroemulsions.

Sustained-release preparations may be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides, copolymers of L-glutamic acid and y ethyl-L-glutamate, non-degradable ethylene-vinyl acetate, degradable lactic acid-glycolic acid copolymers (e.g., injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods.

In some embodiments, pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). The composition may be sterile and should be fluid and of a viscosity that facilitates easy syringeability. It may be stable under the conditions of manufacture and storage and preserved against the contaminating action of microorganisms such as bacteria and fungi. The carrier may be a solvent or dispersion medium containing, for example, water, ethanol, polyol (for example, glycerol, propylene glycol, and liquid polyethylene glycol, and the like), and suitable mixtures thereof. For dispersed particulate compositions, the proper fluidity can be maintained, for example, by the use of a coating on the particles such as lecithin, and by the maintenance of the required particle size in the case of dispersion and by the use of surfactants. In some embodiments, the pharmaceutical compositions may further comprise one or more antibacterial and/or antibacterial and antifungal agents, for example, parabens, chlorobutanol, phenol, ascorbic acid, thimerosal, and the like. In some embodiments, isotonic agents, for example, sugars, polyalcohols such as manitol, sorbitol, and the like, as well as salts, such as, for example, sodium chloride and the like may be included in the composition. Prolonged absorption of the injectable compositions may be brought about by including in the composition an agent that delays absorption, for example, aluminum monostearate and gelatin.

In some embodiments, the pharmaceutical composition may comprise a sterile injectable solution. Sterile injectable solutions may be prepared by incorporating the active compound in the required amount in an appropriate solvent with one or a combination of ingredients enumerated above, as required, followed by filtered sterilization. Generally, dispersions may be prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

In some embodiments, the pharmaceutical composition may comprise an oral composition. Oral compositions may include an inert diluent or an edible carrier. They may be enclosed in gelatin capsules or compressed into tablets. For the purpose of oral therapeutic administration, the active compound may be incorporated with excipients and used in the form of tablets, troches, or capsules. Oral compositions may also be prepared using a fluid carrier for use as a mouthwash, wherein the compound in the fluid carrier is applied orally and swished and expectorated or swallowed. Pharmaceutically compatible binding agents, and/or adjuvant materials may be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

In some embodiments, the pharmaceutical composition may be formulized for administration by inhalation. For example, the compounds may be delivered in the form of an aerosol spray from pressured container or dispenser that contains a suitable propellant, e.g., a gas such as carbon dioxide, or a nebulizer.

In some embodiments, the pharmaceutical composition may be formulized for systemic administration. For example, systemic administration may be by intravenous, as well by transmucosal or transdermal means. For transmucosal or transdermal administration, penetrants appropriate to the barrier to be permeated may be used in the formulation. Such penetrants are generally known in the art, and include, for example, for transmucosal administration, detergents, bile salts, and fusidic acid derivatives. Transmucosal administration may be accomplished through the use of nasal sprays or suppositories. For transdermal administration, the active compounds may be formulated into ointments, salves, gels, or creams as generally known in the art.

In some embodiments, the pharmaceutical composition may be prepared in the form of suppositories (e.g., with conventional suppository bases such as cocoa butter and other glycerides) or retention enemas for rectal delivery.

In one embodiment, the pharmaceutical composition may be prepared with carriers that protect the compound against rapid elimination from the body, such as a controlled release formulation, including implants and microencapsulated delivery systems. Biodegradable, biocompatible polymers may be used, such as ethylene vinyl acetate, polyanhydrides, polyglycolic acid, collagen, polyorthoesters, polylactic-co-glycolic acid and polylactic acid. Methods for preparation of such formulations will be apparent to those skilled in the art.

It may be advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the disclosure may be dictated by and directly dependent on the unique characteristics of the active compound and the particular therapeutic effect to be achieved, and the limitations inherent in the art of compounding such an active compound for the treatment of individuals.

In some embodiments, the compositions (e.g., pharmaceutical compositions) may be included in a container, vial, syringe, injector pen, pack, or dispenser, optionally together with instructions for administration.

Also provided herein are kits that include any of the activatable proteins described herein, any of the compositions that include any of the activatable proteins described herein, or any of the pharmaceutical compositions that include any of the activatable proteins described herein. Also provided are kits that include one or more second therapeutic agent(s) in addition to an activatable protein described herein. The second therapeutic agent(s) may be provided in a dosage administration form that is separate from the activatable proteins. Alternatively, the second therapeutic agent(s) may be formulated together with the activatable proteins.

Any of the kits described herein can include instructions for using any of the compositions (e.g., pharmaceutical compositions) and/or any of the activatable proteins described herein. In some embodiments, the kits can include instructions for performing any of the methods described herein. In some embodiments, the kits can include at least one dose of any of the compositions (e.g., pharmaceutical compositions) described herein. In some embodiments, the kits can provide a syringe for administering any of the pharmaceutical compositions described herein.

Also provided herein are activatable proteins produced by any of the methods described herein. Also provided are compositions (e.g., pharmaceutical compositions) that comprise any of the activatable proteins produced by any of the methods described herein. Also provided herein are kits that include at least one dose of any of the compositions (e.g., pharmaceutical compositions) described herein.

### Methods of Producing Activatable Molecules

Provided herein are methods of producing any activatable molecule (e.g., activatable protein) described herein that include: (a) culturing any of the recombinant host cells described herein in a liquid culture medium under conditions sufficient to produce the activatable molecule; and (b) recovering the activatable molecule from the host cell and/or the liquid culture medium.

Methods of culturing cells are well known in the art. In some embodiments, cells may be maintained in vitro under conditions that favor cell proliferation, cell differentiation and cell growth. For example, the recombinant cells may be cultured by contacting a cell (e.g., any of the cells described herein) with a cell culture medium that includes the necessary growth factors and supplements sufficient to support cell viability and growth.

In some embodiments, the method may further include isolating the recovered activatable protein. The isolation of the activatable protein may be performed using any separation or purification technique for separating protein species, e.g., affinity tag-based protein purification (e.g., polyhistidine (His) tag, glutathione-S-transferase tag, and the like), ammonium sulfate precipitation, polyethylene glycol precipitation, size exclusion chromatography, ligand-affinity chromatography (e.g., Protein A chromatography), ion-exchange chromatography (e.g., anion or cation), hydrophobic interaction chromatography, and the like.

Compositions and methods described herein may involve use of non-reducing or partially-reducing conditions that allow disulfide bonds to form between the MM and the TB of the activatable proteins.

In some embodiments, the method further includes formulating the isolated activatable protein into a pharmaceutical composition. Various formulations are known in the art and are described herein. Any isolated activatable protein described herein can be formulated for any route of administration (e.g., intravenous, intratumoral, subcutaneous, intradermal, oral (e.g., inhalation), transdermal (e.g., topical), transmucosal, or intramuscular).

### Methods of Using Activatable Molecules

In some aspects, the present disclosure further provides methods of using the activatable molecules (e.g., activatable antibodies) herein. In some embodiments, the present disclosure provides methods of the treating a disease (e.g., a cancer (e.g., any of the cancers described herein)) in a subject including administering a therapeutically effective amount of any of the activatable proteins described herein to the subject. In some embodiments, the disclosure provides methods of preventing, delaying the progression of, treating, alleviating a symptom of, or otherwise ameliorating disease in a subject by administering a therapeutically effective amount of an activatable protein described herein to a subject in need thereof. The term “treatment” refers to ameliorating at least one symptom of a disorder. In some embodiments, the disorder being treated may be a cancer or autoimmune disease or to ameliorate at least one symptom of a cancer or autoimmune disease. As used herein, the term “subject” refers to any mammal. In some embodiments, the subject is a feline (e.g., a cat), a canine (e.g., a dog), an equine (e.g., a horse), a rabbit, a pig, a rodent (e.g., a mouse, a rat, a hamster or a guinea pig), a non-human primate (e.g., a simian (e.g., a monkey (e.g., a baboon, a marmoset), or an ape (e.g., a chimpanzee, a gorilla, an orangutan, or a gibbon)), or a human. In some embodiments, the subject is a human. The terms subject and patient are used interchangeably herein. In some embodiments, the subject has been previously identified or diagnosed as having the disease (e.g., cancer (e.g., any of the cancers described herein)).

In some embodiments, a subject can be identified as having a mutation in a HER2 gene that increase the expression and/or activity of HER2 in a mammalian cell (e.g., any of the mammalian cells described herein). For example, a mutation in a HER2 gene that increases the expression and/or activity of HER2 in a mammalian cell can be a gene duplication, a mutation that results in the expression of a HER2 having one or more amino acid substitutions (E.g., one or more amino acid substitutions selected from the group consisting of: G309A, G309E, S310F, R678Q, L755S, L755W, I767M, D769H, D769Y, V777L, Y835F, V842I, R896C, and G1201V) (as compared to the wild type protein). See, e.g., Weigelt and Reis-Filho, Cancer Discov. 2013, 3(2): 145-147.

Non-limiting examples of methods of detecting a HER2 associated disease in a subject include: immunohistochemistry, fluorescent in situ hybridization (FISH), chromogenic in situ hybridization (CISH). See, e.g., Yan et al., Cancer Metastasis Rev. 2015, 34: 157-164.

A therapeutically effective amount of an activatable protein of the disclosure relates generally to the amount needed to achieve a therapeutic objective. As noted above, this may be a binding interaction between the antibody and its target antigens that, in certain cases, interferes with the functioning of the targets. The amount required to be administered will furthermore depend on the binding affinity of the activatable protein for its specific target, and will also depend on the rate at which an administered activatable protein is depleted from the free volume other subject to which it is administered. Common ranges for therapeutically effective dosing of an activatable protein of the disclosure may be, by way of nonlimiting example, from about 0.001, 0.01, 0.1, 0.3, 0.5, 0.7, 0.8, 0.9, 1, 2, 3, 4, 5, 6, 7, 8, 9, 10, 15, 20, 25, 30, 35, 40, 45, 50 mg/kg body weight or higher. The structure of the activatable protein of the present disclosure makes it possible to reduce the dosage of the activatable protein that is administered to a subject compared to conventional activatable antibodies and compared to conventional antibodies. For example, the administered dose on a unit dosage basis or total dosage over a dosage regimen period may be reduced by 10, 20, 30, 40, or 50% compared to the corresponding dose of a corresponding conventional activatable protein or a corresponding conventional antibody.

Common dosing frequencies may range, for example, from once or twice daily, weekly, biweekly, or monthly.

Efficaciousness of treatment is determined in association with any known method for diagnosing or treating the particular disorder. Methods for the screening of activatable proteins that possess the desired specificity include, but are not limited to, enzyme linked immunosorbent assay (ELISA) and other immunologically mediated techniques known within the art.

In another embodiment, an activatable protein directed two or more targets are used in methods known within the art relating to the localization and/or quantitation of the targets (e.g., for use in measuring levels of one or more of the targets within appropriate physiological samples, for use in diagnostic methods, for use in imaging the protein, and the like). In a given embodiment, an activatable protein directed two or more targets, or a derivative, fragment, analog or homolog thereof, that contain the antibody derived antigen binding domain, are utilized as pharmacologically active compounds (referred to hereinafter as “Therapeutics”).

The activatable protein used in any of the embodiments of these methods and uses may be administered at any stage of the disease. For example, such an activatable protein may be administered to a patient suffering cancer of any stage, from early to metastatic. In some embodiments, the activatable protein and formulations thereof may be administered to a subject suffering from or susceptible to a disease or disorder associated with aberrant target expression and/or activity.

A subject suffering from or susceptible to a disease or disorder associated with aberrant target expression and/or activity may be identified using any of a variety of methods known in the art. For example, subjects suffering from cancer or other neoplastic condition may be identified using any of a variety of clinical and/or laboratory tests such as, physical examination and blood, urine and/or stool analysis to evaluate health status. For example, subjects suffering from inflammation and/or an inflammatory disorder may be identified using any of a variety of clinical and/or laboratory tests such as physical examination and/or bodily fluid analysis, e.g., blood, urine and/or stool analysis, to evaluate health status.

In some embodiments, administration of an activatable protein to a patient suffering from a disease or disorder associated with aberrant target expression and/or activity may be considered successful if any of a variety of laboratory or clinical objectives is achieved. For example, administration of an activatable protein to a patient suffering from a disease or disorder associated with aberrant target expression and/or activity may be considered successful if one or more of the symptoms associated with the disease or disorder is alleviated, reduced, inhibited or does not progress to a further, i.e., worse, state. Administration of an activatable protein to a patient suffering from a disease or disorder associated with aberrant target expression and/or activity may be considered successful if the disease or disorder enters remission or does not progress to a further, i.e., worse, state.

As used herein, the term “treat” includes reducing the severity, frequency or the number of one or more (e.g., 1, 2, 3, 4, or 5) symptoms or signs of a disease (e.g., a cancer (e.g., any of the cancers described herein)) in the subject (e.g., any of the subjects described herein). In some embodiments where the disease is cancer, treating results in reducing cancer growth, inhibiting cancer progression, inhibiting cancer metastasis, or reducing the risk of cancer recurrence in a subject having cancer.

In some embodiments, the disease may be a cancer. In some embodiments, the subject may have been identified or diagnosed as having a cancer. Examples of cancer include: solid tumor, hematological tumor, sarcoma, osteosarcoma, glioblastoma, neuroblastoma, melanoma, rhabdomyosarcoma, Ewing sarcoma, osteosarcoma, B-cell neoplasms, multiple myeloma, a lymphoma (e.g., B-cell lymphoma, B-cell non-Hodgkin's lymphoma, Hodgkin's lymphoma, cutaneous T-cell lymphoma), a leukemia (e.g., hairy cell leukemia, chronic lymphocytic leukemia (CLL), acute myeloid leukemia (AML), chronic myeloid leukemia (CML), acute lymphocytic leukemia (ALL)), myelodysplastic syndromes (MDS), Kaposi sarcoma, retinoblastoma, stomach cancer, urothelial carcinoma, lung cancer, renal cell carcinoma, gastric and esophageal cancer, pancreatic cancer, prostate cancer, brain cancer, colon cancer, bone cancer, lung cancer, breast cancer, colorectal cancer, ovarian cancer, nasopharyngeal adenocarcimoa, non-small cell lung carcinoma (NSCLC), squamous cell head and neck carcinoma, endometrial cancer, bladder cancer, cervical cancer, liver cancer, and hepatocellular carcinoma. In some embodiments, the cancer is a lymphoma. In some embodiments, the lymphoma is Burkitt's lymphoma. In some aspects, the subject has been identified or diagnosed as having familial cancer syndromes such as Li Fraumeni Syndrome, Familial Breast-Ovarian Cancer (BRCA1 or BRAC2 mutations) Syndromes, and others. The disclosed methods are also useful in treating non-solid cancers. Exemplary solid tumors include malignancies (e.g., sarcomas, adenocarcinomas, and carcinomas) of the various organ systems, such as those of lung, breast, lymphoid, gastrointestinal (e.g., colon), and genitourinary (e.g., renal, urothelial, or testicular tumors) tracts, pharynx, prostate, and ovary. Exemplary adenocarcinomas include colorectal cancers, renal-cell carcinoma, liver cancer, non-small cell carcinoma of the lung, and cancer of the small intestine. Further examples of cancers that may be treated by the compositions and methods herein include: Acute Lymphoblastic Leukemia, Adult; Acute Lymphoblastic Leukemia, Childhood; Acute Myeloid Leukemia, Adult; Adrenocortical Carcinoma; Adrenocortical Carcinoma, Childhood; AIDS-Related Lymphoma; AIDS-Related Malignancies; Anal Cancer; Astrocytoma, Childhood Cerebellar; Astrocytoma, Childhood Cerebral; Bile Duct Cancer, Extrahepatic; Bladder Cancer; Bladder Cancer, Childhood; Bone Cancer, Osteosarcoma/Malignant Fibrous Histiocytoma; Brain Stem Glioma, Childhood; Brain Tumor, Adult; Brain Tumor, Brain Stem Glioma, Childhood; Brain Tumor, Cerebellar Astrocytoma, Childhood; Brain Tumor, Cerebral Astrocytoma/Malignant Glioma, Childhood; Brain Tumor, Ependymoma, Childhood; Brain Tumor, Medulloblastoma, Childhood; Brain Tumor, Supratentorial Primitive Neuroectodermal Tumors, Childhood; Brain Tumor, Visual Pathway and Hypothalamic Glioma, Childhood; Brain Tumor, Childhood (Other); Breast Cancer; Breast Cancer and Pregnancy; Breast Cancer, Childhood; Breast Cancer, Male; Bronchial Adenomas/Carcinoids, Childhood; Carcinoid Tumor, Childhood; Carcinoid Tumor, Gastrointestinal; Carcinoma, Adrenocortical; Carcinoma, Islet Cell; Carcinoma of Unknown Primary; Central Nervous System Lymphoma, Primary; Cerebellar Astrocytoma, Childhood; Cerebral Astrocytoma/Malignant Glioma, Childhood; Cervical Cancer; Childhood Cancers; Chronic Lymphocytic Leukemia; Chronic Myelogenous Leukemia; Chronic Myeloproliferative Disorders; Clear Cell Sarcoma of Tendon Sheaths; Colon Cancer; Colorectal Cancer, Childhood; Cutaneous T-Cell Lymphoma; Endometrial Cancer; Ependymoma, Childhood; Epithelial Cancer, Ovarian; Esophageal Cancer; Esophageal Cancer, Childhood; Ewing's Family of Tumors; Extracranial Germ Cell Tumor, Childhood; Extragonadal Germ Cell Tumor; Extrahepatic Bile Duct Cancer; Eye Cancer, Intraocular Melanoma; Eye Cancer, Retinoblastoma; Gallbladder Cancer; Gastric (Stomach) Cancer; Gastric (Stomach) Cancer, Childhood; Gastrointestinal Carcinoid Tumor; Germ Cell Tumor, Extracranial, Childhood; Germ Cell Tumor, Extragonadal; Germ Cell Tumor, Ovarian; Gestational Trophoblastic Tumor; Glioma, Childhood Brain Stem; Glioma, Childhood Visual Pathway and Hypothalamic; Hairy Cell Leukemia; Head and Neck Cancer; Hepatocellular (Liver) Cancer, Adult (Primary); Hepatocellular (Liver) Cancer, Childhood (Primary); Hodgkin's Lymphoma, Adult; Hodgkin's Lymphoma, Childhood; Hodgkin's Lymphoma During Pregnancy; Hypopharyngeal Cancer; Hypothalamic and Visual Pathway Glioma, Childhood; Intraocular Melanoma; Islet Cell Carcinoma (Endocrine Pancreas); Kaposi's Sarcoma; Kidney Cancer; Laryngeal Cancer; Laryngeal Cancer, Childhood; Leukemia, Acute Lymphoblastic, Adult; Leukemia, Acute Lymphoblastic, Childhood; Leukemia, Acute Myeloid, Adult; Leukemia, Acute Myeloid, Childhood; Leukemia, Chronic Lymphocytic; Leukemia, Chronic Myelogenous; Leukemia, Hairy Cell; Lip and Oral Cavity Cancer; Liver Cancer, Adult (Primary); Liver Cancer, Childhood (Primary); Lung Cancer, Non-Small Cell; Lung Cancer, Small Cell; Lymphoblastic Leukemia, Adult Acute; Lymphoblastic Leukemia, Childhood Acute; Lymphocytic Leukemia, Chronic; Lymphoma, AIDS-Related; Lymphoma, Central Nervous System (Primary); Lymphoma, Cutaneous T-Cell; Lymphoma, Hodgkin's, Adult; Lymphoma, Hodgkin's, Childhood; Lymphoma, Hodgkin's During Pregnancy; Lymphoma, Non-Hodgkin's, Adult; Lymphoma, Non-Hodgkin's, Childhood; Lymphoma, Non-Hodgkin's During Pregnancy; Lymphoma, Primary Central Nervous System; Macroglobulinemia, Waldenstrom's; Male Breast Cancer; Malignant Mesothelioma, Adult; Malignant Mesothelioma, Childhood; Malignant Thymoma; Medulloblastoma, Childhood; Melanoma; Melanoma, Intraocular; Merkel Cell Carcinoma; Mesothelioma, Malignant; Metastatic Squamous Neck Cancer with Occult Primary; Multiple Endocrine Neoplasia Syndrome, Childhood; Multiple Myeloma/Plasma Cell Neoplasm; Mycosis Fungoides; Myelodysplastic Syndromes; Myelogenous Leukemia, Chronic; Myeloid Leukemia, Childhood Acute; Myeloma, Multiple; Myeloproliferative Disorders, Chronic; Nasal Cavity and Paranasal Sinus Cancer; Nasopharyngeal Cancer; Nasopharyngeal Cancer, Childhood; Neuroblastoma; Non-Hodgkin's Lymphoma, Adult; Non-Hodgkin's Lymphoma, Childhood; Non-Hodgkin's Lymphoma During Pregnancy; Non-Small Cell Lung Cancer; Oral Cancer, Childhood; Oral Cavity and Lip Cancer; Oropharyngeal Cancer; Osteosarcoma/Malignant Fibrous Histiocytoma of Bone; Ovarian Cancer, Childhood; Ovarian Epithelial Cancer; Ovarian Germ Cell Tumor; Ovarian Low Malignant Potential Tumor; Pancreatic Cancer; Pancreatic Cancer, Childhood; Pancreatic Cancer, Islet Cell; Paranasal Sinus and Nasal Cavity Cancer; Parathyroid Cancer; Penile Cancer; Pheochromocytoma; Pineal and Supratentorial Primitive Neuroectodermal Tumors, Childhood; Pituitary Tumor; Plasma Cell Neoplasm/Multiple Myeloma; Pleuropulmonary Blastoma; Pregnancy and Breast Cancer; Pregnancy and Hodgkin's Lymphoma; Pregnancy and Non-Hodgkin's Lymphoma; Primary Central Nervous System Lymphoma; Primary Liver Cancer, Adult; Primary Liver Cancer, Childhood; Prostate Cancer; Rectal Cancer; Renal Cell (Kidney) Cancer; Renal Cell Cancer, Childhood; Renal Pelvis and Ureter, Transitional Cell Cancer; Retinoblastoma; Rhabdomyosarcoma, Childhood; Salivary Gland Cancer; Salivary Gland Cancer, Childhood; Sarcoma, Ewing's Family of Tumors; Sarcoma, Kaposi's; Sarcoma (Osteosarcoma)/Malignant Fibrous Histiocytoma of Bone; Sarcoma, Rhabdomyosarcoma, Childhood; Sarcoma, Soft Tissue, Adult; Sarcoma, Soft Tissue, Childhood; Sezary Syndrome; Skin Cancer; Skin Cancer, Childhood; Skin Cancer (Melanoma); Skin Carcinoma, Merkel Cell; Small Cell Lung Cancer; Small Intestine Cancer; Soft Tissue Sarcoma, Adult; Soft Tissue Sarcoma, Childhood; Squamous Neck Cancer with Occult Primary, Metastatic; Stomach (Gastric) Cancer; Stomach (Gastric) Cancer, Childhood; Supratentorial Primitive Neuroectodermal Tumors, Childhood; T-Cell Lymphoma, Cutaneous; Testicular Cancer; Thymoma, Childhood; Thymoma, Malignant; Thyroid Cancer; Thyroid Cancer, Childhood; Transitional Cell Cancer of the Renal Pelvis and Ureter; Trophoblastic Tumor, Gestational; Unknown Primary Site, Cancer of, Childhood; Unusual Cancers of Childhood; Ureter and Renal Pelvis, Transitional Cell Cancer; Urethral Cancer; Uterine Sarcoma; Vaginal Cancer; Visual Pathway and Hypothalamic Glioma, Childhood; Vulvar Cancer; Waldenstrom's Macro globulinemia; Wilms' Tumor; diffuse large B-cell lymphoma (DLBCL); and mantle cell lymphoma (MCL). Metastases of the aforementioned cancers may also be treated or prevented in accordance with the methods described herein.

In some embodiments, the disease may be an autoimmune disease or condition. In some embodiments, the subject may have been identified or diagnosed as having an autoimmune disease or condition or is at heightened risk of developing an autoimmune disease or condition. Examples of autoimmune diseases include Type 1 diabetes, Rheumatoid arthritis (RA), Psoriasis/psoriatic arthritis, Multiple sclerosis, Systemic lupus erythematosus, Inflammatory bowel disease (e.g., Crohn's disease, ulcerative colitis), Addison's disease, Graves' disease, Sjögren's syndrome, Hashimoto's thyroiditis, Myasthenia gravis, Autoimmune vasculitis, Pernicious anemia, Celiac disease), infectious disease (e.g., Chickenpox, Common cold, Diphtheria, E. coli, Giardiasis, HIV/AIDS, Infectious mononucleosis, Influenza (flu), Lyme disease, Malaria, Measles, Meningitis, Mumps, Poliomyelitis (polio), Pneumonia, Rocky mountain spotted fever, Rubella (German measles), Salmonella infections, Severe acute respiratory syndrome (SARS), Sexually transmitted diseases, Shingles (herpes zoster), Tetanus, Toxic shock syndrome, Tuberculosis, Viral hepatitis, West Nile virus, Whooping cough (pertussis)), chronic inflammation, or transplant rejection (e.g., in kidney, liver, or heart transplantation), autoimmune diseases, infectious disease, chronic inflammation, or transplant rejection.

In some embodiments, the methods herein may result in a reduction in the number, severity, or frequency of one or more symptoms of the cancer in the subject (e.g., as compared to the number, severity, or frequency of the one or more symptoms of the cancer in the subject prior to treatment).

The methods may further comprise administering to a subject one or more additional agents.

In some embodiments, the activatable protein may be administered during and/or after treatment in combination with one or more additional agents. In some embodiments, the activatable protein may be formulated into a single therapeutic composition, and the activatable protein and additional agent(s) may be administered simultaneously. Alternatively, the activatable protein and additional agent(s) may be separate from each other, e.g., each is formulated into a separate therapeutic composition, and the activatable protein and the additional agent are administered simultaneously, or the activatable protein and the additional agent are administered at different times during a treatment regimen. For example, the activatable protein may be administered prior to the administration of the additional agent, subsequent to the administration of the additional agent, or in an alternating fashion. The activatable protein and additional agent(s) may be administered in single doses or in multiple doses.

One of more of the activatable proteins herein may be co-formulated with, and/or co-administered with, one or more anti-inflammatory drugs, immunosuppressants, or metabolic or enzymatic inhibitors. In some embodiments, one or more activatable proteins herein may be combined with one or more activatable proteins of other types (e.g., activatable proteins that do not have EM or activatable proteins whose activated forms comprise an EM).

The present disclosure also provides methods of detecting presence or absence of a cleaving agent and/or the target in a subject or a sample. Such methods may comprise (i) contacting a subject or biological sample with an activatable protein, wherein the activatable protein includes a detectable label that is positioned on a portion of the activatable protein that is released following cleavage of the CM and (ii) measuring a level of activated protein in the subject or biological sample, wherein a detectable level of activated protein in the subject or biological sample indicates that the cleaving agent, the target or both the cleaving agent and the target are absent and/or not sufficiently present in the subject or biological sample, such that the target binding and/or protease cleavage of the activatable protein cannot be detected in the subject or biological sample, and wherein a reduced detectable level of activated protein in the subject or biological sample indicates that the cleaving agent and the target are present in the subject or biological sample.

A reduced level of detectable label may be, for example, a reduction of at least 5%, 10%, 15%, 20%, 25%, 30%, 35%, 40%, 45%, 50%, 55%, 60%, 65%, 70%, 75%, 80%, 85%, 90%, or 95%, or a reduction of substantially 100%. In some embodiments, the detectable label may be conjugated to a component of the activatable protein, e.g., the TB. In some embodiments, measuring the level of activatable protein in the subject or sample may be accomplished using a secondary reagent that specifically binds to the activated protein, wherein the reagent comprises a detectable label. The secondary reagent may be an antibody comprising a detectable label.

In some embodiments, the activatable proteins may also be useful in the detection of the target in patient samples and accordingly are useful as diagnostics. For example, the activatable proteins may be used in in vitro assays, e.g., ELISA, to detect target levels in a patient sample. For example, an activatable protein may be immobilized on a solid support (e.g., the well(s) of a microtiter plate). The immobilized activatable protein may serve as a capture protein for any target that may be present in a test sample. Prior to contacting the immobilized activatable protein with a patient sample, the solid support may be rinsed and treated with a blocking agent such as milk protein or albumin to prevent nonspecific adsorption of the analyte.

In some embodiments, based on the results obtained using the activatable proteins in an in vitro diagnostic assay, the stage of a disease in a subject may be determined based on expression levels of the target protein (e.g., antigen). For a given disease, samples of blood may be taken from subjects diagnosed as being at various stages in the progression of the disease, and/or at various points in the therapeutic treatment of the disease. Using a population of samples that provides statistically significant results for each stage of progression or therapy, a range of concentrations of the target protein (e.g., antigen) that may be considered characteristic of each stage is designated.

Activatable proteins herein may also be used in diagnostic and/or imaging methods. In some embodiments, such methods may be in vitro methods. In some embodiments, such methods may be in vivo methods. In some embodiments, such methods may be in situ methods. In some embodiments, such methods may be ex vivo methods. For example, activatable proteins having a CM may be used to detect the presence or absence of an enzyme capable of cleaving the CM. Such activatable proteins may be used in diagnostics, which can include in vivo detection (e.g., qualitative or quantitative) of enzyme activity (or, in some embodiments, an environment of increased reduction potential such as that which can provide for reduction of a disulfide bond) through measured accumulation of activated antibodies (i.e., antibodies resulting from cleavage of an activatable protein) in a given cell or tissue of a given host organism. Such accumulation of activated proteins indicates not only that the tissue expresses enzymatic activity (or an increased reduction potential depending on the nature of the CM) but also that the tissue expresses target to which the activated protein binds.

For example, the CM may be selected to be a protease substrate for a protease found at the site of a tumor, at the site of a viral or bacterial infection at a biologically confined site (e.g., such as in an abscess, in an organ, and the like), and the like. The TB may be one that binds a target protein (e.g., antigen). Using methods familiar to one skilled in the art, a detectable label (e.g., a fluorescent label or radioactive label or radiotracer) may be conjugated to a TB or other region of an activatable protein. Suitable detectable labels may be discussed in the context of the above screening methods and additional specific examples are provided below. Using a TB specific to a protein or peptide of the disease state, along with a protease whose activity is elevated in the disease tissue of interest, activatable proteins may exhibit an increased rate of binding to disease tissue relative to tissues where the CM specific enzyme is not present at a detectable level or is present at a lower level than in disease tissue or is inactive (e.g., in zymogen form or in complex with an inhibitor). Since small proteins and peptides are rapidly cleared from the blood by the renal filtration system, and because the enzyme specific for the CM is not present at a detectable level (or is present at lower levels in non-disease tissues or is present in inactive conformation), accumulation of activated protein in the disease tissue may be enhanced relative to non-disease tissues.

In some embodiments, the activatable proteins may be useful for in vivo imaging where detection of the fluorescent signal in a subject, e.g., a mammal, including a human, indicates that the disease site contains the target and contains a protease that is specific for the CM of the activatable protein. The in vivo imaging may be used to identify or otherwise refine a patient population suitable for treatment with an activatable protein of the disclosure. For example, patients that test positive for both the target and a protease that cleaves the substrate in the CM of the activatable protein being tested (e.g., accumulate activated proteins at the disease site) are identified as suitable candidates for treatment with such an activatable protein comprising such a CM. Likewise, patients that test negative may be identified as suitable candidates for another form of therapy (i.e., not suitable for treatment with the activatable protein being tested). In some embodiments, such patients that test negative with respect to a first activatable protein can be tested with other activatable proteins comprising different CMs until a suitable activatable protein for treatment is identified (e.g., an activatable protein comprising a CM that is cleaved by the patient at the site of disease).

In some embodiments, in situ imaging may be useful in methods to identify which patients to treat. For example, in in situ imaging, the activatable proteins may be used to screen patient samples to identify those patients having the appropriate protease(s) and target(s) at the appropriate location, e.g., at a tumor site. In some embodiments, in situ imaging is used to identify or otherwise refine a patient population suitable for treatment with an activatable protein of the disclosure. For example, patients that test positive for both the target and a protease that cleaves the substrate in the CM of the activatable protein being tested (e.g., accumulate activated antibodies at the disease site) are identified as suitable candidates for treatment with such an activatable protein comprising such a CM. Likewise, patients that test negative for either or both of the target and the protease that cleaves the CM used in the activatable protein being tested using these methods are identified as suitable candidates for another form of therapy (i.e., not suitable for treatment with the activatable protein being tested). In some embodiments, such patients that test negative with respect to a first activatable protein can be tested with other activatable proteins comprising different CMs until a suitable activatable protein for treatment is identified (e.g., an activatable protein comprising a CM that is cleaved by the patient at the site of disease).

### EXAMPLES

The invention is further described in the following examples, which do not limit the scope of the invention described in the claims and provide proof-of-concept demonstration for the advantageous structure of the activatable macromolecules described in the present disclosure.

### Example 1: Production of Activatable Bispecific Molecules

This example shows the production of exemplary activatable bispecific proteins in which the activated protein does not comprise a half-life extending moiety (e.g., Fc domain). The dually masked activatable bispecific molecules were prepared by recombinant methods. Proteins were prepared by transforming a host cell with three polynucleotides: one having the sequence of SEQ ID NOs: 21 (for ProC1446), 22 (for ProC1447), or 23 (for ProC1448); one having the sequence of SEQ ID NO: 1; and, one having the sequence of SEQ ID NO: 18, followed by cultivation of the resulting recombinant host cells. These proteins comprise a masked Fab that specifically binds HER2 in the activated state (AB1), a masked scFv that specifically binds CD3 in the activated state (AB2), and a pair of knob and hole mutant Fc domains (EM). The structure of these activatable proteins is depicted in FIG. 6A.

The reference molecules ProC306 and ProC531 (unmasked bispecific molecules comprising a Fab that specifically binds HER2; an scFv that specifically binds CD3; and a pair of knob and hole Fc domains, in a different configuration than the exemplary activatable bispecific molecules above) were also prepared by recombinant methods.

### Example 2. Protease Treatment of Activatable Bispecific Molecules

To release the masking peptides, the dually masked activatable bispecific binding molecules prepared in Example 1 were treated overnight at 37° C. with a recombinant human protease such as matrix metalloproteinase (MMP) or uPA. Complete protease treatment was tested by reducing SDS-PAGE. Protein aliquots (2 μg) were denatured for 10 minutes at 75° C. in sample buffer (with reducing agent added, as necessary) and separated on a 4-12% NuPAGE™ Bis-Tris gel (Thermo Fisher Scientific, Waltham, MA, Catalog #NP0321) in MOPS buffer for 1 hour at 175V and visualized after staining with InstantBlue™ for 1 hour followed by destaining in water for at least 4 h.

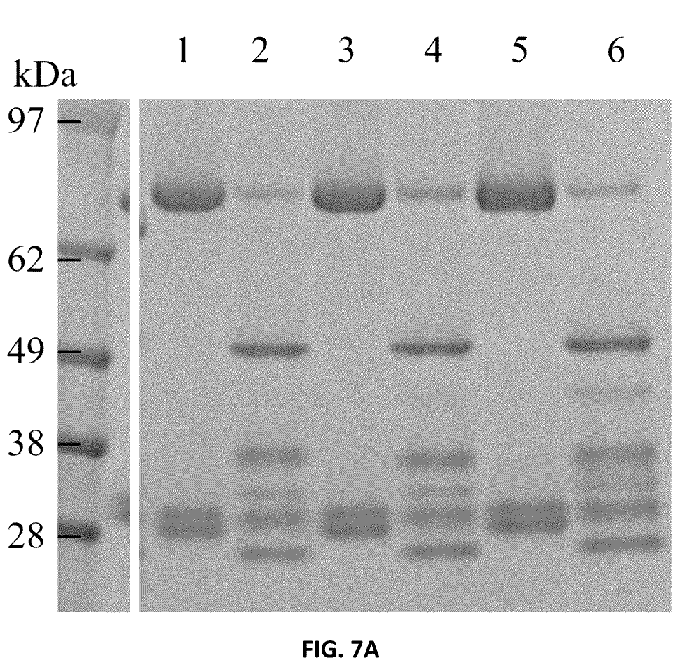

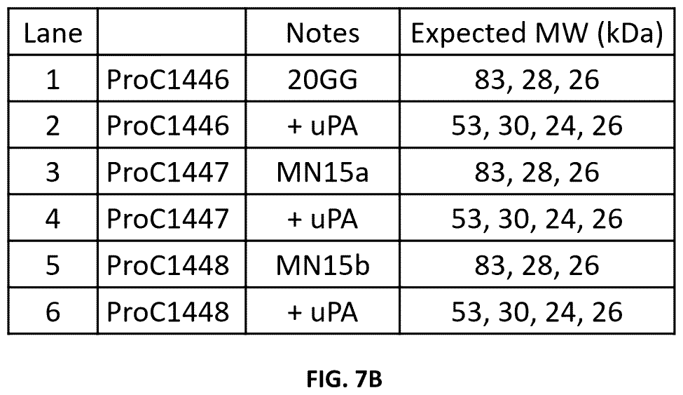

Untreated proteins were confirmed to have all three chains in the reducing gel (FIGS. 7A and 7B). After overnight protease treatment, the activation was incomplete, but a majority of the protease products were of the expected molecular weight.

### Example 3: CD3 Antigen-Binding ELISA

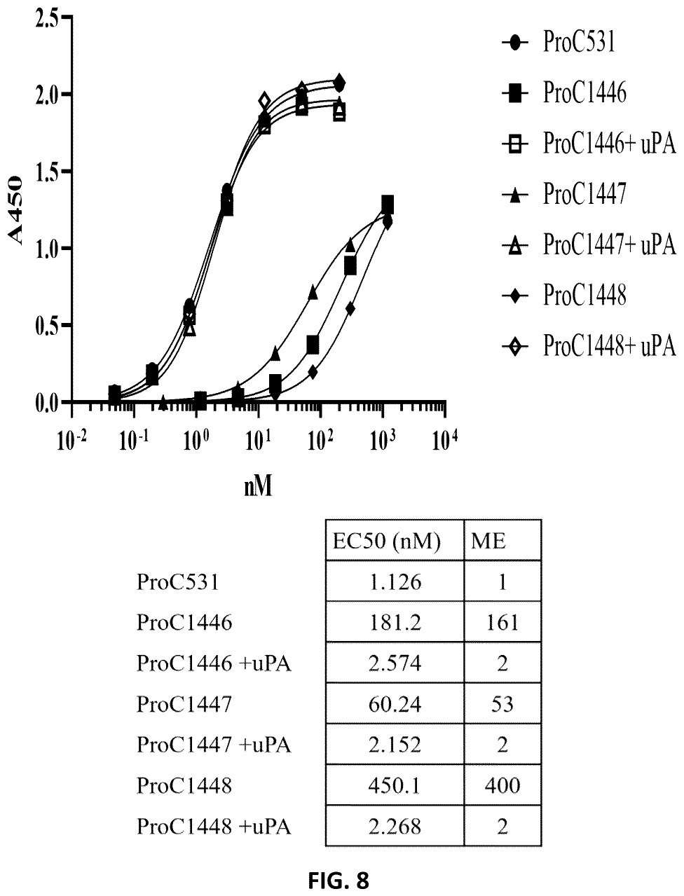

The ability of the dually masked activatable bispecific molecules prepared in Example 1 to bind CD3 antigen was tested with a CD3 binding ELISA. 100 g of CD3e-his antigen (ACRO Biosystems) dissolved in 0.05M carbonate-bicarbonate buffer was adsorbed to the wells of a 96-well micro-titer plate overnight at 4° C. Plates were washed and blocked with blocking buffer (1×PBS, pH 7.4, 0.05% Tween-20, 1% BSA). Four-fold serial dilutions were made of the dually masked activatable bispecific molecules without or with protease treatment along with the unmasked reference protein (ProC531) and applied to the antigen-coated plate. The extent of protein bound to the peptide was measured by anti-human-IgG (Fab-specific) immunodetection. A450 absorbance was measured on the plate reader. Dose-response curves were generated and EC50 values were obtained by sigmoidal fit non-linear regression using Graph Pad Prism software. The results are shown in FIG. 8. These results demonstrate that each of the three dually masked activatable bispecific molecules (ProC1446, ProC1447, and ProC1448) display reduced binding to CD3 compared to both reference bispecifc molecule that does not have the mask (ProC531), and relative to the countepart protease-treated activated molecules. The activity of the dually masked activatable bispecific molecules was recovered upon treatment with uPA to the same or nearly the same level as the counterpart reference bispecific molecule (ProC531).

### Example 4: HER2-Dependent Cytotoxicity of Dually Masked Activatable Bispecific Molecules

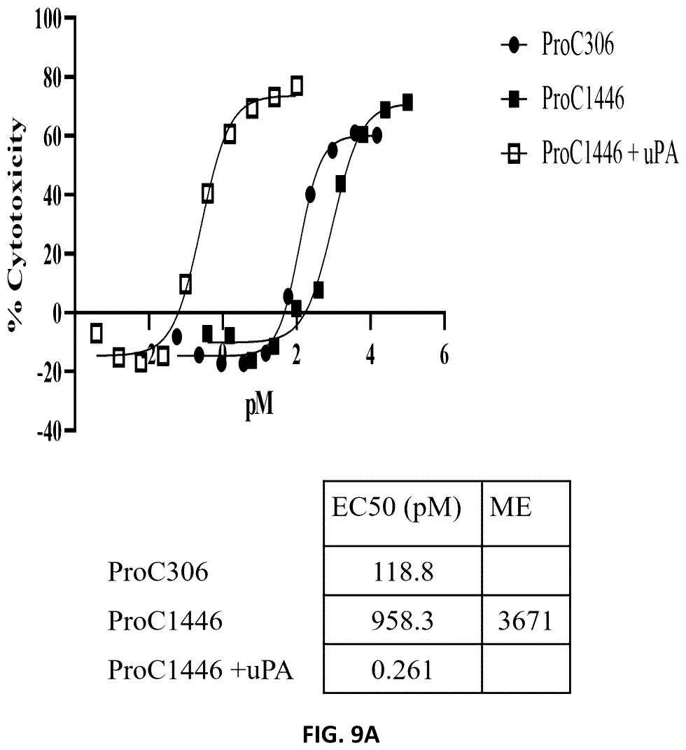

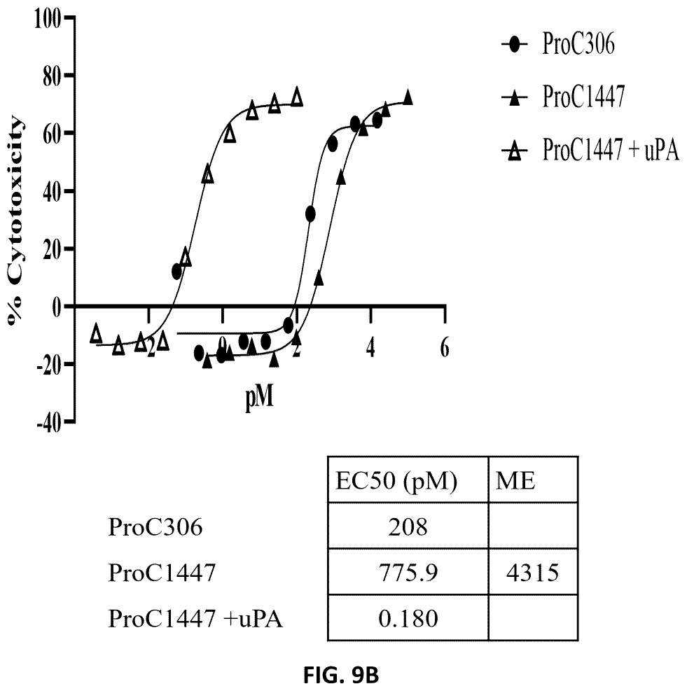

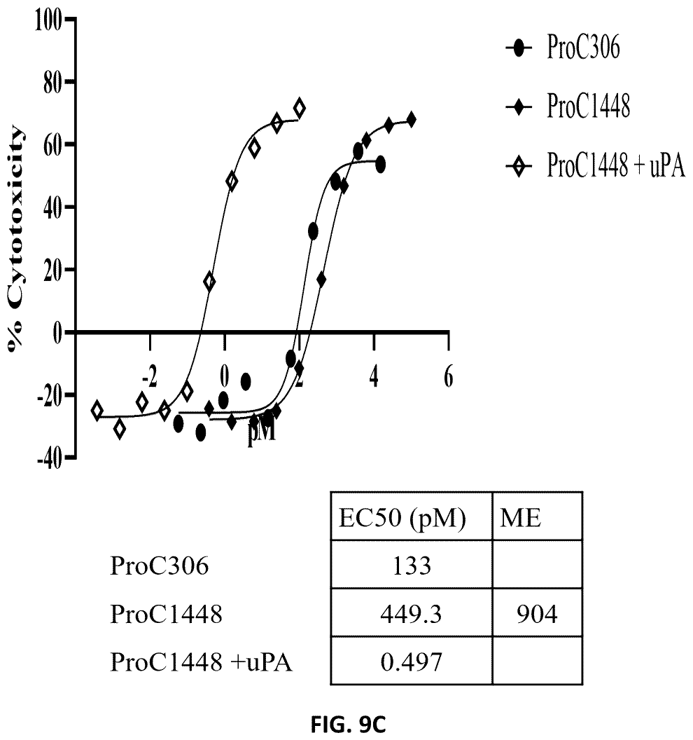

The in vitro potency of the dually-masked activatable bispecific molecules prepared in Example 1 was determined in a cytotoxicity assay. SKOV3-luc2 target cells and human PBMC effector cells (Stemcell technologies) were plated together in a co-culture in RPMI medium (Gibco cat #22400071) supplemented with 5% human serum (MP Bio cat #2930949) at 1:10 Target to Effector cell ratio. To this co-culture, titrations of intact ProC1446, ProC1447 and ProC1448, and their protease activated versions (uPA-treated ProC1446, uPA-treated ProC1447, uPA-treated ProC1448), and the unmasked reference ProC306 were added. The plate was incubated for approximately 48 hours at 37° C. and 5% CO₂. Post incubation, cytotoxicity was evaluated using ONE-Glo™ Luciferase Assay System (Promega cat #E6130) and the luminescence was measured on a plate reader (TECAN). The percent cytotoxicity was calculated as follows: (1−(RLU experimental/average RLU untreated))*100. Using GraphPad PRISM, percent cytotoxicity data was plotted and EC50 values were calculated. The results are shown in FIGS. 9A-9C. These results demonstrate that each of the three dually masked activatable bispecific molecules (ProC1446, ProC1447, and ProC1448) display reduced cytotoxicity compared to both a reference bispecific molecule that does not have the mask (ProC306), and relative to the counterpart protease-treated activated molecules. The cytotoxic activity of the activated molecules following treatment with uPA was more potent than the reference bispecific molecule that does not have the mask (ProC306).

### Example 5: Binding of Dually Masked, Bispecific Antibodies to Her2+ NCI-N87, SKOV3 and CD3ε+ Jurkat Cells

To determine if the described Her2 and CD3c masking peptides could inhibit binding in the context of a dually masked, bispecific, antibody, a flow cytometry-based binding assay was performed.

NCI-N87 (ATCC), SKOV3 (ATCC) and Jurkat (Clone E6-1, ATCC, TIB-152) cells were cultured in RPMI-1640+glutamax (Life Technologies, Catalog 72400-047), 10% Heat Inactivated-Fetal Bovine Serum (HI-FBS, Life Technologies, Catalog 10438-026) and Puromycin in case of NCI-N87 cells (Gibco, catalog A11138-03, @2 ug/ml). The following bispecific antibodies were tested: recombinantly produced activated SHL1-ProC1963, SHL2-ProC1965, ½ TCB ProC306, and their respective dually masked activatable bispecific antibodies, ProC1446 (SHL1), ProC3007 (SHL2), ProC3008 (SHL2) and ProC1441 (1/2 TCB). Two versions of the cleavable moiety (CM) present between the EM (half-life extension moiety) and C terminus were utilized, namely the CM1 in ProC3007 versus the CM2 in ProC3008.

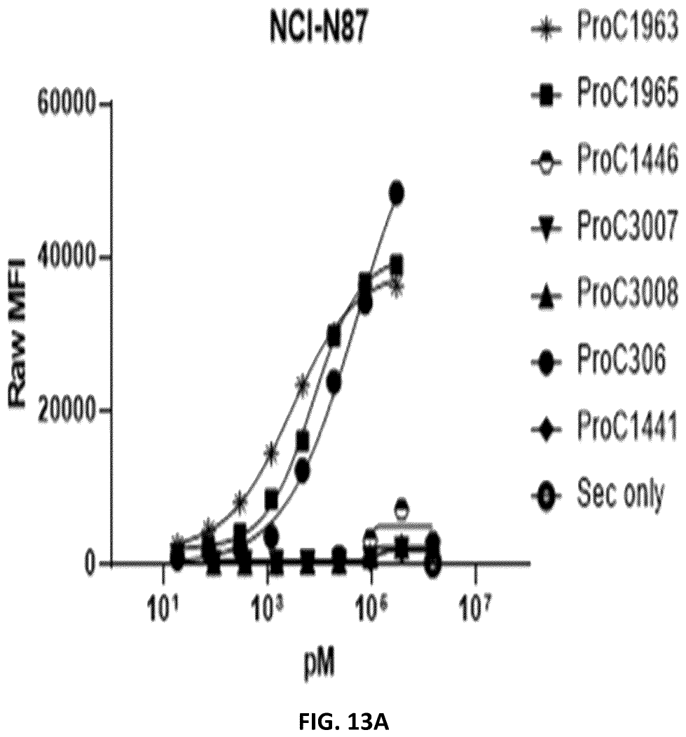

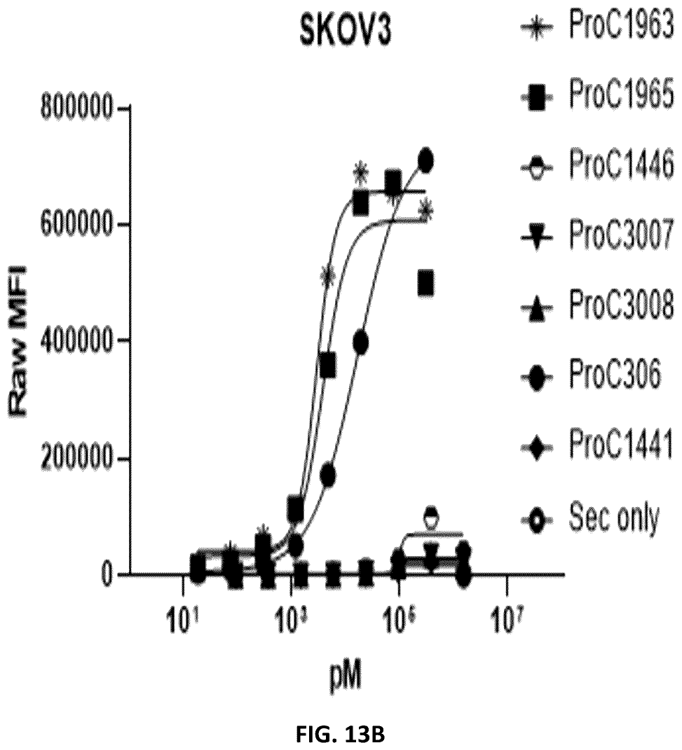

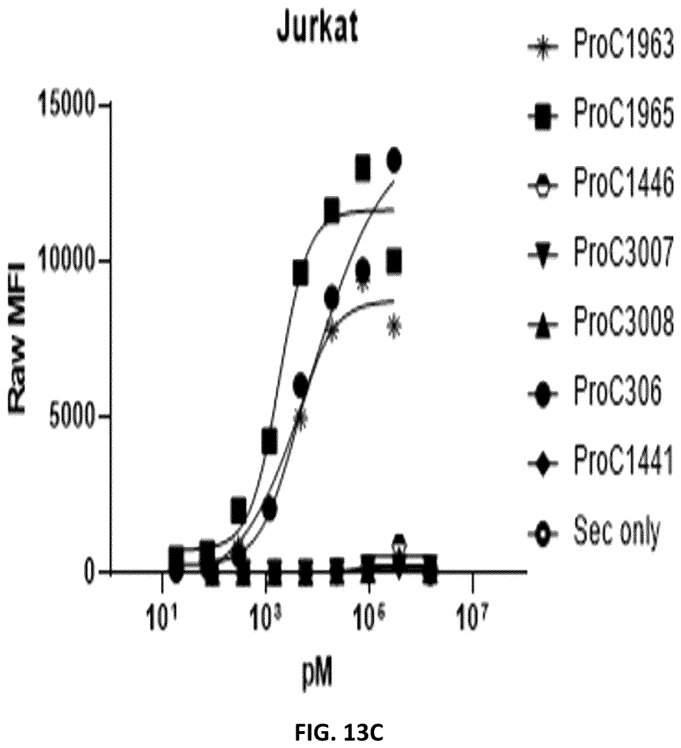

NCI-N87 and SKOV3 cells were detached with Versene™ (Life Technologies, Catalog 15040-066), washed, plated in 96 well plates at 150,000 cells/well, and re-suspended in 50 L of primary antibody (bispecific antibodies). Jurkat cells were counted and plated as described for NCI-N87 and SKOV3. Titrations of primary antibody starting at the concentrations indicated in FIG. 13A-13B followed by 3-fold serial dilutions in FACS Stain Buffer+2% FBS (BD Pharmingen, Catalog 554656) were added to the cells. Cells were incubated at 4° C. with shaking for about 1 hour, harvested, and washed with 2×200 μL of FACS Stain Buffer. Cells were resuspended in 50 μL of Alexa Fluor®647 conjugated Anti-Human IgG, F(ab′)₂ fragment specific antibody (1.5 g/ml, Jackson ImmunoResearch, catalog 109-605-097) and incubated at 4° C. with shaking for about 1 hour. Cells were harvested, washed, and resuspended in a final volume of 200 μL of FACS Stain Buffer containing 2.5 g/ml 7-AAD (BD Biosciences, Catalog 559925). Cells stained with secondary antibody alone were used as a negative control. Data were acquired on an Attune™ NxT Flow Cytometer and the median fluorescence intensity (MFI) of viable cells was calculated using FlowJo® V10.8.1. Raw MFI data was graphed in GraphPad Prism using curve fit analysis.

FIGS. 13A-13B depict binding of masked, activatable short half-life antibodies, ProC1446 (SHL1), ProC3007 (SHL2), ProC3008 (SHL2), and masked antibody, ProC1441 (1/2 TCB, not an activatable short half-life antibody) and unmasked (ProC1963 (SHL1, no mask or Fc), ProC1965, and ProC306) anti-CD3, anti-HER2 bispecific antibodies, as well as secondary antibody (“Sec only”, negative control) to NCI-N87 and SKOV3 cells (i.e., HER2 binding), respectively. FIG. 13C depicts binding of the same molecules to Jurkat cells (i.e., CD3 binding). The results indicate that all of the masked molecules exhibited reduced binding to both HER2 and CD3 relative to their corresponding unmasked forms as represented by a right shift of the binding curves (very low/no binding even at the highest concentration) of the masked molecules. EC50 values were determined from replicate experiments. The average EC50 values are provided below in Table 1.

TABLE 1
Average EC50

| | Molecule | N87 | SKOV3 | Jurkat |
|---|---|---|---|---|
| Molecule ID | Format | EC50 (pM) | EC50 (pM) | EC50 (pM) |
| ProC1965 | SHL2, | 9396.00 | 5369.50 | 2664.67 |
| | unmasked, no Fc | | | |
| ProC1963 | SHL1, | 4524.50 | 3678.50 | 4401.00 |
| | unmasked, no Fc | | | |
| ProC3007 | SHL2 | >1500000 | >1500000 | >1500000 |
| ProC3008 | SHL2 | >1500000 | >1500000 | >1500000 |
| ProC1446 | SHL1 | >1500000 | >1500000 | >1500000 |
| ProC306 | ½ TCB, | 36755.50 | 16478.50 | 9317.67 |
| | Unmasked | | | |
| ProC1441 | ½ TCB | >1500000 | >1500000 | >1500000 |

The results show that unmasked anti-HER2, anti-CD3 TCBs in the activatable short half life formats (SHL1 and SHL2) exhibited comparable CD3 and HER2 binding to the corresponding unmasked ½ TCB format. A modest trend of increased HER2 binding was observed for the activatable short half life formats relative to the ½ TCB format. The masked activatable short half-life molecules exhibited highly attenuated HER2 and CD3 binding, comparable to that observed for the masked ½ TCB molecule. The masked ½ TCB molecule (ProC1441) is a molecule as depicted on the left side of FIG. 12, but lacking the third cleavable moiety (CM3) (1205) between the EM and the Fab, meaning that the molecule is not cleavable so as to release the EM.

### Example 6: Biological Activity of Dually Masked Activable Bispecific Antibodies

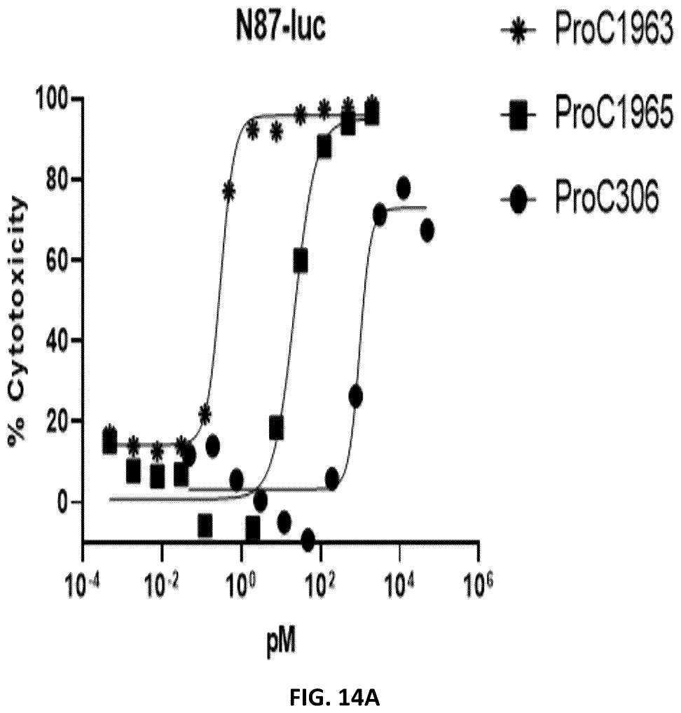

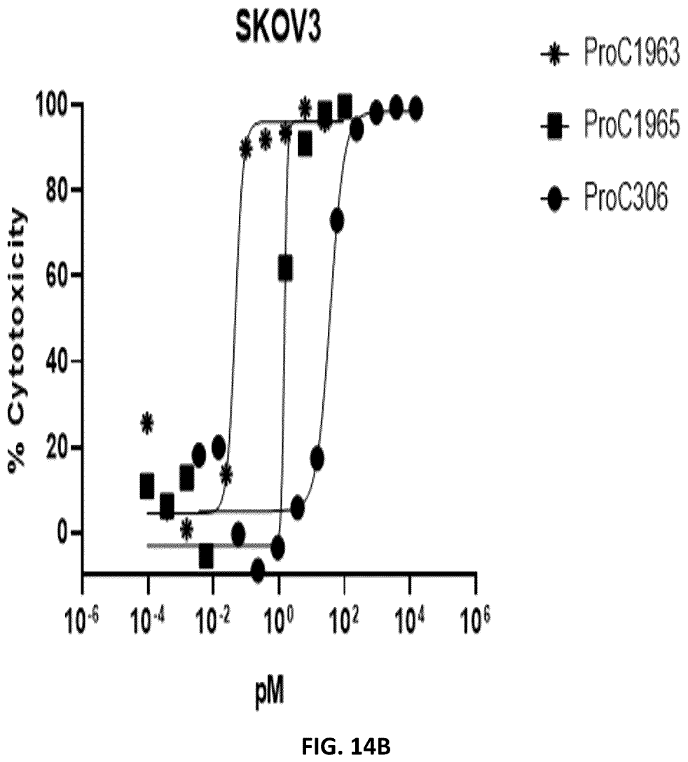

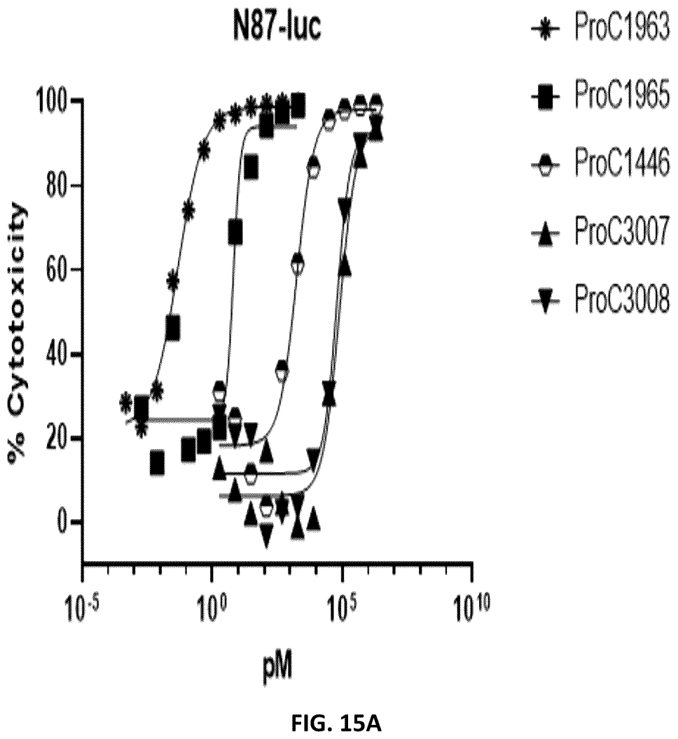

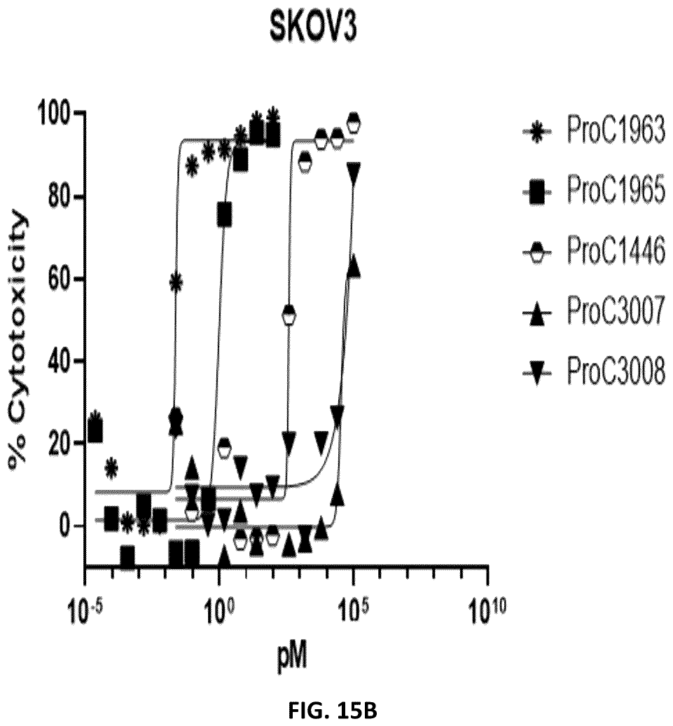

Biological activity of intact activable bispecific and recombinantly produced activated bispecific antibodies was assayed using cytotoxicity assays. Human PBMCs were purchased from HemaCare Inc, Van Nuys, CA) and co-cultured with Her2 expressing cancer cell lines NCI-N87 (ATCC) or SKOV3 (ATCC) at a ratio of 10:1 in RPMI-1640+glutamax supplemented with 5% heat-inactivated human serum (Sigma, Catalog H3667). Dose response at starting concentrations indicated in FIGS. 14A-14B and 15A-15B followed by 3 fold serial dilutions in the co-culture media of intact ProC1446 (SHL1), ProC3007 (SHL2), ProC3008 (SHL2), ProC1441 (½ TCB) and recombinantly produced activated bispecific antibodies, ProC1963 (SHL1), ProC1965 (SHL2), and ProC306 (½ TCB) were tested. After 48 hours, cytotoxicity was evaluated using the ONE-Glo™ Luciferase Assay System (Promega, Madison, WI Catalog E6130). Luminescence was measured on the Infinite® M200 Pro (Tecan Trading AG, Switzerland). Percent cytotoxicity was calculated and plotted in GraphPad PRISM with curve fit analysis.

FIGS. 14A and 14B show that the recombinantly produced activated bispecific ProC1963 (SHL1) and ProC1965 (SHL2) have increased potency, (>1200 and 50 fold lower EC50 respectively) compared to ProC306 as indicated by the left shift of the dose response curve.

FIGS. 15A and 15B, show that the intact bispecific ProC1446, ProC3007, ProC3008 and ProC1446 are strongly masked as indicated by a right shifted dose response curve relative to their recombinantly produced activated versions ProC1963, and ProC1965, in these assays.

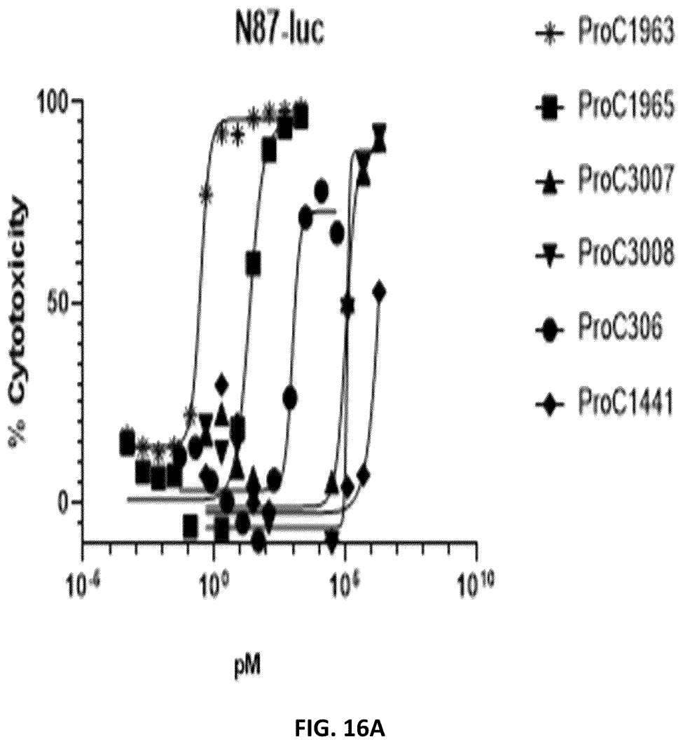

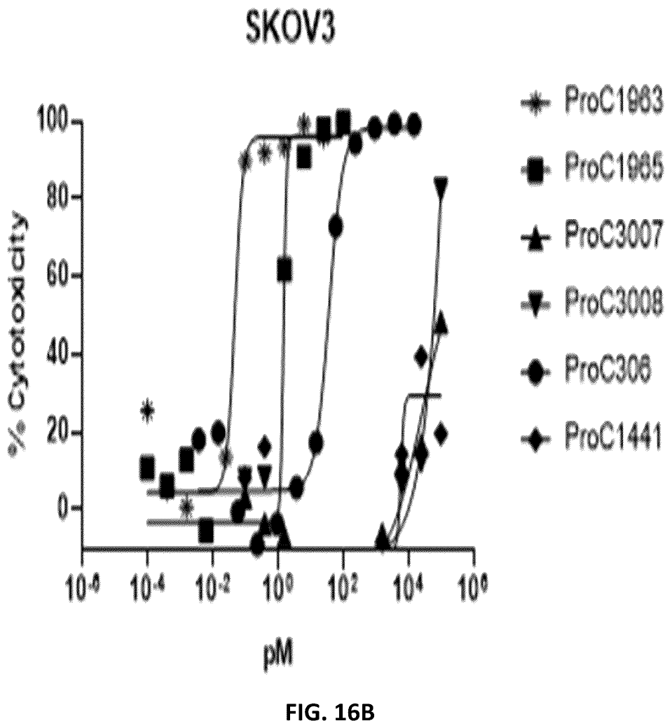

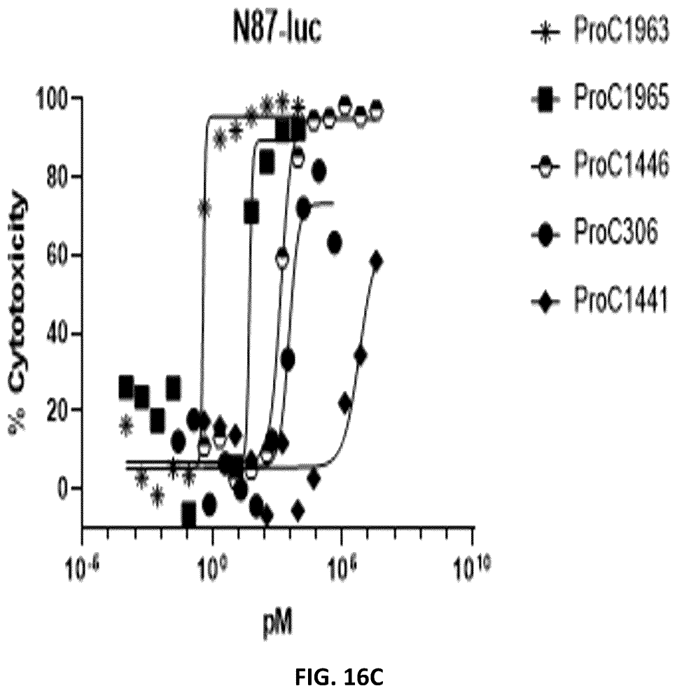

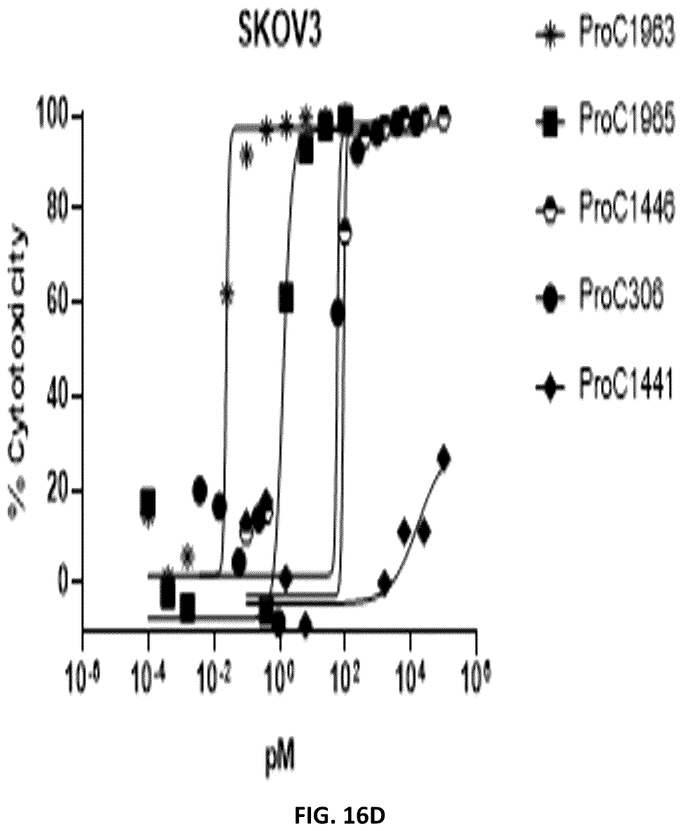

FIGS. 16A-16D show that the intact bispecific ProC1446, ProC3007, ProC3008 and ProC1441 are strongly masked as indicated by a right shifted dose response curve relative to their recombinantly produced activated versions ProC1963, ProC1965, and ProC306, respectively, in these assays.

EC50 values and masking efficiencies (ME) were determined from replicate experiments. The results are provided in Tables 2A and 2B, below.

TABLE 2A
Average EC50 and Masking Efficiency

| Molecule | Molecule | N87 | | SKOV3 | |
|---|---|---|---|---|---|
| ID | Format | EC50 (pM) | ME | EC50 (pM) | ME |
| ProC1965 | SHL2, | 14.46 | N/A | 1.16 | N/A |
| | unmasked, | | | | |
| | no Fc | | | | |
| ProC1963 | SHL1, | 0.26 | N/A | 0.03 | N/A |
| | unmasked, | | | | |
| | no Fc | | | | |
| ProC3007 | SHL2 | 104875.75 | 6859.73 | >100,000 | ND |
| ProC3008 | SHL2 | 87292.25 | 8742.55 | >100,000 | ND |
| ProC1446 | SHL1 | 1104.05 | 18935.31 | 291.03 | 12147.86 |

N/A = Not applicable
ND = Not determined

The EC50 for ProC1963 was ˜200-3000× lower than that of ProC36. The EC50 for ProC1965 was 50× lower than the EC5 for ProC306. The results suggest that the unmasked activatable short-lived TCB formats exhibit greater potency as compared to the control (ProC306), which is not activatable with respect to half-life, i.e., cleavable to release the EM.

TABLE 2B
Average EC50 and Masking Efficiency

| Molecule | Molecule | N87 | | SKOV3 | |
|---|---|---|---|---|---|
| ID | Format | EC50 (pM) | ME | EC50 (pM) | ME |
| ProC1965 | SHL2, | 14.46 | N/A | 1.16 | N/A |
| | unmasked, | | | | |
| | no Fc | | | | |
| ProC1963 | SHL1, | 0.26 | N/A | 0.03 | N/A |
| | unmasked, | | | | |
| | no Fc | | | | |
| ProC3007 | SHL2 | 104875.75 | 6859.73 | >100,000 | ND |
| ProC3008 | SHL2 | 87292.25 | 8742.55 | >100,000 | ND |
| ProC1446 | SHL1 | 1104.05 | 18935.31 | 291.03 | 12147.86 |
| ProC306 | ½ TCB | 736.87 | N/A | 38.4 | N/A |
| | unmasked | | | | |
| ProC1441 | ½ TCB | >2,000,000 | ND | >100,000 | ND |

N/A = Not applicable
ND = Not determined

The results indicate that masking attenuated the activity of all 3 formats: SHL1, SHL2, and ½ TCB.

### Example 7: Dually Masked, Bispecific, Activatable Antibody ProC3007 and its Corresponding Activated Version, ProC1965, Induced Regression of Established NCI-N87 Tumors in Mice

In this example, intact activatable bispecific antibodies ProC3007 (SHL2 TCB), ProC3008 (SHL2 TCB), ProC1441 and recombinantly produced activated bispecific, ProC1965 targeting Her2 and CD3c were analyzed for the ability to induce regression or reduce growth of established NCI-N87 xenograft tumors in human PBMC engrafted NOD scid gamma (NSG) mice.

The human gastric cancer cell line NCI-N87 was obtained from ATCC and was cultured in RPMI+Glutamax+10% FBS according to established procedures. Purified, frozen human PBMCs were obtained from Hemacare Inc, Van Nuys, CA (Donor ID #D163477; Lot #22077550). NSG™ (NOD.Cg-Prkdcscid Il2rgtm1Wjl/SzJ) mice were obtained from The Jackson Laboratories, Bar Harbor, ME.

On day 0, each mouse was inoculated subcutaneously at the right flank with 1×10⁶ NCI-N87 cells in 100 μL RPMI+Glutamax, serum-free medium with Matrigel®. Previously frozen PBMCs from a single donor were thawed and administered (i.p.) on day 7 at a CD3+ T cell to tumor cell ratio of 1:1. When tumor volumes reached ˜125 mm³, mice were randomized, assigned to treatment groups and dosed i.v. according to Table 3. Recombinant bispecific ProC1965 (SHL2 format, no mask and no Fc domain) was dosed 3 times per week to compensate for an expected increase in clearance rate due to lack of half-life extension (Fc) domain. Dose levels of ProC1965 were adjusted to account for the difference in molecular weight. Tumor volume was measured twice weekly. One mouse from the 0.5 mpk cohort was euthanized early. Subsequently, n=7 for that cohort for the day 21 and day 25 time points.

TABLE 3
Groups and doses for NCI-N87 xenograft study C23-005.

| | | | | Dosing |
|---|---|---|---|---|
| Group | Count | Treatment | Dose (mg/kg) | Schedule |
| 1 | 8 | PBS + 6% sucrose | N/A | QW |
| 2 | 8 | ProC1965 | 0.15 | 3x/week |
| 3 | 8 | ProC1965 | 0.5 | 3x/week |
| 4 | 8 | ProC3007 | 0.3 | QW |
| 5 | 8 | ProC3007 | 1.0 | QW |
| 6 | 8 | ProC3008 | 0.3 | QW |
| 7 | 8 | ProC3008 | 1 | QW |
| 8 | 8 | ProC1441 | 1 | QW |

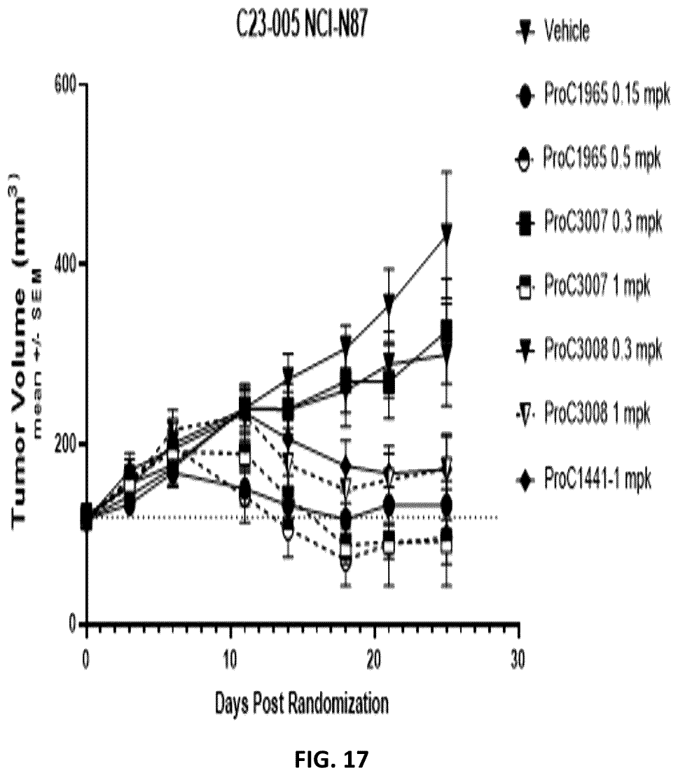

FIG. 17 is a plot of tumor volume versus days post initial treatment dose. The results demonstrate that both intact activable bispecific antibody ProC3007, and recombinantly produced activated bispecific antibody ProC1965 induced regression of NCI-N87 xenograft tumors at 1 and 0.5 mpk, respectively. The results showed that ProC1965 and ProC3007 were more efficacious than ProC1441 in this study. ProC3008 had similar efficacy to ProC1441 at the equivalent dose level.

A second in vivo study was performed as described above but using PBMC from a different donor (Hemacare, Donor ID #D327579; Lot #21070049). In this study, a panel of bispecific activatable antibodies including recombinantly produced activatable short half-life bispecific antibody (ProC1446) and the corresponding recombinantly produced activated version of this activatable molecule (ProC1963, i.e., having the structure of ProC1446 but lacking masks and Fc domains). ProC1446 and ProC1963 were dosed as described in Table 4 and evaluated for their ability to induce regression or reduce growth of established NCI-N87 xenograft tumors in human PBMC engrafted NSG mice. ProC1963 and ProC1446 both appeared to have anti-tumor activity in this study and thus ProC1963 retains the ability to induce tumor regression.

TABLE 4
Dose levels for NCI-N87 xenograft study C22-014

| | | | | Dosing |
|---|---|---|---|---|
| Group | Count | Treatment | Dose (mg/kg) | Schedule |
| 1 | 8 | PBS + 6% sucrose | N/A | QW |
| 2 | 8 | ProC1963 | 0.2 | 3x/week |
| 3 | 8 | ProC1963 | 0.6 | 3x/week |
| 4 | 8 | ProC1446 | 0.3 | QW |
| 5 | 8 | ProC1446 | 1.0 | QW |

N/A = Not applicable

The sequences of the molecules in the examples and other sequences disclosed herein are listed in Table 5 below.

TABLE 5

| SEQ ID NO | NAME/NOTES | SEQUENCES |
|---|---|---|
| 1 | HC179 | SKYGPPCPPCPAPEFEGGPSVFLFPPKPKDTLMISRTP |
| | HC_FcK_IgG4_EN | EVTCVVVDVSQEDPEVQFNWYVDGVEVHNAKTKP |
| | (Fc domain knob | REEQFNSTYRVVSVLTVLHQDWLNGKEYKCKVSNK |
| | mutant) | GLPSSIEKTISKAKGQPREPQVYTLPPCQEEMTKNQV |
| | | SLWCLVKGFYPSDIAVEWESNGQPENNYKTTPPVLD |
| | | SDGSFFLYSRLTVDKSRWQEGNVFSCSVMHEALHN |
| | | HYTQKSLSLSLGX* |
| 2 | Fc domain hole | ESKYGPPCPPCPAPEFEGGPSVFLFPPKPKDTLMISRT |
| | mutant | PEVTCVVVDVSQEDPEVQFNWYVDGVEVHNAKTK |
| | | PREEQFNSTYRVVSVLTVLHQDWLNGKEYKCKVSN |
| | | KGLPSSIEKTISKAKGQPREPQVCTLPPSQEEMTKNQ |
| | | VSLSCAVKGFYPSDIAVEWESNGQPENNYKTTPPVL |
| | | DSDGSFFLVSRLTVDKSRWQEGNVFSCSVMHEALH |
| | | NHYTQKSLSLSLGX* |
| 3 | Human IgG1 | ASTKGPSVFPLAPSSKSTSGGTAALGCLVKDYFPEPV |
| | | TVSWNSGALTSGVHTFPAVLQSSGLYSLSSVVTVPS |
| | | SSLGTQTYICNVNHKPSNTKVDKKVEPKSCDKTHTC |
| | | PPCPAPELLGGPSVFLFPPKPKDTLMISRTPEVTCVV |
| | | VDVSHEDPEVKFNWYVDGVEVHNAKTKPREEQYN |
| | | STYRVVSVLTVLHQDWLNGKEYKCKVSNKALPAPI |
| | | EKTISKAKGQPREPQVYTLPPSREEMTKNQVSLTCL |
| | | VKGFYPSDIAVEWESNGQPENNYKTTPPVLDSDGSF |
| | | FLYSKLTVDKSRWQQGNVFSCSVMHEALHNHYTQ |
| | | KSLSLSPGX |
| 4 | Human IgG2 | ASTKGPSVFPLAPCSRSTSESTAALGCLVKDYFPEPV |
| | | TVSWNSGALTSGVHTFPAVLQSSGLYSLSSVVTVPS |
| | | SNFGTQTYTCNVDHKPSNTKVDKTVERKCCVECPP |
| | | CPAPPVAGPSVFLFPPKPKDTLMISRTPEVTCVVVDV |
| | | SHEDPEVQFNWYVDGVEVHNAKTKPREEQFNSTFR |
| | | VVSVLTVVHQDWLNGKEYKCKVSNKGLPAPIEKTIS |
| | | KTKGQPREPQVYTLPPSREEMTKNQVSLTCLVKGFY |
| | | PSDISVEWESNGQPENNYKTTPPMLDSDGSFFLYSKL |
| | | TVDKSRWQQGNVFSCSVMHEALHNHYTQKSLSLSP |
| | | GX |
| 5 | Human IgG3 | ASTKGPSVFPLAPCSRSTSGGTAALGCLVKDYFPEPV |
| | | TVSWNSGALTSGVHTFPAVLQSSGLYSLSSVVTVPS |
| | | SSLGTQTYTCNVNHKPSNTKVDKRVELKTPLGDTTH |
| | | TCPRCPEPKSCDTPPPCPRCPEPKSCDTPPPCPRCPEP |
| | | KSCDTPPPCPRCPAPELLGGPSVFLFPPKPKDTLMISR |
| | | TPEVTCVVVDVSHEDPEVQFKWYVDGVEVHNAKT |
| | | KPREEQYNSTFRVVSVLTVLHQDWLNGKEYKCKVS |
| | | NKALPAPIEKTISKTKGQPREPQVYTLPPSREEMTKN |
| | | QVSLTCLVKGFYPSDIAVEWESSGQPENNYNTTPPM |
| | | LDSDGSFFLYSKLTVDKSRWQQGNIFSCSVMHEALH |
| | | NRFTQKSLSLSPGX |
| 6 | Human IgG4 | ASTKGPSVFPLAPCSRSTSESTAALGCLVKDYFPEPV |
| | | TVSWNSGALTSGVHTFPAVLQSSGLYSLSSVVTVPS |
| | | SSLGTKTYTCNVDHKPSNTKVDKRVESKYGPPCPSC |
| | | PAPEFLGGPSVFLFPPKPKDTLMISRTPEVTCVVVDV |
| | | SQEDPEVQFNWYVDGVEVHNAKTKPREEQFNSTYR |
| | | VVSVLTVLHQDWLNGKEYKCKVSNKGLPSSIEKTIS |
| | | KAKGQPREPQVYTLPPSQEEMTKNQVSLTCLVKGF |
| | | YPSDIAVEWESNGQPENNYKTTPPVLDSDGSFFLYS |
| | | RLTVDKSRWQEGNVFSCSVMHEALHNHYTQKSLSL |
| | | SLGX* |
| 17 | LC149 LC_Tras | DIQMTQSPSSLSASVGDRVTITCRASQDVNTAVAWY |
| | | QQKPGKAPKLLIYSASFLYSGVPSRFSGSRSGTDFTL |
| | | TISSLQPEDFATYYCQQHYTTPPTFGQGTKVEIKRTV |
| | | AAPSVFIFPPSDEQLKSGTASVVCLLNNFYPREAKVQ |
| | | WKVDNALQSGNSQESVTEQDSKDSTYSLSSTLTLSK |
| | | ADYEKHKVYACEVTHQGLSSPVTKSFNRGEC |
| 18 | LC150 LC_Tras | QGQSGQGALICCSDVSGLCRWCGGGSSGGSISSGLL |
| | 5.17 | SGRSDNHGGGSDIQMTQSPSSLSASVGDRVTITCRAS |
| | | QDVNTAVAWYQQKPGKAPKLLIYSASFLYSGVPSRF |
| | | SGSRSGTDFTLTISSLQPEDFATYYCQQHYTTPPTFG |
| | | QGTKVEIKRTVAAPSVFIFPPSDEQLKSGTASVVCLL |
| | | NNFYPREAKVQWKVDNALQSGNSQESVTEQDSKDS |
| | | TYSLSSTLTLSKADYEKHKVYACEVTHQGLSSPVTK |
| | | SFNRGEC |
| 21 | HC707 HC_Tras-25- | EVQLVESGGGLVQPGGSLRLSCAASGFNIKDTYIHW |
| | v619h-20GG-24- | VRQAPGKGLEWVARIYPTNGYTRYADSVKGRFTISA |
| | H4HEN (in | DTSKNTAYLQMNSLRAEDTAVYYCSRWGGDGFYA |
| | ProC1446) | MDYWGQGTLVTVSSASTKGPSVFPLAPCSRSTSEST |
| | | AALGCLVKDYFPEPVTVSWNSGALTSGVHTFPAVL |
| | | QSSGLYSLSSVVTVPSSSLGTKTYTCNVDHKPSNTK |
| | | VDKRVGGGGSGGGGSGGGGSEVQLVESGGGLVQP |
| | | GGSLKLSCAASGFTFSTYAMNWVRQASGKGLEWV |
| | | GRIRSKYNNYATYYADSVKDRFTISRDDSKNTAYLQ |
| | | MNSLKTEDTAVYYCVRHGNFGNSYVSWWAYWGQ |
| | | GTLVTVSSGGGGSGGGGSGGGGSQTVVTQEPSLTVS |
| | | PGGTVTLTCRSSTGAVTTSNYANWVQQKPGQAPRG |
| | | LIGGTNKRAPGTPARFSGSLIGGKAALTLSGAQPEDE |
| | | AEYYCVLWYSNLWVFGGGTKLTVLGGSGGSSGAS |
| | | GTGTAGGTGSGSGTGSGLSGRSDDHGGGSQGQSGS |
| | | GYLWGCEWNCGGITTGSSGGSGGGGSGGGGSGGG |
| | | GSGGSESKYGPPCPPCPAPEFEGGPSVFLFPPKPKDTL |
| | | MISRTPEVTCVVVDVSQEDPEVQFNWYVDGVEVHN |
| | | AKTKPREEQFNSTYRVVSVLTVLHQDWLNGKEYKC |
| | | KVSNKGLPSSIEKTISKAKGQPREPQVCTLPPSQEEM |
| | | TKNQVSLSCAVKGFYPSDIAVEWESNGQPENNYKTT |
| | | PPVLDSDGSFFLVSRLTVDKSRWQEGNVFSCSVMHE |
| | | ALHNHYTQKSLSLSLGK |
| 22 | HC708 HC_Tras-25- | EVQLVESGGGLVQPGGSLRLSCAASGFNIKDTYIHW |
| | v619h-MN15a-24- | VRQAPGKGLEWVARIYPTNGYTRYADSVKGRFTISA |
| | H4HEN (heavy | DTSKNTAYLQMNSLRAEDTAVYYCSRWGGDGFYA |
| | chain 2 of | MDYWGQGTLVTVSSASTKGPSVFPLAPCSRSTSEST |
| | ProC1447) | AALGCLVKDYFPEPVTVSWNSGALTSGVHTFPAVL |
| | | QSSGLYSLSSVVTVPSSSLGTKTYTCNVDHKPSNTK |
| | | VDKRVGGGGSGGGGSGGGGSEVQLVESGGGLVQP |
| | | GGSLKLSCAASGFTFSTYAMNWVRQASGKGLEWV |
| | | GRIRSKYNNYATYYADSVKDRFTISRDDSKNTAYLQ |
| | | MNSLKTEDTAVYYCVRHGNFGNSYVSWWAYWGQ |
| | | GTLVTVSSGGGGSGGGGSGGGGSQTVVTQEPSLTVS |
| | | PGGTVTLTCRSSTGAVTTSNYANWVQQKPGQAPRG |
| | | LIGGTNKRAPGTPARFSGSLIGGKAALTLSGAQPEDE |
| | | AEYYCVLWYSNLWVFGGGTKLTVLGGSGGSSGAS |
| | | GTGTAGGTGSGSGTGSGLSGRSDDHGGGSQGQSGS |
| | | NAFRCWWDPPCQPMTGSSGGSGGGGSGGGGSGGG |
| | | GSGGSESKYGPPCPPCPAPEFEGGPSVFLFPPKPKDTL |
| | | MISRTPEVTCVVVDVSQEDPEVQFNWYVDGVEVHN |
| | | AKTKPREEQFNSTYRVVSVLTVLHQDWLNGKEYKC |
| | | KVSNKGLPSSIEKTISKAKGQPREPQVCTLPPSQEEM |
| | | TKNQVSLSCAVKGFYPSDIAVEWESNGQPENNYKTT |
| | | PPVLDSDGSFFLVSRLTVDKSRWQEGNVFSCSVMHE |
| | | ALHNHYTQKSLSLSLGK |
| 23 | HC709 HC_Tras-25- | EVQLVESGGGLVQPGGSLRLSCAASGFNIKDTYIHW |
| | v619h-MN15b-24- | VRQAPGKGLEWVARIYPTNGYTRYADSVKGRFTISA |
| | H4HEN (in | DTSKNTAYLQMNSLRAEDTAVYYCSRWGGDGFYA |
| | ProC1448) | MDYWGQGTLVTVSSASTKGPSVFPLAPCSRSTSEST |
| | | AALGCLVKDYFPEPVTVSWNSGALTSGVHTFPAVL |
| | | QSSGLYSLSSVVTVPSSSLGTKTYTCNVDHKPSNTK |
| | | VDKRVGGGGSGGGGSGGGGSEVQLVESGGGLVQP |
| | | GGSLKLSCAASGFTFSTYAMNWVRQASGKGLEWV |
| | | GRIRSKYNNYATYYADSVKDRFTISRDDSKNTAYLQ |
| | | MNSLKTEDTAVYYCVRHGNFGNSYVSWWAYWGQ |
| | | GTLVTVSSGGGGSGGGGSGGGGSQTVVTQEPSLTVS |
| | | PGGTVTLTCRSSTGAVTTSNYANWVQQKPGQAPRG |
| | | LIGGTNKRAPGTPARFSGSLIGGKAALTLSGAQPEDE |
| | | AEYYCVLWYSNLWVFGGGTKLTVLGGSGGSSGAS |
| | | GTGTAGGTGSGSGTGSGLSGRSDDHGGGSQGQSGS |
| | | ARGLCWWDPPCTHDLGSSGGSGGGGSGGGGSGGG |
| | | GSGGSESKYGPPCPPCPAPEFEGGPSVFLFPPKPKDTL |
| | | MISRTPEVTCVVVDVSQEDPEVQFNWYVDGVEVHN |
| | | AKTKPREEQFNSTYRVVSVLTVLHQDWLNGKEYKC |
| | | KVSNKGLPSSIEKTISKAKGQPREPQVCTLPPSQEEM |
| | | TKNQVSLSCAVKGFYPSDIAVEWESNGQPENNYKTT |
| | | PPVLDSDGSFFLVSRLTVDKSRWQEGNVFSCSVMHE |
| | | ALHNHYTQKSLSLSLGK |
| 24 | HC710 HC_Tras-25- | EVQLVESGGGLVQPGGSLRLSCAASGFNIKDTYIHW |
| | v619h-MN15c-24- | VRQAPGKGLEWVARIYPTNGYTRYADSVKGRFTISA |
| | H4HEN (in | DTSKNTAYLQMNSLRAEDTAVYYCSRWGGDGFYA |
| | ProC1449) | MDYWGQGTLVTVSSASTKGPSVFPLAPCSRSTSEST |
| | | AALGCLVKDYFPEPVTVSWNSGALTSGVHTFPAVL |
| | | QSSGLYSLSSVVTVPSSSLGTKTYTCNVDHKPSNTK |
| | | VDKRVGGGGSGGGGSGGGGSEVQLVESGGGLVQP |
| | | GGSLKLSCAASGFTFSTYAMNWVRQASGKGLEWV |
| | | GRIRSKYNNYATYYADSVKDRFTISRDDSKNTAYLQ |
| | | MNSLKTEDTAVYYCVRHGNFGNSYVSWWAYWGQ |
| | | GTLVTVSSGGGGSGGGGSGGGGSQTVVTQEPSLTVS |
| | | PGGTVTLTCRSSTGAVTTSNYANWVQQKPGQAPRG |
| | | LIGGTNKRAPGTPARFSGSLIGGKAALTLSGAQPEDE |
| | | AEYYCVLWYSNLWVFGGGTKLTVLGGSGGSSGAS |
| | | GTGTAGGTGSGSGTGSGLSGRSDDHGGGSQGQSGS |
| | | NHSLCYWDPPCEPSTGSSGGSGGGGSGGGGSGGGG |
| | | SGGSESKYGPPCPPCPAPEFEGGPSVFLFPPKPKDTL |
| | | MISRTPEVTCVVVDVSQEDPEVQFNWYVDGVEVHN |
| | | AKTKPREEQFNSTYRVVSVLTVLHQDWLNGKEYKC |
| | | KVSNKGLPSSIEKTISKAKGQPREPQVCTLPPSQEEM |
| | | TKNQVSLSCAVKGFYPSDIAVEWESNGQPENNYKTT |
| | | PPVLDSDGSFFLVSRLTVDKSRWQEGNVFSCSVMHE |
| | | ALHNHYTQKSLSLSLGK |
| 27 | Heavy chain | EVQLVESGGGLVQPGGSLRLSCAASGFNIKDTYIHW |
| | fragment of | VRQAPGKGLEWVARIYPTNGYTRYADSVKGRFTISA |
| | trastuzumab Fab | DTSKNTAYLQMNSLRAEDTAVYYCSRWGGDGFYA |
| | | MDYWGQGTLVTVSSASTKGPSVFPLAPCSRSTSEST |
| | | AALGCLVKDYFPEPVTVSWNSGALTSGVHTFPAVL |
| | | QSSGLYSLSSVVTVPSSSLGTKTYTCNVDHKPSNTK |
| | | VDKRV |
| 28 | Linker with 15 | GGGGSGGGGSGGGGS |
| | amino acids | |
| 29 | anti-CD3 scFv (in | EVQLVESGGGLVQPGGSLKLSCAASGFTFSTYAMN |
| | ProCs 531, 1446- | WVRQASGKGLEWVGRIRSKYNNYATYYADSVKDR |
| | 1449) | FTISRDDSKNTAYLQMNSLKTEDTAVYYCVRHGNF |
| | | GNSYVSWWAYWGQGTLVTVSSGGGGSGGGGSGG |
| | | GGSQTVVTQEPSLTVSPGGTVTLTCRSSTGAVTTSN |
| | | YANWVQQKPGQAPRGLIGGTNKRAPGTPARFSGSLI |
| | | GGKAALTLSGAQPEDEAEYYCVLWYSNLWVFGGG |
| | | TKLTVL |
| 30 | Heavy chain | EVQLVESGGGLVQPGGSLKLSCAASGFTFSTYAMN |
| | variable region of | WVRQASGKGLEWVGRIRSKYNNYATYYADSVKDR |
| | anti-CD3 scFv (in | FTISRDDSKNTAYLQMNSLKTEDTAVYYCVRHGNF |
| | ProCs 531, 1446- | GNSYVSWWAYWGQGTLVTVSS |
| | 1449) | |
| 31 | Light chain variable | QTVVTQEPSLTVSPGGTVTLTCRSSTGAVTTSNYAN |
| | region of anti-CD3 | WVQQKPGQAPRGLIGGTNKRAPGTPARFSGSLIGGK |
| | scFv (in ProCs 531, | AALTLSGAQPEDEAEYYCVLWYSNLWVFGGGTKLT |
| | 1446-1449) | VL |
| 32 | Linker with 27 | GGSGGSSGASGTGTAGGTGSGSGTGSG |
| | amino acids | |
| 33 | Cleavable moiety | LSGRSDDH |
| | (1204DDH) | |
| 34 | CD3 masking | GYLWGCEWNCGGITT |
| | moiety (20GG, in | |
| | ProC 1466) | |
| 35 | CD3 masking | NAFRCWWDPPCQPMT |
| | moiety (MN15a, in | |
| | ProC 1447) | |
| 36 | CD3 masking | ARGLCWWDPPCTHDL |
| | moiety (MN15b, in | |
| | ProC 1448) | |
| 37 | CD3 masking | NHSLCYWDPPCEPST |
| | moiety (MN15c, in | |
| | ProC 1449) | |
| 38 | Linker with 24 | GSSGGSGGGGSGGGGSGGGGSGGS |
| | amino acids | |
| 40 | F5.17 masking | ALICCSDVSGLCRWC |
| | moiety (mask for | |
| | trastuzumab Fab) | |
| 41 | Cleavable moiety | ISSGLLSGRSDNH |
| | (1490) | |
| 42 | hIgG4 S228P and | ASTKGPSVFPLAPCSRSTSESTAALGCLVKDYFPEPV |
| | L235E CH1-Fc | TVSWNSGALTSGVHTFPAVLQSSGLYSLSSVVTVPS |
| | | SSLGTKTYTCNVDHKPSNTKVDKRVESKYGPPCPPC |
| | | PAPEFEGGPSVFLFPPKPKDTLMISRTPEVTCVVVDV |
| | | SQEDPEVQFNWYVDGVEVHNAKTKPREEQFNSTYR |
| | | VVSVLTVLHQDWLNGKEYKCKVSNKGLPSSIEKTIS |
| | | KAKGQPREPQVYTLPPSQEEMTKNQVSLTCLVKGF |
| | | YPSDIAVEWESNGQPENNYKTTPPVLDSDGSFFLYS |
| | | RLTVDKSRWQEGNVFSCSVMHEALHNHYTQKSLSL |
| | | SLGX* |
| 43 | IgG4-EN KiHSS-H | SKYGPPCPPCPAPEFEGGPSVFLFPPKPKDTLMISRTP |
| | Chain Fc ONLY | EVTCVVVDVSQEDPEVQFNWYVDGVEVHNAKTKP |
| | | REEQFNSTYRVVSVLTVLHQDWLNGKEYKCKVSNK |
| | | GLPSSIEKTISKAKGQPREPQVCTLPPSQEEMTKNQV |
| | | SLSCAVKGFYPSDIAVEWESNGQPENNYKTTPPVLD |
| | | SDGSFFLVSRLTVDKSRWQEGNVFSCSVMHEALHN |
| | | HYTQKSLSLSLGX* |
| 44 | IgG4-EN KiHSS-K | GPPCPPCPAPEFEGGPSVFLFPPKPKDTLMISRTPEVT |
| | Chain-Fc ONLY | CVVVDVSQEDPEVQFNWYVDGVEVHNAKTKPREE |
| | | QFNSTYRVVSVLTVLHQDWLNGKEYKCKVSNKGLP |
| | | SSIEKTISKAKGQPREPQVYTLPPCQEEMTKNQVSL |
| | | WCLVKGFYPSDIAVEWESNGQPENNYKTTPPVLDS |
| | | DGSFFLYSRLTVDKSRWQEGNVFSCSVMHEALHNH |
| | | YTQKSLSLSLGX* |
| 45 | IgG1 KiHSS-H | DKTHTCPPCPAPELLGGPSVFLFPPKPKDTLMISRTPE |
| | Chain Fc ONLY | VTCVVVDVSHEDPEVKFNWYVDGVEVHNAKTKPR |
| | | EEQYNSTYRVVSVLTVLHQDWLNGKEYKCKVSNK |
| | | ALPAPIEKTISKAKGQPREPQVCTLPPSRDELTKNQV |
| | | SLSCAVKGFYPSDIAVEWESNGQPENNYKTTPPVLD |
| | | SDGSFFLVSKLTVDKSRWQQGNVFSCSVMHEALHN |
| | | HYTQKSLSLSPGX |
| 46 | IgG1 KiHSS-K | DKTHTCPPCPAPELLGGPSVFLFPPKPKDTLMISRTPE |
| | Chain-Fc ONLY | VTCVVVDVSHEDPEVKFNWYVDGVEVHNAKTKPR |
| | | EEQYNSTYRVVSVLTVLHQDWLNGKEYKCKVSNK |
| | | ALPAPIEKTISKAKGQPREPQVYTLPPCRDELTKNQV |
| | | SLWCLVKGFYPSDIAVEWESNGQPENNYKTTPPVLD |
| | | SDGSFFLYSKLTVDKSRWQQGNVFSCSVMHEALHN |
| | | HYTQKSLSLSPGX |
| 597 | ProC306: | EVQLVESGGGLVQPGGSLKLSCAASGFTESTYAMN |
| | v619_HLh_Tras 1/2 | WVRQASGKGLEWVGRIRSKYNNYATYYADSVKDR |
| | TCB | FTISRDDSKNTAYLQMNSLKTEDTAVYYCVRHGNF |
| | (v619_HLh) | GNSYVSWWAYWGQGTLVTVSSGGGGSGGGGSGG |
| | | GGSQTVVTQEPSLTVSPGGTVTLTCGSSTGAVTTSN |
| | | YVNWVQQKPGQAPRGLIGGTNKRAPGTPARFSGSLI |
| | | GGKAALTLSGAQPEDEAEYYCVLWYSNRWVFGGG |
| | | TKLTVLGGGGSEVQLVESGGGLVQPGGSLRLSCAAS |
| | | GFNIKDTYIHWVRQAPGKGLEWVARIYPTNGYTRY |
| | | ADSVKGRFTISADTSKNTAYLQMNSLRAEDTAVYY |
| | | CSRWGGDGFYAMDYWGQGTLVTVSSASTKGPSVFP |
| | | LAPCSRSTSESTAALGCLVKDYFPEPVTVSWNSGAL |
| | | TSGVHTFPAVLQSSGLYSLSSVVTVPSSSLGTKTYTC |
| | | NVDHKPSNTKVDKRVESKYGPPCPPCPAPEFEGGPS |
| | | VFLFPPKPKDTLMISRTPEVTCVVVDVSQEDPEVQFN |
| | | WYVDGVEVHNAKTKPREEQFNSTYRVVSVLTVLHQ |
| | | DWLNGKEYKCKVSNKGLPSSIEKTISKAKGQPREPQ |
| | | VCTLPPSQEEMTKNQVSLSCAVKGFYPSDIAVEWES |
| | | NGQPENNYKTTPPVLDSDGSFFLVSRLTVDKSRWQE |
| | | GNVFSCSVMHEALHNHYTQKSLSLSLGX |
| 598 | ProC306: | GSSKYGPPCPPCPAPEFEGGPSVFLFPPKPKDTLMISR |
| | v619_HLh_Tras 1/2 | TPEVTCVVVDVSQEDPEVQFNWYVDGVEVHNAKT |
| | TCB | KPREEQFNSTYRVVSVLTVLHQDWLNGKEYKCKVS |
| | (HC_FcK_IgG4_EN) | NKGLPSSIEKTISKAKGQPREPQVYTLPPCQEEMTKN |
| | | QVSLWCLVKGFYPSDIAVEWESNGQPENNYKTTPP |
| | | VLDSDGSFFLYSRLTVDKSRWQEGNVFSCSVMHEA |
| | | LHNHYTQKSLSLSLGX |
| 599 | ProC306: | DIQMTQSPSSLSASVGDRVTITCRASQDVNTAVAWY |
| | v619_HLh_Tras 1/2 | QQKPGKAPKLLIYSASFLYSGVPSRFSGSRSGTDFTL |
| | TCB (LC_Tras) | TISSLQPEDFATYYCQQHYTTPPTFGQGTKVEIKRTV |
| | | AAPSVFIFPPSDEQLKSGTASVVCLLNNFYPREAKVQ |
| | | WKVDNALQSGNSQESVTEQDSKDSTYSLSSTLTLSK |
| | | ADYEKHKVYACEVTHQGLSSPVTKSFNRGEC |
| 600 | ProC531: | EVQLVESGGGLVQPGGSLKLSCAASGFTFSTYAMN |
| | v619_hh9_Tras 1/2 | WVRQASGKGLEWVGRIRSKYNNYATYYADSVKDR |
| | TCB | FTISRDDSKNTAYLQMNSLKTEDTAVYYCVRHGNF |
| | (HC | GNSYVSWWAYWGQGTLVTVSSGGGGSGGGGSGG |
| | v619_hh9_Tras 1/2 | GGSQTVVTQEPSLTVSPGGTVTLTCRSSTGAVTTSN |
| | TCB) | YANWVQQKPGQAPRGLIGGTNKRAPGTPARFSGSLI |
| | | GGKAALTLSGAQPEDEAEYYCVLWYSNLWVFGGG |
| | | TKLTVLGGGGSEVQLVESGGGLVQPGGSLRLSCAAS |
| | | GFNIKDTYIHWVRQAPGKGLEWVARIYPTNGYTRY |
| | | ADSVKGRFTISADTSKNTAYLQMNSLRAEDTAVYY |
| | | CSRWGGDGFYAMDYWGQGTLVTVSSASTKGPSVFP |
| | | LAPCSRSTSESTAALGCLVKDYFPEPVTVSWNSGAL |
| | | TSGVHTFPAVLQSSGLYSLSSVVTVPSSSLGTKTYTC |
| | | NVDHKPSNTKVDKRVESKYGPPCPPCPAPEFEGGPS |
| | | VFLFPPKPKDTLMISRTPEVTCVVVDVSQEDPEVQFN |
| | | WYVDGVEVHNAKTKPREEQFNSTYRVVSVLTVLHQ |
| | | DWLNGKEYKCKVSNKGLPSSIEKTISKAKGQPREPQ |
| | | VCTLPPSQEEMTKNQVSLSCAVKGFYPSDIAVEWES |
| | | NGQPENNYKTTPPVLDSDGSFFLVSRLTVDKSRWQE |
| | | GNVFSCSVMHEALHNHYTQKSLSLSLG |
| | | X |
| 601 | ProC531: | GSSKYGPPCPPCPAPEFEGGPSVFLFPPKPKDTLMISR |
| | v619_hh9_Tras 1/2 | TPEVTCVVVDVSQEDPEVQFNWYVDGVEVHNAKT |
| | TCB | KPREEQFNSTYRVVSVLTVLHQDWLNGKEYKCKVS |
| | (HC_FcK_IgG4_EN) | NKGLPSSIEKTISKAKGQPREPQVYTLPPCQEEMTKN |
| | | QVSLWCLVKGFYPSDIAVEWESNGQPENNYKTTPP |
| | | VLDSDGSFFLYSRLTVDKSRWQEGNVFSCSVMHEA |
| | | LHNHYTQKSLSLSLG |
| | | X |
| 602 | ProC531: | DIQMTQSPSSLSASVGDRVTITCRASQDVNTAVAWY |
| | v619_hh9_Tras 1/2 | QQKPGKAPKLLIYSASFLYSGVPSRFSGSRSGTDFTL |
| | TCB | TISSLQPEDFATYYCQQHYTTPPTFGQGTKVEIKRTV |
| | (LC_Tras) | AAPSVFIFPPSDEQLKSGTASVVCLLNNFYPREAKVQ |
| | | WKVDNALQSGNSQESVTEQDSKDSTYSLSSTLTLSK |
| | | ADYEKHKVYACEVTHQGLSSPVTKSFNRGEC |
| 603 | ProC1965: v619h-5- | EVQLVESGGGLVQPGGSLKLSCAASGFTFSTYAMN |
| | Tras-noFc (an | WVRQASGKGLEWVGRIRSKYNNYATYYADSVKDR |
| | unmasked, no Fc | FTISRDDSKNTAYLQMNSLKTEDTAVYYCVRHGNF |
| | form of SHL2) | GNSYVSWWAYWGQGTLVTVSSGGGGSGGGGSGG |
| | (PP_v619h-5-Tras- | GGSQTVVTQEPSLTVSPGGTVTLTCGSSTGAVTTSN |
| | no Fc) | YVNWVQQKPGQAPRGLIGGTNKRAPGTPARFSGSLI |
| | | GGKAALTLSGAQPEDEAEYYCVLWYSNRWVFGGG |
| | | TKLTVLGGGGSEVQLVESGGGLVQPGGSLRLSCAAS |
| | | GFNIKDTYIHWVRQAPGKGLEWVARIYPTNGYTRY |
| | | ADSVKGRFTISADTSKNTAYLQMNSLRAEDTAVYY |
| | | CSRWGGDGFYAMDYWGQGTLVTVSSASTKGPSVFP |
| | | LAPCSRSTSESTAALGCLVKDYFPEPVTVSWNSGAL |
| | | TSGVHTFPAVLQSSGLYSLSSVVTVPSSSLGTKTYTC |
| | | NVDHKPSNTKVDKRV |
| 604 | ProC1965: v619h-5- | DIQMTQSPSSLSASVGDRVTITCRASQDVNTAVAWY |
| | Tras-noFc (an | QQKPGKAPKLLIYSASFLYSGVPSRFSGSRSGTDFTL |
| | unmasked, no Fc | TISSLQPEDFATYYCQQHYTTPPTFGQGTKVEIKRTV |
| | form of SHL2) | AAPSVFIFPPSDEQLKSGTASVVCLLNNFYPREAKVQ |
| | (LC_Tras) | WKVDNALQSGNSQESVTEQDSKDSTYSLSSTLTLSK |
| | | ADYEKHKVYACEVTHQGLSSPVTKSFNRGEC |
| 605 | ProC1963: Tras-25- | EVQLVESGGGLVQPGGSLRLSCAASGFNIKDTYIHW |
| | v619h-noFc (an | VRQAPGKGLEWVARIYPTNGYTRYADSVKGRFTISA |
| | unmasked/no Fc | DTSKNTAYLQMNSLRAEDTAVYYCSRWGGDGFYA |
| | form of SHL1) | MDYWGQGTLVTVSSASTKGPSVFPLAPCSRSTSEST |
| | (PP_Tras-25-v619h- | AALGCLVKDYFPEPVTVSWNSGALTSGVHTFPAVL |
| | noFc) | QSSGLYSLSSVVTVPSSSLGTKTYTCNVDHKPSNTK |
| | | VDKRVGGGGSGGGGSGGGGSEVQLVESGGGLVQP |
| | | GGSLKLSCAASGFTFSTYAMNWVRQASGKGLEWV |
| | | GRIRSKYNNYATYYADSVKDRFTISRDDSKNTAYLQ |
| | | MNSLKTEDTAVYYCVRHGNFGNSYVSWWAYWGQ |
| | | GTLVTVSSGGGGSGGGGSGGGGSQTVVTQEPSLTVS |
| | | PGGTVTLTCGSSTGAVTTSNYVNWVQQKPGQAPRG |
| | | LIGGTNKRAPGTPARFSGSLIGGKAALTLSGAQPEDE |
| | | AEYYCVLWYSNRWVFGGGTKLTVL |
| 606 | ProC1963: Tras-25- | DIQMTQSPSSLSASVGDRVTITCRASQDVNTAVAWY |
| | v619h-noFc (an | QQKPGKAPKLLIYSASFLYSGVPSRFSGSRSGTDFTL |
| | unmasked/no Fc | TISSLQPEDFATYYCQQHYTTPPTFGQGTKVEIKRTV |
| | form of SHL1) | AAPSVFIFPPSDEQLKSGTASVVCLLNNFYPREAKVQ |
| | (LC_Tras) | WKVDNALQSGNSQESVTEQDSKDSTYSLSSTLTLSK |
| | | ADYEKHKVYACEVTHQGLSSPVTKSFNRGEC |
| 607 | ProC3007: 20GG- | QGQSGSGYLWGCEWNCGGITTGSSGGSGGSGGLSG |
| | GL1204DDH- | RSDDHGGSEVQLVESGGGLVQPGGSLKLSCAASGFT |
| | v619h-5mr-F5.17- | FSTYAMNWVRQASGKGLEWVGRIRSKYNNYATYY |
| | Tras-1490DNI- | ADSVKDRFTISRDDSKNTAYLQMNSLKTEDTAVYY |
| | SHL2-TCB | CVRHGNFGNSYVSWWAYWGQGTLVTVSSGGGGSG |
| | (HC_20GG- | GGGSGGGGSQTVVTQEPSLTVSPGGTVTLTCGSSTG |
| | GL1204DDH- | AVTTSNYVNWVQQKPGQAPRGLIGGTNKRAPGTPA |
| | v619h-5mr-Tras- | RFSGSLIGGKAALTLSGAQPEDEAEYYCVLWYSNR |
| | 1490DNI-FcH4EN) | WVFGGGTKLTVLGGGGSEVQLVESGGGLVQPGGSL |
| | | RLSCAASGFNIKDTYIHWVRQAPGKGLEWVARIYPT |
| | | NGYTRYADSVKGRFTISADTSKNTAYLQMNSLRAE |
| | | DTAVYYCSRWGGDGFYAMDYWGQGTLVTVSSAST |
| | | KGPSVFPLAPCSRSTSESTAALGCLVKDYFPEPVTVS |
| | | WNSGALTSGVHTFPAVLQSSGLYSLSSVVTVPSSSL |
| | | GTKTYTCNVDHKPSNTKVDKRVESKYGGGSSGGSIS |
| | | SGLLSGRSDNIGGGSGPPCPPCPAPEFEGGPSVFLFPP |
| | | KPKDTLMISRTPEVTCVVVDVSQEDPEVQFNWYVD |
| | | GVEVHNAKTKPREEQFNSTYRVVSVLTVLHQDWLN |
| | | GKEYKCKVSNKGLPSSIEKTISKAKGQPREPQVCTLP |
| | | PSQEEMTKNQVSLSCAVKGFYPSDIAVEWESNGQPE |
| | | NNYKTTPPVLDSDGSFFLVSRLTVDKSRWQEGNVFS |
| | | CSVMHEALHNHYTQKSLSLSLGX |
| 608 | ProC3007: 20GG- | GSSKYGPPCPPCPAPEFEGGPSVFLFPPKPKDTLMISR |
| | GL1204DDH- | TPEVTCVVVDVSQEDPEVQFNWYVDGVEVHNAKT |
| | v619h-5mr-F5.17- | KPREEQFNSTYRVVSVLTVLHQDWLNGKEYKCKVS |
| | Tras-1490DNI- | NKGLPSSIEKTISKAKGQPREPQVYTLPPCQEEMTKN |
| | SHL2-TCB | QVSLWCLVKGFYPSDIAVEWESNGQPENNYKTTPP |
| | (HC_FcK_IgG4_EN) | VLDSDGSFFLYSRLTVDKSRWQEGNVFSCSVMHEA |
| | | LHNHYTQKSLSLSLGX |
| 609 | ProC3007: 20GG- | QGQSGQGALICCSDVSGLCRWCGGGSSGGSISSGLL |
| | GL1204DDH- | SGRSDNHGGGSDIQMTQSPSSLSASVGDRVTITCRAS |
| | v619h-5mr-F5.17- | QDVNTAVAWYQQKPGKAPKLLIYSASFLYSGVPSRF |
| | Tras-1490DNI- | SGSRSGTDFTLTISSLQPEDFATYYCQQHYTTPPTFG |
| | SHL2-TCB | QGTKVEIKRTVAAPSVFIFPPSDEQLKSGTASVVCLL |
| | (LC_Tras 5.17) | NNFYPREAKVQWKVDNALQSGNSQESVTEQDSKDS |
| | | TYSLSSTLTLSKADYEKHKVYACEVTHQGLSSPVTK |
| | | SFNRGEC |
| 610 | ProC3008: 20GG- | QGQSGSGYLWGCEWNCGGITTGSSGGSGGSGGLSG |
| | GL1204DDH- | RSDDHGGSEVQLVESGGGLVQPGGSLKLSCAASGFT |
| | v619h-5mr-F5.17- | FSTYAMNWVRQASGKGLEWVGRIRSKYNNYATYY |
| | Tras-1406-SHL2- | ADSVKDRFTISRDDSKNTAYLQMNSLKTEDTAVYY |
| | TCB | CVRHGNFGNSYVSWWAYWGQGTLVTVSSGGGGSG |
| | (HC_20GG- | GGGSGGGGSQTVVTQEPSLTVSPGGTVTLTCGSSTG |
| | GL1204DDH- | AVTTSNYVNWVQQKPGQAPRGLIGGTNKRAPGTPA |
| | v619h-5mr-Tras- | RFSGSLIGGKAALTLSGAQPEDEAEYYCVLWYSNR |
| | 1406-FcH4EN) | WVFGGGTKLTVLGGGGSEVQLVESGGGLVQPGGSL |
| | | RLSCAASGFNIKDTYIHWVRQAPGKGLEWVARIYPT |
| | | NGYTRYADSVKGRFTISADTSKNTAYLQMNSLRAE |
| | | DTAVYYCSRWGGDGFYAMDYWGQGTLVTVSSAST |
| | | KGPSVFPLAPCSRSTSESTAALGCLVKDYFPEPVTVS |
| | | WNSGALTSGVHTFPAVLQSSGLYSLSSVVTVPSSSL |
| | | GTKTYTCNVDHKPSNTKVDKRVESKYGGGSSGGSA |
| | | PRSALAHGLFGGGSGPPCPPCPAPEFEGGPSVFLFPP |
| | | KPKDTLMISRTPEVTCVVVDVSQEDPEVQFNWYVD |
| | | GVEVHNAKTKPREEQFNSTYRVVSVLTVLHQDWLN |
| | | GKEYKCKVSNKGLPSSIEKTISKAKGQPREPQVCTLP |
| | | PSQEEMTKNQVSLSCAVKGFYPSDIAVEWESNGQPE |
| | | NNYKTTPPVLDSDGSFFLVSRLTVDKSRWQEGNVFS |
| | | CSVMHEALHNHYTQKSLSLSLGX |
| 611 | ProC3008: 20GG- | GSSKYGPPCPPCPAPEFEGGPSVFLFPPKPKDTLMISR |
| | GL1204DDH- | TPEVTCVVVDVSQEDPEVQFNWYVDGVEVHNAKT |
| | v619h-5mr-F5.17- | KPREEQFNSTYRVVSVLTVLHQDWLNGKEYKCKVS |
| | Tras-1406-SHL2- | NKGLPSSIEKTISKAKGQPREPQVYTLPPCQEEMTKN |
| | TCB | QVSLWCLVKGFYPSDIAVEWESNGQPENNYKTTPP |
| | (HC_FcK_IgG4_EN) | VLDSDGSFFLYSRLTVDKSRWQEGNVFSCSVMHEA |
| | | LHNHYTQKSLSLSLGX |
| 612 | ProC3008: 20GG- | QGQSGQGALICCSDVSGLCRWCGGGSSGGSISSGLL |
| | GL1204DDH- | SGRSDNHGGGSDIQMTQSPSSLSASVGDRVTITCRAS |
| | v619h-5mr-F5.17- | QDVNTAVAWYQQKPGKAPKLLIYSASFLYSGVPSRF |
| | Tras-1406-SHL2- | SGSRSGTDFTLTISSLQPEDFATYYCQQHYTTPPTFG |
| | TCB (LC_Tras 5.17) | QGTKVEIKRTVAAPSVFIFPPSDEQLKSGTASVVCLL |
| | | NNFYPREAKVQWKVDNALQSGNSQESVTEQDSKDS |
| | | TYSLSSTLTLSKADYEKHKVYACEVTHQGLSSPVTK |
| | | SFNRGEC |
| 613 | ProC1441: 20GG- | QGQSGSGYLWGCEWNCGGITTGSSGGSGGSGGLSG |
| | v619h-F5.17-Tras- | RSDDHGGSEVQLVESGGGLVQPGGSLKLSCAASGFT |
| | Half TCB | FSTYAMNWVRQASGKGLEWVGRIRSKYNNYATYY |
| | (HC_20GG-v619h- | ADSVKDRFTISRDDSKNTAYLQMNSLKTEDTAVYY |
| | Tras-H4HEN) | CVRHGNFGNSYVSWWAYWGQGTLVTVSSGGGGSG |
| | | GGGSGGGGSQTVVTQEPSLTVSPGGTVTLTCGSSTG |
| | | AVTTSNYVNWVQQKPGQAPRGLIGGTNKRAPGTPA |
| | | RFSGSLIGGKAALTLSGAQPEDEAEYYCVLWYSNR |
| | | WVFGGGTKLTVLGGGGSEVQLVESGGGLVQPGGSL |
| | | RLSCAASGFNIKDTYIHWVRQAPGKGLEWVARIYPT |
| | | NGYTRYADSVKGRFTISADTSKNTAYLQMNSLRAE |
| | | DTAVYYCSRWGGDGFYAMDYWGQGTLVTVSSAST |
| | | KGPSVFPLAPCSRSTSESTAALGCLVKDYFPEPVTVS |
| | | WNSGALTSGVHTFPAVLQSSGLYSLSSVVTVPSSSL |
| | | GTKTYTCNVDHKPSNTKVDKRVESKYGPPCPPCPAP |
| | | EFEGGPSVFLFPPKPKDTLMISRTPEVTCVVVDVSQE |
| | | DPEVQFNWYVDGVEVHNAKTKPREEQFNSTYRVVS |
| | | VLTVLHQDWLNGKEYKCKVSNKGLPSSIEKTISKAK |
| | | GQPREPQVCTLPPSQEEMTKNQVSLSCAVKGFYPSD |
| | | IAVEWESNGQPENNYKTTPPVLDSDGSFFLVSRLTV |
| | | DKSRWQEGNVFSCSVMHEALHNHYTQKSLSLSLGX |
| 614 | ProC1441: 20GG- | GSSKYGPPCPPCPAPEFEGGPSVFLFPPKPKDTLMISR |
| | v619h-F5.17-Tras- | TPEVTCVVVDVSQEDPEVQFNWYVDGVEVHNAKT |
| | Half TCB | KPREEQFNSTYRVVSVLTVLHQDWLNGKEYKCKVS |
| | (HC_FcK_IgG4_EN) | NKGLPSSIEKTISKAKGQPREPQVYTLPPCQEEMTKN |
| | | QVSLWCLVKGFYPSDIAVEWESNGQPENNYKTTPP |
| | | VLDSDGSFFLYSRLTVDKSRWQEGNVFSCSVMHEA |
| | | LHNHYTQKSLSLSLGX |
| 615 | ProC1441: 20GG- | QGQSGQGALICCSDVSGLCRWCGGGSSGGSISSGLL |
| | v619h-F5.17-Tras- | SGRSDNHGGGSDIQMTQSPSSLSASVGDRVTITCRAS |
| | Half TCB | QDVNTAVAWYQQKPGKAPKLLIYSASFLYSGVPSRF |
| | (LC_Tras 5.17) | SGSRSGTDFTLTISSLQPEDFATYYCQQHYTTPPTFG |
| | | QGTKVEIKRTVAAPSVFIFPPSDEQLKSGTASVVCLL |
| | | NNFYPREAKVQWKVDNALQSGNSQESVTEQDSKDS |
| | | TYSLSSTLTLSKADYEKHKVYACEVTHQGLSSPVTK |
| | | SFNRGEC |
| 616 | ProC1446: F5.17- | EVQLVESGGGLVQPGGSLRLSCAASGFNIKDTYIHW |
| | Tras-25-v619h- | VRQAPGKGLEWVARIYPTNGYTRYADSVKGRFTISA |
| | 20GG-24-Half TCB | DTSKNTAYLQMNSLRAEDTAVYYCSRWGGDGFYA |
| | (HC_Tras-25- | MDYWGQGTLVTVSSASTKGPSVFPLAPCSRSTSEST |
| | v619h-20GG-24- | AALGCLVKDYFPEPVTVSWNSGALTSGVHTFPAVL |
| | H4HEN) | QSSGLYSLSSVVTVPSSSLGTKTYTCNVDHKPSNTK |
| | | VDKRVGGGGSGGGGSGGGGSEVQLVESGGGLVQP |
| | | GGSLKLSCAASGFTFSTYAMNWVRQASGKGLEWV |
| | | GRIRSKYNNYATYYADSVKDRFTISRDDSKNTAYLQ |
| | | MNSLKTEDTAVYYCVRHGNFGNSYVSWWAYWGQ |
| | | GTLVTVSSGGGGSGGGGSGGGGSQTVVTQEPSLTVS |
| | | PGGTVTLTCGSSTGAVTTSNYVNWVQQKPGQAPRG |
| | | LIGGTNKRAPGTPARFSGSLIGGKAALTLSGAQPEDE |
| | | AEYYCVLWYSNRWVFGGGTKLTVLGGSGGSSGAS |
| | | GTGTAGGTGSGSGTGSGLSGRSDDHGGGSQGQSGS |
| | | GYLWGCEWNCGGITTGSSGGSGGGGSGGGGSGGG |
| | | GSGGSESKYGPPCPPCPAPEFEGGPSVFLFPPKPKDTL |
| | | MISRTPEVTCVVVDVSQEDPEVQFNWYVDGVEVHN |
| | | AKTKPREEQFNSTYRVVSVLTVLHQDWLNGKEYKC |
| | | KVSNKGLPSSIEKTISKAKGQPREPQVCTLPPSQEEM |
| | | TKNQVSLSCAVKGFYPSDIAVEWESNGQPENNYKTT |
| | | PPVLDSDGSFFLVSRLTVDKSRWQEGNVFSCSVMHE |
| | | ALHNHYTQKSLSLSLGX |
| 617 | ProC1446: F5.17- | GSSKYGPPCPPCPAPEFEGGPSVFLFPPKPKDTLMISR |
| | Tras-25-v619h- | TPEVTCVVVDVSQEDPEVQFNWYVDGVEVHNAKT |
| | 20GG-24-Half TCB | KPREEQFNSTYRVVSVLTVLHQDWLNGKEYKCKVS |
| | (HC_FcK_IgG4_EN) | NKGLPSSIEKTISKAKGQPREPQVYTLPPCQEEMTKN |
| | | QVSLWCLVKGFYPSDIAVEWESNGQPENNYKTTPP |
| | | VLDSDGSFFLYSRLTVDKSRWQEGNVFSCSVMHEA |
| | | LHNHYTQKSLSLSLGX |
| 618 | ProC1446: F5.17- | QGQSGQGALICCSDVSGLCRWCGGGSSGGSISSGLL |
| | Tras-25-v619h- | SGRSDNHGGGSDIQMTQSPSSLSASVGDRVTITCRAS |
| | 20GG-24-Half TCB | QDVNTAVAWYQQKPGKAPKLLIYSASFLYSGVPSRF |
| | (LC_Tras 5.17) | SGSRSGTDFTLTISSLQPEDFATYYCQQHYTTPPTFG |
| | | QGTKVEIKRTVAAPSVFIFPPSDEQLKSGTASVVCLL |
| | | NNFYPREAKVQWKVDNALQSGNSQESVTEQDSKDS |
| | | TYSLSSTLTLSKADYEKHKVYACEVTHQGLSSPVTK |
| | | SFNRGEC |
| 619 | ProC1447: F5.17- | EVQLVESGGGLVQPGGSLRLSCAASGFNIKDTYIHW |
| | Tras-25-v619h- | VRQAPGKGLEWVARIYPTNGYTRYADSVKGRFTISA |
| | MN15a-24-Half | DTSKNTAYLQMNSLRAEDTAVYYCSRWGGDGFYA |
| | TCB | MDYWGQGTLVTVSSASTKGPSVFPLAPCSRSTSEST |
| | (HC_Tras-25- | AALGCLVKDYFPEPVTVSWNSGALTSGVHTFPAVL |
| | v619h-MN15a-24- | QSSGLYSLSSVVTVPSSSLGTKTYTCNVDHKPSNTK |
| | H4HEN) | VDKRVGGGGSGGGGSGGGGSEVQLVESGGGLVQP |
| | | GGSLKLSCAASGFTFSTYAMNWVRQASGKGLEWV |
| | | GRIRSKYNNYATYYADSVKDRFTISRDDSKNTAYLQ |
| | | MNSLKTEDTAVYYCVRHGNFGNSYVSWWAYWGQ |
| | | GTLVTVSSGGGGSGGGGSGGGGSQTVVTQEPSLTVS |
| | | PGGTVTLTCGSSTGAVTTSNYVNWVQQKPGQAPRG |
| | | LIGGTNKRAPGTPARFSGSLIGGKAALTLSGAQPEDE |
| | | AEYYCVLWYSNRWVFGGGTKLTVLGGSGGSSGAS |
| | | GTGTAGGTGSGSGTGSGLSGRSDDHGGGSQGQSGS |
| | | NAFRCWWDPPCQPMTGSSGGSGGGGSGGGGSGGG |
| | | GSGGSESKYGPPCPPCPAPEFEGGPSVFLFPPKPKDTL |
| | | MISRTPEVTCVVVDVSQEDPEVQFNWYVDGVEVHN |
| | | AKTKPREEQFNSTYRVVSVLTVLHQDWLNGKEYKC |
| | | KVSNKGLPSSIEKTISKAKGQPREPQVCTLPPSQEEM |
| | | TKNQVSLSCAVKGFYPSDIAVEWESNGQPENNYKTT |
| | | PPVLDSDGSFFLVSRLTVDKSRWQEGNVFSCSVMHE |
| | | ALHNHYTQKSLSLSLGX |
| 620 | ProC1447: F5.17- | GSSKYGPPCPPCPAPEFEGGPSVFLFPPKPKDTLMISR |
| | Tras-25-v619h- | TPEVTCVVVDVSQEDPEVQFNWYVDGVEVHNAKT |
| | MN15a-24-Half | KPREEQFNSTYRVVSVLTVLHQDWLNGKEYKCKVS |
| | TCB | NKGLPSSIEKTISKAKGQPREPQVYTLPPCQEEMTKN |
| | (HC_FcK_IgG4_EN) | QVSLWCLVKGFYPSDIAVEWESNGQPENNYKTTPP |
| | | VLDSDGSFFLYSRLTVDKSRWQEGNVFSCSVMHEA |
| | | LHNHYTQKSLSLSLGX |
| 621 | ProC1447: F5.17- | QGQSGQGALICCSDVSGLCRWCGGGSSGGSISSGLL |
| | Tras-25-v619h- | SGRSDNHGGGSDIQMTQSPSSLSASVGDRVTITCRAS |
| | MN15a-24-Half | QDVNTAVAWYQQKPGKAPKLLIYSASFLYSGVPSRF |
| | TCB | SGSRSGTDFTLTISSLQPEDFATYYCQQHYTTPPTFG |
| | (LC_Tras 5.17) | QGTKVEIKRTVAAPSVFIFPPSDEQLKSGTASVVCLL |
| | | NNFYPREAKVQWKVDNALQSGNSQESVTEQDSKDS |
| | | TYSLSSTLTLSKADYEKHKVYACEVTHQGLSSPVTK |
| | | SFNRGEC |
| 622 | ProC1448: F5.17- | EVQLVESGGGLVQPGGSLRLSCAASGFNIKDTYIHW |
| | Tras-25-v619h- | VRQAPGKGLEWVARIYPTNGYTRYADSVKGRFTISA |
| | MN15b-24-Half | DTSKNTAYLQMNSLRAEDTAVYYCSRWGGDGFYA |
| | TCB | MDYWGQGTLVTVSSASTKGPSVFPLAPCSRSTSEST |
| | (HC_Tras-25- | AALGCLVKDYFPEPVTVSWNSGALTSGVHTFPAVL |
| | v619h-MN15b-24- | QSSGLYSLSSVVTVPSSSLGTKTYTCNVDHKPSNTK |
| | H4HEN) | VDKRVGGGGSGGGGSGGGGSEVQLVESGGGLVQP |
| | | GGSLKLSCAASGFTFSTYAMNWVRQASGKGLEWV |
| | | GRIRSKYNNYATYYADSVKDRFTISRDDSKNTAYLQ |
| | | MNSLKTEDTAVYYCVRHGNFGNSYVSWWAYWGQ |
| | | GTLVTVSSGGGGSGGGGSGGGGSQTVVTQEPSLTVS |
| | | PGGTVTLTCGSSTGAVTTSNYVNWVQQKPGQAPRG |
| | | LIGGTNKRAPGTPARFSGSLIGGKAALTLSGAQPEDE |
| | | AEYYCVLWYSNRWVFGGGTKLTVLGGSGGSSGAS |
| | | GTGTAGGTGSGSGTGSGLSGRSDDHGGGSQGQSGS |
| | | ARGLCWWDPPCTHDLGSSGGSGGGGSGGGGSGGG |
| | | GSGGSESKYGPPCPPCPAPEFEGGPSVFLFPPKPKDTL |
| | | MISRTPEVTCVVVDVSQEDPEVQFNWYVDGVEVHN |
| | | AKTKPREEQFNSTYRVVSVLTVLHQDWLNGKEYKC |
| | | KVSNKGLPSSIEKTISKAKGQPREPQVCTLPPSQEEM |
| | | TKNQVSLSCAVKGFYPSDIAVEWESNGQPENNYKTT |
| | | PPVLDSDGSFFLVSRLTVDKSRWQEGNVFSCSVMHE |
| | | ALHNHYTQKSLSLSLGX |
| 623 | ProC1448: F5.17- | GSSKYGPPCPPCPAPEFEGGPSVFLFPPKPKDTLMISR |
| | Tras-25-v619h- | TPEVTCVVVDVSQEDPEVQFNWYVDGVEVHNAKT |
| | MN15b-24-Half | KPREEQFNSTYRVVSVLTVLHQDWLNGKEYKCKVS |
| | TCB | NKGLPSSIEKTISKAKGQPREPQVYTLPPCQEEMTKN |
| | (HC_FcK_IgG4_EN) | QVSLWCLVKGFYPSDIAVEWESNGQPENNYKTTPP |
| | | VLDSDGSFFLYSRLTVDKSRWQEGNVFSCSVMHEA |
| | | LHNHYTQKSLSLSLGX |
| 624 | ProC1448: F5.17- | QGQSGQGALICCSDVSGLCRWCGGGSSGGSISSGLL |
| | Tras-25-v619h- | SGRSDNHGGGSDIQMTQSPSSLSASVGDRVTITCRAS |
| | MN15b-24-Half | QDVNTAVAWYQQKPGKAPKLLIYSASFLYSGVPSRF |
| | TCB (LC_Tras 5.17) | SGSRSGTDFTLTISSLQPEDFATYYCQQHYTTPPTFG |
| | | QGTKVEIKRTVAAPSVFIFPPSDEQLKSGTASVVCLL |
| | | NNFYPREAKVQWKVDNALQSGNSQESVTEQDSKDS |
| | | TYSLSSTLTLSKADYEKHKVYACEVTHQGLSSPVTK |
| | | SFNRGEC |
| 625 | ProC306: | GAAGTTCAGCTTGTTGAATCTGGCGGCGGACTGGT |
| | v619_HLh_Tras 1/2 | TCAGCCTGGCGGATCTCTGAAACTGAGCTGTGCCG |
| | TCB | CCAGCGGCTTCACCTTTAGCACCTACGCCATGAAC |
| | (v619_HLh) | TGGGTCCGACAGGCCTCTGGCAAAGGCCTGGAAT |
| | | GGGTCGGACGGATCAGAAGCAAGTACAACAACTA |
| | | CGCCACCTACTACGCCGACAGCGTGAAGGACAGA |
| | | TTCACCATCAGCCGGGACGACAGCAAGAACACCG |
| | | CCTACCTGCAGATGAACAGCCTGAAAACCGAGGA |
| | | CACCGCCGTGTACTACTGTGTGCGGCACGGCAACT |
| | | TCGGCAACAGCTATGTGTCTTGGTGGGCCTATTGG |
| | | GGCCAGGGCACACTGGTTACAGTTAGCTCTGGTG |
| | | GCGGAGGATCTGGCGGAGGTGGAAGCGGCGGAG |
| | | GCGGTTCTCAAACAGTGGTCACACAAGAGCCCAG |
| | | CCTGACCGTTTCTCCTGGCGGAACAGTGACCCTGA |
| | | CATGCGGATCTTCTACAGGCGCCGTGACCACCAG |
| | | CAACTATGTGAATTGGGTGCAGCAGAAGCCCGGA |
| | | CAGGCTCCTAGAGGACTGATCGGCGGCACCAACA |
| | | AAAGAGCCCCTGGAACACCAGCCAGGTTCAGCGG |
| | | CTCTCTGATCGGAGGAAAAGCCGCTCTGACACTGT |
| | | CTGGCGCTCAGCCTGAAGATGAGGCCGAGTACTA |
| | | TTGCGTGCTGTGGTACAGCAACAGATGGGTGTTCG |
| | | GAGGCGGCACAAAGCTGACAGTGCTTGGCGGTGG |
| | | CGGTAGCGAAGTTCAGCTGGTGGAGAGTGGTGGG |
| | | GGGCTGGTTCAGCCTGGCGGTTCGCTCAGGCTGTC |
| | | ATGCGCAGCGAGCGGGTTCAACATCAAGGACACG |
| | | TATATCCATTGGGTGAGACAGGCCCCCGGGAAGG |
| | | GCCTCGAATGGGTTGCAAGGATTTATCCTACTAAC |
| | | GGGTACACAAGGTATGCTGACAGTGTGAAGGGGC |
| | | GGTTCACAATTTCCGCCGATACTTCTAAGAATACT |
| | | GCTTACCTCCAGATGAACTCCCTGAGGGCCGAGG |
| | | ACACTGCCGTTTACTACTGCTCCCGCTGGGGAGGA |
| | | GACGGCTTTTATGCTATGGATTATTGGGGTCAAGG |
| | | CACTCTAGTCACCGTGAGTAGTGCTAGCACAAAG |
| | | GGCCCCAGCGTGTTCCCTCTGGCTCCTTGTAGCAG |
| | | AAGCACCAGCGAGTCTACAGCCGCTCTGGGCTGT |
| | | CTGGTCAAGGACTACTTTCCCGAGCCTGTGACCGT |
| | | GTCCTGGAATAGCGGAGCACTGACCAGCGGCGTG |
| | | CACACATTTCCAGCCGTGCTGCAAAGCAGCGGCC |
| | | TGTACTCTCTGAGCAGCGTGGTCACAGTGCCTAGC |
| | | TCTAGCCTGGGCACCAAGACCTACACCTGTAATGT |
| | | GGACCACAAGCCTAGCAACACCAAGGTGGACAAG |
| | | CGCGTGGAATCTAAGTACGGCCCTCCTTGTCCTCC |
| | | ATGTCCTGCTCCAGAGTTTGAAGGCGGCCCTTCCG |
| | | TGTTCCTGTTTCCTCCAAAGCCTAAGGACACCCTG |
| | | ATGATCAGCAGAACCCCTGAAGTGACCTGCGTGG |
| | | TGGTGGACGTTTCCCAAGAGGACCCTGAGGTGCA |
| | | GTTCAATTGGTACGTGGACGGCGTGGAAGTGCAC |
| | | AACGCCAAGACCAAGCCTAGAGAGGAACAGTTCA |
| | | ACAGCACCTACAGAGTGGTGTCCGTGCTGACAGT |
| | | GCTGCACCAGGATTGGCTGAACGGCAAAGAGTAC |
| | | AAGTGCAAGGTGTCCAACAAGGGCCTGCCTAGCA |
| | | GCATCGAGAAAACCATCAGCAAGGCCAAGGGCCA |
| | | GCCTCGCGAACCTCAAGTCTGTACACTGCCTCCTA |
| | | GCCAAGAGGAAATGACCAAGAACCAGGTGTCCCT |
| | | GAGCTGCGCCGTGAAGGGCTTTTACCCTTCCGATA |
| | | TCGCCGTGGAATGGGAGAGCAATGGCCAGCCAGA |
| | | GAACAACTACAAGACAACCCCTCCTGTGCTGGAC |
| | | AGCGACGGCTCATTCTTCCTGGTGTCCAGACTGAC |
| | | CGTGGACAAGAGCAGATGGCAAGAGGGCAACGT |
| | | GTTCAGCTGCAGCGTGATGCACGAGGCCCTGCAC |
| | | AACCACTACACCCAGAAGTCTCTGAGCCTGTCTCT |
| | | GGGCNNN |
| 626 | ProC306: | GGATCTTCTAAGTACGGACCTCCTTGTCCTCCATG |
| | v619_HLh_Tras 1/2 | TCCTGCTCCAGAGTTTGAAGGCGGCCCTTCCGTGT |
| | TCB | TCCTGTTTCCTCCAAAGCCTAAGGACACCCTGATG |
| | (HC_FcK_IgG4_EN) | ATCAGCAGAACCCCTGAAGTGACCTGCGTGGTGG |
| | | TGGACGTGTCCCAAGAGGATCCTGAGGTGCAGTT |
| | | CAATTGGTACGTGGACGGCGTGGAAGTGCACAAC |
| | | GCCAAGACCAAGCCTAGAGAGGAACAGTTCAACA |
| | | GCACCTACAGAGTGGTGTCCGTGCTGACCGTGCTG |
| | | CACCAGGATTGGCTGAACGGCAAAGAGTACAAGT |
| | | GCAAGGTGTCCAACAAGGGCCTGCCTAGCAGCAT |
| | | CGAGAAAACCATCAGCAAGGCCAAGGGCCAGCCA |
| | | AGAGAACCCCAGGTGTACACACTGCCTCCATGCC |
| | | AAGAGGAAATGACCAAGAACCAGGTGTCCCTGTG |
| | | GTGCCTGGTCAAGGGCTTCTACCCTTCCGATATCG |
| | | CCGTGGAATGGGAGAGCAATGGCCAGCCTGAGAA |
| | | CAACTACAAGACCACACCTCCTGTGCTGGACAGC |
| | | GACGGCTCATTCTTCCTGTACAGCAGACTGACCGT |
| | | GGACAAGAGCAGATGGCAAGAGGGCAACGTGTTC |
| | | AGCTGCAGCGTGATGCACGAGGCCCTGCACAACC |
| | | ACTACACCCAGAAGTCTCTGAGCCTGAGCCTGGG |
| | | CNNN |
| 627 | ProC306: | GATATTCAGATGACACAGAGCCCCAGCAGCCTGT |
| | v619_HLh_Tras 1/2 | CTGCCTCTGTGGGAGACAGAGTGACCATCACCTGT |
| | TCB (LC_Tras) | AGAGCCAGCCAGGACGTGAACACAGCCGTGGCTT |
| | | GGTATCAGCAGAAGCCTGGCAAGGCCCCTAAGCT |
| | | GCTGATCTACAGCGCCAGCTTTCTGTACAGCGGCG |
| | | TGCCCAGCAGATTCAGCGGCTCTAGAAGCGGCAC |
| | | CGACTTCACCCTGACCATAAGCAGTCTGCAGCCCG |
| | | AGGACTTCGCCACCTACTACTGTCAGCAGCACTAC |
| | | ACCACACCTCCAACCTTTGGCCAGGGCACCAAGG |
| | | TGGAAATCAAGCGGACAGTGGCCGCTCCTAGCGT |
| | | GTTCATCTTTCCACCTAGCGACGAGCAGCTGAAGT |
| | | CTGGCACAGCCTCTGTCGTGTGCCTGCTGAACAAC |
| | | TTCTACCCCAGAGAAGCCAAGGTGCAGTGGAAGG |
| | | TGGACAACGCCCTGCAGAGCGGCAATAGCCAAGA |
| | | GAGCGTGACCGAGCAGGACAGCAAGGACTCTACC |
| | | TACAGCCTGAGCAGCACCCTGACACTGAGCAAGG |
| | | CCGACTACGAGAAGCACAAAGTGTACGCCTGCGA |
| | | AGTGACCCACCAGGGCCTTTCTAGCCCTGTGACCA |
| | | AGAGCTTCAACCGGGGCGAATGT |
| 628 | ProC531: | GAAGTTCAGCTTGTTGAATCTGGCGGCGGACTGGT |
| | v619_hh9_Tras 1/2 | TCAGCCTGGCGGATCTCTGAAACTGAGCTGTGCCG |
| | TCB | CCAGCGGCTTCACCTTTAGCACCTACGCCATGAAC |
| | HC | TGGGTCCGACAGGCCTCTGGCAAAGGCCTGGAAT |
| | (v619_hh9_Tras 1/2 | GGGTCGGACGGATCAGAAGCAAGTACAACAACTA |
| | TCB) | CGCCACCTACTACGCCGACAGCGTGAAGGACAGA |
| | | TTCACCATCAGCCGGGACGACAGCAAGAACACCG |
| | | CCTACCTGCAGATGAACAGCCTGAAAACCGAGGA |
| | | CACCGCCGTGTACTACTGTGTGCGGCACGGCAACT |
| | | TCGGCAACAGCTATGTGTCTTGGTGGGCCTATTGG |
| | | GGCCAGGGCACACTGGTTACAGTTAGCTCTGGTG |
| | | GCGGAGGATCTGGCGGAGGTGGAAGCGGCGGAG |
| | | GCGGTTCTCAAACAGTGGTCACACAAGAGCCCAG |
| | | CCTGACCGTTTCTCCTGGCGGAACAGTGACCCTGA |
| | | CCTGTAGATCTTCTACAGGCGCCGTGACCACCAGC |
| | | AATTACGCCAATTGGGTGCAGCAGAAGCCCGGAC |
| | | AGGCTCCTAGAGGACTGATCGGCGGCACCAACAA |
| | | AAGAGCCCCTGGAACACCAGCCAGGTTCAGCGGC |
| | | TCTCTGATCGGAGGAAAAGCCGCTCTGACACTGTC |
| | | TGGCGCTCAGCCTGAAGATGAGGCCGAGTACTAT |
| | | TGCGTGCTGTGGTACAGCAACCTGTGGGTGTTCGG |
| | | AGGCGGCACAAAGCTTACAGTGCTTGGAGGTGGC |
| | | GGCAGCGAGGTGCAACTTGTTGAGTCAGGCGGAG |
| | | GCCTTGTGCAACCTGGTGGTAGCCTGAGACTGTCT |
| | | TGTGCCGCTTCTGGCTTCAACATCAAGGACACCTA |
| | | CATCCACTGGGTTCGACAAGCTCCAGGCAAGGGC |
| | | CTTGAGTGGGTTGCCAGAATCTACCCCACCAACG |
| | | GCTACACCAGATACGCCGACTCTGTGAAAGGCCG |
| | | GTTCACCATCTCTGCCGACACCTCTAAGAATACTG |
| | | CCTATCTCCAAATGAACTCCCTGAGAGCCGAGGA |
| | | TACAGCCGTCTATTACTGCAGCAGATGGGGAGGC |
| | | GACGGCTTTTACGCCATGGATTACTGGGGACAGG |
| | | GAACCCTGGTCACCGTGTCCTCTGCTAGCACAAAG |
| | | GGCCCCAGCGTGTTCCCTCTGGCTCCTTGTAGCAG |
| | | AAGCACCAGCGAGTCTACAGCCGCTCTGGGCTGT |
| | | CTGGTCAAGGACTACTTTCCCGAGCCTGTGACCGT |
| | | GTCCTGGAATAGCGGAGCACTGACCAGCGGCGTG |
| | | CACACATTTCCAGCCGTGCTGCAAAGCAGCGGCC |
| | | TGTACTCTCTGAGCAGCGTGGTCACAGTGCCTAGC |
| | | TCTAGCCTGGGCACCAAGACCTACACCTGTAATGT |
| | | GGACCACAAGCCTAGCAACACCAAGGTGGACAAG |
| | | CGCGTGGAATCTAAGTACGGCCCTCCTTGTCCTCC |
| | | ATGTCCTGCTCCAGAGTTTGAAGGCGGCCCTTCCG |
| | | TGTTCCTGTTTCCTCCAAAGCCTAAGGACACCCTG |
| | | ATGATCAGCAGAACCCCTGAAGTGACCTGCGTGG |
| | | TGGTGGACGTTTCCCAAGAGGACCCTGAGGTGCA |
| | | GTTCAATTGGTACGTGGACGGCGTGGAAGTGCAC |
| | | AACGCCAAGACCAAGCCTAGAGAGGAACAGTTCA |
| | | ACAGCACCTACAGAGTGGTGTCCGTGCTGACAGT |
| | | GCTGCACCAGGATTGGCTGAACGGCAAAGAGTAC |
| | | AAGTGCAAGGTGTCCAACAAGGGCCTGCCTAGCA |
| | | GCATCGAGAAAACCATCAGCAAGGCCAAGGGCCA |
| | | GCCTCGCGAACCTCAAGTCTGTACACTGCCTCCTA |
| | | GCCAAGAGGAAATGACCAAGAACCAGGTGTCCCT |
| | | GAGCTGCGCCGTGAAGGGCTTTTACCCTTCCGATA |
| | | TCGCCGTGGAATGGGAGAGCAATGGCCAGCCAGA |
| | | GAACAACTACAAGACAACCCCTCCTGTGCTGGAC |
| | | AGCGACGGCTCATTCTTCCTGGTGTCCAGACTGAC |
| | | CGTGGACAAGAGCAGATGGCAAGAGGGCAACGT |
| | | GTTCAGCTGCAGCGTGATGCACGAGGCCCTGCAC |
| | | AACCACTACACCCAGAAGTCTCTGAGCCTGTCTCT |
| | | GGGCNNN |
| 629 | ProC531: | GGATCTTCTAAGTACGGACCTCCTTGTCCTCCATG |
| | v619_hh9_Tras 1/2 | TCCTGCTCCAGAGTTTGAAGGCGGCCCTTCCGTGT |
| | TCB | TCCTGTTTCCTCCAAAGCCTAAGGACACCCTGATG |
| | HC | ATCAGCAGAACCCCTGAAGTGACCTGCGTGGTGG |
| | (HC_FcK_IgG4_EN) | TGGACGTGTCCCAAGAGGATCCTGAGGTGCAGTT |
| | | CAATTGGTACGTGGACGGCGTGGAAGTGCACAAC |
| | | GCCAAGACCAAGCCTAGAGAGGAACAGTTCAACA |
| | | GCACCTACAGAGTGGTGTCCGTGCTGACCGTGCTG |
| | | CACCAGGATTGGCTGAACGGCAAAGAGTACAAGT |
| | | GCAAGGTGTCCAACAAGGGCCTGCCTAGCAGCAT |
| | | CGAGAAAACCATCAGCAAGGCCAAGGGCCAGCCA |
| | | AGAGAACCCCAGGTGTACACACTGCCTCCATGCC |
| | | AAGAGGAAATGACCAAGAACCAGGTGTCCCTGTG |
| | | GTGCCTGGTCAAGGGCTTCTACCCTTCCGATATCG |
| | | CCGTGGAATGGGAGAGCAATGGCCAGCCTGAGAA |
| | | CAACTACAAGACCACACCTCCTGTGCTGGACAGC |
| | | GACGGCTCATTCTTCCTGTACAGCAGACTGACCGT |
| | | GGACAAGAGCAGATGGCAAGAGGGCAACGTGTTC |
| | | AGCTGCAGCGTGATGCACGAGGCCCTGCACAACC |
| | | ACTACACCCAGAAGTCTCTGAGCCTGAGCCTGGG |
| | | CNNN |
| 630 | ProC531: | GATATTCAGATGACACAGAGCCCCAGCAGCCTGT |
| | v619_hh9_Tras 1/2 | CTGCCTCTGTGGGAGACAGAGTGACCATCACCTGT |
| | TCB | AGAGCCAGCCAGGACGTGAACACAGCCGTGGCTT |
| | HC (LC_Tras) | GGTATCAGCAGAAGCCTGGCAAGGCCCCTAAGCT |
| | | GCTGATCTACAGCGCCAGCTTTCTGTACAGCGGCG |
| | | TGCCCAGCAGATTCAGCGGCTCTAGAAGCGGCAC |
| | | CGACTTCACCCTGACCATAAGCAGTCTGCAGCCCG |
| | | AGGACTTCGCCACCTACTACTGTCAGCAGCACTAC |
| | | ACCACACCTCCAACCTTTGGCCAGGGCACCAAGG |
| | | TGGAAATCAAGCGGACAGTGGCCGCTCCTAGCGT |
| | | GTTCATCTTTCCACCTAGCGACGAGCAGCTGAAGT |
| | | CTGGCACAGCCTCTGTCGTGTGCCTGCTGAACAAC |
| | | TTCTACCCCAGAGAAGCCAAGGTGCAGTGGAAGG |
| | | TGGACAACGCCCTGCAGAGCGGCAATAGCCAAGA |
| | | GAGCGTGACCGAGCAGGACAGCAAGGACTCTACC |
| | | TACAGCCTGAGCAGCACCCTGACACTGAGCAAGG |
| | | CCGACTACGAGAAGCACAAAGTGTACGCCTGCGA |
| | | AGTGACCCACCAGGGCCTTTCTAGCCCTGTGACCA |
| | | AGAGCTTCAACCGGGGCGAATGT |
| 631 | ProC1965: v619h-5- | GAAGTTCAGCTTGTTGAATCTGGCGGCGGACTGGT |
| | Tras-noFc (an | TCAGCCTGGCGGATCTCTGAAACTGAGCTGTGCCG |
| | unmasked, no Fc | CCAGCGGCTTCACCTTTAGCACCTACGCCATGAAC |
| | form of SHL2) | TGGGTCCGACAGGCCTCTGGCAAAGGCCTGGAAT |
| | (PP_v619h-5-Tras- | GGGTCGGACGGATCAGAAGCAAGTACAACAACTA |
| | noFc) | CGCCACCTACTACGCCGACAGCGTGAAGGACAGA |
| | | TTCACCATCAGCCGGGACGACAGCAAGAACACCG |
| | | CCTACCTGCAGATGAACAGCCTGAAAACCGAGGA |
| | | CACCGCCGTGTACTACTGTGTGCGGCACGGCAACT |
| | | TCGGCAACAGCTATGTGTCTTGGTGGGCCTATTGG |
| | | GGCCAGGGCACACTGGTTACAGTTAGCTCTGGTG |
| | | GCGGAGGATCTGGCGGAGGTGGAAGCGGCGGAG |
| | | GCGGTTCTCAAACAGTGGTCACACAAGAGCCCAG |
| | | CCTGACCGTTTCTCCTGGCGGAACAGTGACCCTGA |
| | | CATGCGGATCTTCTACAGGCGCCGTGACCACCAG |
| | | CAACTATGTGAATTGGGTGCAGCAGAAGCCCGGA |
| | | CAGGCTCCTAGAGGACTGATCGGCGGCACCAACA |
| | | AAAGAGCCCCTGGAACACCAGCCAGGTTCAGCGG |
| | | CTCTCTGATCGGAGGAAAAGCCGCTCTGACACTGT |
| | | CTGGCGCCCAGCCAGAAGATGAGGCCGAGTACTA |
| | | TTGCGTGCTGTGGTACAGCAACAGATGGGTGTTCG |
| | | GAGGCGGCACAAAGCTGACAGTTCTTGGAGGCGG |
| | | AGGAAGTGAAGTGCAGCTGGTGGAAAGCGGTGGT |
| | | GGACTTGTTCAACCAGGCGGCAGCCTGAGACTGT |
| | | CTTGTGCCGCTTCTGGCTTCAACATCAAGGACACC |
| | | TACATCCACTGGGTTCGACAAGCTCCAGGCAAGG |
| | | GCCTTGAGTGGGTTGCCAGAATCTACCCCACCAAC |
| | | GGCTACACCAGATACGCCGACTCTGTGAAGGGCC |
| | | GCTTCACAATCTCTGCCGACACCTCCAAAAACACA |
| | | GCCTATCTCCAAATGAACTCCCTGAGAGCCGAGG |
| | | ATACAGCCGTCTATTACTGCAGCAGATGGGGAGG |
| | | CGACGGCTTTTACGCCATGGATTACTGGGGACAG |
| | | GGAACCCTGGTCACCGTGTCTAGCGCCTCTACAAA |
| | | GGGCCCTAGCGTGTTCCCTCTGGCTCCCTGTAGCA |
| | | GAAGCACCAGCGAATCTACAGCTGCCCTGGGCTG |
| | | CCTGGTCAAGGACTACTTTCCTGAGCCTGTGACCG |
| | | TGTCCTGGAACAGCGGAGCACTGACATCTGGCGT |
| | | GCACACCTTTCCAGCCGTGCTGCAAAGCAGCGGC |
| | | CTGTACTCTCTGAGCAGCGTGGTCACAGTGCCTAG |
| | | CTCTAGCCTGGGCACCAAGACCTACACCTGTAATG |
| | | TGGACCACAAGCCTAGCAACACCAAGGTGGACAA |
| | | GAGAGTC |
| 632 | ProC1965: v619h-5- | GATATTCAGATGACACAGAGCCCCAGCAGCCTGT |
| | Tras-noFc (an | CTGCCTCTGTGGGAGACAGAGTGACCATCACCTGT |
| | unmasked, no Fc | AGAGCCAGCCAGGACGTGAACACAGCCGTGGCTT |
| | form of SHL2) | GGTATCAGCAGAAGCCTGGCAAGGCCCCTAAGCT |
| | (LC_Tras) | GCTGATCTACAGCGCCAGCTTTCTGTACAGCGGCG |
| | | TGCCCAGCAGATTCAGCGGCTCTAGAAGCGGCAC |
| | | CGACTTCACCCTGACCATAAGCAGTCTGCAGCCCG |
| | | AGGACTTCGCCACCTACTACTGTCAGCAGCACTAC |
| | | ACCACACCTCCAACCTTTGGCCAGGGCACCAAGG |
| | | TGGAAATCAAGCGGACAGTGGCCGCTCCTAGCGT |
| | | GTTCATCTTTCCACCTAGCGACGAGCAGCTGAAGT |
| | | CTGGCACAGCCTCTGTCGTGTGCCTGCTGAACAAC |
| | | TTCTACCCCAGAGAAGCCAAGGTGCAGTGGAAGG |
| | | TGGACAACGCCCTGCAGAGCGGCAATAGCCAAGA |
| | | GAGCGTGACCGAGCAGGACAGCAAGGACTCTACC |
| | | TACAGCCTGAGCAGCACCCTGACACTGAGCAAGG |
| | | CCGACTACGAGAAGCACAAAGTGTACGCCTGCGA |
| | | AGTGACCCACCAGGGCCTTTCTAGCCCTGTGACCA |
| | | AGAGCTTCAACCGGGGCGAATGT |
| 633 | ProC1963: Tras-25- | GAAGTTCAGCTTGTTGAATCTGGCGGCGGACTGGT |
| | v619h-noFc (an | TCAGCCTGGCGGATCTCTGAGACTGTCTTGTGCCG |
| | unmasked/no Fc | CCAGCGGCTTCAACATCAAGGACACCTACATCCA |
| | form of SHL1) | CTGGGTCCGACAGGCCCCTGGCAAAGGACTTGAA |
| | (PP_Tras-25-v619h- | TGGGTCGCCAGAATCTACCCCACCAACGGCTACA |
| | noFc) | CCAGATACGCCGACTCTGTGAAGGGCAGATTCAC |
| | | CATCAGCGCCGACACCAGCAAGAACACCGCCTAC |
| | | CTGCAGATGAACAGCCTGAGAGCCGAGGACACCG |
| | | CCGTGTACTACTGTTCTAGATGGGGAGGCGACGG |
| | | CTTCTACGCCATGGATTATTGGGGCCAGGGCACCC |
| | | TGGTCACAGTGTCTAGCGCCTCTACAAAGGGCCCC |
| | | AGCGTTTTCCCACTGGCTCCCTGTAGCAGAAGCAC |
| | | CAGCGAATCTACAGCCGCTCTGGGCTGCCTGGTCA |
| | | AGGACTACTTTCCTGAGCCTGTGACCGTGTCCTGG |
| | | AACTCTGGCGCTCTGACATCTGGCGTGCACACCTT |
| | | TCCAGCCGTGCTGCAAAGCAGCGGCCTGTACTCTC |
| | | TGAGCAGCGTCGTGACAGTGCCTAGCAGCTCTCTG |
| | | GGCACCAAGACCTACACCTGTAATGTGGACCACA |
| | | AGCCTAGCAACACCAAGGTGGACAAGAGAGTTGG |
| | | CGGAGGCGGATCAGGTGGCGGAGGAAGCGGAGG |
| | | CGGCGGATCTGAAGTTCAACTGGTGGAAAGCGGC |
| | | GGAGGCCTTGTGCAACCTGGTGGCAGTCTGAAAC |
| | | TGAGCTGTGCCGCCTCTGGCTTCACCTTCAGCACA |
| | | TACGCCATGAATTGGGTTCGACAAGCCAGCGGCA |
| | | AGGGCCTCGAATGGGTTGGAAGAATCAGAAGCAA |
| | | GTACAACAACTACGCCACCTACTACGCCGACAGC |
| | | GTGAAGGATCGGTTTACCATCAGCCGGGACGACT |
| | | CCAAGAATACTGCCTATCTCCAAATGAACTCCCTC |
| | | AAGACCGAGGATACAGCCGTCTATTACTGCGTGC |
| | | GGCACGGCAACTTCGGCAACAGCTATGTGTCTTG |
| | | GTGGGCCTACTGGGGACAGGGAACACTGGTTACT |
| | | GTGTCCAGTGGCGGTGGTGGAAGTGGCGGCGGAG |
| | | GATCAGGCGGTGGCGGTTCTCAAACAGTGGTCAC |
| | | CCAAGAGCCTAGCCTGACAGTGTCTCCTGGCGGA |
| | | ACCGTGACACTGACATGTGGCAGTTCTACAGGCG |
| | | CCGTGACCACCAGCAACTACGTGAACTGGGTGCA |
| | | GCAGAAGCCAGGCCAGGCTCCTAGAGGACTGATC |
| | | GGAGGCACCAACAAGAGAGCCCCTGGAACACCAG |
| | | CCAGATTCTCCGGCTCTCTGATCGGCGGAAAAGCC |
| | | GCACTGACACTGTCTGGTGCTCAGCCTGAGGATG |
| | | AGGCCGAGTACTATTGCGTGCTGTGGTACAGCAA |
| | | CAGATGGGTGTTCGGCGGAGGGACCAAGCTGACA |
| | | GTGCTT |
| 634 | ProC1963: Tras-25- | GATATTCAGATGACACAGAGCCCCAGCAGCCTGT |
| | v619h-noFc (an | CTGCCTCTGTGGGAGACAGAGTGACCATCACCTGT |
| | unmasked/no Fc | AGAGCCAGCCAGGACGTGAACACAGCCGTGGCTT |
| | form of SHL1) | GGTATCAGCAGAAGCCTGGCAAGGCCCCTAAGCT |
| | (LC_Tras) | GCTGATCTACAGCGCCAGCTTTCTGTACAGCGGCG |
| | | TGCCCAGCAGATTCAGCGGCTCTAGAAGCGGCAC |
| | | CGACTTCACCCTGACCATAAGCAGTCTGCAGCCCG |
| | | AGGACTTCGCCACCTACTACTGTCAGCAGCACTAC |
| | | ACCACACCTCCAACCTTTGGCCAGGGCACCAAGG |
| | | TGGAAATCAAGCGGACAGTGGCCGCTCCTAGCGT |
| | | GTTCATCTTTCCACCTAGCGACGAGCAGCTGAAGT |
| | | CTGGCACAGCCTCTGTCGTGTGCCTGCTGAACAAC |
| | | TTCTACCCCAGAGAAGCCAAGGTGCAGTGGAAGG |
| | | TGGACAACGCCCTGCAGAGCGGCAATAGCCAAGA |
| | | GAGCGTGACCGAGCAGGACAGCAAGGACTCTACC |
| | | TACAGCCTGAGCAGCACCCTGACACTGAGCAAGG |
| | | CCGACTACGAGAAGCACAAAGTGTACGCCTGCGA |
| | | AGTGACCCACCAGGGCCTTTCTAGCCCTGTGACCA |
| | | AGAGCTTCAACCGGGGCGAATGT |
| 635 | ProC3007: 20GG- | CAAGGACAATCTGGCTCTGGCTATCTGTGGGGCTG |
| | GL1204DDH- | CGAGTGGAATTGTGGCGGCATCACAACAGGCTCT |
| | v619h-5mr-F5.17- | AGCGGCGGAAGCGGAGGATCTGGTGGACTGTCTG |
| | Tras-1490DNI- | GCAGATCCGATGATCACGGCGGATCTGAGGTGCA |
| | SHL2-TCB | GCTGGTGGAATCTGGCGGAGGACTTGTTCAGCCT |
| | (HC_20GG- | GGCGGCTCTCTGAAGCTGTCTTGTGCCGCCAGCGG |
| | GL1204DDH- | CTTCACCTTTAGCACCTACGCCATGAACTGGGTCC |
| | v619h-5mr-Tras- | GACAGGCCTCTGGCAAAGGCCTGGAATGGGTCGG |
| | 1490DNI-FcH4EN) | ACGGATCAGAAGCAAGTACAACAACTACGCCACC |
| | | TACTACGCCGACAGCGTGAAGGACAGATTCACCA |
| | | TCAGCCGGGACGACAGCAAGAACACCGCCTACCT |
| | | GCAGATGAACAGCCTGAAAACCGAGGACACCGCC |
| | | GTGTACTACTGTGTGCGGCACGGCAACTTCGGCA |
| | | ACAGCTATGTGTCTTGGTGGGCCTATTGGGGCCAG |
| | | GGCACACTGGTTACAGTTAGCAGTGGCGGCGGAG |
| | | GTAGTGGCGGAGGTGGCAGCGGAGGCGGTGGATC |
| | | TCAAACAGTGGTCACCCAAGAGCCTAGCCTGACC |
| | | GTTTCTCCTGGCGGAACCGTGACACTGACATGCGG |
| | | ATCTTCTACAGGCGCCGTGACCACCAGCAACTATG |
| | | TGAATTGGGTGCAGCAGAAGCCCGGACAGGCTCC |
| | | TAGAGGACTGATCGGAGGCACCAACAAGAGAGCC |
| | | CCTGGAACACCAGCCAGATTCAGCGGATCTCTGA |
| | | TCGGCGGAAAGGCCGCTCTGACACTTTCTGGTGCC |
| | | CAGCCTGAAGATGAGGCCGAGTACTATTGCGTGC |
| | | TGTGGTACAGCAACAGATGGGTGTTCGGAGGCGG |
| | | CACCAAGCTGACAGTTCTTGGAGGCGGAGGCAGC |
| | | GAAGTCCAGCTGGTTGAAAGCGGTGGTGGCCTGG |
| | | TCCAACCAGGTGGAAGTCTGAGACTGAGCTGTGC |
| | | CGCTTCCGGCTTCAACATCAAGGACACCTACATCC |
| | | ACTGGGTTCGACAAGCTCCAGGCAAGGGCCTTGA |
| | | GTGGGTTGCCAGAATCTACCCCACCAACGGCTAC |
| | | ACCAGATACGCCGACTCTGTGAAAGGCCGGTTCA |
| | | CCATCTCTGCCGACACCTCCAAAAACACAGCCTAT |
| | | CTCCAAATGAACTCCCTGAGAGCCGAGGATACAG |
| | | CCGTCTATTACTGCAGCAGATGGGGAGGCGACGG |
| | | CTTTTACGCCATGGATTACTGGGGACAGGGAACC |
| | | CTGGTCACCGTGTCTAGCGCCTCTACAAAGGGCCC |
| | | TAGCGTGTTCCCTCTGGCTCCCTGTAGCAGAAGCA |
| | | CCAGCGAATCTACAGCCGCTCTGGGCTGCCTGGTC |
| | | AAGGACTACTTTCCTGAGCCTGTGACCGTGTCCTG |
| | | GAACTCTGGCGCTCTTACAAGCGGCGTGCACACCT |
| | | TTCCAGCCGTGCTGCAAAGCAGCGGCCTGTACTCT |
| | | CTGAGCAGCGTGGTCACAGTGCCTAGCTCTAGCCT |
| | | GGGCACAAAGACCTACACCTGTAATGTGGACCAC |
| | | AAGCCTAGCAACACCAAGGTGGACAAGCGCGTGG |
| | | AATCTAAGTACGGCGGAGGAAGCTCTGGCGGCAG |
| | | CATCTCTTCTGGACTGCTGAGCGGCAGAAGCGAC |
| | | AATATCGGCGGAGGCTCTGGACCTCCTTGTCCTCC |
| | | ATGTCCTGCTCCAGAGTTTGAAGGCGGCCCTTCCG |
| | | TGTTCCTGTTTCCTCCAAAGCCTAAGGACACCCTG |
| | | ATGATCAGCAGAACCCCTGAAGTGACCTGCGTGG |
| | | TGGTGGACGTGTCCCAAGAGGATCCAGAGGTGCA |
| | | GTTCAATTGGTACGTGGACGGCGTGGAAGTGCAC |
| | | AACGCCAAGACCAAGCCTAGAGAGGAACAGTTCA |
| | | ACAGCACCTACAGAGTGGTGTCCGTGCTGACAGT |
| | | GCTGCACCAGGATTGGCTGAACGGCAAAGAGTAC |
| | | AAGTGCAAGGTGTCCAACAAGGGCCTGCCTAGCA |
| | | GCATCGAGAAAACCATCAGCAAGGCCAAGGGCCA |
| | | GCCTCGCGAACCTCAAGTCTGTACACTGCCTCCTA |
| | | GCCAAGAGGAAATGACCAAGAACCAGGTGTCCCT |
| | | GAGCTGCGCCGTGAAGGGCTTTTACCCTTCCGATA |
| | | TCGCCGTGGAATGGGAGAGCAATGGCCAGCCAGA |
| | | GAACAACTACAAGACAACCCCTCCTGTGCTGGAC |
| | | AGCGACGGCTCATTCTTCCTGGTGTCCAGACTGAC |
| | | CGTGGACAAGAGCAGATGGCAAGAGGGCAACGT |
| | | GTTCAGCTGCAGCGTGATGCACGAGGCCCTGCAC |
| | | AACCACTACACCCAGAAGTCTCTGAGCCTGTCTCT |
| | | GGGCNNN |
| 636 | ProC3007: 20GG- | GGATCTTCTAAGTACGGACCTCCTTGTCCTCCATG |
| | GL1204DDH- | TCCTGCTCCAGAGTTTGAAGGCGGCCCTTCCGTGT |
| | v619h-5mr-F5.17- | TCCTGTTTCCTCCAAAGCCTAAGGACACCCTGATG |
| | Tras-1490DNI- | ATCAGCAGAACCCCTGAAGTGACCTGCGTGGTGG |
| | SHL2-TCB | TGGACGTGTCCCAAGAGGATCCTGAGGTGCAGTT |
| | (HC_FcK_IgG4_EN) | CAATTGGTACGTGGACGGCGTGGAAGTGCACAAC |
| | | GCCAAGACCAAGCCTAGAGAGGAACAGTTCAACA |
| | | GCACCTACAGAGTGGTGTCCGTGCTGACCGTGCTG |
| | | CACCAGGATTGGCTGAACGGCAAAGAGTACAAGT |
| | | GCAAGGTGTCCAACAAGGGCCTGCCTAGCAGCAT |
| | | CGAGAAAACCATCAGCAAGGCCAAGGGCCAGCCA |
| | | AGAGAACCCCAGGTGTACACACTGCCTCCATGCC |
| | | AAGAGGAAATGACCAAGAACCAGGTGTCCCTGTG |
| | | GTGCCTGGTCAAGGGCTTCTACCCTTCCGATATCG |
| | | CCGTGGAATGGGAGAGCAATGGCCAGCCTGAGAA |
| | | CAACTACAAGACCACACCTCCTGTGCTGGACAGC |
| | | GACGGCTCATTCTTCCTGTACAGCAGACTGACCGT |
| | | GGACAAGAGCAGATGGCAAGAGGGCAACGTGTTC |
| | | AGCTGCAGCGTGATGCACGAGGCCCTGCACAACC |
| | | ACTACACCCAGAAGTCTCTGAGCCTGAGCCTGGG |
| | | CNNN |
| 637 | ProC3007: 20GG- | CAAGGACAATCTGGACAAGGCGCCCTGATCTGCT |
| | GL1204DDH- | GTTCCGATGTGTCTGGACTGTGCCGTTGGTGCGGC |
| | v619h-5mr-F5.17- | GGAGGATCTTCTGGCGGCTCTATCTCTTCTGGCCT |
| | Tras-1490DNI- | GCTGAGCGGCAGAAGCGACAATCACGGCGGAGGC |
| | SHL2-TCB | AGCGACATCCAGATGACACAGAGCCCTTCTAGCC |
| | (LC_Tras 5.17) | TGAGCGCCAGCGTGGGAGACAGAGTGACCATCAC |
| | | ATGTAGAGCCAGCCAGGACGTGAACACAGCCGTG |
| | | GCTTGGTATCAGCAGAAGCCTGGCAAGGCCCCTA |
| | | AGCTGCTGATCTACAGCGCCAGCTTTCTGTACAGC |
| | | GGCGTGCCCAGCAGATTCAGCGGCTCTAGAAGCG |
| | | GCACCGACTTCACCCTGACCATAAGCAGTCTGCA |
| | | GCCCGAGGACTTCGCCACCTACTACTGTCAGCAGC |
| | | ACTACACCACACCTCCAACCTTTGGCCAGGGCACC |
| | | AAGGTGGAAATCAAGCGGACAGTGGCCGCTCCTA |
| | | GCGTGTTCATCTTTCCACCTAGCGACGAGCAGCTG |
| | | AAGTCTGGCACAGCCTCTGTCGTGTGCCTGCTGAA |
| | | CAACTTCTACCCCAGAGAAGCCAAGGTGCAGTGG |
| | | AAGGTGGACAACGCCCTGCAGTCCGGCAATAGCC |
| | | AAGAGAGCGTGACCGAGCAGGACAGCAAGGACT |
| | | CTACCTACAGCCTGAGCAGCACCCTGACACTGAG |
| | | CAAGGCCGACTACGAGAAGCACAAAGTGTACGCC |
| | | TGCGAAGTGACCCACCAGGGCCTTTCTAGCCCTGT |
| | | GACCAAGAGCTTCAACCGGGGCGAATGT |
| 638 | ProC3008: 20GG- | CAAGGACAATCTGGCTCTGGCTATCTGTGGGGCTG |
| | GL1204DDH- | CGAGTGGAATTGTGGCGGCATCACAACAGGCTCT |
| | v619h-5mr-F5.17- | AGCGGCGGAAGCGGAGGATCTGGTGGACTGTCTG |
| | Tras-1406-SHL2- | GCAGATCCGATGATCACGGCGGATCTGAGGTGCA |
| | TCB | GCTGGTGGAATCTGGCGGAGGACTTGTTCAGCCT |
| | (HC_20GG- | GGCGGCTCTCTGAAGCTGTCTTGTGCCGCCAGCGG |
| | GL1204DDH- | CTTCACCTTTAGCACCTACGCCATGAACTGGGTCC |
| | v619h-5mr-Tras- | GACAGGCCTCTGGCAAAGGCCTGGAATGGGTCGG |
| | 1406-FcH4EN) | ACGGATCAGAAGCAAGTACAACAACTACGCCACC |
| | | TACTACGCCGACAGCGTGAAGGACAGATTCACCA |
| | | TCAGCCGGGACGACAGCAAGAACACCGCCTACCT |
| | | GCAGATGAACAGCCTGAAAACCGAGGACACCGCC |
| | | GTGTACTACTGTGTGCGGCACGGCAACTTCGGCA |
| | | ACAGCTATGTGTCTTGGTGGGCCTATTGGGGCCAG |
| | | GGCACACTGGTTACAGTTAGCAGTGGCGGCGGAG |
| | | GTAGTGGCGGAGGTGGCAGCGGAGGCGGTGGATC |
| | | TCAAACAGTGGTCACCCAAGAGCCTAGCCTGACC |
| | | GTTTCTCCTGGCGGAACCGTGACACTGACATGCGG |
| | | ATCTTCTACAGGCGCCGTGACCACCAGCAACTATG |
| | | TGAATTGGGTGCAGCAGAAGCCCGGACAGGCTCC |
| | | TAGAGGACTGATCGGAGGCACCAACAAGAGAGCC |
| | | CCTGGAACACCAGCCAGATTCAGCGGATCTCTGA |
| | | TCGGCGGAAAGGCCGCTCTGACACTTTCTGGTGCC |
| | | CAGCCTGAAGATGAGGCCGAGTACTATTGCGTGC |
| | | TGTGGTACAGCAACAGATGGGTGTTCGGAGGCGG |
| | | CACCAAGCTGACAGTTCTTGGAGGCGGAGGCAGC |
| | | GAAGTCCAGCTGGTTGAAAGCGGTGGTGGCCTGG |
| | | TCCAACCAGGTGGAAGTCTGAGACTGAGCTGTGC |
| | | CGCTTCCGGCTTCAACATCAAGGACACCTACATCC |
| | | ACTGGGTTCGACAAGCTCCAGGCAAGGGCCTTGA |
| | | GTGGGTTGCCAGAATCTACCCCACCAACGGCTAC |
| | | ACCAGATACGCCGACTCTGTGAAAGGCCGGTTCA |
| | | CCATCTCTGCCGACACCTCCAAAAACACAGCCTAT |
| | | CTCCAAATGAACTCCCTGAGAGCCGAGGATACAG |
| | | CCGTCTATTACTGCAGCAGATGGGGAGGCGACGG |
| | | CTTTTACGCCATGGATTACTGGGGACAGGGAACC |
| | | CTGGTCACCGTGTCTAGCGCCTCTACAAAGGGCCC |
| | | TAGCGTGTTCCCTCTGGCTCCCTGTAGCAGAAGCA |
| | | CCAGCGAATCTACAGCCGCTCTGGGCTGCCTGGTC |
| | | AAGGACTACTTTCCTGAGCCTGTGACCGTGTCCTG |
| | | GAACTCTGGCGCTCTTACAAGCGGCGTGCACACCT |
| | | TTCCAGCCGTGCTGCAAAGCAGCGGCCTGTACTCT |
| | | CTGAGCAGCGTGGTCACAGTGCCTAGCTCTAGCCT |
| | | GGGCACAAAGACCTACACCTGTAATGTGGACCAC |
| | | AAGCCTAGCAACACCAAGGTGGACAAGCGCGTGG |
| | | AATCTAAGTACGGCGGAGGAAGCTCTGGCGGCAG |
| | | CATCTCTTCTGGACTGCTGAGCGGCAGAAGCGAC |
| | | AATATCGGCGGAGGCTCTGGACCTCCTTGTCCTCC |
| | | ATGTCCTGCTCCAGAGTTTGAAGGCGGCCCTTCCG |
| | | TGTTCCTGTTTCCTCCAAAGCCTAAGGACACCCTG |
| | | ATGATCAGCAGAACCCCTGAAGTGACCTGCGTGG |
| | | TGGTGGACGTGTCCCAAGAGGATCCAGAGGTGCA |
| | | GTTCAATTGGTACGTGGACGGCGTGGAAGTGCAC |
| | | AACGCCAAGACCAAGCCTAGAGAGGAACAGTTCA |
| | | ACAGCACCTACAGAGTGGTGTCCGTGCTGACAGT |
| | | GCTGCACCAGGATTGGCTGAACGGCAAAGAGTAC |
| | | AAGTGCAAGGTGTCCAACAAGGGCCTGCCTAGCA |
| | | GCATCGAGAAAACCATCAGCAAGGCCAAGGGCCA |
| | | GCCTCGCGAACCTCAAGTCTGTACACTGCCTCCTA |
| | | GCCAAGAGGAAATGACCAAGAACCAGGTGTCCCT |
| | | GAGCTGCGCCGTGAAGGGCTTTTACCCTTCCGATA |
| | | TCGCCGTGGAATGGGAGAGCAATGGCCAGCCAGA |
| | | GAACAACTACAAGACAACCCCTCCTGTGCTGGAC |
| | | AGCGACGGCTCATTCTTCCTGGTGTCCAGACTGAC |
| | | CGTGGACAAGAGCAGATGGCAAGAGGGCAACGT |
| | | GTTCAGCTGCAGCGTGATGCACGAGGCCCTGCAC |
| | | AACCACTACACCCAGAAGTCTCTGAGCCTGTCTCT |
| | | GGGCNNN |
| 639 | ProC3008: 20GG- | CAAGGACAATCTGGCTCTGGCTATCTGTGGGGCTG |
| | GL1204DDH- | CGAGTGGAATTGTGGCGGCATCACAACAGGCTCT |
| | v619h-5mr-F5.17- | AGCGGCGGAAGCGGAGGATCTGGTGGACTGTCTG |
| | Tras-1406-SHL2- | GCAGATCCGATGATCACGGCGGATCTGAGGTGCA |
| | TCB | GCTGGTGGAATCTGGCGGAGGACTTGTTCAGCCT |
| | (HC_FcK_IgG4_EN) | GGCGGCTCTCTGAAGCTGTCTTGTGCCGCCAGCGG |
| | | CTTCACCTTTAGCACCTACGCCATGAACTGGGTCC |
| | | GACAGGCCTCTGGCAAAGGCCTGGAATGGGTCGG |
| | | ACGGATCAGAAGCAAGTACAACAACTACGCCACC |
| | | TACTACGCCGACAGCGTGAAGGACAGATTCACCA |
| | | TCAGCCGGGACGACAGCAAGAACACCGCCTACCT |
| | | GCAGATGAACAGCCTGAAAACCGAGGACACCGCC |
| | | GTGTACTACTGTGTGCGGCACGGCAACTTCGGCA |
| | | ACAGCTATGTGTCTTGGTGGGCCTATTGGGGCCAG |
| | | GGCACACTGGTTACAGTTAGCAGTGGCGGCGGAG |
| | | GTAGTGGCGGAGGTGGCAGCGGAGGCGGTGGATC |
| | | TCAAACAGTGGTCACCCAAGAGCCTAGCCTGACC |
| | | GTTTCTCCTGGCGGAACCGTGACACTGACATGCGG |
| | | ATCTTCTACAGGCGCCGTGACCACCAGCAACTATG |
| | | TGAATTGGGTGCAGCAGAAGCCCGGACAGGCTCC |
| | | TAGAGGACTGATCGGAGGCACCAACAAGAGAGCC |
| | | CCTGGAACACCAGCCAGATTCAGCGGATCTCTGA |
| | | TCGGCGGAAAGGCCGCTCTGACACTTTCTGGTGCC |
| | | CAGCCTGAAGATGAGGCCGAGTACTATTGCGTGC |
| | | TGTGGTACAGCAACAGATGGGTGTTCGGAGGCGG |
| | | CACCAAGCTGACAGTTCTTGGAGGCGGAGGCAGC |
| | | GAAGTCCAGCTGGTTGAAAGCGGTGGTGGCCTGG |
| | | TCCAACCAGGTGGAAGTCTGAGACTGAGCTGTGC |
| | | CGCTTCCGGCTTCAACATCAAGGACACCTACATCC |
| | | ACTGGGTTCGACAAGCTCCAGGCAAGGGCCTTGA |
| | | GTGGGTTGCCAGAATCTACCCCACCAACGGCTAC |
| | | ACCAGATACGCCGACTCTGTGAAAGGCCGGTTCA |
| | | CCATCTCTGCCGACACCTCCAAAAACACAGCCTAT |
| | | CTCCAAATGAACTCCCTGAGAGCCGAGGATACAG |
| | | CCGTCTATTACTGCAGCAGATGGGGAGGCGACGG |
| | | CTTTTACGCCATGGATTACTGGGGACAGGGAACC |
| | | CTGGTCACCGTGTCTAGCGCCTCTACAAAGGGCCC |
| | | TAGCGTGTTCCCTCTGGCTCCCTGTAGCAGAAGCA |
| | | CCAGCGAATCTACAGCCGCTCTGGGCTGCCTGGTC |
| | | AAGGACTACTTTCCTGAGCCTGTGACCGTGTCCTG |
| | | GAACTCTGGCGCTCTTACAAGCGGCGTGCACACCT |
| | | TTCCAGCCGTGCTGCAAAGCAGCGGCCTGTACTCT |
| | | CTGAGCAGCGTGGTCACAGTGCCTAGCTCTAGCCT |
| | | GGGCACAAAGACCTACACCTGTAATGTGGACCAC |
| | | AAGCCTAGCAACACCAAGGTGGACAAGCGCGTGG |
| | | AATCTAAGTACGGCGGAGGAAGCTCAGGCGGCTC |
| | | TGCTCCTAGATCTGCTCTGGCCCATGGACTGTTTG |
| | | GCGGAGGCTCTGGACCTCCTTGTCCTCCATGTCCT |
| | | GCTCCAGAGTTTGAAGGCGGCCCTTCCGTGTTCCT |
| | | GTTTCCTCCAAAGCCTAAGGACACCCTGATGATCA |
| | | GCAGAACCCCTGAAGTGACCTGCGTGGTGGTGGA |
| | | CGTGTCCCAAGAGGATCCAGAGGTGCAGTTCAAT |
| | | TGGTACGTGGACGGCGTGGAAGTGCACAACGCCA |
| | | AGACCAAGCCTAGAGAGGAACAGTTCAACAGCAC |
| | | CTACAGAGTGGTGTCCGTGCTGACAGTGCTGCACC |
| | | AGGATTGGCTGAACGGCAAAGAGTACAAGTGCAA |
| | | GGTGTCCAACAAGGGCCTGCCTAGCAGCATCGAG |
| | | AAAACCATCAGCAAGGCCAAGGGCCAGCCTCGCG |
| | | AACCTCAAGTCTGTACACTGCCTCCTAGCCAAGAG |
| | | GAAATGACCAAGAACCAGGTGTCCCTGAGCTGCG |
| | | CCGTGAAGGGCTTTTACCCTTCCGATATCGCCGTG |
| | | GAATGGGAGAGCAATGGCCAGCCAGAGAACAACT |
| | | ACAAGACAACCCCTCCTGTGCTGGACAGCGACGG |
| | | CTCATTCTTCCTGGTGTCCAGACTGACCGTGGACA |
| | | AGAGCAGATGGCAAGAGGGCAACGTGTTCAGCTG |
| | | CAGCGTGATGCACGAGGCCCTGCACAACCACTAC |
| | | ACCCAGAAGTCTCTGAGCCTGTCTCTGGGCNNN |
| 640 | ProC3008: 20GG- | CAAGGACAATCTGGACAAGGCGCCCTGATCTGCT |
| | GL1204DDH- | GTTCCGATGTGTCTGGACTGTGCCGTTGGTGCGGC |
| | v619h-5mr-F5.17- | GGAGGATCTTCTGGCGGCTCTATCTCTTCTGGCCT |
| | Tras-1406-SHL2- | GCTGAGCGGCAGAAGCGACAATCACGGCGGAGGC |
| | TCB | AGCGACATCCAGATGACACAGAGCCCTTCTAGCC |
| | (LC_Tras 5.17) | TGAGCGCCAGCGTGGGAGACAGAGTGACCATCAC |
| | | ATGTAGAGCCAGCCAGGACGTGAACACAGCCGTG |
| | | GCTTGGTATCAGCAGAAGCCTGGCAAGGCCCCTA |
| | | AGCTGCTGATCTACAGCGCCAGCTTTCTGTACAGC |
| | | GGCGTGCCCAGCAGATTCAGCGGCTCTAGAAGCG |
| | | GCACCGACTTCACCCTGACCATAAGCAGTCTGCA |
| | | GCCCGAGGACTTCGCCACCTACTACTGTCAGCAGC |
| | | ACTACACCACACCTCCAACCTTTGGCCAGGGCACC |
| | | AAGGTGGAAATCAAGCGGACAGTGGCCGCTCCTA |
| | | GCGTGTTCATCTTTCCACCTAGCGACGAGCAGCTG |
| | | AAGTCTGGCACAGCCTCTGTCGTGTGCCTGCTGAA |
| | | CAACTTCTACCCCAGAGAAGCCAAGGTGCAGTGG |
| | | AAGGTGGACAACGCCCTGCAGTCCGGCAATAGCC |
| | | AAGAGAGCGTGACCGAGCAGGACAGCAAGGACT |
| | | CTACCTACAGCCTGAGCAGCACCCTGACACTGAG |
| | | CAAGGCCGACTACGAGAAGCACAAAGTGTACGCC |
| | | TGCGAAGTGACCCACCAGGGCCTTTCTAGCCCTGT |
| | | GACCAAGAGCTTCAACCGGGGCGAATGT |
| 641 | ProC1441: 20GG- | CAAGGACAATCTGGCTCTGGCTATCTGTGGGGCTG |
| | v619h-F5.17-Tras- | CGAGTGGAATTGTGGCGGCATCACAACAGGCTCT |
| | Half TCB | AGCGGCGGAAGCGGAGGATCTGGTGGACTGTCTG |
| | (HC_20GG-v619h- | GCAGATCCGATGATCACGGCGGATCTGAGGTGCA |
| | Tras-H4HEN) | GCTGGTGGAATCTGGCGGAGGACTTGTTCAGCCT |
| | | GGCGGCTCTCTGAAGCTGTCTTGTGCCGCCAGCGG |
| | | CTTCACCTTTAGCACCTACGCCATGAACTGGGTCC |
| | | GACAGGCCTCTGGCAAAGGCCTGGAATGGGTCGG |
| | | ACGGATCAGAAGCAAGTACAACAACTACGCCACC |
| | | TACTACGCCGACAGCGTGAAGGACAGATTCACCA |
| | | TCAGCCGGGACGACAGCAAGAACACCGCCTACCT |
| | | GCAGATGAACAGCCTGAAAACCGAGGACACCGCC |
| | | GTGTACTACTGTGTGCGGCACGGCAACTTCGGCA |
| | | ACAGCTATGTGTCTTGGTGGGCCTATTGGGGCCAG |
| | | GGCACACTGGTTACAGTTAGCAGTGGCGGCGGAG |
| | | GTAGTGGCGGAGGTGGCAGCGGAGGCGGTGGATC |
| | | TCAAACAGTGGTCACCCAAGAGCCTAGCCTGACC |
| | | GTTTCTCCTGGCGGAACCGTGACACTGACATGCGG |
| | | ATCTTCTACAGGCGCCGTGACCACCAGCAACTATG |
| | | TGAATTGGGTGCAGCAGAAGCCCGGACAGGCTCC |
| | | TAGAGGACTGATCGGAGGCACCAACAAGAGAGCC |
| | | CCTGGAACACCAGCCAGATTCAGCGGATCTCTGA |
| | | TCGGCGGAAAGGCCGCTCTGACACTTTCTGGTGCC |
| | | CAGCCTGAAGATGAGGCCGAGTACTATTGCGTGC |
| | | TGTGGTACAGCAACAGATGGGTGTTCGGAGGCGG |
| | | CACCAAGCTGACAGTTCTTGGAGGCGGAGGCAGC |
| | | GAAGTCCAGCTGGTTGAAAGCGGTGGTGGCCTGG |
| | | TCCAACCAGGTGGAAGTCTGAGACTGAGCTGTGC |
| | | CGCTTCCGGCTTCAACATCAAGGACACCTACATCC |
| | | ACTGGGTTCGACAAGCTCCAGGCAAGGGCCTTGA |
| | | GTGGGTTGCCAGAATCTACCCCACCAACGGCTAC |
| | | ACCAGATACGCCGACTCTGTGAAAGGCCGGTTCA |
| | | CCATCTCTGCCGACACCTCCAAAAACACAGCCTAT |
| | | CTCCAAATGAACTCCCTGAGAGCCGAGGATACAG |
| | | CCGTCTATTACTGCAGCAGATGGGGAGGCGACGG |
| | | CTTTTACGCCATGGATTACTGGGGACAGGGAACC |
| | | CTGGTCACCGTGTCTAGCGCCTCTACAAAGGGCCC |
| | | TAGCGTGTTCCCTCTGGCTCCCTGTAGCAGAAGCA |
| | | CCAGCGAATCTACAGCCGCTCTGGGCTGCCTGGTC |
| | | AAGGACTACTTTCCTGAGCCTGTGACCGTGTCCTG |
| | | GAACTCTGGCGCTCTTACAAGCGGCGTGCACACCT |
| | | TTCCAGCCGTGCTGCAAAGCAGCGGCCTGTACTCT |
| | | CTGAGCAGCGTGGTCACAGTGCCTAGCTCTAGCCT |
| | | GGGCACAAAGACCTACACCTGTAATGTGGACCAC |
| | | AAGCCTAGCAACACCAAGGTGGACAAGCGCGTGG |
| | | AATCTAAGTACGGCCCTCCTTGTCCTCCATGTCCT |
| | | GCTCCAGAGTTTGAAGGCGGCCCTTCCGTGTTCCT |
| | | GTTTCCTCCAAAGCCTAAGGACACCCTGATGATCA |
| | | GCAGAACCCCTGAAGTGACCTGCGTGGTGGTGGA |
| | | CGTTTCCCAAGAGGACCCCGAGGTGCAGTTCAATT |
| | | GGTACGTGGACGGCGTGGAAGTGCACAACGCCAA |
| | | GACCAAGCCTAGAGAGGAACAGTTCAACAGCACC |
| | | TACAGAGTGGTGTCCGTGCTGACAGTGCTGCACC |
| | | AGGATTGGCTGAACGGCAAAGAGTACAAGTGCAA |
| | | GGTGTCCAACAAGGGCCTGCCTAGCAGCATCGAG |
| | | AAAACCATCAGCAAGGCCAAGGGCCAGCCTCGCG |
| | | AACCTCAAGTCTGTACACTGCCTCCTAGCCAAGAG |
| | | GAAATGACCAAGAACCAGGTGTCCCTGAGCTGCG |
| | | CCGTGAAGGGCTTTTACCCTTCCGATATCGCCGTG |
| | | GAATGGGAGAGCAATGGCCAGCCTGAGAACAACT |
| | | ACAAGACCACACCTCCTGTGCTGGACAGCGACGG |
| | | CTCATTCTTCCTGGTGTCCAGACTGACCGTGGACA |
| | | AGAGCAGATGGCAAGAGGGCAACGTGTTCAGCTG |
| | | CAGCGTGATGCACGAGGCCCTGCACAACCACTAC |
| | | ACCCAGAAGTCTCTGTCTCTGTCCCTGGGCNNN |
| 642 | ProC1441: 20GG- | GGATCTTCTAAGTACGGACCTCCTTGTCCTCCATG |
| | v619h-F5.17-Tras- | TCCTGCTCCAGAGTTTGAAGGCGGCCCTTCCGTGT |
| | Half TCB | TCCTGTTTCCTCCAAAGCCTAAGGACACCCTGATG |
| | (HC_FcK_IgG4_EN) | ATCAGCAGAACCCCTGAAGTGACCTGCGTGGTGG |
| | | TGGACGTGTCCCAAGAGGATCCTGAGGTGCAGTT |
| | | CAATTGGTACGTGGACGGCGTGGAAGTGCACAAC |
| | | GCCAAGACCAAGCCTAGAGAGGAACAGTTCAACA |
| | | GCACCTACAGAGTGGTGTCCGTGCTGACCGTGCTG |
| | | CACCAGGATTGGCTGAACGGCAAAGAGTACAAGT |
| | | GCAAGGTGTCCAACAAGGGCCTGCCTAGCAGCAT |
| | | CGAGAAAACCATCAGCAAGGCCAAGGGCCAGCCA |
| | | AGAGAACCCCAGGTGTACACACTGCCTCCATGCC |
| | | AAGAGGAAATGACCAAGAACCAGGTGTCCCTGTG |
| | | GTGCCTGGTCAAGGGCTTCTACCCTTCCGATATCG |
| | | CCGTGGAATGGGAGAGCAATGGCCAGCCTGAGAA |
| | | CAACTACAAGACCACACCTCCTGTGCTGGACAGC |
| | | GACGGCTCATTCTTCCTGTACAGCAGACTGACCGT |
| | | GGACAAGAGCAGATGGCAAGAGGGCAACGTGTTC |
| | | AGCTGCAGCGTGATGCACGAGGCCCTGCACAACC |
| | | ACTACACCCAGAAGTCTCTGAGCCTGAGCCTGGG |
| | | CNNN |
| 643 | ProC1441: 20GG- | CAAGGACAATCTGGACAAGGCGCCCTGATCTGCT |
| | v619h-F5.17-Tras- | GTTCCGATGTGTCTGGACTGTGCCGTTGGTGCGGC |
| | Half TCB (LC_Tras | GGAGGATCTTCTGGCGGCTCTATCTCTTCTGGCCT |
| | 5.17) | GCTGAGCGGCAGAAGCGACAATCACGGCGGAGGC |
| | | AGCGACATCCAGATGACACAGAGCCCTTCTAGCC |
| | | TGAGCGCCAGCGTGGGAGACAGAGTGACCATCAC |
| | | ATGTAGAGCCAGCCAGGACGTGAACACAGCCGTG |
| | | GCTTGGTATCAGCAGAAGCCTGGCAAGGCCCCTA |
| | | AGCTGCTGATCTACAGCGCCAGCTTTCTGTACAGC |
| | | GGCGTGCCCAGCAGATTCAGCGGCTCTAGAAGCG |
| | | GCACCGACTTCACCCTGACCATAAGCAGTCTGCA |
| | | GCCCGAGGACTTCGCCACCTACTACTGTCAGCAGC |
| | | ACTACACCACACCTCCAACCTTTGGCCAGGGCACC |
| | | AAGGTGGAAATCAAGCGGACAGTGGCCGCTCCTA |
| | | GCGTGTTCATCTTTCCACCTAGCGACGAGCAGCTG |
| | | AAGTCTGGCACAGCCTCTGTCGTGTGCCTGCTGAA |
| | | CAACTTCTACCCCAGAGAAGCCAAGGTGCAGTGG |
| | | AAGGTGGACAACGCCCTGCAGTCCGGCAATAGCC |
| | | AAGAGAGCGTGACCGAGCAGGACAGCAAGGACT |
| | | CTACCTACAGCCTGAGCAGCACCCTGACACTGAG |
| | | CAAGGCCGACTACGAGAAGCACAAAGTGTACGCC |
| | | TGCGAAGTGACCCACCAGGGCCTTTCTAGCCCTGT |
| | | GACCAAGAGCTTCAACCGGGGCGAATGT |
| 644 | ProC1446: F5.17- | GAAGTTCAGCTTGTTGAATCTGGCGGCGGACTGGT |
| | Tras-25-v619h- | TCAGCCTGGCGGATCTCTGAGACTGTCTTGTGCCG |
| | 20GG-24-Half TCB | CCAGCGGCTTCAACATCAAGGACACCTACATCCA |
| | (HC_Tras-25- | CTGGGTCCGACAGGCCCCTGGCAAAGGACTTGAA |
| | v619h-20GG-24- | TGGGTCGCCAGAATCTACCCCACCAACGGCTACA |
| | H4HEN) | CCAGATACGCCGACTCTGTGAAGGGCAGATTCAC |
| | | CATCAGCGCCGACACCAGCAAGAACACCGCCTAC |
| | | CTGCAGATGAACAGCCTGAGAGCCGAGGACACCG |
| | | CCGTGTACTACTGTTCTAGATGGGGAGGCGACGG |
| | | CTTCTACGCCATGGATTATTGGGGCCAGGGCACCC |
| | | TGGTCACAGTGTCTAGCGCCTCTACAAAGGGCCCC |
| | | AGCGTTTTCCCACTGGCTCCCTGTAGCAGAAGCAC |
| | | CAGCGAATCTACAGCCGCTCTGGGCTGCCTGGTCA |
| | | AGGACTACTTTCCTGAGCCTGTGACCGTGTCCTGG |
| | | AACTCTGGCGCTCTGACATCTGGCGTGCACACCTT |
| | | TCCAGCCGTGCTGCAAAGCAGCGGCCTGTACTCTC |
| | | TGAGCAGCGTCGTGACAGTGCCTAGCAGCTCTCTG |
| | | GGCACCAAGACCTACACCTGTAATGTGGACCACA |
| | | AGCCTAGCAACACCAAGGTGGACAAGAGAGTTGG |
| | | CGGAGGCGGATCAGGTGGCGGAGGAAGCGGAGG |
| | | CGGCGGATCTGAAGTGCAACTGGTTGAAAGCGGC |
| | | GGAGGCCTTGTGCAACCTGGCGGAAGCCTGAAAC |
| | | TGAGCTGTGCCGCCTCTGGCTTCACCTTCAGCACA |
| | | TACGCCATGAATTGGGTTCGACAAGCCAGCGGCA |
| | | AGGGCCTCGAATGGGTTGGAAGAATCAGAAGCAA |
| | | GTACAACAACTACGCCACCTACTACGCCGACAGC |
| | | GTGAAGGATCGGTTTACCATCAGCCGGGACGACT |
| | | CCAAGAATACTGCCTATCTCCAAATGAACTCCCTC |
| | | AAGACCGAGGATACAGCCGTCTATTACTGCGTGC |
| | | GGCACGGCAACTTCGGCAACAGCTATGTGTCTTG |
| | | GTGGGCCTACTGGGGACAGGGAACACTGGTTACT |
| | | GTGTCCAGTGGCGGTGGTGGAAGTGGCGGCGGAG |
| | | GATCAGGCGGTGGCGGTTCTCAAACAGTGGTCAC |
| | | CCAAGAGCCTAGCCTGACAGTGTCTCCAGGCGGA |
| | | ACCGTGACACTGACATGTGGCAGTTCTACAGGCG |
| | | CCGTGACCACCAGCAACTACGTGAACTGGGTGCA |
| | | GCAGAAGCCAGGCCAGGCTCCTAGAGGACTGATC |
| | | GGAGGCACCAACAAGAGAGCCCCTGGAACACCAG |
| | | CCAGATTCTCCGGCTCTCTGATCGGCGGAAAAGCC |
| | | GCACTGACACTGTCTGGTGCTCAGCCTGAGGATG |
| | | AGGCCGAGTACTATTGCGTGCTGTGGTACAGCAA |
| | | CAGATGGGTGTTCGGCGGAGGGACCAAGCTGACA |
| | | GTGCTTGGAGGTTCTGGTGGCAGCAGCGGCGCTTC |
| | | TGGAACTGGAACAGCTGGCGGAACAGGCTCTGGA |
| | | TCTGGCACAGGATCTGGCCTGAGCGGCAGATCCG |
| | | ATGATCATGGCGGAGGTTCTCAGGGCCAGTCTGG |
| | | CAGTGGATATCTGTGGGGCTGCGAGTGGAATTGC |
| | | GGCGGCATCACAACAGGATCTAGCGGAGGCTCAG |
| | | GTGGTGGTGGATCTGGTGGTGGCGGCTCAGGCGG |
| | | AGGTGGTAGCGGTGGTTCTGAGTCTAAGTACGGC |
| | | CCTCCTTGTCCTCCATGTCCTGCTCCAGAGTTTGA |
| | | AGGCGGCCCTTCCGTGTTCCTGTTTCCTCCAAAGC |
| | | CTAAGGACACCCTGATGATCAGCAGAACCCCTGA |
| | | AGTGACCTGCGTGGTGGTCGACGTTTCCCAAGAG |
| | | GACCCTGAGGTGCAGTTCAATTGGTACGTGGACG |
| | | GCGTGGAAGTGCACAACGCCAAGACCAAGCCTAG |
| | | AGAGGAACAGTTCAACAGCACCTACAGAGTGGTG |
| | | TCCGTGCTGACTGTGCTGCACCAGGATTGGCTGAA |
| | | CGGCAAAGAGTACAAGTGCAAGGTGTCCAACAAG |
| | | GGCCTGCCAAGCAGCATCGAGAAAACCATCAGCA |
| | | AGGCCAAGGGCCAGCCTCGCGAACCTCAAGTCTG |
| | | TACACTGCCTCCTAGCCAAGAGGAAATGACCAAG |
| | | AACCAGGTGTCCCTGAGCTGCGCCGTGAAGGGCT |
| | | TTTACCCTTCCGATATCGCCGTGGAATGGGAGAGC |
| | | AATGGCCAGCCAGAGAACAACTACAAGACAACCC |
| | | CTCCTGTGCTGGACAGCGACGGCTCATTTTTCCTG |
| | | GTGTCCAGACTGACCGTGGACAAGTCCAGATGGC |
| | | AAGAGGGCAACGTGTTCAGCTGCAGCGTGATGCA |
| | | CGAGGCCCTGCACAACCACTACACCCAGAAGTCT |
| | | CTGTCTCTGAGCCTGGGCNNN |
| 645 | ProC1446: F5.17- | GGATCTTCTAAGTACGGACCTCCTTGTCCTCCATG |
| | Tras-25-v619h- | TCCTGCTCCAGAGTTTGAAGGCGGCCCTTCCGTGT |
| | 20GG-24-Half TCB | TCCTGTTTCCTCCAAAGCCTAAGGACACCCTGATG |
| | (HC_FcK_IgG4_EN) | ATCAGCAGAACCCCTGAAGTGACCTGCGTGGTGG |
| | | TGGACGTGTCCCAAGAGGATCCTGAGGTGCAGTT |
| | | CAATTGGTACGTGGACGGCGTGGAAGTGCACAAC |
| | | GCCAAGACCAAGCCTAGAGAGGAACAGTTCAACA |
| | | GCACCTACAGAGTGGTGTCCGTGCTGACCGTGCTG |
| | | CACCAGGATTGGCTGAACGGCAAAGAGTACAAGT |
| | | GCAAGGTGTCCAACAAGGGCCTGCCTAGCAGCAT |
| | | CGAGAAAACCATCAGCAAGGCCAAGGGCCAGCCA |
| | | AGAGAACCCCAGGTGTACACACTGCCTCCATGCC |
| | | AAGAGGAAATGACCAAGAACCAGGTGTCCCTGTG |
| | | GTGCCTGGTCAAGGGCTTCTACCCTTCCGATATCG |
| | | CCGTGGAATGGGAGAGCAATGGCCAGCCTGAGAA |
| | | CAACTACAAGACCACACCTCCTGTGCTGGACAGC |
| | | GACGGCTCATTCTTCCTGTACAGCAGACTGACCGT |
| | | GGACAAGAGCAGATGGCAAGAGGGCAACGTGTTC |
| | | AGCTGCAGCGTGATGCACGAGGCCCTGCACAACC |
| | | ACTACACCCAGAAGTCTCTGAGCCTGAGCCTGGG |
| | | CNNN |
| 646 | ProC1446: F5.17- | CAAGGACAATCTGGACAAGGCGCCCTGATCTGCT |
| | Tras-25-v619h- | GTTCCGATGTGTCTGGACTGTGCCGTTGGTGCGGC |
| | 20GG-24-Half TCB | GGAGGATCTTCTGGCGGCTCTATCTCTTCTGGCCT |
| | (LC_Tras 5.17) | GCTGAGCGGCAGAAGCGACAATCACGGCGGAGGC |
| | | AGCGACATCCAGATGACACAGAGCCCTTCTAGCC |
| | | TGAGCGCCAGCGTGGGAGACAGAGTGACCATCAC |
| | | ATGTAGAGCCAGCCAGGACGTGAACACAGCCGTG |
| | | GCTTGGTATCAGCAGAAGCCTGGCAAGGCCCCTA |
| | | AGCTGCTGATCTACAGCGCCAGCTTTCTGTACAGC |
| | | GGCGTGCCCAGCAGATTCAGCGGCTCTAGAAGCG |
| | | GCACCGACTTCACCCTGACCATAAGCAGTCTGCA |
| | | GCCCGAGGACTTCGCCACCTACTACTGTCAGCAGC |
| | | ACTACACCACACCTCCAACCTTTGGCCAGGGCACC |
| | | AAGGTGGAAATCAAGCGGACAGTGGCCGCTCCTA |
| | | GCGTGTTCATCTTTCCACCTAGCGACGAGCAGCTG |
| | | AAGTCTGGCACAGCCTCTGTCGTGTGCCTGCTGAA |
| | | CAACTTCTACCCCAGAGAAGCCAAGGTGCAGTGG |
| | | AAGGTGGACAACGCCCTGCAGTCCGGCAATAGCC |
| | | AAGAGAGCGTGACCGAGCAGGACAGCAAGGACT |
| | | CTACCTACAGCCTGAGCAGCACCCTGACACTGAG |
| | | CAAGGCCGACTACGAGAAGCACAAAGTGTACGCC |
| | | TGCGAAGTGACCCACCAGGGCCTTTCTAGCCCTGT |
| | | GACCAAGAGCTTCAACCGGGGCGAATGT |
| 647 | ProC1447: F5.17- | GAAGTTCAGCTTGTTGAATCTGGCGGCGGACTGGT |
| | Tras-25-v619h- | TCAGCCTGGCGGATCTCTGAGACTGTCTTGTGCCG |
| | MN15a-24-Half | CCAGCGGCTTCAACATCAAGGACACCTACATCCA |
| | TCB | CTGGGTCCGACAGGCCCCTGGCAAAGGACTTGAA |
| | (HC_Tras-25- | TGGGTCGCCAGAATCTACCCCACCAACGGCTACA |
| | v619h-MN15a-24- | CCAGATACGCCGACTCTGTGAAGGGCAGATTCAC |
| | H4HEN) | CATCAGCGCCGACACCAGCAAGAACACCGCCTAC |
| | | CTGCAGATGAACAGCCTGAGAGCCGAGGACACCG |
| | | CCGTGTACTACTGTTCTAGATGGGGAGGCGACGG |
| | | CTTCTACGCCATGGATTATTGGGGCCAGGGCACCC |
| | | TGGTCACAGTGTCTAGCGCCTCTACAAAGGGCCCC |
| | | AGCGTTTTCCCACTGGCTCCCTGTAGCAGAAGCAC |
| | | CAGCGAATCTACAGCCGCTCTGGGCTGCCTGGTCA |
| | | AGGACTACTTTCCTGAGCCTGTGACCGTGTCCTGG |
| | | AACTCTGGCGCTCTGACATCTGGCGTGCACACCTT |
| | | TCCAGCCGTGCTGCAAAGCAGCGGCCTGTACTCTC |
| | | TGAGCAGCGTCGTGACAGTGCCTAGCAGCTCTCTG |
| | | GGCACCAAGACCTACACCTGTAATGTGGACCACA |
| | | AGCCTAGCAACACCAAGGTGGACAAGAGAGTTGG |
| | | CGGAGGCGGATCAGGTGGCGGAGGAAGCGGAGG |
| | | CGGCGGATCTGAAGTGCAACTGGTTGAAAGCGGC |
| | | GGAGGCCTTGTGCAACCTGGCGGAAGCCTGAAAC |
| | | TGAGCTGTGCCGCCTCTGGCTTCACCTTCAGCACA |
| | | TACGCCATGAATTGGGTTCGACAAGCCAGCGGCA |
| | | AGGGCCTCGAATGGGTTGGAAGAATCAGAAGCAA |
| | | GTACAACAACTACGCCACCTACTACGCCGACAGC |
| | | GTGAAGGATCGGTTTACCATCAGCCGGGACGACT |
| | | CCAAGAATACTGCCTATCTCCAAATGAACTCCCTC |
| | | AAGACCGAGGATACAGCCGTCTATTACTGCGTGC |
| | | GGCACGGCAACTTCGGCAACAGCTATGTGTCTTG |
| | | GTGGGCCTACTGGGGACAGGGAACACTGGTTACT |
| | | GTGTCCAGTGGCGGTGGTGGAAGTGGCGGCGGAG |
| | | GATCAGGCGGTGGCGGTTCTCAAACAGTGGTCAC |
| | | CCAAGAGCCTAGCCTGACAGTGTCTCCAGGCGGA |
| | | ACCGTGACACTGACATGTGGCAGTTCTACAGGCG |
| | | CCGTGACCACCAGCAACTACGTGAACTGGGTGCA |
| | | GCAGAAGCCAGGCCAGGCTCCTAGAGGACTGATC |
| | | GGAGGCACCAACAAGAGAGCCCCTGGAACACCAG |
| | | CCAGATTCTCCGGCTCTCTGATCGGCGGAAAAGCC |
| | | GCACTGACACTGTCTGGTGCTCAGCCTGAGGATG |
| | | AGGCCGAGTACTATTGCGTGCTGTGGTACAGCAA |
| | | CAGATGGGTGTTCGGCGGAGGGACCAAGCTGACA |
| | | GTGCTTGGAGGTTCTGGTGGCAGCAGCGGCGCTTC |
| | | TGGAACTGGAACAGCTGGCGGAACAGGCTCTGGA |
| | | TCTGGCACAGGATCTGGCCTGAGCGGCAGATCCG |
| | | ATGATCATGGCGGAGGTTCTCAGGGCCAGTCTGG |
| | | CAGCAATGCCTTCAGATGTTGGTGGGACCCTCCTT |
| | | GCCAGCCTATGACAGGATCTAGCGGAGGCTCAGG |
| | | TGGCGGTGGATCTGGCGGAGGTGGTAGCGGAGGT |
| | | GGTGGAAGCGGAGGATCTGAGTCTAAGTACGGCC |
| | | CTCCTTGTCCTCCATGTCCTGCTCCAGAGTTTGAA |
| | | GGCGGCCCTTCCGTGTTCCTGTTTCCTCCAAAGCC |
| | | TAAGGACACCCTGATGATCAGCAGAACCCCTGAA |
| | | GTGACCTGCGTGGTGGTGGACGTTTCCCAAGAGG |
| | | ACCCTGAGGTGCAGTTCAATTGGTACGTGGACGG |
| | | CGTGGAAGTGCACAACGCCAAGACCAAGCCTAGA |
| | | GAGGAACAGTTCAACAGCACCTACAGAGTGGTGT |
| | | CCGTGCTGACTGTGCTGCACCAGGATTGGCTGAAC |
| | | GGCAAAGAGTACAAGTGCAAGGTGTCCAACAAGG |
| | | GCCTGCCAAGCAGCATCGAGAAAACCATCAGCAA |
| | | GGCCAAGGGCCAGCCTCGCGAACCTCAAGTCTGT |
| | | ACACTGCCTCCTAGCCAAGAGGAAATGACCAAGA |
| | | ACCAGGTGTCCCTGAGCTGCGCCGTGAAGGGCTTT |
| | | TACCCTTCCGATATCGCCGTGGAATGGGAGAGCA |
| | | ATGGCCAGCCAGAGAACAACTACAAGACAACCCC |
| | | TCCTGTGCTGGACAGCGACGGCTCATTTTTCCTGG |
| | | TGTCCAGACTGACCGTGGACAAGTCCAGATGGCA |
| | | AGAGGGCAACGTGTTCAGCTGCAGCGTGATGCAC |
| | | GAGGCCCTGCACAACCACTACACCCAGAAGTCTC |
| | | TGTCTCTGAGCCTGGGCNNN |
| 648 | ProC1447: F5.17- | GGATCTTCTAAGTACGGACCTCCTTGTCCTCCATG |
| | Tras-25-v619h- | TCCTGCTCCAGAGTTTGAAGGCGGCCCTTCCGTGT |
| | MN15a-24-Half | TCCTGTTTCCTCCAAAGCCTAAGGACACCCTGATG |
| | TCB | ATCAGCAGAACCCCTGAAGTGACCTGCGTGGTGG |
| | (HC_FcK_IgG4_EN) | TGGACGTGTCCCAAGAGGATCCTGAGGTGCAGTT |
| | | CAATTGGTACGTGGACGGCGTGGAAGTGCACAAC |
| | | GCCAAGACCAAGCCTAGAGAGGAACAGTTCAACA |
| | | GCACCTACAGAGTGGTGTCCGTGCTGACCGTGCTG |
| | | CACCAGGATTGGCTGAACGGCAAAGAGTACAAGT |
| | | GCAAGGTGTCCAACAAGGGCCTGCCTAGCAGCAT |
| | | CGAGAAAACCATCAGCAAGGCCAAGGGCCAGCCA |
| | | AGAGAACCCCAGGTGTACACACTGCCTCCATGCC |
| | | AAGAGGAAATGACCAAGAACCAGGTGTCCCTGTG |
| | | GTGCCTGGTCAAGGGCTTCTACCCTTCCGATATCG |
| | | CCGTGGAATGGGAGAGCAATGGCCAGCCTGAGAA |
| | | CAACTACAAGACCACACCTCCTGTGCTGGACAGC |
| | | GACGGCTCATTCTTCCTGTACAGCAGACTGACCGT |
| | | GGACAAGAGCAGATGGCAAGAGGGCAACGTGTTC |
| | | AGCTGCAGCGTGATGCACGAGGCCCTGCACAACC |
| | | ACTACACCCAGAAGTCTCTGAGCCTGAGCCTGGG |
| | | CNNN |
| 649 | ProC1447: F5.17- | CAAGGACAATCTGGACAAGGCGCCCTGATCTGCT |
| | Tras-25-v619h- | GTTCCGATGTGTCTGGACTGTGCCGTTGGTGCGGC |
| | MN15a-24-Half | GGAGGATCTTCTGGCGGCTCTATCTCTTCTGGCCT |
| | TCB (LC_Tras 5.17) | GCTGAGCGGCAGAAGCGACAATCACGGCGGAGGC |
| | | AGCGACATCCAGATGACACAGAGCCCTTCTAGCC |
| | | TGAGCGCCAGCGTGGGAGACAGAGTGACCATCAC |
| | | ATGTAGAGCCAGCCAGGACGTGAACACAGCCGTG |
| | | GCTTGGTATCAGCAGAAGCCTGGCAAGGCCCCTA |
| | | AGCTGCTGATCTACAGCGCCAGCTTTCTGTACAGC |
| | | GGCGTGCCCAGCAGATTCAGCGGCTCTAGAAGCG |
| | | GCACCGACTTCACCCTGACCATAAGCAGTCTGCA |
| | | GCCCGAGGACTTCGCCACCTACTACTGTCAGCAGC |
| | | ACTACACCACACCTCCAACCTTTGGCCAGGGCACC |
| | | AAGGTGGAAATCAAGCGGACAGTGGCCGCTCCTA |
| | | GCGTGTTCATCTTTCCACCTAGCGACGAGCAGCTG |
| | | AAGTCTGGCACAGCCTCTGTCGTGTGCCTGCTGAA |
| | | CAACTTCTACCCCAGAGAAGCCAAGGTGCAGTGG |
| | | AAGGTGGACAACGCCCTGCAGTCCGGCAATAGCC |
| | | AAGAGAGCGTGACCGAGCAGGACAGCAAGGACT |
| | | CTACCTACAGCCTGAGCAGCACCCTGACACTGAG |
| | | CAAGGCCGACTACGAGAAGCACAAAGTGTACGCC |
| | | TGCGAAGTGACCCACCAGGGCCTTTCTAGCCCTGT |
| | | GACCAAGAGCTTCAACCGGGGCGAATGT |
| 650 | ProC1448: F5.17- | GAAGTTCAGCTTGTTGAATCTGGCGGCGGACTGGT |
| | Tras-25-v619h- | TCAGCCTGGCGGATCTCTGAGACTGTCTTGTGCCG |
| | MN15b-24-Half | CCAGCGGCTTCAACATCAAGGACACCTACATCCA |
| | TCB | CTGGGTCCGACAGGCCCCTGGCAAAGGACTTGAA |
| | (HC_Tras-25- | TGGGTCGCCAGAATCTACCCCACCAACGGCTACA |
| | v619h-MN15b-24- | CCAGATACGCCGACTCTGTGAAGGGCAGATTCAC |
| | H4HEN) | CATCAGCGCCGACACCAGCAAGAACACCGCCTAC |
| | | CTGCAGATGAACAGCCTGAGAGCCGAGGACACCG |
| | | CCGTGTACTACTGTTCTAGATGGGGAGGCGACGG |
| | | CTTCTACGCCATGGATTATTGGGGCCAGGGCACCC |
| | | TGGTCACAGTGTCTAGCGCCTCTACAAAGGGCCCC |
| | | AGCGTTTTCCCACTGGCTCCCTGTAGCAGAAGCAC |
| | | CAGCGAATCTACAGCCGCTCTGGGCTGCCTGGTCA |
| | | AGGACTACTTTCCTGAGCCTGTGACCGTGTCCTGG |
| | | AACTCTGGCGCTCTGACATCTGGCGTGCACACCTT |
| | | TCCAGCCGTGCTGCAAAGCAGCGGCCTGTACTCTC |
| | | TGAGCAGCGTCGTGACAGTGCCTAGCAGCTCTCTG |
| | | GGCACCAAGACCTACACCTGTAATGTGGACCACA |
| | | AGCCTAGCAACACCAAGGTGGACAAGAGAGTTGG |
| | | CGGAGGCGGATCAGGTGGCGGAGGAAGCGGAGG |
| | | CGGCGGATCTGAAGTGCAACTGGTTGAAAGCGGC |
| | | GGAGGCCTTGTGCAACCTGGCGGAAGCCTGAAAC |
| | | TGAGCTGTGCCGCCTCTGGCTTCACCTTCAGCACA |
| | | TACGCCATGAATTGGGTTCGACAAGCCAGCGGCA |
| | | AGGGCCTCGAATGGGTTGGAAGAATCAGAAGCAA |
| | | GTACAACAACTACGCCACCTACTACGCCGACAGC |
| | | GTGAAGGATCGGTTTACCATCAGCCGGGACGACT |
| | | CCAAGAATACTGCCTATCTCCAAATGAACTCCCTC |
| | | AAGACCGAGGATACAGCCGTCTATTACTGCGTGC |
| | | GGCACGGCAACTTCGGCAACAGCTATGTGTCTTG |
| | | GTGGGCCTACTGGGGACAGGGAACACTGGTTACT |
| | | GTGTCCAGTGGCGGTGGTGGAAGTGGCGGCGGAG |
| | | GATCAGGCGGTGGCGGTTCTCAAACAGTGGTCAC |
| | | CCAAGAGCCTAGCCTGACAGTGTCTCCAGGCGGA |
| | | ACCGTGACACTGACATGTGGCAGTTCTACAGGCG |
| | | CCGTGACCACCAGCAACTACGTGAACTGGGTGCA |
| | | GCAGAAGCCAGGCCAGGCTCCTAGAGGACTGATC |
| | | GGAGGCACCAACAAGAGAGCCCCTGGAACACCAG |
| | | CCAGATTCTCCGGCTCTCTGATCGGCGGAAAAGCC |
| | | GCACTGACACTGTCTGGTGCTCAGCCTGAGGATG |
| | | AGGCCGAGTACTATTGCGTGCTGTGGTACAGCAA |
| | | CAGATGGGTGTTCGGCGGAGGGACCAAGCTGACA |
| | | GTGCTTGGAGGTTCTGGTGGCAGCAGCGGCGCTTC |
| | | TGGAACTGGAACAGCTGGCGGAACAGGCTCTGGA |
| | | TCTGGCACAGGATCTGGCCTGAGCGGCAGATCCG |
| | | ATGATCATGGCGGAGGTTCTCAGGGCCAGTCTGG |
| | | CTCTGCAAGAGGACTGTGTTGGTGGGACCCTCCTT |
| | | GCACACACGATCTGGGATCTTCAGGCGGATCTGG |
| | | CGGAGGTGGTAGCGGTGGTGGTGGTAGCGGAGGC |
| | | GGAGGTAGTGGTGGAAGCGAGTCTAAGTACGGCC |
| | | CTCCTTGTCCTCCATGTCCTGCTCCAGAGTTTGAA |
| | | GGCGGCCCTTCCGTGTTCCTGTTTCCTCCAAAGCC |
| | | TAAGGACACCCTGATGATCAGCAGAACCCCTGAA |
| | | GTGACCTGCGTGGTGGTCGACGTTTCCCAAGAGG |
| | | ACCCTGAGGTGCAGTTCAATTGGTACGTGGACGG |
| | | CGTGGAAGTGCACAACGCCAAGACCAAGCCTAGA |
| | | GAGGAACAGTTCAACAGCACCTACAGAGTGGTGT |
| | | CCGTGCTGACTGTGCTGCACCAGGATTGGCTGAAC |
| | | GGCAAAGAGTACAAGTGCAAGGTGTCCAACAAGG |
| | | GCCTGCCAAGCAGCATCGAGAAAACCATCAGCAA |
| | | GGCCAAGGGCCAGCCTCGCGAACCTCAAGTCTGT |
| | | ACACTGCCTCCTAGCCAAGAGGAAATGACCAAGA |
| | | ACCAGGTGTCCCTGAGCTGCGCCGTGAAGGGCTTT |
| | | TACCCTTCCGATATCGCCGTGGAATGGGAGAGCA |
| | | ATGGCCAGCCAGAGAACAACTACAAGACAACCCC |
| | | TCCTGTGCTGGACAGCGACGGCTCATTTTTCCTGG |
| | | TGTCCAGACTGACCGTGGACAAGTCCAGATGGCA |
| | | AGAGGGCAACGTGTTCAGCTGCAGCGTGATGCAC |
| | | GAGGCCCTGCACAACCACTACACCCAGAAGTCTC |
| | | TGTCTCTGAGCCTGGGCNNN |
| 651 | ProC1448: F5.17- | GGATCTTCTAAGTACGGACCTCCTTGTCCTCCATG |
| | Tras-25-v619h- | TCCTGCTCCAGAGTTTGAAGGCGGCCCTTCCGTGT |
| | MN15b-24-Half | TCCTGTTTCCTCCAAAGCCTAAGGACACCCTGATG |
| | TCB | ATCAGCAGAACCCCTGAAGTGACCTGCGTGGTGG |
| | (HC_FcK_IgG4_EN) | TGGACGTGTCCCAAGAGGATCCTGAGGTGCAGTT |
| | | CAATTGGTACGTGGACGGCGTGGAAGTGCACAAC |
| | | GCCAAGACCAAGCCTAGAGAGGAACAGTTCAACA |
| | | GCACCTACAGAGTGGTGTCCGTGCTGACCGTGCTG |
| | | CACCAGGATTGGCTGAACGGCAAAGAGTACAAGT |
| | | GCAAGGTGTCCAACAAGGGCCTGCCTAGCAGCAT |
| | | CGAGAAAACCATCAGCAAGGCCAAGGGCCAGCCA |
| | | AGAGAACCCCAGGTGTACACACTGCCTCCATGCC |
| | | AAGAGGAAATGACCAAGAACCAGGTGTCCCTGTG |
| | | GTGCCTGGTCAAGGGCTTCTACCCTTCCGATATCG |
| | | CCGTGGAATGGGAGAGCAATGGCCAGCCTGAGAA |
| | | CAACTACAAGACCACACCTCCTGTGCTGGACAGC |
| | | GACGGCTCATTCTTCCTGTACAGCAGACTGACCGT |
| | | GGACAAGAGCAGATGGCAAGAGGGCAACGTGTTC |
| | | AGCTGCAGCGTGATGCACGAGGCCCTGCACAACC |
| | | ACTACACCCAGAAGTCTCTGAGCCTGAGCCTGGG |
| | | CNNN |
| 652 | ProC1448: F5.17- | CAAGGACAATCTGGACAAGGCGCCCTGATCTGCT |
| | Tras-25-v619h- | GTTCCGATGTGTCTGGACTGTGCCGTTGGTGCGGC |
| | MN15b-24-Half | GGAGGATCTTCTGGCGGCTCTATCTCTTCTGGCCT |
| | TCB (LC_Tras 5.17) | GCTGAGCGGCAGAAGCGACAATCACGGCGGAGGC |
| | | AGCGACATCCAGATGACACAGAGCCCTTCTAGCC |
| | | TGAGCGCCAGCGTGGGAGACAGAGTGACCATCAC |
| | | ATGTAGAGCCAGCCAGGACGTGAACACAGCCGTG |
| | | GCTTGGTATCAGCAGAAGCCTGGCAAGGCCCCTA |
| | | AGCTGCTGATCTACAGCGCCAGCTTTCTGTACAGC |
| | | GGCGTGCCCAGCAGATTCAGCGGCTCTAGAAGCG |
| | | GCACCGACTTCACCCTGACCATAAGCAGTCTGCA |
| | | GCCCGAGGACTTCGCCACCTACTACTGTCAGCAGC |
| | | ACTACACCACACCTCCAACCTTTGGCCAGGGCACC |
| | | AAGGTGGAAATCAAGCGGACAGTGGCCGCTCCTA |
| | | GCGTGTTCATCTTTCCACCTAGCGACGAGCAGCTG |
| | | AAGTCTGGCACAGCCTCTGTCGTGTGCCTGCTGAA |
| | | CAACTTCTACCCCAGAGAAGCCAAGGTGCAGTGG |
| | | AAGGTGGACAACGCCCTGCAGTCCGGCAATAGCC |
| | | AAGAGAGCGTGACCGAGCAGGACAGCAAGGACT |
| | | CTACCTACAGCCTGAGCAGCACCCTGACACTGAG |
| | | CAAGGCCGACTACGAGAAGCACAAAGTGTACGCC |
| | | TGCGAAGTGACCCACCAGGGCCTTTCTAGCCCTGT |
| | | GACCAAGAGCTTCAACCGGGGCGAATGT |

*Note: X is a lysine residue or absent at the C-terminus.
**Note: N is either adenine forming a codon for lysine or each N is absent so that the codon for lysine is not present.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

All publications, patent applications, patents, and other references mentioned herein are incorporated by reference in their entirety. In case of conflict, the present specification, including definitions, will control. In addition section headings, the materials, methods, and examples are illustrative only and not intended to be limiting.

What is claimed is:

1. An activatable protein comprising:
a first antigen-binding domain (AB1) that specifically binds to a first target, wherein the AB1 comprises a first heavy chain variable domain (HVD1) and a first light chain variable domain (LVD1);
a second antigen-binding domain (AB2) that specifically binds to a second target, wherein the AB2 comprises a second heavy chain variable domain (HVD2) and a second light chain variable domain (LVD2), and the AB2 is directly or indirectly coupled to a C-terminus of the HVD1 or to a C-terminus of the LVD1;
a first masking moiety (MM1) coupled to the AB1 via a first cleavable moiety (CM1), either directly or indirectly, wherein the MM1 inhibits the binding of the AB1 to the first target;
a half-life extending moiety (EM) directly or indirectly coupled to a second masking moiety (MM2),
wherein the EM is coupled to the AB1 or to the AB2 via a second cleavable moiety (CM2), either directly or indirectly, and
wherein the MM2 inhibits the binding of the AB2 to the second target.

2. The activatable protein of claim 1, wherein the EM is a dimer formed by a first fragment crystallizable (Fc) domain and a second Fc domain.

3. The activatable protein of claim 1 or claim 2, wherein the EM is C-terminal to the AB1 or the AB2 to which the EM is coupled via the CM2, either directly or indirectly, and wherein MM1 is N-terminal to the AB1.

4. An activatable protein comprising:
a first antigen-binding domain (AB1) that specifically binds to a first target, wherein the AB1 comprises a first heavy chain variable domain (HVD1) and a first light chain variable domain (LVD1);
a second antigen-binding domain (AB2) that specifically binds to a second target, wherein the AB2 comprises a second heavy chain variable domain (HVD2) and a second light chain variable domain (LVD2), and the AB2 is directly or indirectly coupled to a C-terminus of the HVD1 or the LVD1;
a first masking moiety (MM1) coupled to the AB1 via a first cleavable moiety (CM1) and optionally one or more linkers, wherein the MM1 inhibits the binding of the AB1 to the first target; and
a half-life extending moiety (EM) directly or indirectly coupled to a second masking moiety (MM2), wherein the EM and the MM2 are coupled to the AB1 or to the AB2 via a second cleavable moiety (CM2) and optionally one or more linkers, and
wherein the MM2 inhibits the binding of the AB2 to the second target.

5. The activatable protein of claim 4, wherein the CM2 is N-terminal of the MM2 and the MM2 is N-terminal of the EM and the CM2 is C-terminal of the AB1 or the AB2 to which the EM and the MM2 are coupled via the CM2 and optionally one or more linkers.

6. An activatable protein comprising:
a first antigen-binding domain (AB1) that specifically binds to a first target, wherein the AB1 comprises a first heavy chain variable domain (HVD1) and a first light chain variable domain (LVD1);
a second antigen-binding domain (AB2) that specifically binds to a second target, wherein the AB2 comprises a second heavy chain variable domain (HVD2) and a second light chain variable domain (LVD2), and the AB2 is directly or indirectly coupled to a C-terminus of the HVD1 or the LVD1;
a first masking moiety (MM1) coupled to the AB1 via a first cleavable moiety (CM1) and optionally one or more linkers, wherein the MM1 inhibits the binding of the AB1 to the first target; and
a half-life extending moiety (EM) comprising a dimer of a first half-life extending moiety (EM1) and a second half-life extending moiety (EM2),
wherein the EM1 is coupled to the AB1 via a second cleavable moiety (CM2) and optionally one or more linkers, and
wherein the EM2 is directly or indirectly coupled to a second masking moiety (MM2),
wherein the MM2 inhibits the binding of the AB2 to the second target.

7. The activatable protein of claim 6, wherein the CM2 is N-terminal of the EM1 and the CM2 is C-terminal of the AB1.

8. The activatable protein of any one of claims 1-7, wherein the activatable protein comprises at least a first polypeptide and a second polypeptide.

9. The activatable protein of claim 8, wherein the first polypeptide comprises, in order from N-terminus to C-terminus, the MM1, the CM1, and the VLD1.

10. The activatable protein of any one of claims 8-9, wherein the second polypeptide comprises the VHD1, the VHD2, the VLD2, the CM2, the MM2 and a first Fc domain, and wherein the activatable protein further comprises a third polypeptide comprising a second Fc domain.

11. The activatable protein of claim 10, wherein the second polypeptide comprises, in order from N-terminus to C-terminus, the VHD1, the VHD2, the VLD2, the CM2, the MM2, and a first Fc domain.

12. The activatable protein of claim 9, wherein the second polypeptide comprises, in order from N-terminus to C-terminus, the VHD1, the CM2, the MM2, and a first Fc domain.

13. The activatable protein of claim 9, wherein the second polypeptide comprises, in order from N-terminus to C-terminus, the VHD1, the CM2, and a first Fc domain.

14. The activatable protein of any one of claim 9 or 12-13, wherein the first polypeptide comprises the MM1, the CM1, the VLD1, the VHD2, and the VLD2.

15. The activatable protein of claim 13, wherein the first polypeptide comprises, in order from N-terminus to C-terminus, the MM1, the CM1 the VLD1, the VHD2, and the VLD2.

16. The activatable protein of claim 13, wherein the first polypeptide comprises, in order from N-terminus to C-terminus, the MM1, the CM1 the VLD1, the VLD2, and the VHD2.

17. The activatable protein of any of claims 13-16, wherein the protein comprises a third polypeptide, and wherein the third polypeptide comprises a second Fc domain and the MM2.

18. The activatable protein of claim 17, wherein the MM2 is linked to the C-terminus of the second Fc domain via a linker.

19. The activatable protein of claim 17, wherein the MM2 is linked to the N-terminus of the second Fc domain via a linker.

20. The activatable protein of any of claims 1-19, further comprising a linker coupling:
(i) MM1 and CM1,
(ii) CM1 and VLD1,
(iii) VHD1 and VLD2,
(iv) VHD1 and VHD2,
(v) VHD1 and CM2,
(vi) VLD2 and VHD2,
(vii) CM2 and MM2,
(viii) CM2 and EM,
(ix) EM and MM2,
(x) VLD1 and VHD2, and/or
(xi) VLD1 and VLD2.

21. The activatable protein of any one of claims 18-20, wherein the linker is a peptide having a length of 5 to 30, 6 to 29, 7 to 28, 8 to 27, 9 to 26, 10 to 25, 11, 12, 13, 14, 15, 16, 17, 18, 19, 20, 21, 22, 23, or 24 amino acids.

22. The activatable protein of any one of claims 2 and 10-19, wherein the first Fc domain is a Fc domain hole mutant and the second Fc domain is a Fc domain knob mutant.

23. The activatable protein of claim 22, wherein the Fc domain hole mutant comprises a sequence of SEQ ID NO: 2 and the Fc domain knob mutant comprises a sequence of SEQ ID NO: 1.

24. The activatable protein of claim 1, wherein the CM2 is directly or indirectly coupled to a C-terminus of the AB1 or a C-terminus of the AB2.

25. The activatable protein of claim 24, wherein the MM2 is directly or indirectly coupled to a C-terminus of the CM2 and the MM2 is directly or indirectly coupled to a N-terminus of the EM.

26. The activatable protein of claim 1 or claim 24, wherein the EM comprises a dimer of a first half-life extending moiety (EM1) and a second half-life extending moiety (EM2).

27. The activatable protein of claim 26, wherein the CM2 is directly or indirectly coupled to a N-terminus of the EM1.

28. The activatable protein of claim 26, wherein the CM2 is directly or indirectly coupled to a N-terminus of the MM2 and the MM2 is directly or indirectly coupled to a N-terminus of the EM1.

29. The activatable protein of any one of claim 26, wherein the MM2 is directly or indirectly coupled to a N-terminus of the EM2.

30. The activatable protein of any one of claims 1-29, wherein the first target or epitope is a tumor associated antigen.

31. The activatable protein of claim 30, wherein the tumor associated antigen is human epidermal growth factor receptor 2 (HER2).

32. The activatable protein of claim 31, wherein the AB1 is a Fab of trastuzumab.

33. The activatable of claim 31, wherein the HVD1 comprises a sequence of SEQ ID NO: 27 and the LVD1 comprises a sequence of SEQ ID NO: 17.

34. The activatable protein of any one of claims 1-33, wherein AB2 is:
an immune effector cell engaging scFv;
a leukocyte engaging scFv;
a T-cell engaging scFv;
a NK-cell engaging scFv;
a macrophage engaging scFv; or
a mononuclear cell engaging scFv.

35. The activatable protein of any one of claims 1-34, wherein AB2 is or is derived from an anti-CD3 epsilon scFv or an anti-CTLA-4 scFv.

36. The activatable protein of claim 35, wherein the AB2 is or is derived from an anti-CD3 epsilon scFv.

37. The activatable protein of claim 36, wherein the HVD2 comprises a sequence of SEQ ID NO: 30 and the LVD2 comprises a sequence of SEQ ID NO: 31.

38. The activatable protein of any one of claims 1-37, wherein AB1 is or is derived from an anti-HER2 antibody.

39. The activatable protein of any one of claims 1-38, wherein CM1 and CM2 each independently comprises a substrate for a protease that is upregulated in a tumor microenvironment.

40. The activatable protein of any one of claims 1-39, wherein AB1 is a Fragment antigen binding (Fab).

41. The activatable protein of any one of claims 1-40, wherein the second target is a co-stimulatory molecule.

42. The activatable protein of claim 41, wherein the co-stimulatory molecule is CD3.

43. The activatable protein of any one of claims 1-42, wherein each of the CM1 and the CM2 comprises a substrate for the same protease.

44. The activatable protein of any one of claims 1-42, wherein the CM1 and the CM2 comprise substrates for different proteases.

45. An activatable molecule comprising:
a first target-binding domain (TB1) that specifically binds to a first target;
a second target-binding domain (TB2) that specifically binds to a second target, wherein the TB2 is directly or indirectly coupled to the TB1;
a first masking moiety (MM1) directly or indirectly coupled to the TB1 via a first cleavable moiety (CM1), wherein the MM1 inhibits the binding of the TB1 to the first target;
a half-life extending moiety (EM) and a second masking moiety (MM2) directly or indirectly coupled to the TB1 or to the TB2 via a second cleavable moiety (CM2), wherein the MM2 inhibits the binding of the TB2 to the second target,
wherein the components of the activatable molecule are configured such that cleavage of the CM1 and the CM2 releases the MM1, the MM2, and the EM from the TB1 that is directly or indirectly coupled to the TB2, and
wherein optionally the TB1 is an antigen-binding molecule (AB1) comprising a HVD1 and an LVD1, and optionally the TB2 is an antigen-binding molecule (AB2) comprising a HVD2 and an LVD2.

46. The activatable protein of any one of claims 1-45, wherein each of the CM1 and the CM2 independently comprises a substrate for a protease selected from the group consisting of ADAMS, ADAMTS, ADAM8, ADAM9, ADAM10, ADAM12, ADAM15, ADAM17/TACE, ADAMDEC1, ADAMTS1, ADAMTS4, ADAMTS5, Aspartate proteases, BACE, Renin, Aspartic cathepsins, Cathepsin D, Cathepsin E, Caspases, Caspase 1, Caspase 2, Caspase 3, Caspase 4, Caspase 5, Caspase 6, Caspase 7, Caspase 8, Caspase 9, Caspase 10, Caspase 14, Cysteine cathepsins, Cathepsin B, Cathepsin C, Cathepsin K, Cathepsin L, Cathepsin S, Cathepsin V/L2, Cathepsin X/Z/P, Cysteine proteinases, Cruzipain, Legumain, Otubain-2, KLKs, KLK4, KLK5, KLK6, KLK7, KLK8, KLK10, KLK11, KLK13, KLK14, Metallo proteinases, Meprin, Neprilysin, PSMA, BMP-1, MMPs, MMP1, MMP2, MMP3, MMP7, MMP8, MMP9, MMP10, MMP11, MMP12, MMP13, MMP14, MMP15, MMP16, MMP17, MMP19, MMP20, MMP23, MMP24, MMP26, MMP27, Serine proteases, activated protein C, Cathepsin A, Cathepsin G, Chymase, coagulation factor proteases, FVIIa, FIXa, FXa, FXIa, FXIIa, Elastase, Granzyme B, Guanidinobenzoatase, HtrA1, Human Neutrophil Elastase, Lactoferrin, Marapsin, NS3/4A, PACE4, Plasmin, PSA, tPA, Thrombin, Tryptase, uPA, Type II Transmembrane, Serine Proteases, TTSPs, DESC1, DPP-4, FAP, Hepsin, Matriptase-2, MT-SP1/Matriptase, TMPRSS2, TMPRSS3, and TMPRSS4.

47. The activatable protein of any one of claims 1-46, wherein the MM1 is a peptide of from 2 to 40 amino acids in length.

48. The activatable protein of any one of claims 1-47, wherein the MM2 is a peptide of from 2 to 40 amino acids in length.

49. The activatable protein of claim 47 or 48, wherein
the heavy chain variable region of the AB2 is directly or indirectly coupled to a C-terminus of the heavy chain fragment of the AB1,
an N-terminus of the MM2 is directly or indirectly coupled to a C-terminus of a light chain variable region of the AB2 via the CM2, and
the EM comprises a dimer of a first Fc domain and a second Fc domain, and a C-terminus of the MM2 is directly or indirectly coupled to an N-terminus of the first Fc domain of the EM.

50. The activatable protein of claim 47 or 48, wherein
the heavy chain variable region of the AB2 is directly or indirectly coupled to a C-terminus of the light chain fragment of the AB1,
an N-terminus of the MM2 is directly or indirectly coupled to a C-terminus of the heavy chain fragment of the AB1 via the CM2, and
the EM comprises a dimer of a first Fc domain and a second Fc domain, and a C-terminus of the MM2 is directly or indirectly coupled to an N-terminus of the first Fc domain of the EM.

51. The activatable protein claim 47 or 48, wherein
the heavy chain variable region of the AB2 is directly or indirectly coupled to a C-terminus of the light chain fragment of the AB1,
the EM comprises a dimer of a first Fc domain and a second Fc domain, and an N-terminus of the first Fc domain is directly or indirectly coupled to a C-terminus of the heavy chain fragment of the AB1 via the CM2, and
an N-terminus of the MM2 is directly or indirectly coupled to an C-terminus of the second Fc domain.

52. The activatable protein of claim 47 or 48, wherein
the heavy chain variable region of the AB2 is directly or indirectly coupled to a C-terminus of the light chain fragment of the AB1,
the EM comprises a dimer of a first Fc domain and a second Fc domain, and an N-terminus of the first Fc domain is directly or indirectly coupled to a C-terminus of the heavy chain fragment of the AB1 via the CM2, and
a C-terminus of the MM2 is directly or indirectly coupled to an N-terminus of the second Fc domain.

53. The activatable protein of any one of claims 49-52, further comprising a linker between the MM2 and the first or second Fc domain directly or indirectly coupled to the MM2.

54. The activatable protein of any one of claims 1-53, wherein the MM1 comprises a sequence of SEQ ID NO: 40 and the MM2 comprises a sequence of any one of SEQ ID NO: 34-37, or 66-70.

55. The activatable protein of any one of claims 1-54, wherein the MM1 has a dissociation constant for binding to the AB1 that is greater than a dissociation constant of the AB1 for binding to the first target or epitope, and the MM2 has a dissociation constant for binding to the AB2 that is greater than a dissociation constant of the AB2 for binding to the second target or epitope.

56. The activatable protein of any one of claims 1-55, wherein the components of the activatable protein are configured such that cleavage of the CM1 and the CM2 severs the MM1, the MM2, and the EM from the activatable protein thereby generating an activated protein having a shorter half-life compared to a counterpart molecule comprising the TB1, TB2, and EM.

57. The activatable protein of any one of claims 1-55, wherein the components of the activatable protein are configured such that cleavage of the CM1 and the CM2 severs the MM1, the MM2, and the EM from the activatable protein thereby generating an activated protein that has a higher target-binding activity compared to a counterpart molecule comprising the TB1, TB2, and the EM.

58. The activatable protein of any one of claims 1-57, wherein the second polypeptide further comprises a linker (L2) between the MM2 and the AB2.

59. The activatable protein of claim 58, wherein L2 is a peptide having a length of 5 to 30, 6 to 29, 7 to 28, 8 to 27, 9 to 26, 10 to 25, 11, 12, 13, 14, 15, 16, 17, 18, 19, 20, 21, 22, 23, 24, 25, 26, or 27 amino acids.

60. The activatable protein of any of claims 1-59, wherein the second polypeptide further comprises a linker (L3) between the AB2 and the AB1.

61. The activatable protein of claim 60, wherein L3 is a peptide having a length of 5 to 30, 6 to 29, 7 to 28, 8 to 27, 9 to 26, 10 to 25, 11, 12, 13, 14, 15, 16, 17, 18, 19, 20, 21, 22, 23, 24, 25, 26, or 27 amino acids.

62. The activatable molecule of claim 45, further comprising the features of any one or more of claims 1-44.

63. An activatable protein comprising:
a first antigen-binding domain (AB1) that specifically binds to a first target, wherein the AB1 comprises a first heavy chain variable domain (HVD1) and a first light chain variable domain (LVD1);
a second antigen-binding domain (AB2) that specifically binds to a second target, wherein the AB2 comprises a second heavy chain variable domain (HVD2) and a second light chain variable domain (LVD2), wherein the AB2 is directly or indirectly coupled to an N-terminus of the HVD1 or to an N-terminus of the LVD1;
a first masking moiety (MM1) coupled to the AB1 via a first cleavable moiety (CM1) and optionally one or more linkers, wherein the MM1 inhibits the binding of the AB1 to the first target;
a second masking moiety (MM2) coupled to the AB1 via a second cleavable moiety (CM2) and optionally one or more linkers, wherein the MM2 inhibits the binding of the AB2 to the second target;
a half-life extending moiety (EM) directly or indirectly coupled to a C-terminus of the HVD1 or to a C-terminus of the LVD1 via a third cleavable moiety (CM3) and optionally one or more linkers.

64. The activatable molecule of claim 63, further comprising the features of any one or more of claims 1-61.

65. A composition comprising the activatable protein of any one of claims 1 to 64 and a carrier.

66. The composition of claim 65, wherein the composition is a pharmaceutical composition, wherein the carrier is a pharmaceutically acceptable carrier.

67. A container, vial, syringe, injector pen, or kit comprising at least one dose of the composition of claim 66.

68. A nucleic acid comprising a sequence encoding the second polypeptide of any of claims 8, 10-13, 58, or 60.

69. A vector comprising the nucleic acid of claim 68.

70. A cell comprising the nucleic acid of claim 68 or the vector of claim 69.

71. A conjugated activatable protein comprising the activatable protein of any one of claims 1 to 64 conjugated to an agent.

72. The conjugated activatable protein of claim 71, wherein the agent is a therapeutic agent, an antineoplastic agent, a toxin, a diagnostic agent, a therapeutic macromolecule, a targeting moiety, or a detectable moiety.

73. The conjugated activatable protein of claim 71 or claim 72, wherein the agent is conjugated to the antibody via a linker.

74. The conjugated activatable protein of claim 72, wherein the linker is a cleavable linker.

75. The conjugated activatable protein of claim 72, wherein the linker is a non-cleavable linker.

76. A method of treating a subject in need thereof comprising administering to the subject a therapeutically effective amount of the activatable protein of any one of claims 1 to 64 the composition of claim 65 or 66, or the conjugated activatable protein of any one claims 71-75.

77. The method of claim 76, wherein the subject has been identified or diagnosed as having a cancer.

78. A method of producing an activatable protein, comprising:
culturing the cell of claim 70 in a culture medium under a condition sufficient to produce the activatable protein; and
recovering the activatable protein from the cell or the culture medium.

79. The method of claim 78, further comprising isolating the activatable protein recovered from the cell or the culture medium.

80. The method of claim 79, wherein isolating the activatable protein is performed using a protein purification tag and/or size exclusion chromatography.

81. The method of any of claims 78-80, further comprising formulating the activatable protein into a pharmaceutical composition.


SEQUENCE LISTING (652 sequences)

SEQ ID NO: 1 - protein, 228 aa, synthetic construct
  VARIANT 228: note = X is lysine or absent
SKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGX

SEQ ID NO: 2 - protein, 229 aa, synthetic construct
  VARIANT 229: note = X is lysine or absent
ESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGX

SEQ ID NO: 3 - protein, 330 aa, synthetic construct
  VARIANT 330: note = X is lysine or absent
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGX

SEQ ID NO: 4 - protein, 326 aa, synthetic construct
  VARIANT 326: note = X is lysine or absent
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGX

SEQ ID NO: 5 - protein, 377 aa, synthetic construct
  VARIANT 377: note = X is lysine or absent
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGX

SEQ ID NO: 6 - protein, 327 aa, synthetic construct
  VARIANT 327: note = X is lysine or absent
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGX

SEQ ID NO: 7 - protein, 14 aa, synthetic construct
RSSTGAVTTSNYAN

SEQ ID NO: 8 - protein, 7 aa, synthetic construct
GTNKRAP

SEQ ID NO: 9 - protein, 9 aa, synthetic construct
ALWYSNLWV

SEQ ID NO: 10 - protein, 5 aa, synthetic construct
TYAMN

SEQ ID NO: 11 - protein, 19 aa, synthetic construct
RIRSKYNNYATYYADSVKD

SEQ ID NO: 12 - protein, 14 aa, synthetic construct
HGNFGNSYVSWFAY

SEQ ID NO: 13 - protein, 14 aa, synthetic construct
HGNFGNSYVSWWAY

SEQ ID NO: 14 - protein, 14 aa, synthetic construct
RSSTGAVTTSNYPN

SEQ ID NO: 15 - protein, 9 aa, synthetic construct
VLWYSNRWV

SEQ ID NO: 16 - protein, 109 aa, synthetic construct
QTVVTQEPSLTVSPGGTVTLTCRSSTGAVTTSNYPNWVQQKPGQAPRGLIGGTNKRAPGTPARFSGSLLGGKAALTLSGVQPEDEAEYYCVLWYSNRWVFGGGTKLTVL

SEQ ID NO: 17 - protein, 214 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - protein, 261 aa, synthetic construct
QGQSGQGALICCSDVSGLCRWCGGGSSGGSISSGLLSGRSDNHGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 249 aa, synthetic construct
EVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCVRHGNFGNSYVSWWAYWGQGTLVTVSSGGGGSGGGGSGGGGSQTVVTQEPSLTVSPGGTVTLTCRSSTGAVTTSNYPNWVQQKPGQAPRGLIGGTNKRAPGTPARFSGSLLGGKAALTLSGVQPEDEAEYYCVLWYSNRWVFGGGTKLTVL

SEQ ID NO: 20 - protein, 14 aa, synthetic construct
GSSTGAVTTSNYVN

SEQ ID NO: 21 - protein, 795 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVGGGGSGGGGSGGGGSEVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCVRHGNFGNSYVSWWAYWGQGTLVTVSSGGGGSGGGGSGGGGSQTVVTQEPSLTVSPGGTVTLTCRSSTGAVTTSNYANWVQQKPGQAPRGLIGGTNKRAPGTPARFSGSLIGGKAALTLSGAQPEDEAEYYCVLWYSNLWVFGGGTKLTVLGGSGGSSGASGTGTAGGTGSGSGTGSGLSGRSDDHGGGSQGQSGSGYLWGCEWNCGGITTGSSGGSGGGGSGGGGSGGGGSGGSESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 22 - protein, 795 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVGGGGSGGGGSGGGGSEVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCVRHGNFGNSYVSWWAYWGQGTLVTVSSGGGGSGGGGSGGGGSQTVVTQEPSLTVSPGGTVTLTCRSSTGAVTTSNYANWVQQKPGQAPRGLIGGTNKRAPGTPARFSGSLIGGKAALTLSGAQPEDEAEYYCVLWYSNLWVFGGGTKLTVLGGSGGSSGASGTGTAGGTGSGSGTGSGLSGRSDDHGGGSQGQSGSNAFRCWWDPPCQPMTGSSGGSGGGGSGGGGSGGGGSGGSESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 23 - protein, 795 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVGGGGSGGGGSGGGGSEVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCVRHGNFGNSYVSWWAYWGQGTLVTVSSGGGGSGGGGSGGGGSQTVVTQEPSLTVSPGGTVTLTCRSSTGAVTTSNYANWVQQKPGQAPRGLIGGTNKRAPGTPARFSGSLIGGKAALTLSGAQPEDEAEYYCVLWYSNLWVFGGGTKLTVLGGSGGSSGASGTGTAGGTGSGSGTGSGLSGRSDDHGGGSQGQSGSARGLCWWDPPCTHDLGSSGGSGGGGSGGGGSGGGGSGGSESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 24 - protein, 795 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVGGGGSGGGGSGGGGSEVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCVRHGNFGNSYVSWWAYWGQGTLVTVSSGGGGSGGGGSGGGGSQTVVTQEPSLTVSPGGTVTLTCRSSTGAVTTSNYANWVQQKPGQAPRGLIGGTNKRAPGTPARFSGSLIGGKAALTLSGAQPEDEAEYYCVLWYSNLWVFGGGTKLTVLGGSGGSSGASGTGTAGGTGSGSGTGSGLSGRSDDHGGGSQGQSGSNHSLCYWDPPCEPSTGSSGGSGGGGSGGGGSGGGGSGGSESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 25 - protein, 109 aa, synthetic construct
QTVVTQEPSLTVSPGGTVTLTCGSSTGAVTTSNYVNWVQQKPGQAPRGLIGGTNKRAPGTPARFSGSLIGGKAALTLSGAQPEDEAEYYCVLWYSNRWVFGGGTKLTVL

SEQ ID NO: 26 - protein, 249 aa, synthetic construct
EVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCVRHGNFGNSYVSWWAYWGQGTLVTVSSGGGGSGGGGSGGGGSQTVVTQEPSLTVSPGGTVTLTCGSSTGAVTTSNYVNWVQQKPGQAPRGLIGGTNKRAPGTPARFSGSLIGGKAALTLSGAQPEDEAEYYCVLWYSNRWVFGGGTKLTVL

SEQ ID NO: 27 - protein, 218 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRV

SEQ ID NO: 28 - protein, 15 aa, synthetic construct
GGGGSGGGGSGGGGS

SEQ ID NO: 29 - protein, 249 aa, synthetic construct
EVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCVRHGNFGNSYVSWWAYWGQGTLVTVSSGGGGSGGGGSGGGGSQTVVTQEPSLTVSPGGTVTLTCRSSTGAVTTSNYANWVQQKPGQAPRGLIGGTNKRAPGTPARFSGSLIGGKAALTLSGAQPEDEAEYYCVLWYSNLWVFGGGTKLTVL

SEQ ID NO: 30 - protein, 125 aa, synthetic construct
EVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCVRHGNFGNSYVSWWAYWGQGTLVTVSS

SEQ ID NO: 31 - protein, 109 aa, synthetic construct
QTVVTQEPSLTVSPGGTVTLTCRSSTGAVTTSNYANWVQQKPGQAPRGLIGGTNKRAPGTPARFSGSLIGGKAALTLSGAQPEDEAEYYCVLWYSNLWVFGGGTKLTVL

SEQ ID NO: 32 - protein, 27 aa, synthetic construct
GGSGGSSGASGTGTAGGTGSGSGTGSG

SEQ ID NO: 33 - protein, 8 aa, synthetic construct
LSGRSDDH

SEQ ID NO: 34 - protein, 15 aa, synthetic construct
GYLWGCEWNCGGITT

SEQ ID NO: 35 - protein, 15 aa, synthetic construct
NAFRCWWDPPCQPMT

SEQ ID NO: 36 - protein, 15 aa, synthetic construct
ARGLCWWDPPCTHDL

SEQ ID NO: 37 - protein, 15 aa, synthetic construct
NHSLCYWDPPCEPST

SEQ ID NO: 38 - protein, 24 aa, synthetic construct
GSSGGSGGGGSGGGGSGGGGSGGS

SEQ ID NO: 39 - protein, 9 aa, synthetic construct
VLWYSNLWV

SEQ ID NO: 40 - protein, 15 aa, synthetic construct
ALICCSDVSGLCRWC

SEQ ID NO: 41 - protein, 13 aa, synthetic construct
ISSGLLSGRSDNH

SEQ ID NO: 42 - protein, 327 aa, synthetic construct
  VARIANT 327: note = X is lysine or absent
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGX

SEQ ID NO: 43 - protein, 228 aa, synthetic construct
  VARIANT 228: note = X is lysine or absent
SKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGX

SEQ ID NO: 44 - protein, 225 aa, synthetic construct
  VARIANT 225: note = X is lysine or absent
GPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGX

SEQ ID NO: 45 - protein, 227 aa, synthetic construct
  VARIANT 227: note = X is lysine or absent
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGX

SEQ ID NO: 46 - protein, 227 aa, synthetic construct
  VARIANT 227: note = X is lysine or absent
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGX

SEQ ID NO: 47 - protein, 109 aa, synthetic construct
QTVVTQEPSFSVSPGGTVTLTCRSSTGAVTTSNYANWVQQTPGQAPRGLIGGTNKRAPGVPDRFSGSILGNKAALTITGAQADDESDYYCALWYSNLWVFGGGTKLTVL

SEQ ID NO: 48 - protein, 125 aa, synthetic construct
EVQLVESGGGLVQPGGSLRLSCAASGFTFSTYAMNWVRQAPGKGLEWVARIRSKYNNYATYYADSVKDRFTISRDDSKNSLYLQMNSLKTEDTAVYYCVRHGNFGNSYVSWFAYWGQGTLVTVSS

SEQ ID NO: 49 - protein, 249 aa, synthetic construct
QTVVTQEPSFSVSPGGTVTLTCRSSTGAVTTSNYANWVQQTPGQAPRGLIGGTNKRAPGVPDRFSGSILGNKAALTITGAQADDESDYYCALWYSNLWVFGGGTKLTVLGGGGSGGGGSGGGGSEVQLVESGGGLVQPGGSLRLSCAASGFTFSTYAMNWVRQAPGKGLEWVARIRSKYNNYATYYADSVKDRFTISRDDSKNSLYLQMNSLKTEDTAVYYCVRHGNFGNSYVSWFAYWGQGTLVTVSS

SEQ ID NO: 50 - protein, 125 aa, synthetic construct
EVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCTRHGNFGNSYVSWFAYWGQGTLVTVSS

SEQ ID NO: 51 - protein, 249 aa, synthetic construct
QTVVTQEPSFSVSPGGTVTLTCRSSTGAVTTSNYANWVQQTPGQAPRGLIGGTNKRAPGVPDRFSGSILGNKAALTITGAQADDESDYYCALWYSNLWVFGGGTKLTVLGGGGSGGGGSGGGGSEVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCTRHGNFGNSYVSWFAYWGQGTLVTVSS

SEQ ID NO: 52 - protein, 109 aa, synthetic construct
QAVVTQEPSLTVSPGGTVTLTCRSSTGAVTTSNYANWVQQKPGQAPRGLIGGTNKRAPGTPARFSGSLIGGKAALTLSGAQPEDEAEYYCALWYSNLWVFGGGTKLTVL

SEQ ID NO: 53 - protein, 249 aa, synthetic construct
QAVVTQEPSLTVSPGGTVTLTCRSSTGAVTTSNYANWVQQKPGQAPRGLIGGTNKRAPGTPARFSGSLIGGKAALTLSGAQPEDEAEYYCALWYSNLWVFGGGTKLTVLGGGGSGGGGSGGGGSEVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCTRHGNFGNSYVSWFAYWGQGTLVTVSS

SEQ ID NO: 54 - protein, 249 aa, synthetic construct
QAVVTQEPSLTVSPGGTVTLTCRSSTGAVTTSNYANWVQQKPGQAPRGLIGGTNKRAPGTPARFSGSLIGGKAALTLSGAQPEDEAEYYCALWYSNLWVFGGGTKLTVLGGGGSGGGGSGGGGSEVQLVESGGGLVQPGGSLRLSCAASGFTFSTYAMNWVRQAPGKGLEWVARIRSKYNNYATYYADSVKDRFTISRDDSKNSLYLQMNSLKTEDTAVYYCVRHGNFGNSYVSWFAYWGQGTLVTVSS

SEQ ID NO: 55 - protein, 125 aa, synthetic construct
EVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCVRHGNFGNSYVSWFAYWGCGTLVTVSS

SEQ ID NO: 56 - protein, 109 aa, synthetic construct
QTVVTQEPSLTVSPGGTVTLTCGSSTGAVTTSNYVNWVQQKPGCAPRGLIGGTNKRAPGTPARFSGSLIGGKAALTLSGAQPEDEAEYYCVLWYSNRWVFGGGTKLTVL

SEQ ID NO: 57 - protein, 249 aa, synthetic construct
EVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCVRHGNFGNSYVSWFAYWGCGTLVTVSSGGGGSGGGGSGGGGSQTVVTQEPSLTVSPGGTVTLTCGSSTGAVTTSNYVNWVQQKPGCAPRGLIGGTNKRAPGTPARFSGSLIGGKAALTLSGAQPEDEAEYYCVLWYSNRWVFGGGTKLTVL

SEQ ID NO: 58 - protein, 109 aa, synthetic construct
QTVVTQEPSLTVSPGGTVTLTCGSSTGAVTTSNYVNWVQQKPGQAPRGLIGGTNKRAPGTPARFSGSLIGGKAALTLSGAQPEDEAEYYCVLWYSNLWVFGGGTKLTVL

SEQ ID NO: 59 - protein, 249 aa, synthetic construct
EVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCVRHGNFGNSYVSWWAYWGQGTLVTVSSGGGGSGGGGSGGGGSQTVVTQEPSLTVSPGGTVTLTCGSSTGAVTTSNYVNWVQQKPGQAPRGLIGGTNKRAPGTPARFSGSLIGGKAALTLSGAQPEDEAEYYCVLWYSNLWVFGGGTKLTVL

SEQ ID NO: 60 - protein, 14 aa, synthetic construct
RSSTGAVTTSNYVN

SEQ ID NO: 61 - protein, 9 aa, synthetic construct
ILWYSNRWV

SEQ ID NO: 62 - protein, 109 aa, synthetic construct
QTVVTQEPSLTVSPGGTVTLTCRSSTGAVTTSNYVNWVQQKPGQAPRGLIGGTNKRAPGTPARFSGSLIGGKAALTLSGAQPEDEAEYYCILWYSNRWVFGGGTKLTVL

SEQ ID NO: 63 - protein, 249 aa, synthetic construct
EVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCVRHGNFGNSYVSWWAYWGQGTLVTVSSGGGGSGGGGSGGGGSQTVVTQEPSLTVSPGGTVTLTCRSSTGAVTTSNYVNWVQQKPGQAPRGLIGGTNKRAPGTPARFSGSLIGGKAALTLSGAQPEDEAEYYCILWYSNRWVFGGGTKLTVL

SEQ ID NO: 64 - protein, 109 aa, synthetic construct
QTVVTQEPSLTVSPGGTVTLTCRSSTGAVTTSNYVNWVQQKPGQAPRGLIGGTNKRAPGTPARFSGSLIGGKAALTLSGAQPEDEAEYYCVLWYSNRWVFGGGTKLTVL

SEQ ID NO: 65 - protein, 249 aa, synthetic construct
EVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCVRHGNFGNSYVSWWAYWGQGTLVTVSSGGGGSGGGGSGGGGSQTVVTQEPSLTVSPGGTVTLTCRSSTGAVTTSNYVNWVQQKPGQAPRGLIGGTNKRAPGTPARFSGSLIGGKAALTLSGAQPEDEAEYYCVLWYSNRWVFGGGTKLTVL

SEQ ID NO: 66 - protein, 15 aa, synthetic construct
MMYCGGNEVLCGPRV

SEQ ID NO: 67 - protein, 15 aa, unidentified
GYRWGCEWNCGGITT

SEQ ID NO: 68 - protein, 15 aa, synthetic construct
MMYCGGNEIFCEPRG

SEQ ID NO: 69 - protein, 15 aa, synthetic construct
GYGWGCEWNCGGSSP

SEQ ID NO: 70 - protein, 15 aa, synthetic construct
MMYCGGNEIFCGPRG

SEQ ID NO: 71 - protein, 4 aa, synthetic construct
GGSG

SEQ ID NO: 72 - protein, 5 aa, synthetic construct
GGSGG

SEQ ID NO: 73 - protein, 5 aa, synthetic construct
GSGSG

SEQ ID NO: 74 - protein, 5 aa, synthetic construct
GSGGG

SEQ ID NO: 75 - protein, 5 aa, synthetic construct
GGGSG

SEQ ID NO: 76 - protein, 5 aa, synthetic construct
GSSSG

SEQ ID NO: 77 - protein, 11 aa, synthetic construct
GSSGGSGGSGG

SEQ ID NO: 78 - protein, 4 aa, synthetic construct
GGGS

SEQ ID NO: 79 - protein, 8 aa, synthetic construct
GGGSGGGS

SEQ ID NO: 80 - protein, 12 aa, synthetic construct
GGGSGGGSGGGS

SEQ ID NO: 81 - protein, 15 aa, synthetic construct
GGGGSGGGGSGGGGS

SEQ ID NO: 82 - protein, 25 aa, synthetic construct
GGGGSGGGGSGGGGSGGGGSGGGGS

SEQ ID NO: 83 - protein, 10 aa, synthetic construct
GGGGSGGGGS

SEQ ID NO: 84 - protein, 5 aa, synthetic construct
GGGGS

SEQ ID NO: 85 - protein, 7 aa, synthetic construct
GGGGSGS

SEQ ID NO: 86 - protein, 17 aa, synthetic construct
GGGGSGGGGSGGGGSGS

SEQ ID NO: 87 - protein, 12 aa, synthetic construct
GGSLDPKGGGGS

SEQ ID NO: 88 - protein, 20 aa, synthetic construct
PKSCDKTHTCPPCPAPELLG

SEQ ID NO: 89 - protein, 17 aa, synthetic construct
SKYGPPCPPCPAPEFLG

SEQ ID NO: 90 - protein, 12 aa, synthetic construct
GKSSGSGSESKS

SEQ ID NO: 91 - protein, 14 aa, synthetic construct
GSTSGSGKSSEGKG

SEQ ID NO: 92 - protein, 18 aa, synthetic construct
GSTSGSGKSSEGSGSTKG

SEQ ID NO: 93 - protein, 18 aa, synthetic construct
GSTSGSGKPGSGEGSTKG

SEQ ID NO: 94 - protein, 16 aa, synthetic construct
GSTSGSGKPGSSEGST

SEQ ID NO: 95 - protein, 8 aa, synthetic construct
GGGSSGGS

SEQ ID NO: 96 - protein, 11 aa, synthetic construct
GGGGSGGGGSS

SEQ ID NO: 97 - protein, 15 aa, synthetic construct
GGGSSGGSGGSSGGS

SEQ ID NO: 98 - protein, 16 aa, synthetic construct
GSTSGSGKPGSSEGST

SEQ ID NO: 99 - protein, 10 aa, synthetic construct
AANALAHGLF

SEQ ID NO: 100 - protein, 4 aa, synthetic construct
AANL

SEQ ID NO: 101 - protein, 10 aa, synthetic construct
AANLGSGGSS

SEQ ID NO: 102 - protein, 5 aa, synthetic construct
AAPRS

SEQ ID NO: 103 - protein, 6 aa, synthetic construct
AAPRSF

SEQ ID NO: 104 - protein, 8 aa, synthetic construct
AARGPAIH

SEQ ID NO: 105 - protein, 8 aa, synthetic construct
AAYHLVSQ

SEQ ID NO: 106 - protein, 10 aa, synthetic construct
AFPDMRSVRS

SEQ ID NO: 107 - protein, 7 aa, synthetic construct
AFQALRM

SEQ ID NO: 108 - protein, 6 aa, synthetic construct
AFRHLR

SEQ ID NO: 109 - protein, 8 aa, synthetic construct
AGLGISST

SEQ ID NO: 110 - protein, 8 aa, synthetic construct
AGLGVVER

SEQ ID NO: 111 - protein, 4 aa, synthetic construct
AGPR

SEQ ID NO: 112 - protein, 4 aa, synthetic construct
AHGL

SEQ ID NO: 113 - protein, 5 aa, synthetic construct
AHGLF

SEQ ID NO: 114 - protein, 7 aa, synthetic construct
AHQALRM

SEQ ID NO: 115 - protein, 10 aa, synthetic construct
AIPRVRLFDV

SEQ ID NO: 116 - protein, 5 aa, synthetic construct
ALAHG

SEQ ID NO: 117 - protein, 6 aa, synthetic construct
ALAHGL

SEQ ID NO: 118 - protein, 7 aa, synthetic construct
ALAHGLF

SEQ ID NO: 119 - protein, 12 aa, synthetic construct
ALAHGLFAPRSF

SEQ ID NO: 120 - protein, 13 aa, synthetic construct
ALAHGLFSGRSAN

SEQ ID NO: 121 - protein, 12 aa, synthetic construct
ALAHGLPTFVHL

SEQ ID NO: 122 - protein, 8 aa, synthetic construct
ALGLLRLP

SEQ ID NO: 123 - protein, 10 aa, synthetic construct
ALPSVKMVSE

SEQ ID NO: 124 - protein, 5 aa, synthetic construct
ALRAP

SEQ ID NO: 125 - protein, 7 aa, synthetic construct
ANQALRM

SEQ ID NO: 126 - protein, 8 aa, synthetic construct
ANQALRMA

SEQ ID NO: 127 - protein, 10 aa, synthetic construct
APPLVKSMVV

SEQ ID NO: 128 - protein, 10 aa, synthetic construct
APPSFKLVNA

SEQ ID NO: 129 - protein, 4 aa, synthetic construct
APRS

SEQ ID NO: 130 - protein, 11 aa, synthetic construct
APRSALAHGLF

SEQ ID NO: 131 - protein, 5 aa, synthetic construct
APRSF

SEQ ID NO: 132 - protein, 8 aa, synthetic construct
AQFVLTEG

SEQ ID NO: 133 - protein, 8 aa, synthetic construct
AQNLLGMV

SEQ ID NO: 134 - protein, 4 aa, synthetic construct
ARGP

SEQ ID NO: 135 - protein, 5 aa, synthetic construct
ARGPS

SEQ ID NO: 136 - protein, 6 aa, synthetic construct
ARGPSF

SEQ ID NO: 137 - protein, 7 aa, synthetic construct
ARGPSFK

SEQ ID NO: 138 - protein, 8 aa, synthetic construct
ASGLLRFP

SEQ ID NO: 139 - protein, 10 aa, synthetic construct
ASPTMKTVGL

SEQ ID NO: 140 - protein, 8 aa, synthetic construct
AVGLLAPP

SEQ ID NO: 141 - protein, 18 aa, synthetic construct
AVGLLAPPGGLSGRSANI

SEQ ID NO: 142 - protein, 18 aa, synthetic construct
AVGLLAPPGGLSGRSANP

SEQ ID NO: 143 - protein, 18 aa, synthetic construct
AVGLLAPPGGLSGRSDDH

SEQ ID NO: 144 - protein, 18 aa, synthetic construct
AVGLLAPPGGLSGRSDIH

SEQ ID NO: 145 - protein, 18 aa, synthetic construct
AVGLLAPPGGLSGRSDNH

SEQ ID NO: 146 - protein, 18 aa, synthetic construct
AVGLLAPPGGLSGRSDNI

SEQ ID NO: 147 - protein, 18 aa, synthetic construct
AVGLLAPPGGLSGRSDNP

SEQ ID NO: 148 - protein, 18 aa, synthetic construct
AVGLLAPPGGLSGRSDQH

SEQ ID NO: 149 - protein, 18 aa, synthetic construct
AVGLLAPPGGLSGRSDTH

SEQ ID NO: 150 - protein, 18 aa, synthetic construct
AVGLLAPPGGLSGRSDYH

SEQ ID NO: 151 - protein, 17 aa, synthetic construct
AVGLLAPPGGLSGRSNI

SEQ ID NO: 152 - protein, 18 aa, synthetic construct
AVGLLAPPGGLSGRSNIG

SEQ ID NO: 153 - protein, 19 aa, synthetic construct
AVGLLAPPGGLSGRSNIGS

SEQ ID NO: 154 - protein, 22 aa, synthetic construct
AVGLLAPPGGTSTSGRSANPRG

SEQ ID NO: 155 - protein, 17 aa, synthetic construct
AVGLLAPPSGRSANPRG

SEQ ID NO: 156 - protein, 18 aa, synthetic construct
AVGLLAPPTSGRSANPRG

SEQ ID NO: 157 - protein, 10 aa, synthetic construct
AVPKVRVVPE

SEQ ID NO: 158 - protein, 7 aa, synthetic construct
CGPPLGR

SEQ ID NO: 159 - protein, 7 aa, synthetic construct
CSPPLGR

SEQ ID NO: 160 - protein, 10 aa, synthetic construct
DEVDGSGGSS

SEQ ID NO: 161 - protein, 8 aa, synthetic construct
DISHWRRS

SEQ ID NO: 162 - protein, 7 aa, synthetic construct
DLAHPLL

SEQ ID NO: 163 - protein, 10 aa, synthetic construct
DLPLVKSLPS

SEQ ID NO: 164 - protein, 12 aa, synthetic construct
DRLSGRSANHKK

SEQ ID NO: 165 - protein, 12 aa, synthetic construct
DRLSGRSDNHKK

SEQ ID NO: 166 - protein, 10 aa, synthetic construct
DRPEMKSLSG

SEQ ID NO: 167 - protein, 10 aa, synthetic construct
DRPKVKTMDF

SEQ ID NO: 168 - protein, 8 aa, synthetic construct
DVAQFVLT

SEQ ID NO: 169 - protein, 10 aa, synthetic construct
DVPPMKTLRP

SEQ ID NO: 170 - protein, 8 aa, synthetic construct
DWLYWMGI

SEQ ID NO: 171 - protein, 8 aa, synthetic construct
DWLYWMSI

SEQ ID NO: 172 - protein, 8 aa, synthetic construct
DWLYWPGI

SEQ ID NO: 173 - protein, 8 aa, synthetic construct
DWLYWPSI

SEQ ID NO: 174 - protein, 10 aa, synthetic construct
EAPKVKALPK

SEQ ID NO: 175 - protein, 10 aa, synthetic construct
EHPRVKVVSE

SEQ ID NO: 176 - protein, 10 aa, synthetic construct
EKPRMKLFQG

SEQ ID NO: 177 - protein, 8 aa, synthetic construct
EPQALAMS

SEQ ID NO: 178 - protein, 10 aa, synthetic construct
EQPEVKMVKG

SEQ ID NO: 179 - protein, 10 aa, synthetic construct
ERPGVKSLVL

SEQ ID NO: 180 - protein, 8 aa, synthetic construct
ESLPVVAV

SEQ ID NO: 181 - protein, 10 aa, synthetic construct
ESPVMKSMAL

SEQ ID NO: 182 - protein, 5 aa, synthetic construct
ESRRW

SEQ ID NO: 183 - protein, 6 aa, synthetic construct
ESRRWM

SEQ ID NO: 184 - protein, 7 aa, synthetic construct
ESRRWMP

SEQ ID NO: 185 - protein, 10 aa, synthetic construct
ETPSVKTMGR

SEQ ID NO: 186 - protein, 12 aa, synthetic construct
ETPSVKTMGRSS

SEQ ID NO: 187 - protein, 10 aa, synthetic construct
FPRPLGITGL

SEQ ID NO: 188 - protein, 8 aa, synthetic construct
FRLLDWQW

SEQ ID NO: 189 - protein, 8 aa, synthetic construct
GCGPPLGR

SEQ ID NO: 190 - protein, 8 aa, synthetic construct
GCSPPLGR

SEQ ID NO: 191 - protein, 10 aa, synthetic construct
GFPHMKTFQH

SEQ ID NO: 192 - protein, 12 aa, synthetic construct
GFPHMKTFQHSS

SEQ ID NO: 193 - protein, 8 aa, synthetic construct
GGGPPLGR

SEQ ID NO: 194 - protein, 7 aa, synthetic construct
GGPPLGR

SEQ ID NO: 195 - protein, 10 aa, synthetic construct
GGQPSGMWGW

SEQ ID NO: 196 - protein, 7 aa, synthetic construct
GGSIDGR

SEQ ID NO: 197 - protein, 8 aa, synthetic construct
GGSPPLGR

SEQ ID NO: 198 - protein, 8 aa, synthetic construct
GGWHTGRN

SEQ ID NO: 199 - protein, 5 aa, synthetic construct
GIAGQ

SEQ ID NO: 200 - protein, 10 aa, synthetic construct
GLGTPRGLFA

SEQ ID NO: 201 - protein, 6 aa, synthetic construct
GLPTFV

SEQ ID NO: 202 - protein, 7 aa, synthetic construct
GLPTFVH

SEQ ID NO: 203 - protein, 8 aa, synthetic construct
GLPTFVHL

SEQ ID NO: 204 - protein, 12 aa, synthetic construct
GLPTFVHLPRQV

SEQ ID NO: 205 - protein, 12 aa, synthetic construct
GLSGRSDNHGSS

SEQ ID NO: 206 - protein, 8 aa, synthetic construct
GPEGLRVG

SEQ ID NO: 207 - protein, 8 aa, synthetic construct
GPLGIAGI

SEQ ID NO: 208 - protein, 12 aa, synthetic construct
GPLNGRSDNHKA

SEQ ID NO: 209 - protein, 12 aa, synthetic construct
GPLNGRSDNHKK

SEQ ID NO: 210 - protein, 12 aa, synthetic construct
GPLNGRSDNHKR

SEQ ID NO: 211 - protein, 12 aa, synthetic construct
GPLNGRSDNHQA

SEQ ID NO: 212 - protein, 12 aa, synthetic construct
GPLNGRSDNHQK

SEQ ID NO: 213 - protein, 12 aa, synthetic construct
GPLNGRSDNHQR

SEQ ID NO: 214 - protein, 12 aa, synthetic construct
GPLNGRSDNHRA

SEQ ID NO: 215 - protein, 12 aa, synthetic construct
GPLNGRSDNHRK

SEQ ID NO: 216 - protein, 12 aa, synthetic construct
GPLNGRSDNHRR

SEQ ID NO: 217 - protein, 12 aa, synthetic construct
GPLSGRSDNHKA

SEQ ID NO: 218 - protein, 12 aa, synthetic construct
GPLSGRSDNHKK

SEQ ID NO: 219 - protein, 12 aa, synthetic construct
GPLSGRSDNHKR

SEQ ID NO: 220 - protein, 12 aa, synthetic construct
GPLSGRSDNHQA

SEQ ID NO: 221 - protein, 12 aa, synthetic construct
GPLSGRSDNHQK

SEQ ID NO: 222 - protein, 12 aa, synthetic construct
GPLSGRSDNHQR

SEQ ID NO: 223 - protein, 12 aa, synthetic construct
GPLSGRSDNHRA

SEQ ID NO: 224 - protein, 12 aa, synthetic construct
GPLSGRSDNHRK

SEQ ID NO: 225 - protein, 12 aa, synthetic construct
GPLSGRSDNHRR

SEQ ID NO: 226 - protein, 6 aa, synthetic construct
GPPLGR

SEQ ID NO: 227 - protein, 8 aa, synthetic construct
GPQGIAGQ

SEQ ID NO: 228 - protein, 8 aa, synthetic construct
GPQGLLGA

SEQ ID NO: 229 - protein, 6 aa, synthetic construct
GPRSFG

SEQ ID NO: 230 - protein, 7 aa, synthetic construct
GPRSFGL

SEQ ID NO: 231 - protein, 8 aa, synthetic construct
GPSHLVLT

SEQ ID NO: 232 - protein, 4 aa, synthetic construct
GPTN

SEQ ID NO: 233 - protein, 11 aa, synthetic construct
GPTNALAHGLF

SEQ ID NO: 234 - protein, 5 aa, synthetic construct
GRSML

SEQ ID NO: 235 - protein, 6 aa, synthetic construct
GRSMLL

SEQ ID NO: 236 - protein, 7 aa, synthetic construct
GRSMLLG

SEQ ID NO: 237 - protein, 8 aa, synthetic construct
GRSMLLGG

SEQ ID NO: 238 - protein, 8 aa, synthetic construct
GRSMLLGP

SEQ ID NO: 239 - protein, 8 aa, synthetic construct
GRSMLLGS

SEQ ID NO: 240 - protein, 7 aa, synthetic construct
GRSMLLP

SEQ ID NO: 241 - protein, 8 aa, synthetic construct
GRSMLLPG

SEQ ID NO: 242 - protein, 8 aa, synthetic construct
GRSMLLPP

SEQ ID NO: 243 - protein, 8 aa, synthetic construct
GRSMLLPS

SEQ ID NO: 244 - protein, 7 aa, synthetic construct
GRSMLLS

SEQ ID NO: 245 - protein, 8 aa, synthetic construct
GRSMLLSG

SEQ ID NO: 246 - protein, 8 aa, synthetic construct
GRSMLLSP

SEQ ID NO: 247 - protein, 8 aa, synthetic construct
GRSMLLSS

SEQ ID NO: 248 - protein, 6 aa, synthetic construct
GRSMLM

SEQ ID NO: 249 - protein, 7 aa, synthetic construct
GRSMLMG

SEQ ID NO: 250 - protein, 8 aa, synthetic construct
GRSMLMGG

SEQ ID NO: 251 - protein, 8 aa, synthetic construct
GRSMLMGP

SEQ ID NO: 252 - protein, 8 aa, synthetic construct
GRSMLMGS

SEQ ID NO: 253 - protein, 7 aa, synthetic construct
GRSMLMP

SEQ ID NO: 254 - protein, 8 aa, synthetic construct
GRSMLMPG

SEQ ID NO: 255 - protein, 8 aa, synthetic construct
GRSMLMPP

SEQ ID NO: 256 - protein, 8 aa, synthetic construct
GRSMLMPS

SEQ ID NO: 257 - protein, 7 aa, synthetic construct
GRSMLMS

SEQ ID NO: 258 - protein, 8 aa, synthetic construct
GRSMLMSG

SEQ ID NO: 259 - protein, 8 aa, synthetic construct
GRSMLMSP

SEQ ID NO: 260 - protein, 8 aa, synthetic construct
GRSMLMSS

SEQ ID NO: 261 - protein, 8 aa, synthetic construct
GSGPPLGR

SEQ ID NO: 262 - protein, 7 aa, synthetic construct
GSPPLGR

SEQ ID NO: 263 - protein, 8 aa, synthetic construct
GSSPPLGR

SEQ ID NO: 264 - protein, 12 aa, synthetic construct
GTGRGPSWVGSS

SEQ ID NO: 265 - protein, 4 aa, synthetic construct
PTNL

SEQ ID NO: 266 - protein, 10 aa, synthetic construct
PTNLGSGGSS

SEQ ID NO: 267 - protein, 8 aa, synthetic construct
PVGYTSSL

SEQ ID NO: 268 - protein, 10 aa, synthetic construct
PVPRLKLIKD

SEQ ID NO: 269 - protein, 8 aa, synthetic construct
PVQPIGPQ

SEQ ID NO: 270 - protein, 8 aa, synthetic construct
QALAMSAI

SEQ ID NO: 271 - protein, 7 aa, synthetic construct
QFQALRM

SEQ ID NO: 272 - protein, 10 aa, synthetic construct
QGPMFKSLWD

SEQ ID NO: 273 - protein, 8 aa, synthetic construct
QGRAITFI

SEQ ID NO: 274 - protein, 7 aa, synthetic construct
QHQALRM

SEQ ID NO: 275 - protein, 8 aa, synthetic construct
QNQALRIA

SEQ ID NO: 276 - protein, 7 aa, synthetic construct
QNQALRM

SEQ ID NO: 277 - protein, 8 aa, synthetic construct
QNQALRMA

SEQ ID NO: 278 - protein, 22 aa, synthetic construct
QNQALRMAGGSGGSLSGRSDNH

SEQ ID NO: 279 - protein, 22 aa, synthetic construct
QNQALRMAGGSGGSLSGRSGNH

SEQ ID NO: 280 - protein, 6 aa, synthetic construct
QSRRVP

SEQ ID NO: 281 - protein, 7 aa, synthetic construct
QSRRVPL

SEQ ID NO: 282 - protein, 7 aa, synthetic construct
QSRRVPV

SEQ ID NO: 283 - protein, 6 aa, synthetic construct
QTRRVP

SEQ ID NO: 284 - protein, 7 aa, synthetic construct
QTRRVPL

SEQ ID NO: 285 - protein, 7 aa, synthetic construct
QTRRVPV

SEQ ID NO: 286 - protein, 8 aa, synthetic construct
QYIVSRSA

SEQ ID NO: 287 - protein, 6 aa, synthetic construct
RALRAP

SEQ ID NO: 288 - protein, 10 aa, synthetic construct
REPFMKSLPW

SEQ ID NO: 289 - protein, 6 aa, synthetic construct
RFPLKV

SEQ ID NO: 290 - protein, 10 aa, synthetic construct
RFPSLKSFPL

SEQ ID NO: 291 - protein, 7 aa, synthetic construct
RFPYGVW

SEQ ID NO: 292 - protein, 8 aa, synthetic construct
RFYRNQFF

SEQ ID NO: 293 - protein, 4 aa, synthetic construct
RGPA

SEQ ID NO: 294 - protein, 8 aa, synthetic construct
RGPAFNPM

SEQ ID NO: 295 - protein, 8 aa, synthetic construct
RGPATPIM

SEQ ID NO: 296 - protein, 7 aa, synthetic construct
RGPKLYW

SEQ ID NO: 297 - protein, 6 aa, synthetic construct
RHLAKL

SEQ ID NO: 298 - protein, 8 aa, synthetic construct
RIGRSDNH

SEQ ID NO: 299 - protein, 8 aa, synthetic construct
RKMPNITV

SEQ ID NO: 300 - protein, 14 aa, synthetic construct
RKSSIIIRMRDVVL

SEQ ID NO: 301 - protein, 8 aa, synthetic construct
RKTVQHWW

SEQ ID NO: 302 - protein, 8 aa, synthetic construct
RLGRSDNN

SEQ ID NO: 303 - protein, 8 aa, synthetic construct
RMHLRSLG

SEQ ID NO: 304 - protein, 8 aa, synthetic construct
RPLARAGI

SEQ ID NO: 305 - protein, 8 aa, synthetic construct
RPLARAGL

SEQ ID NO: 306 - protein, 12 aa, synthetic construct
RPLNGRSDNHKA

SEQ ID NO: 307 - protein, 12 aa, synthetic construct
RPLNGRSDNHKK

SEQ ID NO: 308 - protein, 12 aa, synthetic construct
RPLNGRSDNHKR

SEQ ID NO: 309 - protein, 12 aa, synthetic construct
RPLNGRSDNHQA

SEQ ID NO: 310 - protein, 12 aa, synthetic construct
RPLNGRSDNHQK

SEQ ID NO: 311 - protein, 12 aa, synthetic construct
RPLNGRSDNHQR

SEQ ID NO: 312 - protein, 12 aa, synthetic construct
RPLNGRSDNHRA

SEQ ID NO: 313 - protein, 12 aa, synthetic construct
RPLNGRSDNHRK

SEQ ID NO: 314 - protein, 12 aa, synthetic construct
RPLNGRSDNHRR

SEQ ID NO: 315 - protein, 12 aa, synthetic construct
RPLSGRSDNHKA

SEQ ID NO: 316 - protein, 12 aa, synthetic construct
RPLSGRSDNHKK

SEQ ID NO: 317 - protein, 12 aa, synthetic construct
RPLSGRSDNHKR

SEQ ID NO: 318 - protein, 12 aa, synthetic construct
RPLSGRSDNHQA

SEQ ID NO: 319 - protein, 12 aa, synthetic construct
RPLSGRSDNHQK

SEQ ID NO: 320 - protein, 12 aa, synthetic construct
RPLSGRSDNHQR

SEQ ID NO: 321 - protein, 12 aa, synthetic construct
RPLSGRSDNHRA

SEQ ID NO: 322 - protein, 12 aa, synthetic construct
RPLSGRSDNHRK

SEQ ID NO: 323 - protein, 12 aa, synthetic construct
RPLSGRSDNHRR

SEQ ID NO: 324 - protein, 8 aa, synthetic construct
RPSPMWAY

SEQ ID NO: 325 - protein, 8 aa, synthetic construct
RRHDGLRA

SEQ ID NO: 326 - protein, 8 aa, synthetic construct
RRHDGLRS

SEQ ID NO: 327 - protein, 8 aa, synthetic construct
RSLVFAPI

SEQ ID NO: 328 - protein, 8 aa, synthetic construct
RSPSRLKC

SEQ ID NO: 329 - protein, 8 aa, synthetic construct
VAGRSMRP

SEQ ID NO: 330 - protein, 10 aa, synthetic construct
VAPQLKSLVP

SEQ ID NO: 331 - protein, 8 aa, synthetic construct
VAQFVLTE

SEQ ID NO: 332 - protein, 10 aa, synthetic construct
VHMPLGFLGP

SEQ ID NO: 333 - protein, 20 aa, synthetic construct
VHMPLGFLGPGGLSGRSDNH

SEQ ID NO: 334 - protein, 24 aa, synthetic construct
VHMPLGFLGPGGTSTSGRSANPRG

SEQ ID NO: 335 - protein, 10 aa, synthetic construct
VLPELRSVFS

SEQ ID NO: 336 - protein, 8 aa, synthetic construct
VLSKQMSF

SEQ ID NO: 337 - protein, 7 aa, synthetic construct
VPAGRRS

SEQ ID NO: 338 - protein, 8 aa, synthetic construct
VPAGRRSL

SEQ ID NO: 339 - protein, 4 aa, synthetic construct
VPRQ

SEQ ID NO: 340 - protein, 5 aa, synthetic construct
VPRQV

SEQ ID NO: 341 - protein, 5 aa, synthetic construct
VSRSA

SEQ ID NO: 342 - protein, 8 aa, synthetic construct
VVPEGRRS

SEQ ID NO: 343 - protein, 8 aa, synthetic construct
WATPRPMR

SEQ ID NO: 344 - protein, 8 aa, synthetic construct
WDHPISLL

SEQ ID NO: 345 - protein, 10 aa, synthetic construct
YDPZVKVVLA

SEQ ID NO: 346 - protein, 8 aa, synthetic construct
YGAGLGVV

SEQ ID NO: 347 - protein, 7 aa, synthetic construct
YIVSRSA

SEQ ID NO: 348 - protein, 8 aa, synthetic construct
YKKFVGSL

SEQ ID NO: 349 - protein, 10 aa, synthetic construct
YVPRVKALEM

SEQ ID NO: 350 - protein, 8 aa, synthetic construct
HMMQYARH

SEQ ID NO: 351 - protein, 8 aa, synthetic construct
HTGRSGAL

SEQ ID NO: 352 - protein, 5 aa, synthetic construct
HVPRQ

SEQ ID NO: 353 - protein, 6 aa, Synthecium elegans
HVPRQV

SEQ ID NO: 354 - protein, 11 aa, synthetic construct
HVPRQVAPRSF

SEQ ID NO: 355 - protein, 11 aa, synthetic construct
HVPRQVLSGRS

SEQ ID NO: 356 - protein, 13 aa, synthetic construct
HVPRQVLSGRSAN

SEQ ID NO: 357 - protein, 8 aa, synthetic construct
HWHLGPPT

SEQ ID NO: 358 - protein, 8 aa, synthetic construct
IANLLSMV

SEQ ID NO: 359 - protein, 4 aa, synthetic construct
IDGR

SEQ ID NO: 360 - protein, 4 aa, synthetic construct
IEGR

SEQ ID NO: 361 - protein, 8 aa, synthetic construct
ILNLLSMV

SEQ ID NO: 362 - protein, 8 aa, synthetic construct
ILPRSPAF

SEQ ID NO: 363 - protein, 8 aa, synthetic construct
IPFSWSRF

SEQ ID NO: 364 - protein, 8 aa, synthetic construct
IQNLLSMV

SEQ ID NO: 365 - protein, 5 aa, synthetic construct
ISSGL

SEQ ID NO: 366 - protein, 6 aa, synthetic construct
ISSGLL

SEQ ID NO: 367 - protein, 7 aa, synthetic construct
ISSGLLS

SEQ ID NO: 368 - protein, 13 aa, synthetic construct
ISSGLLSGRSANI

SEQ ID NO: 369 - protein, 13 aa, synthetic construct
ISSGLLSGRSANP

SEQ ID NO: 370 - protein, 15 aa, synthetic construct
ISSGLLSGRSANPRG

SEQ ID NO: 371 - protein, 13 aa, synthetic construct
ISSGLLSGRSDDH

SEQ ID NO: 372 - protein, 13 aa, synthetic construct
ISSGLLSGRSDIH

SEQ ID NO: 373 - protein, 13 aa, synthetic construct
ISSGLLSGRSDNI

SEQ ID NO: 374 - protein, 13 aa, synthetic construct
ISSGLLSGRSDNP

SEQ ID NO: 375 - protein, 13 aa, synthetic construct
ISSGLLSGRSDQH

SEQ ID NO: 376 - protein, 13 aa, synthetic construct
ISSGLLSGRSDTH

SEQ ID NO: 377 - protein, 13 aa, synthetic construct
ISSGLLSGRSDYH

SEQ ID NO: 378 - protein, 13 aa, synthetic construct
ISSGLLSGRSGNH

SEQ ID NO: 379 - protein, 12 aa, synthetic construct
ISSGLLSGRSNI

SEQ ID NO: 380 - protein, 13 aa, synthetic construct
ISSGLLSGRSNIG

SEQ ID NO: 381 - protein, 14 aa, synthetic construct
ISSGLLSGRSNIGS

SEQ ID NO: 382 - protein, 8 aa, synthetic construct
ISSGLLSS

SEQ ID NO: 383 - protein, 22 aa, synthetic construct
ISSGLLSSGGSGGSLSGRSDNH

SEQ ID NO: 384 - protein, 22 aa, synthetic construct
ISSGLLSSGGSGGSLSGRSGNH

SEQ ID NO: 385 - protein, 7 aa, synthetic construct
ISSGLSS

SEQ ID NO: 386 - protein, 6 aa, synthetic construct
IVSRSA

SEQ ID NO: 387 - protein, 12 aa, synthetic construct
KGLTGRSDRHQA

SEQ ID NO: 388 - protein, 10 aa, synthetic construct
KGPKVKVVTL

SEQ ID NO: 389 - protein, 12 aa, synthetic construct
KNLYGRSENNGN

SEQ ID NO: 390 - protein, 8 aa, synthetic construct
KRMPVQFL

SEQ ID NO: 391 - protein, 8 aa, synthetic construct
LAAPLGLL

SEQ ID NO: 392 - protein, 4 aa, synthetic construct
LAHG

SEQ ID NO: 393 - protein, 5 aa, synthetic construct
LAHGL

SEQ ID NO: 394 - protein, 6 aa, synthetic construct
LAHGLF

SEQ ID NO: 395 - protein, 9 aa, synthetic construct
LAPLGLQRR

SEQ ID NO: 396 - protein, 5 aa, synthetic construct
LARAG

SEQ ID NO: 397 - protein, 6 aa, synthetic construct
LARAGI

SEQ ID NO: 398 - protein, 6 aa, synthetic construct
LARAGL

SEQ ID NO: 399 - protein, 8 aa, synthetic construct
LKAAPRWA

SEQ ID NO: 400 - protein, 8 aa, synthetic construct
LKAAPRWF

SEQ ID NO: 401 - protein, 8 aa, synthetic construct
LKAAPVWA

SEQ ID NO: 402 - protein, 8 aa, synthetic construct
LKAAPVWF

SEQ ID NO: 403 - protein, 8 aa, synthetic construct
LKGRSYYY

SEQ ID NO: 404 - protein, 8 aa, synthetic construct
LLAPSHRA

SEQ ID NO: 405 - protein, 8 aa, synthetic construct
LLEALRAL

SEQ ID NO: 406 - protein, 8 aa, synthetic construct
LLESLRAL

SEQ ID NO: 407 - protein, 8 aa, synthetic construct
LLLPAHGG

SEQ ID NO: 408 - protein, 8 aa, synthetic construct
LLLPLLGS

SEQ ID NO: 409 - protein, 8 aa, synthetic construct
LLNALRAL

SEQ ID NO: 410 - protein, 8 aa, synthetic construct
LLNSLRAL

SEQ ID NO: 411 - protein, 8 aa, synthetic construct
LLQALRAL

SEQ ID NO: 412 - protein, 8 aa, synthetic construct
LLQSLRAL

SEQ ID NO: 413 - protein, 8 aa, synthetic construct
LLSALRAL

SEQ ID NO: 414 - protein, 8 aa, synthetic construct
LLSSLRAL

SEQ ID NO: 415 - protein, 8 aa, synthetic construct
LNGRSDNH

SEQ ID NO: 416 - protein, 7 aa, synthetic construct
LPAGLLL

SEQ ID NO: 417 - protein, 8 aa, synthetic construct
LPAGLLLR

SEQ ID NO: 418 - protein, 8 aa, synthetic construct
LPAHLVLL

SEQ ID NO: 419 - protein, 8 aa, synthetic construct
LPAHLVLV

SEQ ID NO: 420 - protein, 8 aa, synthetic construct
LPGGLSPW

SEQ ID NO: 421 - protein, 8 aa, synthetic construct
LPSHLVLL

SEQ ID NO: 422 - protein, 8 aa, synthetic construct
LPSHLVLV

SEQ ID NO: 423 - protein, 5 aa, synthetic construct
LPTFV

SEQ ID NO: 424 - protein, 6 aa, synthetic construct
LPTFVH

SEQ ID NO: 425 - protein, 7 aa, synthetic construct
LPTFVHL

SEQ ID NO: 426 - protein, 5 aa, synthetic construct
LRSGW

SEQ ID NO: 427 - protein, 4 aa, synthetic construct
LSGR

SEQ ID NO: 428 - protein, 5 aa, synthetic construct
LSGRS

SEQ ID NO: 429 - protein, 6 aa, synthetic construct
LSGRSA

SEQ ID NO: 430 - protein, 12 aa, synthetic construct
LSGRSALAHGLF

SEQ ID NO: 431 - protein, 7 aa, synthetic construct
LSGRSAN

SEQ ID NO: 432 - protein, 8 aa, synthetic construct
LSGRSANI

SEQ ID NO: 433 - protein, 8 aa, synthetic construct
LSGRSANP

SEQ ID NO: 434 - protein, 6 aa, synthetic construct
LSGRSD

SEQ ID NO: 435 - protein, 7 aa, synthetic construct
LSGRSDD

SEQ ID NO: 436 - protein, 7 aa, synthetic construct
LSGRSDI

SEQ ID NO: 437 - protein, 8 aa, synthetic construct
LSGRSDIH

SEQ ID NO: 438 - protein, 7 aa, synthetic construct
LSGRSDN

SEQ ID NO: 439 - protein, 8 aa, synthetic construct
LSGRSDNH

SEQ ID NO: 440 - protein, 18 aa, synthetic construct
LSGRSDNHGGAVGLLAPP

SEQ ID NO: 441 - protein, 22 aa, synthetic construct
LSGRSDNHGGSGGSISSGLLSS

SEQ ID NO: 442 - protein, 22 aa, synthetic construct
LSGRSDNHGGSGGSQNQALRMA

SEQ ID NO: 443 - protein, 20 aa, synthetic construct
LSGRSDNHGGVHMPLGFLGP

SEQ ID NO: 444 - protein, 8 aa, synthetic construct
LSGRSDNI

SEQ ID NO: 445 - protein, 8 aa, synthetic construct
LSGRSDNP

SEQ ID NO: 446 - protein, 7 aa, synthetic construct
LSGRSDQ

SEQ ID NO: 447 - protein, 8 aa, synthetic construct
LSGRSDQH

SEQ ID NO: 448 - protein, 7 aa, synthetic construct
LSGRSDT

SEQ ID NO: 449 - protein, 8 aa, synthetic construct
LSGRSDTH

SEQ ID NO: 450 - protein, 7 aa, synthetic construct
LSGRSDY

SEQ ID NO: 451 - protein, 8 aa, synthetic construct
LSGRSDYH

SEQ ID NO: 452 - protein, 8 aa, synthetic construct
LSGRSENH

SEQ ID NO: 453 - protein, 6 aa, synthetic construct
LSGRSG

SEQ ID NO: 454 - protein, 7 aa, synthetic construct
LSGRSGN

SEQ ID NO: 455 - protein, 8 aa, synthetic construct
LSGRSGNH

SEQ ID NO: 456 - protein, 22 aa, synthetic construct
LSGRSGNHGGSGGSISSGLLSS

SEQ ID NO: 457 - protein, 22 aa, synthetic construct
LSGRSGNHGGSGGSQNQALRMA

SEQ ID NO: 458 - protein, 8 aa, synthetic construct
LSGRSGNP

SEQ ID NO: 459 - protein, 8 aa, synthetic construct
LSGRSVTQ

SEQ ID NO: 460 - protein, 8 aa, synthetic construct
LSQARWRK

SEQ ID NO: 461 - protein, 8 aa, synthetic construct
LTFPTYIF

SEQ ID NO: 462 - protein, 8 aa, synthetic construct
LTFPTYWF

SEQ ID NO: 463 - protein, 8 aa, synthetic construct
LTGRSDRH

SEQ ID NO: 464 - protein, 7 aa, synthetic construct
LTGRSGA

SEQ ID NO: 465 - protein, 7 aa, synthetic construct
LTGRSGA

SEQ ID NO: 466 - protein, 8 aa, synthetic construct
LYAAPRWA

SEQ ID NO: 467 - protein, 8 aa, synthetic construct
LYAAPRWF

SEQ ID NO: 468 - protein, 8 aa, synthetic construct
LYAAPVWA

SEQ ID NO: 469 - protein, 8 aa, synthetic construct
LYAAPVWF

SEQ ID NO: 470 - protein, 8 aa, synthetic construct
LYGRSENN

SEQ ID NO: 471 - protein, 8 aa, synthetic construct
MDAFLESS

SEQ ID NO: 472 - protein, 8 aa, synthetic construct
MGLFSEAG

SEQ ID NO: 473 - protein, 6 aa, synthetic construct
MGPWFM

SEQ ID NO: 474 - protein, 8 aa, synthetic construct
MIAPVAYR

SEQ ID NO: 475 - protein, 6 aa, synthetic construct
MLRSGW

SEQ ID NO: 476 - protein, 7 aa, synthetic construct
MLRSGWR

SEQ ID NO: 477 - protein, 8 aa, synthetic construct
MLRSGWRG

SEQ ID NO: 478 - protein, 8 aa, synthetic construct
MLRSGWRL

SEQ ID NO: 479 - protein, 8 aa, synthetic construct
MLRSGWRS

SEQ ID NO: 480 - protein, 8 aa, synthetic construct
MTFPTYIF

SEQ ID NO: 481 - protein, 8 aa, synthetic construct
MTFPTYWF

SEQ ID NO: 482 - protein, 12 aa, synthetic construct
NHRIGRSDNHRR

SEQ ID NO: 483 - protein, 10 aa, synthetic construct
NMPSFKLVTG

SEQ ID NO: 484 - protein, 12 aa, synthetic construct
NTLSGRSENHSG

SEQ ID NO: 485 - protein, 12 aa, synthetic construct
NTLSGRSGNHGS

SEQ ID NO: 486 - protein, 10 aa, synthetic construct
NZPRVRLVLP

SEQ ID NO: 487 - protein, 8 aa, synthetic construct
PAGLWLDP

SEQ ID NO: 488 - protein, 5 aa, synthetic construct
PAGRR

SEQ ID NO: 489 - protein, 6 aa, synthetic construct
PAGRRS

SEQ ID NO: 490 - protein, 7 aa, synthetic construct
PAGRRSL

SEQ ID NO: 491 - protein, 8 aa, synthetic construct
PASLWYTQ

SEQ ID NO: 492 - protein, 8 aa, synthetic construct
PESRRWMP

SEQ ID NO: 493 - protein, 6 aa, synthetic construct
PFHLSR

SEQ ID NO: 494 - protein, 6 aa, synthetic construct
PHGFFQ

SEQ ID NO: 495 - protein, 7 aa, synthetic construct
PLARAGI

SEQ ID NO: 496 - protein, 7 aa, synthetic construct
PLARAGL

SEQ ID NO: 497 - protein, 4 aa, synthetic construct
PLGL

SEQ ID NO: 498 - protein, 6 aa, synthetic construct
PLGLAG

SEQ ID NO: 499 - protein, 6 aa, synthetic construct
PLGLWA

SEQ ID NO: 500 - protein, 7 aa, synthetic construct
PLGVRGK

SEQ ID NO: 501 - protein, 8 aa, synthetic construct
PLTGRSGG

SEQ ID NO: 502 - protein, 12 aa, synthetic construct
PLTGRSGGGGSS

SEQ ID NO: 503 - protein, 5 aa, synthetic construct
PPLGR

SEQ ID NO: 504 - protein, 10 aa, synthetic construct
PPPDMKLFPG

SEQ ID NO: 505 - protein, 10 aa, synthetic construct
PPPEVRSFSV

SEQ ID NO: 506 - protein, 10 aa, synthetic construct
PPPVLKLLEW

SEQ ID NO: 507 - protein, 12 aa, synthetic construct
PPSIARSDNLAN

SEQ ID NO: 508 - protein, 8 aa, synthetic construct
PQHRIVSF

SEQ ID NO: 509 - protein, 8 aa, synthetic construct
PRFKIIGG

SEQ ID NO: 510 - protein, 8 aa, synthetic construct
PRFRIIGG

SEQ ID NO: 511 - protein, 10 aa, synthetic construct
PRPFVKSVDQ

SEQ ID NO: 512 - protein, 4 aa, synthetic construct
PRQV

SEQ ID NO: 513 - protein, 4 aa, synthetic construct
PRSF

SEQ ID NO: 514 - protein, 10 aa, synthetic construct
PSPPVKMMPE

SEQ ID NO: 515 - protein, 10 aa, synthetic construct
PTNGGSGGSS

SEQ ID NO: 516 - protein, 10 aa, synthetic construct
RVPKVKVMLD

SEQ ID NO: 517 - protein, 8 aa, synthetic construct
SAGFSLPA

SEQ ID NO: 518 - protein, 8 aa, synthetic construct
SAPAVESE

SEQ ID NO: 519 - protein, 10 aa, synthetic construct
SAPYFRMMDM

SEQ ID NO: 520 - protein, 8 aa, synthetic construct
SARGPSRW

SEQ ID NO: 521 - protein, 8 aa, synthetic construct
SCGPPLGR

SEQ ID NO: 522 - protein, 8 aa, synthetic construct
SCSPPLGR

SEQ ID NO: 523 - protein, 8 aa, synthetic construct
SGGPLGVR

SEQ ID NO: 524 - protein, 8 aa, synthetic construct
SGGPPLGR

SEQ ID NO: 525 - protein, 7 aa, synthetic construct
SGPPLGR

SEQ ID NO: 526 - protein, 4 aa, synthetic construct
SGRS

SEQ ID NO: 527 - protein, 5 aa, synthetic construct
SGRSA

SEQ ID NO: 528 - protein, 6 aa, synthetic construct
SGRSAN

SEQ ID NO: 529 - protein, 7 aa, synthetic construct
SGRSANI

SEQ ID NO: 530 - protein, 7 aa, synthetic construct
SGRSANP

SEQ ID NO: 531 - protein, 9 aa, synthetic construct
SGRSANPRG

SEQ ID NO: 532 - protein, 5 aa, synthetic construct
SGRSD

SEQ ID NO: 533 - protein, 6 aa, synthetic construct
SGRSDD

SEQ ID NO: 534 - protein, 7 aa, synthetic construct
SGRSDDH

SEQ ID NO: 535 - protein, 6 aa, synthetic construct
SGRSDI

SEQ ID NO: 536 - protein, 7 aa, synthetic construct
SGRSDIH

SEQ ID NO: 537 - protein, 6 aa, synthetic construct
SGRSDN

SEQ ID NO: 538 - protein, 7 aa, synthetic construct
SGRSDNI

SEQ ID NO: 539 - protein, 7 aa, synthetic construct
SGRSDNP

SEQ ID NO: 540 - protein, 6 aa, synthetic construct
SGRSDQ

SEQ ID NO: 541 - protein, 7 aa, synthetic construct
SGRSDQH

SEQ ID NO: 542 - protein, 6 aa, synthetic construct
SGRSDT

SEQ ID NO: 543 - protein, 7 aa, synthetic construct
SGRSDTH

SEQ ID NO: 544 - protein, 6 aa, synthetic construct
SGRSDY

SEQ ID NO: 545 - protein, 7 aa, synthetic construct
SGRSDYH

SEQ ID NO: 546 - protein, 5 aa, synthetic construct
SGRSG

SEQ ID NO: 547 - protein, 6 aa, synthetic construct
SGRSGN

SEQ ID NO: 548 - protein, 7 aa, synthetic construct
SGRSGNH

SEQ ID NO: 549 - protein, 8 aa, synthetic construct
SGSPPLGR

SEQ ID NO: 550 - protein, 8 aa, synthetic construct
SIARSDNL

SEQ ID NO: 551 - protein, 14 aa, synthetic construct
SISSGLLSGRSDNI

SEQ ID NO: 552 - protein, 6 aa, synthetic construct
SNPFKY

SEQ ID NO: 553 - protein, 8 aa, synthetic construct
SPLPLRVP

SEQ ID NO: 554 - protein, 8 aa, synthetic construct
SPLTGRSG

SEQ ID NO: 555 - protein, 6 aa, synthetic construct
SPPLGR

SEQ ID NO: 556 - protein, 5 aa, synthetic construct
SRRVP

SEQ ID NO: 557 - protein, 6 aa, synthetic construct
SRRVPL

SEQ ID NO: 558 - protein, 6 aa, synthetic construct
SRRVPV

SEQ ID NO: 559 - protein, 8 aa, synthetic construct
SSGPPLGR

SEQ ID NO: 560 - protein, 7 aa, synthetic construct
SSPPLGR

SEQ ID NO: 561 - protein, 8 aa, synthetic construct
SSRGPAYL

SEQ ID NO: 562 - protein, 9 aa, synthetic construct
SSRHRRALD

SEQ ID NO: 563 - protein, 15 aa, synthetic construct
SSSFDKGKYKKGDDA

SEQ ID NO: 564 - protein, 15 aa, synthetic construct
SSSFDKGKYKRGDDA

SEQ ID NO: 565 - protein, 8 aa, synthetic construct
SSSPPLGR

SEQ ID NO: 566 - protein, 8 aa, synthetic construct
STFPFGMF

SEQ ID NO: 567 - protein, 6 aa, synthetic construct
STVFHM

SEQ ID NO: 568 - protein, 6 aa, synthetic construct
SVHHLI

SEQ ID NO: 569 - protein, 8 aa, synthetic construct
SVSGLLSH

SEQ ID NO: 570 - protein, 8 aa, synthetic construct
SVSGLLSS

SEQ ID NO: 571 - protein, 8 aa, synthetic construct
SVSGLRSH

SEQ ID NO: 572 - protein, 8 aa, synthetic construct
SVSGLRSS

SEQ ID NO: 573 - protein, 4 aa, synthetic construct
TARG

SEQ ID NO: 574 - protein, 5 aa, synthetic construct
TARGP

SEQ ID NO: 575 - protein, 12 aa, synthetic construct
TARGPALAHGLF

SEQ ID NO: 576 - protein, 6 aa, synthetic construct
TARGPS

SEQ ID NO: 577 - protein, 7 aa, synthetic construct
TARGPSF

SEQ ID NO: 578 - protein, 8 aa, synthetic construct
TARGPSFK

SEQ ID NO: 579 - protein, 7 aa, synthetic construct
TARGPSW

SEQ ID NO: 580 - protein, 7 aa, synthetic construct
TARGPSW

SEQ ID NO: 581 - protein, 10 aa, synthetic construct
TARGPVPRQV

SEQ ID NO: 582 - protein, 4 aa, synthetic construct
TFVH

SEQ ID NO: 583 - protein, 12 aa, synthetic construct
TGLSGRSVTQTS

SEQ ID NO: 584 - protein, 8 aa, synthetic construct
TGRGPSWV

SEQ ID NO: 585 - protein, 12 aa, synthetic construct
TLRLGRSDNNKN

SEQ ID NO: 586 - protein, 8 aa, synthetic construct
TLSGLRSP

SEQ ID NO: 587 - protein, 8 aa, synthetic construct
TSGRSANP

SEQ ID NO: 588 - protein, 8 aa, synthetic construct
TSGRSGNP

SEQ ID NO: 589 - protein, 12 aa, synthetic construct
TSLSGRSANPRG

SEQ ID NO: 590 - protein, 12 aa, synthetic construct
TSLSGRSGNPRG

SEQ ID NO: 591 - protein, 8 aa, synthetic construct
TSSGLRSP

SEQ ID NO: 592 - protein, 12 aa, synthetic construct
TSTSGRSANPRG

SEQ ID NO: 593 - protein, 22 aa, synthetic construct
TSTSGRSANPRGGGAVGLLAPP

SEQ ID NO: 594 - protein, 24 aa, synthetic construct
TSTSGRSANPRGGGVHMPLGFLGP

SEQ ID NO: 595 - protein, 12 aa, synthetic construct
TSTSGRSGNPRG

SEQ ID NO: 596 - protein, 8 aa, synthetic construct
TVSGLRSP

SEQ ID NO: 597 - protein, 701 aa, synthetic construct
  VARIANT 701: note = X is lysine or absent
EVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCVRHGNFGNSYVSWWAYWGQGTLVTVSSGGGGSGGGGSGGGGSQTVVTQEPSLTVSPGGTVTLTCGSSTGAVTTSNYVNWVQQKPGQAPRGLIGGTNKRAPGTPARFSGSLIGGKAALTLSGAQPEDEAEYYCVLWYSNRWVFGGGTKLTVLGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGX

SEQ ID NO: 598 - protein, 230 aa, synthetic construct
  VARIANT 230: note = X is lysine or absent
GSSKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGX

SEQ ID NO: 599 - protein, 214 aa, synthetic construct; identical to SEQ ID NO: 17

SEQ ID NO: 600 - protein, 701 aa, synthetic construct
  VARIANT 701: note = X is lysine or absent
EVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCVRHGNFGNSYVSWWAYWGQGTLVTVSSGGGGSGGGGSGGGGSQTVVTQEPSLTVSPGGTVTLTCRSSTGAVTTSNYANWVQQKPGQAPRGLIGGTNKRAPGTPARFSGSLIGGKAALTLSGAQPEDEAEYYCVLWYSNLWVFGGGTKLTVLGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGX

SEQ ID NO: 601 - protein, 230 aa, synthetic construct; identical to SEQ ID NO: 598
  VARIANT 230: note = X is lysine or absent

SEQ ID NO: 602 - protein, 214 aa, synthetic construct; identical to SEQ ID NO: 17

SEQ ID NO: 603 - protein, 472 aa, synthetic construct
EVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCVRHGNFGNSYVSWWAYWGQGTLVTVSSGGGGSGGGGSGGGGSQTVVTQEPSLTVSPGGTVTLTCGSSTGAVTTSNYVNWVQQKPGQAPRGLIGGTNKRAPGTPARFSGSLIGGKAALTLSGAQPEDEAEYYCVLWYSNRWVFGGGTKLTVLGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRV

SEQ ID NO: 604 - protein, 214 aa, synthetic construct; identical to SEQ ID NO: 17

SEQ ID NO: 605 - protein, 482 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVGGGGSGGGGSGGGGSEVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCVRHGNFGNSYVSWWAYWGQGTLVTVSSGGGGSGGGGSGGGGSQTVVTQEPSLTVSPGGTVTLTCGSSTGAVTTSNYVNWVQQKPGQAPRGLIGGTNKRAPGTPARFSGSLIGGKAALTLSGAQPEDEAEYYCVLWYSNRWVFGGGTKLTVL

SEQ ID NO: 606 - protein, 214 aa, synthetic construct; identical to SEQ ID NO: 17

SEQ ID NO: 607 - protein, 769 aa, synthetic construct
  VARIANT 769: note = X is absent or lysine
QGQSGSGYLWGCEWNCGGITTGSSGGSGGSGGLSGRSDDHGGSEVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCVRHGNFGNSYVSWWAYWGQGTLVTVSSGGGGSGGGGSGGGGSQTVVTQEPSLTVSPGGTVTLTCGSSTGAVTTSNYVNWVQQKPGQAPRGLIGGTNKRAPGTPARFSGSLIGGKAALTLSGAQPEDEAEYYCVLWYSNRWVFGGGTKLTVLGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGGGSSGGSISSGLLSGRSDNIGGGSGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGX

SEQ ID NO: 608 - protein, 230 aa, synthetic construct; identical to SEQ ID NO: 598
  VARIANT 230: note = X is lysine or absent

SEQ ID NO: 609 - protein, 261 aa, synthetic construct; identical to SEQ ID NO: 18

SEQ ID NO: 610 - protein, 767 aa, synthetic construct
  VARIANT 767: note = X is lysine or absent
QGQSGSGYLWGCEWNCGGITTGSSGGSGGSGGLSGRSDDHGGSEVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCVRHGNFGNSYVSWWAYWGQGTLVTVSSGGGGSGGGGSGGGGSQTVVTQEPSLTVSPGGTVTLTCGSSTGAVTTSNYVNWVQQKPGQAPRGLIGGTNKRAPGTPARFSGSLIGGKAALTLSGAQPEDEAEYYCVLWYSNRWVFGGGTKLTVLGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGGGSSGGSAPRSALAHGLFGGGSGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGX

SEQ ID NO: 611 - protein, 230 aa, synthetic construct; identical to SEQ ID NO: 598
  VARIANT 230: note = X is lysine or absent

SEQ ID NO: 612 - protein, 261 aa, synthetic construct; identical to SEQ ID NO: 18

SEQ ID NO: 613 - protein, 744 aa, synthetic construct
  VARIANT 744: note = X is lysine or absent
QGQSGSGYLWGCEWNCGGITTGSSGGSGGSGGLSGRSDDHGGSEVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCVRHGNFGNSYVSWWAYWGQGTLVTVSSGGGGSGGGGSGGGGSQTVVTQEPSLTVSPGGTVTLTCGSSTGAVTTSNYVNWVQQKPGQAPRGLIGGTNKRAPGTPARFSGSLIGGKAALTLSGAQPEDEAEYYCVLWYSNRWVFGGGTKLTVLGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGX

SEQ ID NO: 614 - protein, 230 aa, synthetic construct; identical to SEQ ID NO: 598
  VARIANT 230: note = X is lysine or absent

SEQ ID NO: 615 - protein, 261 aa, synthetic construct; identical to SEQ ID NO: 18

SEQ ID NO: 616 - protein, 795 aa, synthetic construct
  VARIANT 795: note = X is lysine or absent
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVGGGGSGGGGSGGGGSEVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCVRHGNFGNSYVSWWAYWGQGTLVTVSSGGGGSGGGGSGGGGSQTVVTQEPSLTVSPGGTVTLTCGSSTGAVTTSNYVNWVQQKPGQAPRGLIGGTNKRAPGTPARFSGSLIGGKAALTLSGAQPEDEAEYYCVLWYSNRWVFGGGTKLTVLGGSGGSSGASGTGTAGGTGSGSGTGSGLSGRSDDHGGGSQGQSGSGYLWGCEWNCGGITTGSSGGSGGGGSGGGGSGGGGSGGSESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGX

SEQ ID NO: 617 - protein, 230 aa, synthetic construct; identical to SEQ ID NO: 598
  VARIANT 230: note = X is lysine or absent

SEQ ID NO: 618 - protein, 261 aa, synthetic construct; identical to SEQ ID NO: 18

SEQ ID NO: 619 - protein, 795 aa, synthetic construct
  VARIANT 795: note = X is lysine or absent
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVGGGGSGGGGSGGGGSEVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCVRHGNFGNSYVSWWAYWGQGTLVTVSSGGGGSGGGGSGGGGSQTVVTQEPSLTVSPGGTVTLTCGSSTGAVTTSNYVNWVQQKPGQAPRGLIGGTNKRAPGTPARFSGSLIGGKAALTLSGAQPEDEAEYYCVLWYSNRWVFGGGTKLTVLGGSGGSSGASGTGTAGGTGSGSGTGSGLSGRSDDHGGGSQGQSGSNAFRCWWDPPCQPMTGSSGGSGGGGSGGGGSGGGGSGGSESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGX

SEQ ID NO: 620 - protein, 230 aa, synthetic construct; identical to SEQ ID NO: 598
  VARIANT 230: note = X is lysine or absent

SEQ ID NO: 621 - protein, 261 aa, synthetic construct; identical to SEQ ID NO: 18

SEQ ID NO: 622 - protein, 795 aa, synthetic construct
  VARIANT 795: note = X is lysine or absent
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVGGGGSGGGGSGGGGSEVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCVRHGNFGNSYVSWWAYWGQGTLVTVSSGGGGSGGGGSGGGGSQTVVTQEPSLTVSPGGTVTLTCGSSTGAVTTSNYVNWVQQKPGQAPRGLIGGTNKRAPGTPARFSGSLIGGKAALTLSGAQPEDEAEYYCVLWYSNRWVFGGGTKLTVLGGSGGSSGASGTGTAGGTGSGSGTGSGLSGRSDDHGGGSQGQSGSARGLCWWDPPCTHDLGSSGGSGGGGSGGGGSGGGGSGGSESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGX

SEQ ID NO: 623 - protein, 230 aa, synthetic construct; identical to SEQ ID NO: 598
  VARIANT 230: note = X is lysine or absent

SEQ ID NO: 624 - protein, 261 aa, synthetic construct; identical to SEQ ID NO: 18

SEQ ID NO: 625 - DNA, 2103 bp, synthetic construct
  source 1..2103: mol_type = other DNA
  variation 2101..2103: note = n is adenine or absent
gaagttcagcttgttgaatctggcggcggactggttcagcctggcggatctctgaaactgagctgtgccgccagcggcttcacctttagcacctacgccatgaactgggtccgacaggcctctggcaaaggcctggaatgggtcggacggatcagaagcaagtacaacaactacgccacctactacgccgacagcgtgaaggacagattcaccatcagccgggacgacagcaagaacaccgcctacctgcagatgaacagcctgaaaaccgaggacaccgccgtgtactactgtgtgcggcacggcaacttcggcaacagctatgtgtcttggtgggcctattggggccagggcacactggttacagttagctctggtggcggaggatctggcggaggtggaagcggcggaggcggttctcaaacagtggtcacacaagagcccagcctgaccgtttctcctggcggaacagtgaccctgacatgcggatcttctacaggcgccgtgaccaccagcaactatgtgaattgggtgcagcagaagcccggacaggctcctagaggactgatcggcggcaccaacaaaagagcccctggaacaccagccaggttcagcggctctctgatcggaggaaaagccgctctgacactgtctggcgctcagcctgaagatgaggccgagtactattgcgtgctgtggtacagcaacagatgggtgttcggaggcggcacaaagctgacagtgcttggcggtggcggtagcgaagttcagctggtggagagtggtggggggctggttcagcctggcggttcgctcaggctgtcatgcgcagcgagcgggttcaacatcaaggacacgtatatccattgggtgagacaggcccccgggaagggcctcgaatgggttgcaaggatttatcctactaacgggtacacaaggtatgctgacagtgtgaaggggcggttcacaatttccgccgatacttctaagaatactgcttacctccagatgaactccctgagggccgaggacactgccgtttactactgctcccgctggggaggagacggcttttatgctatggattattggggtcaaggcactctagtcaccgtgagtagtgctagcacaaagggccccagcgtgttccctctggctccttgtagcagaagcaccagcgagtctacagccgctctgggctgtctggtcaaggactactttcccgagcctgtgaccgtgtcctggaatagcggagcactgaccagcggcgtgcacacatttccagccgtgctgcaaagcagcggcctgtactctctgagcagcgtggtcacagtgcctagctctagcctgggcaccaagacctacacctgtaatgtggaccacaagcctagcaacaccaaggtggacaagcgcgtggaatctaagtacggccctccttgtcctccatgtcctgctccagagtttgaaggcggcccttccgtgttcctgtttcctccaaagcctaaggacaccctgatgatcagcagaacccctgaagtgacctgcgtggtggtggacgtttcccaagaggaccctgaggtgcagttcaattggtacgtggacggcgtggaagtgcacaacgccaagaccaagcctagagaggaacagttcaacagcacctacagagtggtgtccgtgctgacagtgctgcaccaggattggctgaacggcaaagagtacaagtgcaaggtgtccaacaagggcctgcctagcagcatcgagaaaaccatcagcaaggccaagggccagcctcgcgaacctcaagtctgtacactgcctcctagccaagaggaaatgaccaagaaccaggtgtccctgagctgcgccgtgaagggcttttacccttccgatatcgccgtggaatgggagagcaatggccagccagagaacaactacaagacaacccctcctgtgctggacagcgacggctcattcttcctggtgtccagactgaccgtggacaagagcagatggcaagagggcaacgtgttcagctgcagcgtgatgcacgaggccctgcacaaccactacacccagaagtctctgagcctgtctctgggcnnn

SEQ ID NO: 626 - DNA, 690 bp, synthetic construct
  source 1..690: mol_type = other DNA
  variation 688..690: note = n is adenine or absent
ggatcttctaagtacggacctccttgtcctccatgtcctgctccagagtttgaaggcggcccttccgtgttcctgtttcctccaaagcctaaggacaccctgatgatcagcagaacccctgaagtgacctgcgtggtggtggacgtgtcccaagaggatcctgaggtgcagttcaattggtacgtggacggcgtggaagtgcacaacgccaagaccaagcctagagaggaacagttcaacagcacctacagagtggtgtccgtgctgaccgtgctgcaccaggattggctgaacggcaaagagtacaagtgcaaggtgtccaacaagggcctgcctagcagcatcgagaaaaccatcagcaaggccaagggccagccaagagaaccccaggtgtacacactgcctccatgccaagaggaaatgaccaagaaccaggtgtccctgtggtgcctggtcaagggcttctacccttccgatatcgccgtggaatgggagagcaatggccagcctgagaacaactacaagaccacacctcctgtgctggacagcgacggctcattcttcctgtacagcagactgaccgtggacaagagcagatggcaagagggcaacgtgttcagctgcagcgtgatgcacgaggccctgcacaaccactacacccagaagtctctgagcctgagcctgggcnnn

SEQ ID NO: 627 - DNA, 642 bp, synthetic construct
  source 1..642: mol_type = other DNA
gatattcagatgacacagagccccagcagcctgtctgcctctgtgggagacagagtgaccatcacctgtagagccagccaggacgtgaacacagccgtggcttggtatcagcagaagcctggcaaggcccctaagctgctgatctacagcgccagctttctgtacagcggcgtgcccagcagattcagcggctctagaagcggcaccgacttcaccctgaccataagcagtctgcagcccgaggacttcgccacctactactgtcagcagcactacaccacacctccaacctttggccagggcaccaaggtggaaatcaagcggacagtggccgctcctagcgtgttcatctttccacctagcgacgagcagctgaagtctggcacagcctctgtcgtgtgcctgctgaacaacttctaccccagagaagccaaggtgcagtggaaggtggacaacgccctgcagagcggcaatagccaagagagcgtgaccgagcaggacagcaaggactctacctacagcctgagcagcaccctgacactgagcaaggccgactacgagaagcacaaagtgtacgcctgcgaagtgacccaccagggcctttctagccctgtgaccaagagcttcaaccggggcgaatgt

SEQ ID NO: 628 - DNA, 2103 bp, synthetic construct
  source 1..2103: mol_type = other DNA
  variation 2101..2103: note = n is adenine or absent
gaagttcagcttgttgaatctggcggcggactggttcagcctggcggatctctgaaactgagctgtgccgccagcggcttcacctttagcacctacgccatgaactgggtccgacaggcctctggcaaaggcctggaatgggtcggacggatcagaagcaagtacaacaactacgccacctactacgccgacagcgtgaaggacagattcaccatcagccgggacgacagcaagaacaccgcctacctgcagatgaacagcctgaaaaccgaggacaccgccgtgtactactgtgtgcggcacggcaacttcggcaacagctatgtgtcttggtgggcctattggggccagggcacactggttacagttagctctggtggcggaggatctggcggaggtggaagcggcggaggcggttctcaaacagtggtcacacaagagcccagcctgaccgtttctcctggcggaacagtgaccctgacctgtagatcttctacaggcgccgtgaccaccagcaattacgccaattgggtgcagcagaagcccggacaggctcctagaggactgatcggcggcaccaacaaaagagcccctggaacaccagccaggttcagcggctctctgatcggaggaaaagccgctctgacactgtctggcgctcagcctgaagatgaggccgagtactattgcgtgctgtggtacagcaacctgtgggtgttcggaggcggcacaaagcttacagtgcttggaggtggcggcagcgaggtgcaacttgttgagtcaggcggaggccttgtgcaacctggtggtagcctgagactgtcttgtgccgcttctggcttcaacatcaaggacacctacatccactgggttcgacaagctccaggcaagggccttgagtgggttgccagaatctaccccaccaacggctacaccagatacgccgactctgtgaaaggccggttcaccatctctgccgacacctctaagaatactgcctatctccaaatgaactccctgagagccgaggatacagccgtctattactgcagcagatggggaggcgacggcttttacgccatggattactggggacagggaaccctggtcaccgtgtcctctgctagcacaaagggccccagcgtgttccctctggctccttgtagcagaagcaccagcgagtctacagccgctctgggctgtctggtcaaggactactttcccgagcctgtgaccgtgtcctggaatagcggagcactgaccagcggcgtgcacacatttccagccgtgctgcaaagcagcggcctgtactctctgagcagcgtggtcacagtgcctagctctagcctgggcaccaagacctacacctgtaatgtggaccacaagcctagcaacaccaaggtggacaagcgcgtggaatctaagtacggccctccttgtcctccatgtcctgctccagagtttgaaggcggcccttccgtgttcctgtttcctccaaagcctaaggacaccctgatgatcagcagaacccctgaagtgacctgcgtggtggtggacgtttcccaagaggaccctgaggtgcagttcaattggtacgtggacggcgtggaagtgcacaacgccaagaccaagcctagagaggaacagttcaacagcacctacagagtggtgtccgtgctgacagtgctgcaccaggattggctgaacggcaaagagtacaagtgcaaggtgtccaacaagggcctgcctagcagcatcgagaaaaccatcagcaaggccaagggccagcctcgcgaacctcaagtctgtacactgcctcctagccaagaggaaatgaccaagaaccaggtgtccctgagctgcgccgtgaagggcttttacccttccgatatcgccgtggaatgggagagcaatggccagccagagaacaactacaagacaacccctcctgtgctggacagcgacggctcattcttcctggtgtccagactgaccgtggacaagagcagatggcaagagggcaacgtgttcagctgcagcgtgatgcacgaggccctgcacaaccactacacccagaagtctctgagcctgtctctgggcnnn

SEQ ID NO: 629 - DNA, 690 bp, synthetic construct; identical to SEQ ID NO: 626
  source 1..690: mol_type = other DNA
  variation 688..690: note = n is adenine or absent

SEQ ID NO: 630 - DNA, 642 bp, synthetic construct; identical to SEQ ID NO: 627
  source 1..642: mol_type = other DNA

SEQ ID NO: 631 - DNA, 1416 bp, synthetic construct
  source 1..1416: mol_type = other DNA
gaagttcagcttgttgaatctggcggcggactggttcagcctggcggatctctgaaactgagctgtgccgccagcggcttcacctttagcacctacgccatgaactgggtccgacaggcctctggcaaaggcctggaatgggtcggacggatcagaagcaagtacaacaactacgccacctactacgccgacagcgtgaaggacagattcaccatcagccgggacgacagcaagaacaccgcctacctgcagatgaacagcctgaaaaccgaggacaccgccgtgtactactgtgtgcggcacggcaacttcggcaacagctatgtgtcttggtgggcctattggggccagggcacactggttacagttagctctggtggcggaggatctggcggaggtggaagcggcggaggcggttctcaaacagtggtcacacaagagcccagcctgaccgtttctcctggcggaacagtgaccctgacatgcggatcttctacaggcgccgtgaccaccagcaactatgtgaattgggtgcagcagaagcccggacaggctcctagaggactgatcggcggcaccaacaaaagagcccctggaacaccagccaggttcagcggctctctgatcggaggaaaagccgctctgacactgtctggcgcccagccagaagatgaggccgagtactattgcgtgctgtggtacagcaacagatgggtgttcggaggcggcacaaagctgacagttcttggaggcggaggaagtgaagtgcagctggtggaaagcggtggtggacttgttcaaccaggcggcagcctgagactgtcttgtgccgcttctggcttcaacatcaaggacacctacatccactgggttcgacaagctccaggcaagggccttgagtgggttgccagaatctaccccaccaacggctacaccagatacgccgactctgtgaagggccgcttcacaatctctgccgacacctccaaaaacacagcctatctccaaatgaactccctgagagccgaggatacagccgtctattactgcagcagatggggaggcgacggcttttacgccatggattactggggacagggaaccctggtcaccgtgtctagcgcctctacaaagggccctagcgtgttccctctggctccctgtagcagaagcaccagcgaatctacagctgccctgggctgcctggtcaaggactactttcctgagcctgtgaccgtgtcctggaacagcggagcactgacatctggcgtgcacacctttccagccgtgctgcaaagcagcggcctgtactctctgagcagcgtggtcacagtgcctagctctagcctgggcaccaagacctacacctgtaatgtggaccacaagcctagcaacaccaaggtggacaagagagtc

SEQ ID NO: 632 - DNA, 642 bp, synthetic construct; identical to SEQ ID NO: 627
  source 1..642: mol_type = other DNA

SEQ ID NO: 633 - DNA, 1446 bp, synthetic construct
  source 1..1446: mol_type = other DNA
gaagttcagcttgttgaatctggcggcggactggttcagcctggcggatctctgagactgtcttgtgccgccagcggcttcaacatcaaggacacctacatccactgggtccgacaggcccctggcaaaggacttgaatgggtcgccagaatctaccccaccaacggctacaccagatacgccgactctgtgaagggcagattcaccatcagcgccgacaccagcaagaacaccgcctacctgcagatgaacagcctgagagccgaggacaccgccgtgtactactgttctagatggggaggcgacggcttctacgccatggattattggggccagggcaccctggtcacagtgtctagcgcctctacaaagggccccagcgttttcccactggctccctgtagcagaagcaccagcgaatctacagccgctctgggctgcctggtcaaggactactttcctgagcctgtgaccgtgtcctggaactctggcgctctgacatctggcgtgcacacctttccagccgtgctgcaaagcagcggcctgtactctctgagcagcgtcgtgacagtgcctagcagctctctgggcaccaagacctacacctgtaatgtggaccacaagcctagcaacaccaaggtggacaagagagttggcggaggcggatcaggtggcggaggaagcggaggcggcggatctgaagttcaactggtggaaagcggcggaggccttgtgcaacctggtggcagtctgaaactgagctgtgccgcctctggcttcaccttcagcacatacgccatgaattgggttcgacaagccagcggcaagggcctcgaatgggttggaagaatcagaagcaagtacaacaactacgccacctactacgccgacagcgtgaaggatcggtttaccatcagccgggacgactccaagaatactgcctatctccaaatgaactccctcaagaccgaggatacagccgtctattactgcgtgcggcacggcaacttcggcaacagctatgtgtcttggtgggcctactggggacagggaacactggttactgtgtccagtggcggtggtggaagtggcggcggaggatcaggcggtggcggttctcaaacagtggtcacccaagagcctagcctgacagtgtctcctggcggaaccgtgacactgacatgtggcagttctacaggcgccgtgaccaccagcaactacgtgaactgggtgcagcagaagccaggccaggctcctagaggactgatcggaggcaccaacaagagagcccctggaacaccagccagattctccggctctctgatcggcggaaaagccgcactgacactgtctggtgctcagcctgaggatgaggccgagtactattgcgtgctgtggtacagcaacagatgggtgttcggcggagggaccaagctgacagtgctt

SEQ ID NO: 634 - protein, 642 aa, synthetic construct
GATATTCAGATGACACAGAGCCCCAGCAGCCTGTCTGCCTCTGTGGGAGACAGAGTGACCATCACCTGTAGAGCCAGCCAGGACGTGAACACAGCCGTGGCTTGGTATCAGCAGAAGCCTGGCAAGGCCCCTAAGCTGCTGATCTACAGCGCCAGCTTTCTGTACAGCGGCGTGCCCAGCAGATTCAGCGGCTCTAGAAGCGGCACCGACTTCACCCTGACCATAAGCAGTCTGCAGCCCGAGGACTTCGCCACCTACTACTGTCAGCAGCACTACACCACACCTCCAACCTTTGGCCAGGGCACCAAGGTGGAAATCAAGCGGACAGTGGCCGCTCCTAGCGTGTTCATCTTTCCACCTAGCGACGAGCAGCTGAAGTCTGGCACAGCCTCTGTCGTGTGCCTGCTGAACAACTTCTACCCCAGAGAAGCCAAGGTGCAGTGGAAGGTGGACAACGCCCTGCAGAGCGGCAATAGCCAAGAGAGCGTGACCGAGCAGGACAGCAAGGACTCTACCTACAGCCTGAGCAGCACCCTGACACTGAGCAAGGCCGACTACGAGAAGCACAAAGTGTACGCCTGCGAAGTGACCCACCAGGGCCTTTCTAGCCCTGTGACCAAGAGCTTCAACCGGGGCGAATGT

SEQ ID NO: 635 - DNA, 2307 bp, synthetic construct
  source 1..2307: mol_type = other DNA
  variation 2305..2307: note = n is adenine or absent
caaggacaatctggctctggctatctgtggggctgcgagtggaattgtggcggcatcacaacaggctctagcggcggaagcggaggatctggtggactgtctggcagatccgatgatcacggcggatctgaggtgcagctggtggaatctggcggaggacttgttcagcctggcggctctctgaagctgtcttgtgccgccagcggcttcacctttagcacctacgccatgaactgggtccgacaggcctctggcaaaggcctggaatgggtcggacggatcagaagcaagtacaacaactacgccacctactacgccgacagcgtgaaggacagattcaccatcagccgggacgacagcaagaacaccgcctacctgcagatgaacagcctgaaaaccgaggacaccgccgtgtactactgtgtgcggcacggcaacttcggcaacagctatgtgtcttggtgggcctattggggccagggcacactggttacagttagcagtggcggcggaggtagtggcggaggtggcagcggaggcggtggatctcaaacagtggtcacccaagagcctagcctgaccgtttctcctggcggaaccgtgacactgacatgcggatcttctacaggcgccgtgaccaccagcaactatgtgaattgggtgcagcagaagcccggacaggctcctagaggactgatcggaggcaccaacaagagagcccctggaacaccagccagattcagcggatctctgatcggcggaaaggccgctctgacactttctggtgcccagcctgaagatgaggccgagtactattgcgtgctgtggtacagcaacagatgggtgttcggaggcggcaccaagctgacagttcttggaggcggaggcagcgaagtccagctggttgaaagcggtggtggcctggtccaaccaggtggaagtctgagactgagctgtgccgcttccggcttcaacatcaaggacacctacatccactgggttcgacaagctccaggcaagggccttgagtgggttgccagaatctaccccaccaacggctacaccagatacgccgactctgtgaaaggccggttcaccatctctgccgacacctccaaaaacacagcctatctccaaatgaactccctgagagccgaggatacagccgtctattactgcagcagatggggaggcgacggcttttacgccatggattactggggacagggaaccctggtcaccgtgtctagcgcctctacaaagggccctagcgtgttccctctggctccctgtagcagaagcaccagcgaatctacagccgctctgggctgcctggtcaaggactactttcctgagcctgtgaccgtgtcctggaactctggcgctcttacaagcggcgtgcacacctttccagccgtgctgcaaagcagcggcctgtactctctgagcagcgtggtcacagtgcctagctctagcctgggcacaaagacctacacctgtaatgtggaccacaagcctagcaacaccaaggtggacaagcgcgtggaatctaagtacggcggaggaagctctggcggcagcatctcttctggactgctgagcggcagaagcgacaatatcggcggaggctctggacctccttgtcctccatgtcctgctccagagtttgaaggcggcccttccgtgttcctgtttcctccaaagcctaaggacaccctgatgatcagcagaacccctgaagtgacctgcgtggtggtggacgtgtcccaagaggatccagaggtgcagttcaattggtacgtggacggcgtggaagtgcacaacgccaagaccaagcctagagaggaacagttcaacagcacctacagagtggtgtccgtgctgacagtgctgcaccaggattggctgaacggcaaagagtacaagtgcaaggtgtccaacaagggcctgcctagcagcatcgagaaaaccatcagcaaggccaagggccagcctcgcgaacctcaagtctgtacactgcctcctagccaagaggaaatgaccaagaaccaggtgtccctgagctgcgccgtgaagggcttttacccttccgatatcgccgtggaatgggagagcaatggccagccagagaacaactacaagacaacccctcctgtgctggacagcgacggctcattcttcctggtgtccagactgaccgtggacaagagcagatggcaagagggcaacgtgttcagctgcagcgtgatgcacgaggccctgcacaaccactacacccagaagtctctgagcctgtctctgggcnnn

SEQ ID NO: 636 - DNA, 690 bp, synthetic construct; identical to SEQ ID NO: 626
  source 1..690: mol_type = other DNA
  variation 688..690: note = n is adenine or absent

SEQ ID NO: 637 - DNA, 783 bp, synthetic construct
  source 1..783: mol_type = other DNA
caaggacaatctggacaaggcgccctgatctgctgttccgatgtgtctggactgtgccgttggtgcggcggaggatcttctggcggctctatctcttctggcctgctgagcggcagaagcgacaatcacggcggaggcagcgacatccagatgacacagagcccttctagcctgagcgccagcgtgggagacagagtgaccatcacatgtagagccagccaggacgtgaacacagccgtggcttggtatcagcagaagcctggcaaggcccctaagctgctgatctacagcgccagctttctgtacagcggcgtgcccagcagattcagcggctctagaagcggcaccgacttcaccctgaccataagcagtctgcagcccgaggacttcgccacctactactgtcagcagcactacaccacacctccaacctttggccagggcaccaaggtggaaatcaagcggacagtggccgctcctagcgtgttcatctttccacctagcgacgagcagctgaagtctggcacagcctctgtcgtgtgcctgctgaacaacttctaccccagagaagccaaggtgcagtggaaggtggacaacgccctgcagtccggcaatagccaagagagcgtgaccgagcaggacagcaaggactctacctacagcctgagcagcaccctgacactgagcaaggccgactacgagaagcacaaagtgtacgcctgcgaagtgacccaccagggcctttctagccctgtgaccaagagcttcaaccggggcgaatgt

SEQ ID NO: 638 - DNA, 2307 bp, synthetic construct; identical to SEQ ID NO: 635
  source 1..2307: mol_type = other DNA
  variation 2305..2307: note = n is adenine or absent

SEQ ID NO: 639 - DNA, 2301 bp, synthetic construct
  source 1..2301: mol_type = other DNA
  variation 2299..2301: note = n is adenine or absent
caaggacaatctggctctggctatctgtggggctgcgagtggaattgtggcggcatcacaacaggctctagcggcggaagcggaggatctggtggactgtctggcagatccgatgatcacggcggatctgaggtgcagctggtggaatctggcggaggacttgttcagcctggcggctctctgaagctgtcttgtgccgccagcggcttcacctttagcacctacgccatgaactgggtccgacaggcctctggcaaaggcctggaatgggtcggacggatcagaagcaagtacaacaactacgccacctactacgccgacagcgtgaaggacagattcaccatcagccgggacgacagcaagaacaccgcctacctgcagatgaacagcctgaaaaccgaggacaccgccgtgtactactgtgtgcggcacggcaacttcggcaacagctatgtgtcttggtgggcctattggggccagggcacactggttacagttagcagtggcggcggaggtagtggcggaggtggcagcggaggcggtggatctcaaacagtggtcacccaagagcctagcctgaccgtttctcctggcggaaccgtgacactgacatgcggatcttctacaggcgccgtgaccaccagcaactatgtgaattgggtgcagcagaagcccggacaggctcctagaggactgatcggaggcaccaacaagagagcccctggaacaccagccagattcagcggatctctgatcggcggaaaggccgctctgacactttctggtgcccagcctgaagatgaggccgagtactattgcgtgctgtggtacagcaacagatgggtgttcggaggcggcaccaagctgacagttcttggaggcggaggcagcgaagtccagctggttgaaagcggtggtggcctggtccaaccaggtggaagtctgagactgagctgtgccgcttccggcttcaacatcaaggacacctacatccactgggttcgacaagctccaggcaagggccttgagtgggttgccagaatctaccccaccaacggctacaccagatacgccgactctgtgaaaggccggttcaccatctctgccgacacctccaaaaacacagcctatctccaaatgaactccctgagagccgaggatacagccgtctattactgcagcagatggggaggcgacggcttttacgccatggattactggggacagggaaccctggtcaccgtgtctagcgcctctacaaagggccctagcgtgttccctctggctccctgtagcagaagcaccagcgaatctacagccgctctgggctgcctggtcaaggactactttcctgagcctgtgaccgtgtcctggaactctggcgctcttacaagcggcgtgcacacctttccagccgtgctgcaaagcagcggcctgtactctctgagcagcgtggtcacagtgcctagctctagcctgggcacaaagacctacacctgtaatgtggaccacaagcctagcaacaccaaggtggacaagcgcgtggaatctaagtacggcggaggaagctcaggcggctctgctcctagatctgctctggcccatggactgtttggcggaggctctggacctccttgtcctccatgtcctgctccagagtttgaaggcggcccttccgtgttcctgtttcctccaaagcctaaggacaccctgatgatcagcagaacccctgaagtgacctgcgtggtggtggacgtgtcccaagaggatccagaggtgcagttcaattggtacgtggacggcgtggaagtgcacaacgccaagaccaagcctagagaggaacagttcaacagcacctacagagtggtgtccgtgctgacagtgctgcaccaggattggctgaacggcaaagagtacaagtgcaaggtgtccaacaagggcctgcctagcagcatcgagaaaaccatcagcaaggccaagggccagcctcgcgaacctcaagtctgtacactgcctcctagccaagaggaaatgaccaagaaccaggtgtccctgagctgcgccgtgaagggcttttacccttccgatatcgccgtggaatgggagagcaatggccagccagagaacaactacaagacaacccctcctgtgctggacagcgacggctcattcttcctggtgtccagactgaccgtggacaagagcagatggcaagagggcaacgtgttcagctgcagcgtgatgcacgaggccctgcacaaccactacacccagaagtctctgagcctgtctctgggcnnn

SEQ ID NO: 640 - DNA, 783 bp, synthetic construct; identical to SEQ ID NO: 637
  source 1..783: mol_type = other DNA

SEQ ID NO: 641 - DNA, 2232 bp, synthetic construct
  source 1..2232: mol_type = other DNA
  variation 2230..2232: note = n is adenine or absent
caaggacaatctggctctggctatctgtggggctgcgagtggaattgtggcggcatcacaacaggctctagcggcggaagcggaggatctggtggactgtctggcagatccgatgatcacggcggatctgaggtgcagctggtggaatctggcggaggacttgttcagcctggcggctctctgaagctgtcttgtgccgccagcggcttcacctttagcacctacgccatgaactgggtccgacaggcctctggcaaaggcctggaatgggtcggacggatcagaagcaagtacaacaactacgccacctactacgccgacagcgtgaaggacagattcaccatcagccgggacgacagcaagaacaccgcctacctgcagatgaacagcctgaaaaccgaggacaccgccgtgtactactgtgtgcggcacggcaacttcggcaacagctatgtgtcttggtgggcctattggggccagggcacactggttacagttagcagtggcggcggaggtagtggcggaggtggcagcggaggcggtggatctcaaacagtggtcacccaagagcctagcctgaccgtttctcctggcggaaccgtgacactgacatgcggatcttctacaggcgccgtgaccaccagcaactatgtgaattgggtgcagcagaagcccggacaggctcctagaggactgatcggaggcaccaacaagagagcccctggaacaccagccagattcagcggatctctgatcggcggaaaggccgctctgacactttctggtgcccagcctgaagatgaggccgagtactattgcgtgctgtggtacagcaacagatgggtgttcggaggcggcaccaagctgacagttcttggaggcggaggcagcgaagtccagctggttgaaagcggtggtggcctggtccaaccaggtggaagtctgagactgagctgtgccgcttccggcttcaacatcaaggacacctacatccactgggttcgacaagctccaggcaagggccttgagtgggttgccagaatctaccccaccaacggctacaccagatacgccgactctgtgaaaggccggttcaccatctctgccgacacctccaaaaacacagcctatctccaaatgaactccctgagagccgaggatacagccgtctattactgcagcagatggggaggcgacggcttttacgccatggattactggggacagggaaccctggtcaccgtgtctagcgcctctacaaagggccctagcgtgttccctctggctccctgtagcagaagcaccagcgaatctacagccgctctgggctgcctggtcaaggactactttcctgagcctgtgaccgtgtcctggaactctggcgctcttacaagcggcgtgcacacctttccagccgtgctgcaaagcagcggcctgtactctctgagcagcgtggtcacagtgcctagctctagcctgggcacaaagacctacacctgtaatgtggaccacaagcctagcaacaccaaggtggacaagcgcgtggaatctaagtacggccctccttgtcctccatgtcctgctccagagtttgaaggcggcccttccgtgttcctgtttcctccaaagcctaaggacaccctgatgatcagcagaacccctgaagtgacctgcgtggtggtggacgtttcccaagaggaccccgaggtgcagttcaattggtacgtggacggcgtggaagtgcacaacgccaagaccaagcctagagaggaacagttcaacagcacctacagagtggtgtccgtgctgacagtgctgcaccaggattggctgaacggcaaagagtacaagtgcaaggtgtccaacaagggcctgcctagcagcatcgagaaaaccatcagcaaggccaagggccagcctcgcgaacctcaagtctgtacactgcctcctagccaagaggaaatgaccaagaaccaggtgtccctgagctgcgccgtgaagggcttttacccttccgatatcgccgtggaatgggagagcaatggccagcctgagaacaactacaagaccacacctcctgtgctggacagcgacggctcattcttcctggtgtccagactgaccgtggacaagagcagatggcaagagggcaacgtgttcagctgcagcgtgatgcacgaggccctgcacaaccactacacccagaagtctctgtctctgtccctgggcnnn

SEQ ID NO: 642 - DNA, 690 bp, synthetic construct; identical to SEQ ID NO: 626
  source 1..690: mol_type = other DNA
  variation 688..690: note = n is adenine or absent

SEQ ID NO: 643 - DNA, 783 bp, synthetic construct; identical to SEQ ID NO: 637
  source 1..783: mol_type = other DNA

SEQ ID NO: 644 - DNA, 2385 bp, synthetic construct
  source 1..2385: mol_type = other DNA
  variation 2383..2385: note = n is adenine or absent
gaagttcagcttgttgaatctggcggcggactggttcagcctggcggatctctgagactgtcttgtgccgccagcggcttcaacatcaaggacacctacatccactgggtccgacaggcccctggcaaaggacttgaatgggtcgccagaatctaccccaccaacggctacaccagatacgccgactctgtgaagggcagattcaccatcagcgccgacaccagcaagaacaccgcctacctgcagatgaacagcctgagagccgaggacaccgccgtgtactactgttctagatggggaggcgacggcttctacgccatggattattggggccagggcaccctggtcacagtgtctagcgcctctacaaagggccccagcgttttcccactggctccctgtagcagaagcaccagcgaatctacagccgctctgggctgcctggtcaaggactactttcctgagcctgtgaccgtgtcctggaactctggcgctctgacatctggcgtgcacacctttccagccgtgctgcaaagcagcggcctgtactctctgagcagcgtcgtgacagtgcctagcagctctctgggcaccaagacctacacctgtaatgtggaccacaagcctagcaacaccaaggtggacaagagagttggcggaggcggatcaggtggcggaggaagcggaggcggcggatctgaagtgcaactggttgaaagcggcggaggccttgtgcaacctggcggaagcctgaaactgagctgtgccgcctctggcttcaccttcagcacatacgccatgaattgggttcgacaagccagcggcaagggcctcgaatgggttggaagaatcagaagcaagtacaacaactacgccacctactacgccgacagcgtgaaggatcggtttaccatcagccgggacgactccaagaatactgcctatctccaaatgaactccctcaagaccgaggatacagccgtctattactgcgtgcggcacggcaacttcggcaacagctatgtgtcttggtgggcctactggggacagggaacactggttactgtgtccagtggcggtggtggaagtggcggcggaggatcaggcggtggcggttctcaaacagtggtcacccaagagcctagcctgacagtgtctccaggcggaaccgtgacactgacatgtggcagttctacaggcgccgtgaccaccagcaactacgtgaactgggtgcagcagaagccaggccaggctcctagaggactgatcggaggcaccaacaagagagcccctggaacaccagccagattctccggctctctgatcggcggaaaagccgcactgacactgtctggtgctcagcctgaggatgaggccgagtactattgcgtgctgtggtacagcaacagatgggtgttcggcggagggaccaagctgacagtgcttggaggttctggtggcagcagcggcgcttctggaactggaacagctggcggaacaggctctggatctggcacaggatctggcctgagcggcagatccgatgatcatggcggaggttctcagggccagtctggcagtggatatctgtggggctgcgagtggaattgcggcggcatcacaacaggatctagcggaggctcaggtggtggtggatctggtggtggcggctcaggcggaggtggtagcggtggttctgagtctaagtacggccctccttgtcctccatgtcctgctccagagtttgaaggcggcccttccgtgttcctgtttcctccaaagcctaaggacaccctgatgatcagcagaacccctgaagtgacctgcgtggtggtcgacgtttcccaagaggaccctgaggtgcagttcaattggtacgtggacggcgtggaagtgcacaacgccaagaccaagcctagagaggaacagttcaacagcacctacagagtggtgtccgtgctgactgtgctgcaccaggattggctgaacggcaaagagtacaagtgcaaggtgtccaacaagggcctgccaagcagcatcgagaaaaccatcagcaaggccaagggccagcctcgcgaacctcaagtctgtacactgcctcctagccaagaggaaatgaccaagaaccaggtgtccctgagctgcgccgtgaagggcttttacccttccgatatcgccgtggaatgggagagcaatggccagccagagaacaactacaagacaacccctcctgtgctggacagcgacggctcatttttcctggtgtccagactgaccgtggacaagtccagatggcaagagggcaacgtgttcagctgcagcgtgatgcacgaggccctgcacaaccactacacccagaagtctctgtctctgagcctgggcnnn

SEQ ID NO: 645 - DNA, 690 bp, synthetic construct; identical to SEQ ID NO: 626
  source 1..690: mol_type = other DNA
  variation 688..690: note = n is adenine or absent

SEQ ID NO: 646 - DNA, 783 bp, synthetic construct; identical to SEQ ID NO: 637
  source 1..783: mol_type = other DNA

SEQ ID NO: 647 - DNA, 2385 bp, synthetic construct
  source 1..2385: mol_type = other DNA
  variation 2383..2385: note = n is adenine or absent
gaagttcagcttgttgaatctggcggcggactggttcagcctggcggatctctgagactgtcttgtgccgccagcggcttcaacatcaaggacacctacatccactgggtccgacaggcccctggcaaaggacttgaatgggtcgccagaatctaccccaccaacggctacaccagatacgccgactctgtgaagggcagattcaccatcagcgccgacaccagcaagaacaccgcctacctgcagatgaacagcctgagagccgaggacaccgccgtgtactactgttctagatggggaggcgacggcttctacgccatggattattggggccagggcaccctggtcacagtgtctagcgcctctacaaagggccccagcgttttcccactggctccctgtagcagaagcaccagcgaatctacagccgctctgggctgcctggtcaaggactactttcctgagcctgtgaccgtgtcctggaactctggcgctctgacatctggcgtgcacacctttccagccgtgctgcaaagcagcggcctgtactctctgagcagcgtcgtgacagtgcctagcagctctctgggcaccaagacctacacctgtaatgtggaccacaagcctagcaacaccaaggtggacaagagagttggcggaggcggatcaggtggcggaggaagcggaggcggcggatctgaagtgcaactggttgaaagcggcggaggccttgtgcaacctggcggaagcctgaaactgagctgtgccgcctctggcttcaccttcagcacatacgccatgaattgggttcgacaagccagcggcaagggcctcgaatgggttggaagaatcagaagcaagtacaacaactacgccacctactacgccgacagcgtgaaggatcggtttaccatcagccgggacgactccaagaatactgcctatctccaaatgaactccctcaagaccgaggatacagccgtctattactgcgtgcggcacggcaacttcggcaacagctatgtgtcttggtgggcctactggggacagggaacactggttactgtgtccagtggcggtggtggaagtggcggcggaggatcaggcggtggcggttctcaaacagtggtcacccaagagcctagcctgacagtgtctccaggcggaaccgtgacactgacatgtggcagttctacaggcgccgtgaccaccagcaactacgtgaactgggtgcagcagaagccaggccaggctcctagaggactgatcggaggcaccaacaagagagcccctggaacaccagccagattctccggctctctgatcggcggaaaagccgcactgacactgtctggtgctcagcctgaggatgaggccgagtactattgcgtgctgtggtacagcaacagatgggtgttcggcggagggaccaagctgacagtgcttggaggttctggtggcagcagcggcgcttctggaactggaacagctggcggaacaggctctggatctggcacaggatctggcctgagcggcagatccgatgatcatggcggaggttctcagggccagtctggcagcaatgccttcagatgttggtgggaccctccttgccagcctatgacaggatctagcggaggctcaggtggcggtggatctggcggaggtggtagcggaggtggtggaagcggaggatctgagtctaagtacggccctccttgtcctccatgtcctgctccagagtttgaaggcggcccttccgtgttcctgtttcctccaaagcctaaggacaccctgatgatcagcagaacccctgaagtgacctgcgtggtggtggacgtttcccaagaggaccctgaggtgcagttcaattggtacgtggacggcgtggaagtgcacaacgccaagaccaagcctagagaggaacagttcaacagcacctacagagtggtgtccgtgctgactgtgctgcaccaggattggctgaacggcaaagagtacaagtgcaaggtgtccaacaagggcctgccaagcagcatcgagaaaaccatcagcaaggccaagggccagcctcgcgaacctcaagtctgtacactgcctcctagccaagaggaaatgaccaagaaccaggtgtccctgagctgcgccgtgaagggcttttacccttccgatatcgccgtggaatgggagagcaatggccagccagagaacaactacaagacaacccctcctgtgctggacagcgacggctcatttttcctggtgtccagactgaccgtggacaagtccagatggcaagagggcaacgtgttcagctgcagcgtgatgcacgaggccctgcacaaccactacacccagaagtctctgtctctgagcctgggcnnn

SEQ ID NO: 648 - DNA, 690 bp, synthetic construct; identical to SEQ ID NO: 626
  source 1..690: mol_type = other DNA
  variation 688..690: note = n is adenine or absent

SEQ ID NO: 649 - DNA, 783 bp, synthetic construct; identical to SEQ ID NO: 637
  source 1..783: mol_type = other DNA

SEQ ID NO: 650 - DNA, 2385 bp, synthetic construct
  source 1..2385: mol_type = other DNA
  variation 2383..2385: note = n is adenine or absent
gaagttcagcttgttgaatctggcggcggactggttcagcctggcggatctctgagactgtcttgtgccgccagcggcttcaacatcaaggacacctacatccactgggtccgacaggcccctggcaaaggacttgaatgggtcgccagaatctaccccaccaacggctacaccagatacgccgactctgtgaagggcagattcaccatcagcgccgacaccagcaagaacaccgcctacctgcagatgaacagcctgagagccgaggacaccgccgtgtactactgttctagatggggaggcgacggcttctacgccatggattattggggccagggcaccctggtcacagtgtctagcgcctctacaaagggccccagcgttttcccactggctccctgtagcagaagcaccagcgaatctacagccgctctgggctgcctggtcaaggactactttcctgagcctgtgaccgtgtcctggaactctggcgctctgacatctggcgtgcacacctttccagccgtgctgcaaagcagcggcctgtactctctgagcagcgtcgtgacagtgcctagcagctctctgggcaccaagacctacacctgtaatgtggaccacaagcctagcaacaccaaggtggacaagagagttggcggaggcggatcaggtggcggaggaagcggaggcggcggatctgaagtgcaactggttgaaagcggcggaggccttgtgcaacctggcggaagcctgaaactgagctgtgccgcctctggcttcaccttcagcacatacgccatgaattgggttcgacaagccagcggcaagggcctcgaatgggttggaagaatcagaagcaagtacaacaactacgccacctactacgccgacagcgtgaaggatcggtttaccatcagccgggacgactccaagaatactgcctatctccaaatgaactccctcaagaccgaggatacagccgtctattactgcgtgcggcacggcaacttcggcaacagctatgtgtcttggtgggcctactggggacagggaacactggttactgtgtccagtggcggtggtggaagtggcggcggaggatcaggcggtggcggttctcaaacagtggtcacccaagagcctagcctgacagtgtctccaggcggaaccgtgacactgacatgtggcagttctacaggcgccgtgaccaccagcaactacgtgaactgggtgcagcagaagccaggccaggctcctagaggactgatcggaggcaccaacaagagagcccctggaacaccagccagattctccggctctctgatcggcggaaaagccgcactgacactgtctggtgctcagcctgaggatgaggccgagtactattgcgtgctgtggtacagcaacagatgggtgttcggcggagggaccaagctgacagtgcttggaggttctggtggcagcagcggcgcttctggaactggaacagctggcggaacaggctctggatctggcacaggatctggcctgagcggcagatccgatgatcatggcggaggttctcagggccagtctggctctgcaagaggactgtgttggtgggaccctccttgcacacacgatctgggatcttcaggcggatctggcggaggtggtagcggtggtggtggtagcggaggcggaggtagtggtggaagcgagtctaagtacggccctccttgtcctccatgtcctgctccagagtttgaaggcggcccttccgtgttcctgtttcctccaaagcctaaggacaccctgatgatcagcagaacccctgaagtgacctgcgtggtggtcgacgtttcccaagaggaccctgaggtgcagttcaattggtacgtggacggcgtggaagtgcacaacgccaagaccaagcctagagaggaacagttcaacagcacctacagagtggtgtccgtgctgactgtgctgcaccaggattggctgaacggcaaagagtacaagtgcaaggtgtccaacaagggcctgccaagcagcatcgagaaaaccatcagcaaggccaagggccagcctcgcgaacctcaagtctgtacactgcctcctagccaagaggaaatgaccaagaaccaggtgtccctgagctgcgccgtgaagggcttttacccttccgatatcgccgtggaatgggagagcaatggccagccagagaacaactacaagacaacccctcctgtgctggacagcgacggctcatttttcctggtgtccagactgaccgtggacaagtccagatggcaagagggcaacgtgttcagctgcagcgtgatgcacgaggccctgcacaaccactacacccagaagtctctgtctctgagcctgggcnnn

SEQ ID NO: 651 - DNA, 690 bp, synthetic construct; identical to SEQ ID NO: 626
  source 1..690: mol_type = other DNA
  variation 688..690: note = n is adenine or absent

SEQ ID NO: 652 - DNA, 783 bp, synthetic construct; identical to SEQ ID NO: 637
  source 1..783: mol_type = other DNA